(19) United States
(12) Patent Application Publication
(10) Pub. No.: US 2021/0188775 A1
(43) Pub. Date: Jun. 24, 2021

(54) CARBOXAMIDE AND SULFONAMIDE DERIVATIVES USEFUL AS TEAD MODULATORS

(71) Applicant: Genentech, Inc., South San Francisco, CA (US)

(72) Inventors: Christian CUNNINGHAM, San Mateo, CA (US); Paul Powell BEROZA, San Carlos, CA (US); James John CRAWFORD, San Carlos, CA (US); Wendy Lee, South San Francisco, CA (US); Olivier RENE, South San Francisco, CA (US); Jason Robert ZBIEG, Montara, CA (US); Jiangpeng LIAO, Shanghai (CN); Tao WANG, Shanghai (CN); Chen YU, Shanghai (CN)

(73) Assignee: Genentech, Inc., South San Francisco, CA (US)

(21) Appl. No.: 17/249,457

(22) Filed: Mar. 2, 2021

Related U.S. Application Data
(63) Continuation of application No. PCT/US2019/049255, filed on Sep. 2, 2019.

(30) Foreign Application Priority Data
Sep. 3, 2018 (CN) PCT/CN2018/103789
Nov. 22, 2018 (CN) PCT/CN2018/116897

(51) Int. Cl. C07D 213/76; C07C 311/08; C07C 303/40; C07C 235/46; C07C 231/14; C07D 213/82; C07D 209/08; C07D 237/24

(52) U.S. Cl. CPC C07D 213/76; C07C 311/08; C07C 303/40; C07D 237/24; C07C 231/14; C07D 213/82; C07D 209/08; C07C 235/46

(57) ABSTRACT

The invention is concerned with the compounds of formula (I) and formula (II):

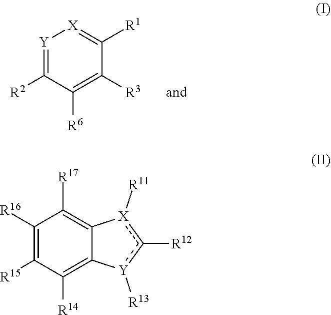

and pharmaceutically acceptable salts thereof. In addition, the present invention relates to methods of using the compounds of formula (I) and formula (II) as well as pharmaceutical compositions containing such compounds. The compounds are useful in treating diseases and conditions mediated by TEAD, such as cancer.

### CROSS-REFERENCE TO RELATED APPLICATIONS

This application is a continuation of International Application No. PCT/US2019/049255 having an International filing date of Sep. 2, 2019 which claims priority to PCT/CN2018/103789 filed on Sep. 3, 2018 and to PCT/CN2018/116897 filed on Nov. 22, 2018, both of which are incorporated herein in their entirety

### SEQUENCE LISTING

The instant application contains a Sequence Listing which has been submitted electronically in ASCII format and is hereby incorporated by reference in its entirety. Said ASCII copy, created on Mar. 1, 2021, is named P35012-US_Sequence_Listing.TXT and is 33,652 bytes in size.

### FIELD OF THE DISCLOSURE

The present disclosure relates to organic compounds of formula (I) and formula (II) useful for therapy and/or prophylaxis in a mammal, and in particular as inhibitors of TEAD useful for treating cancer.

### BRIEF DESCRIPTION

The Hippo pathway is a signaling pathway that regulates cell proliferation and cell death and determines organ size. The pathway is believed to play a role as a tumor suppressor in mammals, and disorders of the pathway are often detected in human cancers. The pathway is involved in and/or may regulate the self-renewal and differentiation of stem cells and progenitor cells. In addition, the Hippo pathway may be involved in wound healing and tissue regeneration. Furthermore, it is believed that as the Hippo pathway cross-talks with other signaling pathways such as Wnt, Notch, Hedgehog, and MAPK/ERK, it may influence a wide variety of biological events, and that its dysfunction could be involved in many human diseases in addition to cancer. For reviews, see, for example, Halder et al., 2011, Development 138:9-22; Zhao et al., 2011, Nature Cell Biology 13:877-883; Bao et al., 2011, J. Biochem. 149:361-379; Zhao at al., 2010, J. Cell Sci. 123:4001-4006.

The Hippo signaling pathway is conserved from drosophila to mammals (Vassilev et al., Genes and Development, 2001, 15, 1229-1241; Zeng and Hong, Cancer Cell, 2008, 13, 188-192). The core of the pathway consists of a cascade of kinases (Hippo-MST1-2 being upstream of Lats 1-2 and NDRI-2) leading to the phosphorylation of two transcriptional co-activators, YAP (Yes-Associated Protein) and TAZ (Transcription co-activator with PDZ binding motif or tafazzin; Zhao et al., Cancer Res., 2009, 69, 1089-1098; Lei et al., Mol. Cell. Biol., 2008, 28, 2426-2436).

Because the Hippo signaling pathway is a regulator of animal development, organ size control and stem cell regulation, it has been implicated in cancer development (Review in Harvey et al., Nat. Rev. Cancer, 2013, 13, 246-257; Zhao et al., Genes Dev. 2010, 24, 862-874). In vitro, the overexpression of YAP or TAZ in mammary epithelial cells induces cell transformation, through interaction of both proteins with the TEAD family of transcription factors. Increased YAP/TAZ transcriptional activity induces oncogenic properties such as epithelial-mesenchymal transition and was also shown to confer stem cells properties to breast cancer cells. In vivo, in mouse liver, the overexpression of YAP or the genetic knockout of its upstream regulators MST1-2 triggers the development of hepatocellular carcinomas. Furthermore, when the tumor suppressor NF2 is inactivated in the mouse liver, the development of hepatocellular carcinomas can be blocked completely by the co-inactivation of YAP.

It is believed that deregulation of the Hippo tumor suppressor pathway is a major event in the development of a wide range of malignancies, including with no limitations, lung cancer (NSCLC; Zhou et al., Oncogene, 2011, 30, 2181-2186; Wang et al., Cancer Sci., 2010, 101, 1279-1285), breast cancer (Chan et al., Cancer Res., 2008, 68, 2592-2598; Lamar et al., Proc. Natl. Acad. Sci, USA, 2012; 109, E2441-E2250; Wang et al., Eur. J. Cancer, 2012, 48, 1227-1234), head and neck cancer (Gasparotto et al., Oncotarget., 2011, 2, 1165-1175; Steinmann et al., Oncol. Rep., 2009, 22, 1519-1526), colon cancer (Angela et al., Hum. Pathol., 2008, 39, 1582-1589; Yuen et al., PLoS One, 2013, 8, e54211; Avruch et al., Cell Cycle, 2012, 11, 1090-1096), ovarian cancer (Angela et al., Hum. Pathol., 2008, 39, 1582-1589; Chad et al., Cancer Res., 2010, 70, 8517-8525; Hall et al., Cancer Res., 2010, 70, 8517-8525), liver cancer (Jie et al., Gastroenterol. Res. Pract., 2013, 2013, 187070; Ahn et al., Mol. Cancer. Res., 2013, 11, 748-758; Liu et al., Expert. Opin. Ther. Targets, 2012, 16, 243-247), brain cancer (Orr et al., J Neuropathol. Exp. Neurol. 2011, 70, 568-577; Baia et al., Mol. Cancer Res., 2012, 10, 904-913; Striedinger et al., Neoplasia, 2008, 10, 1204-1212) and prostate cancer (Zhao et al., Genes Dev., 2012, 26, 54-68; Zhao et al., Genes Dev., 2007, 21, 2747-2761), mesotheliomas (Fujii et al., J. Exp. Med., 2012, 209, 479-494; Mizuno et al., Oncogene, 2012, 31, 5117-5122; Sekido Y., Pathol. Int., 2011, 61, 331-344), sarcomas (Seidel et al., Mol. Carcinog., 2007, 46, 865-871) and leukemia (Jimenez-Velasco et al., Leukemia, 2005, 19, 2347-2350).

Two of the core components of the mammalian Hippo pathway are Lats1 and Lats2, which are nuclear Dbf2-related (NDR) family protein kinases homologous to Drosophila Warts (Wts). The Lats1/2 proteins are activated by association with the scaffold proteins Mob1A/B (Mps one binder kinase activator-like 1A and 1), which are homologous to Drosophila Mats. Lats1/2 proteins are also activated by phosphorylation by the STE20 family protein kinases Mst1 and Mst2, which are homologous to Drosophila Hippo. Lats1/2 kinases phosphorylate the downstream effectors YAP (Yes-associated protein) and TAZ (transcriptional coactivator with PDZ-binding motif, WWTR1), which are homologous to Drosophila Yorkie. The phosphorylation of YAP and TAZ by Lats1/2 are crucial events within the Hippo signaling pathway. Lats1/2 phosphorylates YAP at multiple sites, but phosphorylation of Ser127 is critical for YAP inhibition. Phosphorylation of YAP generates a protein-binding motif for the 14-3-3 family of proteins, which upon binding of a 14-3-3 protein, leads to retention and/or sequestration of YAP in the cell cytoplasm. Likewise, Lats1/2 phosphorylates TAZ at multiple sites, but phosphorylation of Ser89 is critical for TAZ inhibition. Phosphorylation of TAZ leads to retention and/or sequestration of TAZ in the cell cytoplasm. In addition, phosphorylation of YAP and TAZ is believed to destabilize these proteins by activating phosphorylation-dependent degradation catalyzed by YAP or TAZ ubiquitination. Thus, when the Hippo pathway is “on”, YAP and/or TAZ is phosphorylated, inactive, and generally sequestered in the cytoplasm; in contrast, when the Hippo pathway is “off”, YAP and/or TAZ is non-phosphorylated, active, and generally found in the nucleus.

Non-phosphorylated, activated YAP is translocated into the cell nucleus where its major target transcription factors are the four proteins of the TEAD-domain-containing family (TEAD1-TEAD4, collectively “TEAD”). YAP together with TEAD (or other transcription factors such as Smad1, RUNX, ErbB4 and p73) has been shown to induce the expression of a variety of genes, including connective tissue growth factor (CTGF), Gli2, Birc5, Birc2, fibroblast growth factor 1 (FGF1), and amphiregulin (AREG). Like YAP, non-phosphorylated TAZ is translocated into the cell nucleus where it interacts with multiple DNA-binding transcription factors, such as peroxisome proliferator-activated receptor γ (PPARγ), thyroid transcription factor-1 (TTF-1), Pax3, TBX5, RUNX, TEAD1 and Smad2/3/4. Many of the genes activated by YAP/TAZ-transcription factor complexes mediate cell survival and proliferation. Therefore, under some conditions YAP and/or TAZ acts as an oncogene and the Hippo pathway acts as a tumor suppressor.

Hence, pharmacological targeting of the Hippo cascade through inhibition of TEAD would be valuable approach for the treatment of cancers that harbor functional alterations of this pathway.

### SUMMARY OF THE DISCLOSURE

In some aspects, a compound or a pharmaceutically acceptable salt thereof of the following formula (I) is provided:

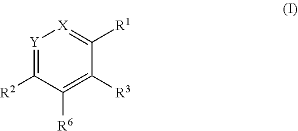

R¹ is selected from —C₁₋₆ alkyl, —C₃₋₈ cycloalkyl, —C₁₋₆ alkyl-C₃₋₈ cycloalkyl, —C₁₋₆ haloalkyl, —O—C₁₋₆ alkyl, —O—C₃₋₈ cycloalkyl, —O—C₁₋₆ alkyl-C₃₋₈ cycloalkyl and —O—C₁₋₆ haloalkyl.

R² is selected from —C(O)—N(R^{a})(R^{b}) and —N(R^{c})—S(O)₂(R^{d}). Each R^{a}, R^{b}, R^{c} and R^{d} is independently selected from —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₁₋₆ alkyl-C₅₋₂₀ aryl, —C₃₋₈ heterocyclyl, —C₆₋₂₀ aryl and —C₁₋₂₀ heteroaryl, wherein each —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₁₋₆ alkyl-C₅₋₂₀ aryl, —C₃₋₈ heterocyclyl, —C₆₋₂₀ aryl, and —C₁₋₂₀ heteroaryl is independently optionally substituted with at least one of oxo, —CN, —C₁₋₁₂ alkyl, —C₁₋₁₂ haloalkyl, halo, —NO₂, —N(R^{e})(R^{f}), —C₁₋₆ alkyl-C(O)—N(R^{e})(R^{f}), and —OR^{e}. Each R^{a}, R^{b} and R^{c} may further optionally independently be H. Each R^{e} and R^{f} is independently selected from hydrogen, —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃₋₈ heterocyclyl, —C₆₋₂₀ aryl and —C₁₋₂₀ heteroaryl, wherein each —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl is independently optionally substituted with at least one of oxo, —CN, —C₁₋₁₂ alkyl, —C₁₋₁₂ haloalkyl, halo, —NO₂, —O—C₁₋₁₂ alkyl and —OH.

R³ is -(A)ₙ-R⁵. A is selected from optionally substituted —C₁₋₁₂ alkyl-, —C₃₋₈ cycloalkyl- and —C₂₋₁₂ alkenyl-. R⁵ is selected from hydrogen, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl, and —C₅₋₁₃ spirocycle, wherein for A and R⁵, each —C₁₋₁₂ alkyl-, —C₃₋₈ cycloalkyl-, —C₂₋₁₂ alkenyl-, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl and —C₅₋₁₃ spirocycle is independently optionally substituted with at least one of oxo, —CN, —C₁₋₁₂ alkyl, —C₁₋₁₂ haloalkyl, C₃₋₈ cycloalkyl, halo, —NO₂, —N(R^{e})(R^{f}), and —OR^{e}. n is 0 or 1.

Each X and Y is independently selected from CR⁴ and N. R⁴ and R⁶ is independently selected from hydrogen, halogen, —C₁₋₆ haloalkyl and CN.

When X and Y are each CR⁴ and when R² is —C(O)—N(R^{a})(R^{b}), A is selected from optionally substituted —C₁₋₁₂ alkyl-, —C₃₋₈ cycloalkyl- and —C₃₋₁₂ alkenyl- and R⁵ is selected from hydrogen, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl, and —C₅₋₁₃ spirocycle. For A and R⁵, each —C₁₋₁₂ alkyl-, —C₃₋₈ cycloalkyl-, —C₃₋₁₂ alkenyl, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl and —C₅₋₁₃ spirocycle is independently optionally substituted with at least one of oxo, —CN, —C₁₋₁₂ alkyl, —C₁₋₁₂ haloalkyl, C-₃₋₈ cycloalkyl, halo, —NO₂, —N(R^{e})(R^{f}), and —OR^{e}.

In some aspects, the compound or a pharmaceutically acceptable salt thereof of formula (I) is selected from compounds 1 to 21, 25 to 52 and 54 to 58:

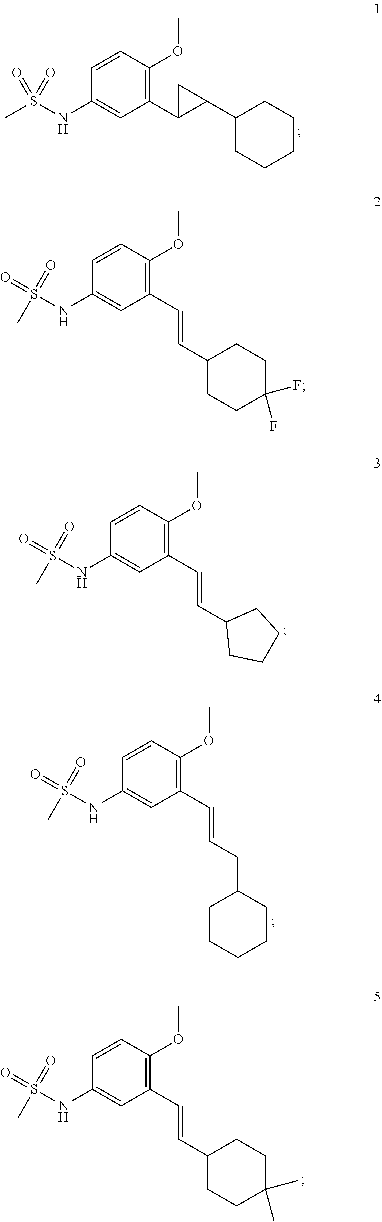 [SMILES: COc1ccc(NS(C)(=O)=O)cc1C1CC1C1CCCCC1 ; COc1ccc(NS(C)(=O)=O)cc1/C=C/C1CCC(F)(F)CC1 ; COc1ccc(NS(C)(=O)=O)cc1/C=C/C1CCCC1 ; COc1ccc(NS(C)(=O)=O)cc1/C=C/CC1CCCCC1 ; COc1ccc(NS(C)(=O)=O)cc1/C=C/C1CCC(C)(C)CC1]

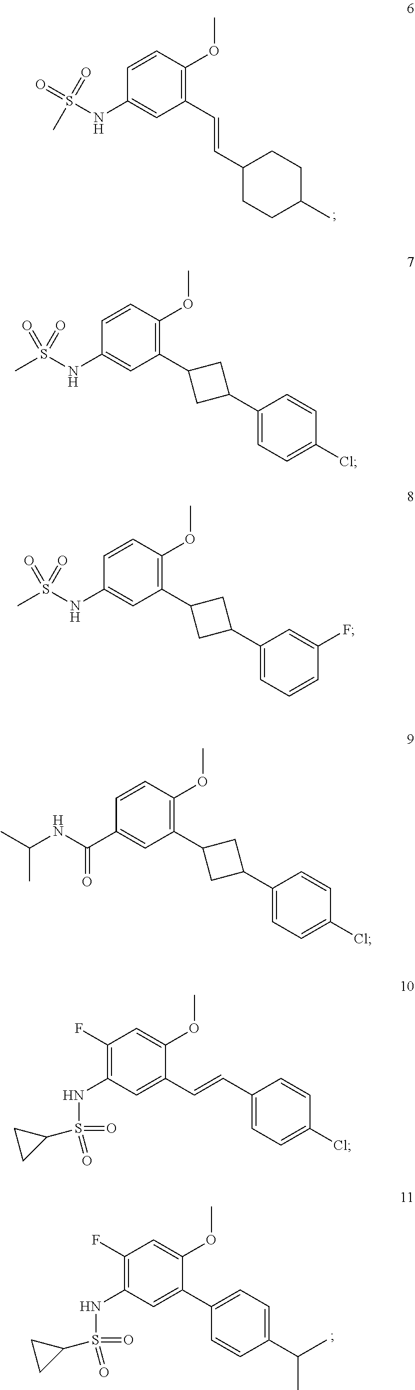

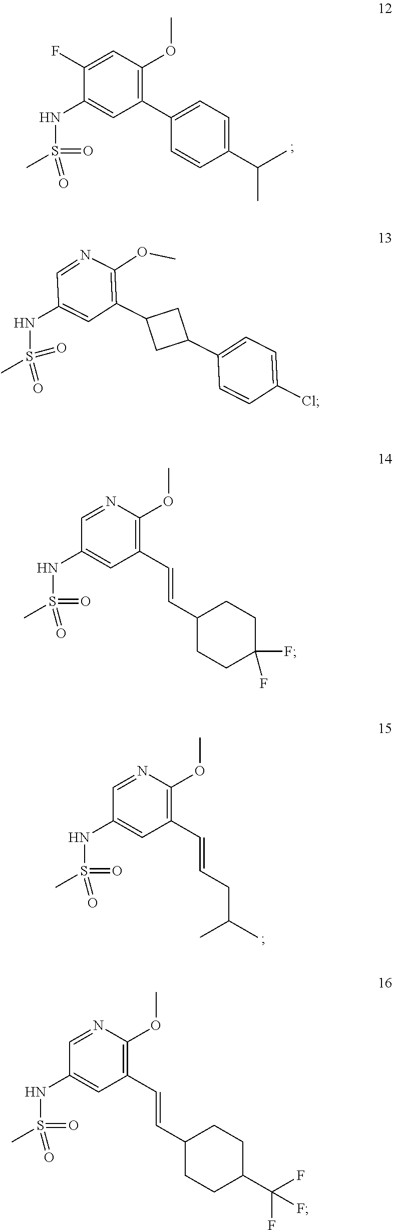

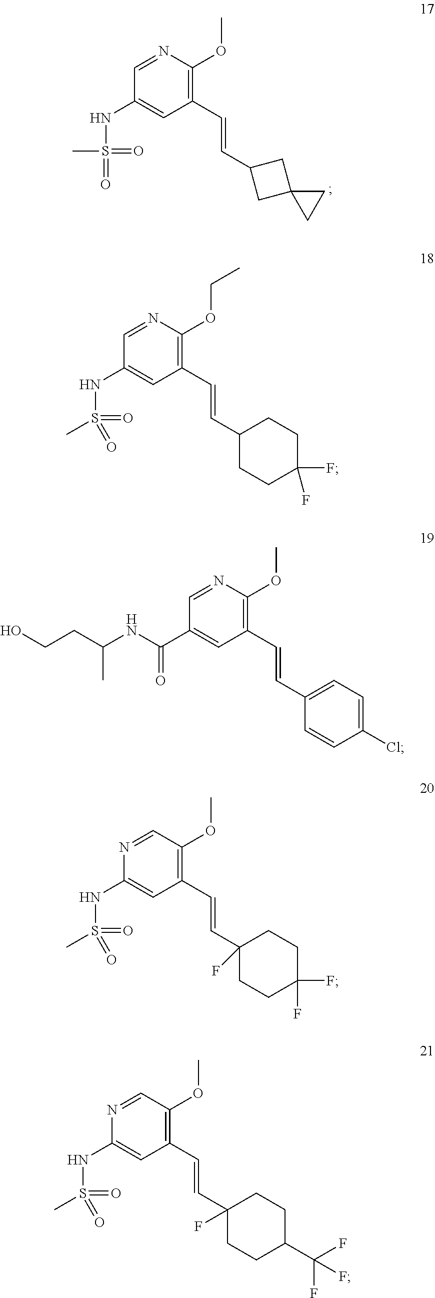 [SMILES: COc1ncc(NS(C)(=O)=O)cc1/C=C/C1CC2(CC2)C1 ; CCOc1ncc(NS(C)(=O)=O)cc1/C=C/C1CCC(F)(F)CC1 ; COc1ncc(C(=O)NC(C)CCO)cc1/C=C/c1ccc(Cl)cc1 ; COc1cnc(NS(C)(=O)=O)cc1/C=C/C1(F)CCC(F)(F)CC1 ; COc1cnc(NS(C)(=O)=O)cc1/C=C/C1(F)CCC(C(F)(F)F)CC1]

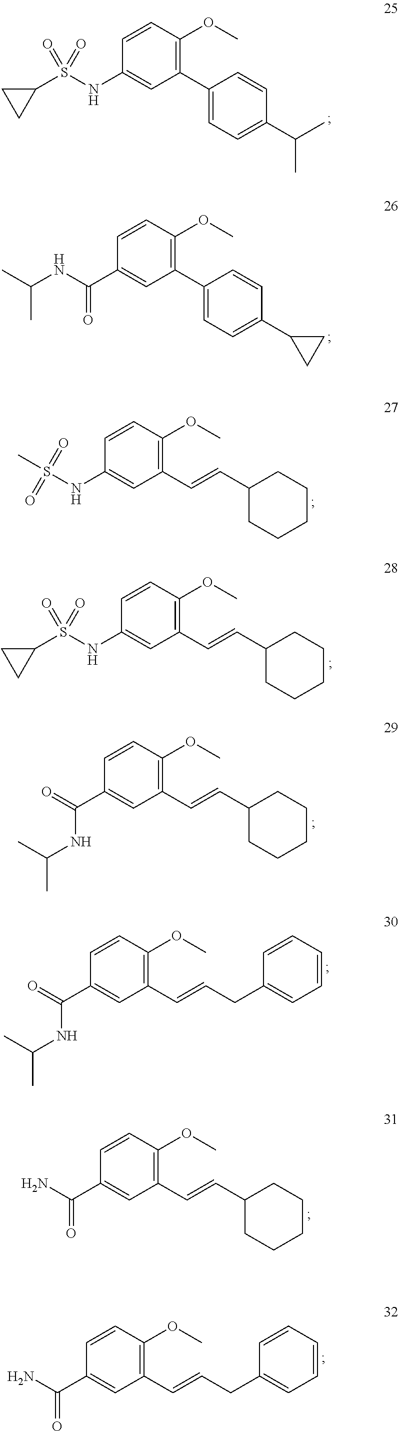

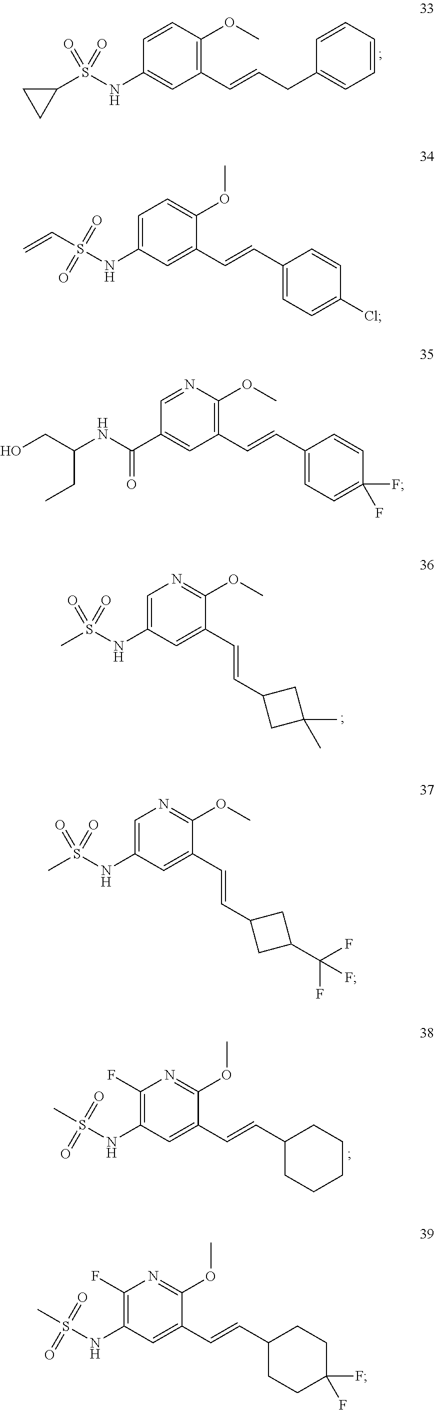

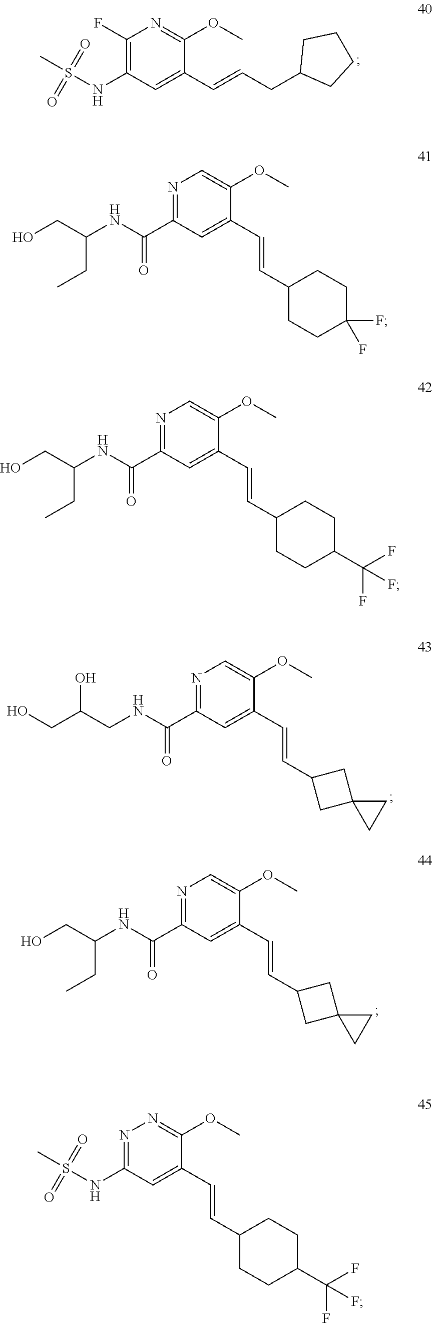 [SMILES: COc1nc(F)c(NS(C)(=O)=O)cc1/C=C/CC1CCCC1 ; CCC(CO)NC(=O)c1cc(/C=C/C2CCC(F)(F)CC2)c(OC)cn1 ; CCC(CO)NC(=O)c1cc(/C=C/C2CCC(C(F)(F)F)CC2)c(OC)cn1 ; COc1cnc(C(=O)NCC(O)CO)cc1/C=C/C1CC2(CC2)C1 ; CCC(CO)NC(=O)c1cc(/C=C/C2CC3(CC3)C2)c(OC)cn1 ; COc1nnc(NS(C)(=O)=O)cc1/C=C/C1CCC(C(F)(F)F)CC1]

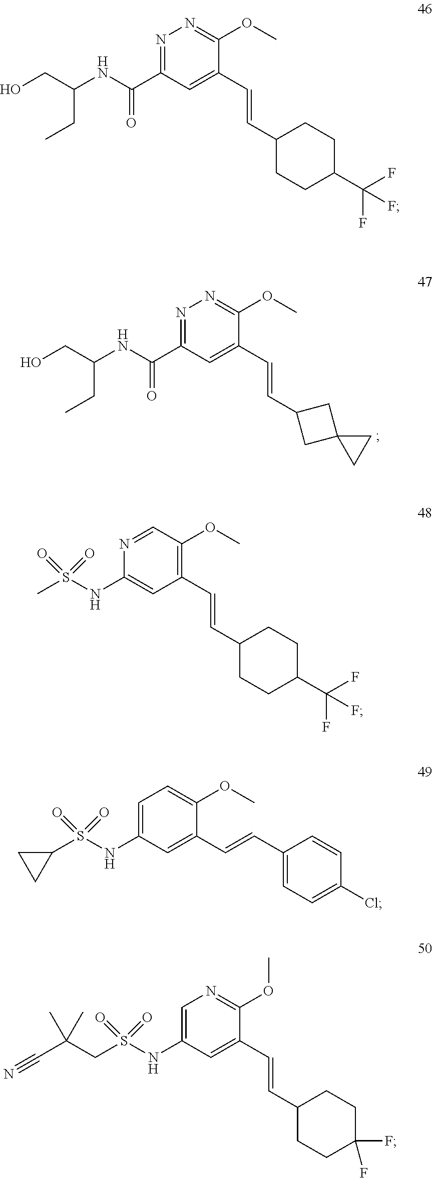 [SMILES: CCC(CO)NC(=O)c1cc(/C=C/C2CCC(C(F)(F)F)CC2)c(OC)nn1 ; CCC(CO)NC(=O)c1cc(/C=C/C2CC3(CC3)C2)c(OC)nn1 ; COc1cnc(NS(C)(=O)=O)cc1/C=C/C1CCC(C(F)(F)F)CC1 ; COc1ccc(NS(=O)(=O)C2CC2)cc1/C=C/c1ccc(Cl)cc1 ; COc1ncc(NS(=O)(=O)CC(C)(C)C#N)cc1/C=C/C1CCC(F)(F)CC1]

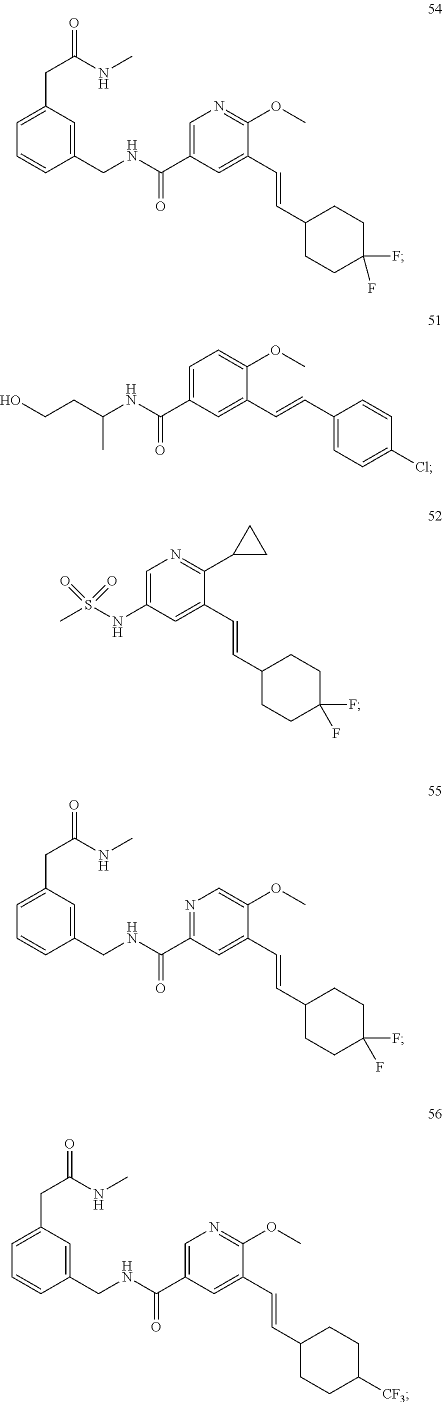 [SMILES: CNC(=O)Cc1cccc(CNC(=O)c2cnc(OC)c(/C=C/C3CCC(F)(F)CC3)c2)c1 ; COc1ccc(C(=O)NC(C)CCO)cc1/C=C/c1ccc(Cl)cc1 ; CS(=O)(=O)Nc1cnc(C2CC2)c(/C=C/C2CCC(F)(F)CC2)c1 ; CNC(=O)Cc1cccc(CNC(=O)c2cc(/C=C/C3CCC(F)(F)CC3)c(OC)cn2)c1 ; CNC(=O)Cc1cccc(CNC(=O)c2cnc(OC)c(/C=C/C3CCC(C(F)(F)F)CC3)c2)c1]

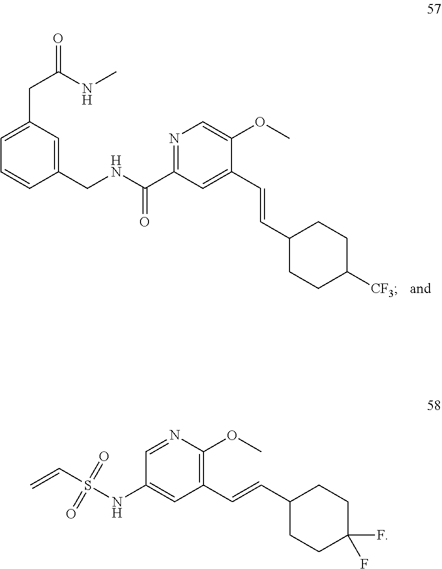 [SMILES: CNC(=O)Cc1cccc(CNC(=O)c2cc(/C=C/C3CCC(C(F)(F)F)CC3)c(OC)cn2)c1 ; "and" ; C=CS(=O)(=O)Nc1cnc(OC)c(/C=C/C2CCC(F)(F)CC2)c1]

In some aspects, a compound or a pharmaceutically acceptable salt thereof of the following formula (II) is provided:

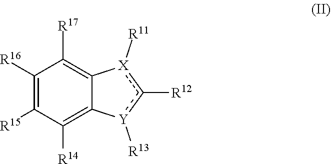

R¹¹ is selected from hydrogen, —C₁₋₆ alkyl, —C₃₋₈ cycloalkyl, —C₁₋₆ alkyl-C₃₋₈ cycloalkyl, and —C₁₋₆ haloalkyl.

R¹⁵ is —C(O)—N(R^{g})(R^{h}) or —N(Rⁱ)—S(O)₂(R^{j}). Each R^{g}, R^{h}, Rⁱ and R^{j} is independently selected from —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃₋₈ heterocyclyl, —C₆₋₂₀ aryl and —C₁₋₂₀ heteroaryl, and wherein each —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃₋₈ heterocyclyl, —C₆₋₂₀ aryl and —C₁₋₂₀ heteroaryl is independently optionally substituted with at least one of oxo, —CN, —C₁₋₁₂ alkyl, —C₁₋₁₂ haloalkyl, halo, —NO₂, —N(R^{k})(R^{l}), and —OR^{k}. Each of R^{g}, R^{h} and Rⁱ may be further optionally substituted with H. Each R^{k} and R^{l} is independently selected from hydrogen, —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃₋₈ heterocyclyl, —C₆₋₂₀ aryl and —C₁₋₂₀ heteroaryl, wherein each —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃— heterocyclyl, —C₆₋₂₀ aryl, —C₁₋₂₀ heteroaryl is independently optionally substituted with at least one of oxo, —CN, —C₁₋₁₂ alkyl, —C₁₋₁₂ haloalkyl, halo, —NO₂, —O—C₁₋₁₂ alkyl and —OH.

R¹³ is -(A)ₙ-R¹⁸. A is selected from —C₁₋₁₂ alkyl-, —C₃₋₈ cycloalkyl- and —C₂₋₁₂ alkenyl-. R¹⁸ is selected from hydrogen, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl and —C₅₋₁₃ spirocycle, wherein for A and R¹⁸ each —C₁₋₁₂ alkyl-, —C₃₋₈ cycloalkyl-, —C₂₋₁₂ alkenyl-, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl and —C₅₋₁₃ spirocycle is independently optionally substituted with at least one of oxo, —CN, —C₁₋₁₂ alkyl, —C₁₋₁₂ haloalkyl, —C₃₋₈ cycloalkyl, halo, —NO₂, —N(R^{k})(R^{l}), and —OR^{k}. n is 0 or 1.

The dashed lines represent optional double bonds wherein (a) X is C, Y is N, the bond between X and the ring carbon atom bearing R¹² is a double bond, and the bond between Y and the ring carbon atom bearing R¹² is a single bond, or (b) X is N, and Y is C, the bond between X and the ring carbon atom bearing R¹² is a single bond, and the bond between Y and the ring carbon atom bearing R¹² is a double bond.

Each R¹², R¹⁴, R¹⁶ and R¹⁷ is independently selected from hydrogen, halogen, —C₁₋₆ alkyl and —C₁₋₆ haloalkyl.

In some aspects, a compound or a pharmaceutically acceptable salt thereof of formula (II) is selected compounds 22-24:

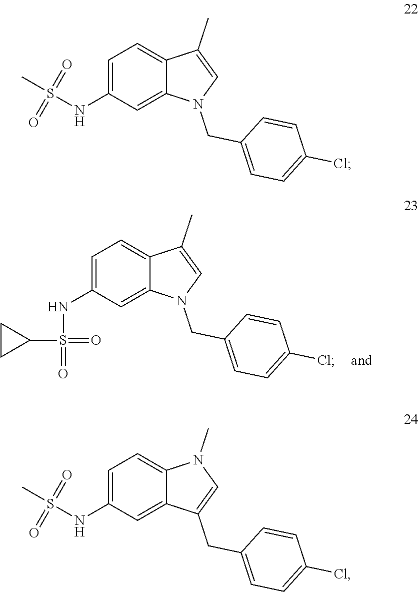 [SMILES: Cc1cn(Cc2ccc(Cl)cc2)c2cc(NS(C)(=O)=O)ccc12 ; Cc1cn(Cc2ccc(Cl)cc2)c2cc(NS(=O)(=O)C3CC3)ccc12 ; Cn1cc(Cc2ccc(Cl)cc2)c2cc(NS(C)(=O)=O)ccc21]

In some aspects, the compound or a pharmaceutically acceptable salt thereof of formula (I) is selected from the following stereoisomers:

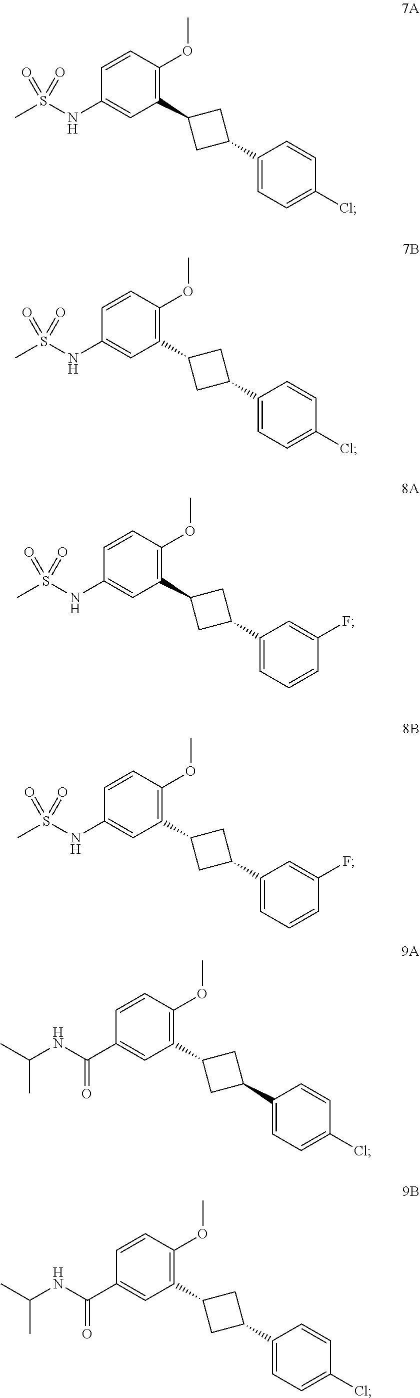 [SMILES: COc1ccc(NS(C)(=O)=O)cc1[C@H]1C[C@H](c2ccc(Cl)cc2)C1 ; COc1ccc(NS(C)(=O)=O)cc1[C@H]1C[C@@H](c2ccc(Cl)cc2)C1 ; COc1ccc(NS(C)(=O)=O)cc1[C@H]1C[C@H](c2cccc(F)c2)C1 ; COc1ccc(NS(C)(=O)=O)cc1[C@H]1C[C@@H](c2cccc(F)c2)C1 ; COc1ccc(C(=O)NC(C)C)cc1[C@H]1C[C@H](c2ccc(Cl)cc2)C1 ; COc1ccc(C(=O)NC(C)C)cc1[C@H]1C[C@@H](c2ccc(Cl)cc2)C1]

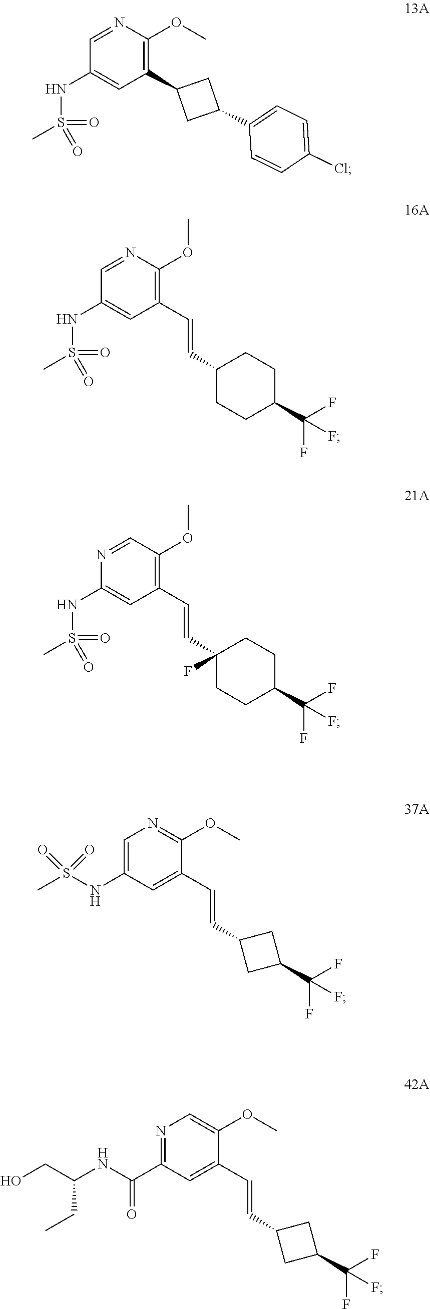 [SMILES: COc1ncc(NS(C)(=O)=O)cc1[C@H]1C[C@H](c2ccc(Cl)cc2)C1 ; COc1ncc(NS(C)(=O)=O)cc1/C=C/[C@H]1CC[C@H](C(F)(F)F)CC1 ; COc1cnc(NS(C)(=O)=O)cc1/C=C/[C@]1(F)CC[C@@H](C(F)(F)F)CC1 ; COc1ncc(NS(C)(=O)=O)cc1/C=C/[C@H]1C[C@H](C(F)(F)F)C1 ; CC[C@H](CO)NC(=O)c1cc(/C=C/[C@H]2C[C@H](C(F)(F)F)C2)c(OC)cn1]

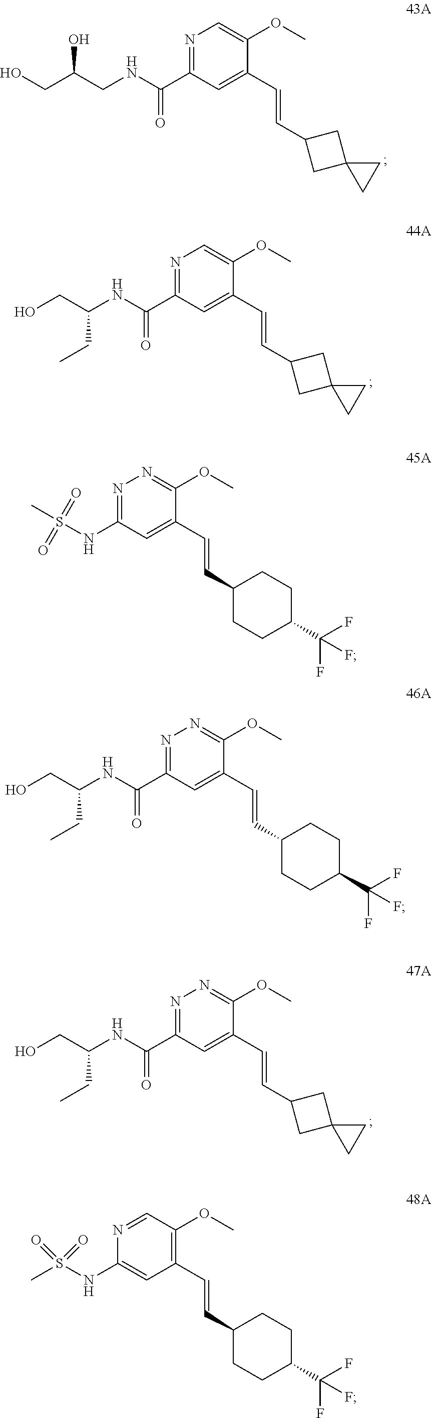 [SMILES: COc1cnc(C(=O)NC[C@H](O)CO)cc1/C=C/C1CC2(CC2)C1 ; CC[C@H](CO)NC(=O)c1cc(/C=C/C2CC3(CC3)C2)c(OC)cn1 ; COc1nnc(NS(C)(=O)=O)cc1/C=C/[C@H]1CC[C@H](C(F)(F)F)CC1 ; CC[C@H](CO)NC(=O)c1cc(/C=C/[C@H]2CC[C@H](C(F)(F)F)CC2)c(OC)nn1 ; CC[C@H](CO)NC(=O)c1cc(/C=C/C2CC3(CC3)C2)c(OC)nn1 ; COc1cnc(NS(C)(=O)=O)cc1/C=C/[C@H]1CC[C@H](C(F)(F)F)CC1]

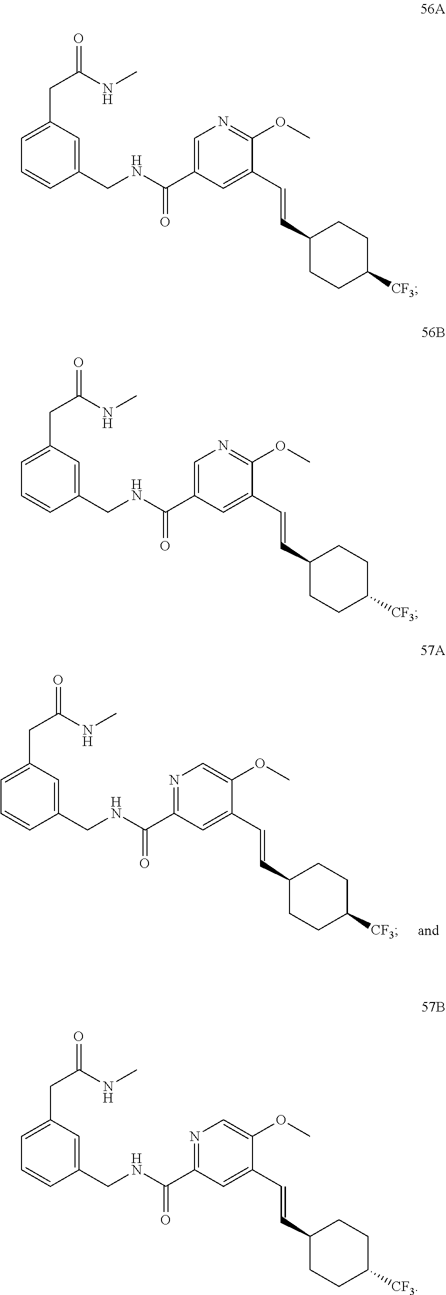 [SMILES: CNC(=O)Cc1cccc(CNC(=O)c2cnc(OC)c(/C=C/[C@H]3CC[C@@H](C(F)(F)F)CC3)c2)c1 ; CNC(=O)Cc1cccc(CNC(=O)c2cnc(OC)c(/C=C/[C@H]3CC[C@H](C(F)(F)F)CC3)c2)c1 ; CNC(=O)Cc1cccc(CNC(=O)c2cc(/C=C/[C@H]3CC[C@@H](C(F)(F)F)CC3)c(OC)cn2)c1 ; "and" ; CNC(=O)Cc1cccc(CNC(=O)c2cc(/C=C/[C@H]3CC[C@H](C(F)(F)F)CC3)c(OC)cn2)c1]

In some aspects, a pharmaceutical composition comprising a compound of formula (I) or formula (II) or a pharmaceutically acceptable salt thereof, and a pharmaceutically acceptable carrier, diluent or excipient is provided.

In some aspects, a compound of formula (I) or formula (II) or a pharmaceutically acceptable salt thereof is provided for use in medical therapy.

In some aspects, a compound of formula (I) or formula (II) or a pharmaceutically acceptable salt thereof is provided for the treatment or prophylaxis of cancer, mesothelioma, sarcoma, or leukemia.

In some aspects, a compound of formula (I) formula (II) or a pharmaceutically acceptable salt thereof is provided for the preparation of a medicament for the treatment or prophylaxis of cancer, mesothelioma, sarcoma, or leukemia.

In some aspects, a method for treating cancer, mesothelioma, sarcoma, or leukemia in a mammal is provided, the method comprising, administering a compound of formula (I) or formula (II) or a pharmaceutically acceptable salt thereof to the mammal.

In some aspects, a compound of formula (I) or formula (II) or a pharmaceutically acceptable salt thereof is provided for modulating TEAD activity.

In some aspects, a compound of formula (I) or formula (II) or a pharmaceutically acceptable salt thereof is provided for the treatment or prophylaxis of a disease or condition mediated by TEAD activity.

In some aspects, a compound of formula (I) or formula (II) or a pharmaceutically acceptable salt thereof is provided for use for the preparation of a medicament for the treatment or prophylaxis of a disease or condition that is mediated by TEAD activity.

In some aspects, a method for modulating TEAD activity is provided, the method comprising contacting TEAD with a compound of formula (I) or formula (II) or a pharmaceutically acceptable salt thereof.

In some aspects, a method for treating a disease or condition mediated by TEAD activity in a mammal is provided, the method comprising administering a compound of formula (I) or formula (II) or a pharmaceutically acceptable salt thereof to the mammal.

### DETAILED DESCRIPTION

### Definitions

Unless otherwise indicated, the following specific terms and phrases used in the description and claims are defined as follows.

The term “moiety” refers to an atom or group of chemically bonded atoms that is attached to another atom or molecule by one or more chemical bonds thereby forming part of a molecule.

The term “substituted” refers to the fact that at least one of the hydrogen atoms of that moiety is replaced by another substituent or moiety.

The term “alkyl” refers to an aliphatic straight-chain or branched-chain saturated hydrocarbon moiety having 1 to 20 carbon atoms, such as 1 to 12 carbon atoms, or 1 to 6 carbon atoms. Alkyl groups may be optionally substituted.

The term “cycloalkyl” means a saturated or partially unsaturated carbocyclic moiety having mono- or bicyclic (including bridged bicyclic) rings and 3 to 10 carbon atoms in the ring. In particular aspects, cycloalkyl may contain from 3 to 8 carbon atoms (i.e., (C₃-C₈)cycloalkyl). In other particular aspects cycloalkyl may contain from 3 to 6 carbon atoms (i.e., (C₃-C₆)cycloalkyl). Examples of cycloalkyl moieties include, but are not limited to, cyclopropyl, cyclobutyl, cyclopentyl, cyclohexyl, cycloheptyl, and partially unsaturated (cycloalkenyl) derivatives thereof (e.g. cyclopentenyl, cyclohexenyl, and cycloheptenyl). The cycloalkyl moiety can be attached in a spirocycle fashion such as spirocyclopropyl:

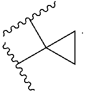 [SMILES: *C1(*)CC1]

The term “haloalkyl” refers to an alkyl group wherein one or more of the hydrogen atoms of the alkyl group has been replaced by the same or different halogen atoms, such as fluoro atoms. Examples of haloalkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, or trifluoromethyl. Haloalkyl groups may be optionally substituted.

The term “alkenyl” refers to a straight or branched chain alkyl or substituted alkyl group as defined elsewhere herein having at least one carbon-carbon double bond. Alkenyl groups may be optionally substituted.

The term “alkynyl” refers to a straight or branched chain alkyl or substituted alkyl group as defined elsewhere herein having at least one carbon-carbon triple bond. Alkynyl groups may be optionally substituted.

The terms “heterocyclyl” and “heterocycle” refer to a 4, 5, 6 and 7-membered monocyclic or 7, 8, 9 and 10-membered bicyclic (including bridged bicyclic) heterocyclic moiety that is saturated or partially unsaturated, and has one or more (e.g., 1, 2, 3 or 4) heteroatoms selected from oxygen, nitrogen and sulfur in the ring with the remaining ring atoms being carbon. When used in reference to a ring atom of a heterocycle, a nitrogen or sulfur may also be in an oxidized form, and a nitrogen may be substituted. The heterocycle can be attached to its pendant group at any heteroatom or carbon atom that results in a stable structure and any of the ring atoms can be optionally substituted. Examples of such saturated or partially unsaturated heterocycles include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The term the term heterocycle also includes groups in which a heterocycle is fused to one or more aryl, heteroaryl, or cycloalkyl rings, such as indolinyl, 3H-indolyl, chromanyl, 2-azabicyclo[2.2.1]heptanyl, octahydroindolyl, or tetrahydroquinolinyl. Heterocyclyl groups may be optionally substituted.

The term “aryl” refers to a cyclic aromatic hydrocarbon moiety having a mono-, bi- or tricyclic aromatic ring of 5 to 20 carbon ring atoms. Examples of aryl moieties include, but are not limited to, phenyl, naphthyl, benzyl, and the like. The term “aryl” also includes partially hydrogenated derivatives of the cyclic aromatic hydrocarbon moiety provided that at least one ring of the cyclic aromatic hydrocarbon moiety is aromatic, each being optionally substituted. In some aspects, monocyclic aryl rings may have 5 or 6 carbon ring atoms. Aryl groups may be optionally substituted.

The term “heteroaryl” refers an aromatic heterocyclic mono- or bicyclic ring system of 1 to 20 ring atoms, comprising 1, 2, 3 or 4 heteroatoms selected from N, O and S, the remaining ring atoms being carbon. Examples of heteroaryl moieties include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, or quinoxalinyl. Heteroaryl groups may be optionally substituted.

The terms “halo” and “halogen” refer fluoro, chloro, bromo and iodo. In some aspects, halo is fluoro or chloro.

The term “oxo” refers to the ═O moiety.

The term “spirocycle” refers to carbogenic bicyclic ring systems comprising between 5 and 15 carbon atoms with both rings connected through a single atom. The rings can be different in size and nature, or identical in size and nature. Examples include spiropentane, spirohexane, spiroheptane, spirooctane, spirononane, or spirodecane. One or more of the carbon atoms in the spirocycle can be substituted with a heteroatom (e.g., O, N, S, or P), wherein in such aspects the spirocycle may comprise between 3 and 14 carbon atoms. Spirocycle groups may be optionally substituted.

The term “pharmaceutically acceptable salts” refers to those salts which retain the biological effectiveness and properties of the free bases or free acids, which are not biologically or otherwise undesirable. Salts may be formed with inorganic acids such as hydrochloric acid, hydrobromic acid, sulfuric acid, nitric acid, phosphoric acid and the like, preferably hydrochloric acid, and organic acids such as acetic acid, propionic acid, glycolic acid, pyruvic acid, oxalic acid, maleic acid, malonic acid, salicylic acid, succinic acid, fumaric acid, tartaric acid, citric acid, benzoic acid, cinnamic acid, mandelic acid, methanesulfonic acid, ethanesulfonic acid, p-toluenesulfonic acid, N-acetylcystein and the like. In addition, salts may be prepared by the addition of an inorganic base or an organic base to the free acid. Salts derived from an inorganic base include, but are not limited to, the sodium, potassium, lithium, ammonium, calcium, and magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins and the like.

The term “prodrug” refers to those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

In some prodrug aspects, prodrugs include compounds wherein an amino acid residue, or a polypeptide chain of two or more (e.g., two, three or four) amino acid residues, is covalently joined through an amide or ester bond to a free amino, hydroxy or carboxylic acid group of a compound of the present disclosure. The amino acid residues include but are not limited to the 20 naturally occurring amino acids commonly designated by three letter symbols and also includes phosphoserine, phosphothreonine, phosphotyrosine, 4-hydroxyproline, hydroxylysine, demosine, isodemosine, gamma-carboxyglutamate, hippuric acid, octahydroindole-2-carboxylic acid, statine, 1,2,3,4-tetrahydroisoquinoline-3-carboxylic acid, penicillamine, ornithine, 3-methylhistidine, norvaline, beta-alanine, gamma-aminobutyric acid, citrulline, homocysteine, homoserine, methyl-alanine, para-benzoylphenylalanine, phenylglycine, propargylglycine, sarcosine, methionine sulfone and tert-butylglycine.

In some other prodrug aspects, a free carboxyl group of a compound of the disclosure can be derivatized as an amide or alkyl ester. In yet other prodrug aspects, prodrugs comprising free hydroxy groups can be derivatized as prodrugs by converting the hydroxy group into a group such as, but not limited to, a phosphate ester, hemisuccinate, dimethylaminoacetate, or phosphoryloxymethyloxycarbonyl group, as outlined in Fleisher, D. et al., (1996) Improved oral drug delivery: solubility limitations overcome by the use of prodrugs Advanced Drug Delivery Reviews, 19:115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers, wherein the acyl group can be an alkyl ester optionally substituted with groups including, but not limited to, ether, amine and carboxylic acid functionalities, or where the acyl group is an amino acid ester as described above, are also encompassed. Prodrugs of this type are described in J. Med. Chem., (1996), 39:10. More specific examples include replacement of the hydrogen atom of the alcohol group with a group such as (C₁₋₆)alkanoyloxymethyl, 1-((C₁₋₆)alkanoyloxy)ethyl, 1-methyl-((C₁₋₆)alkanoyloxy)ethyl, (C₁₋₆)alkoxycarbonyloxymethyl, N—(C₁₋₆)alkoxycarbonylaminomethyl, succinoyl, (C₁₋₆)alkanoyl, alpha-amino(C₁₋₄)alkanoyl, arylacyl and alpha-aminoacyl, or alpha-aminoacyl-alpha-aminoacyl, where each alpha-aminoacyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate).

For additional examples of prodrug derivatives, see, for example, a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs,” by H. Bundgaard p. 113-191 (1991); c) H. Bundgaard, Advanced Drug Delivery Reviews, 8:1-38 (1992); d) H. Bundgaard, et al., Journal of Pharmaceutical Sciences, 77:285 (1988); and e) N. Kakeya, et al., Chem. Pharm. Bull., 32:692 (1984), each of which is specifically incorporated herein by reference.

Additionally, the present disclosure provides for metabolites of compounds of the disclosure. As used herein, a “metabolite” refers to a product produced through metabolism in the body of a specified compound or salt thereof. Such products can result for example from the oxidation, reduction, hydrolysis, amidation, deamidation, esterification, deesterification, enzymatic cleavage, and the like, of the administered compound.

Metabolite products typically are identified by preparing a radiolabeled (e.g., ¹⁴C or ³H) isotope of a compound of the disclosure, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolite products, so long as they are not otherwise found in vivo, are useful in diagnostic assays for therapeutic dosing of the compounds of the disclosure.

Certain compounds of the present disclosure can exist in unsolvated forms as well as solvated forms, including hydrated forms. In general, the solvated forms are equivalent to unsolvated forms and are intended to be encompassed within the scope of the present disclosure. Certain compounds of the present disclosure can exist in multiple crystalline or amorphous forms. In general, all physical forms are equivalent for the uses contemplated by the present disclosure and are intended to be within the scope of the present disclosure.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Diastereomers are stereoisomers with opposite configuration at one or more chiral centers which are not enantiomers. Stereoisomers bearing one or more asymmetric centers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center or centers and is described by the R- and S-sequencing rules of Cahn, Ingold and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. In certain aspects the compound is enriched by at least about 90% by weight with a single diastereomer or enantiomer. In other aspects the compound is enriched by at least about 95%, 98%, or 99% by weight with a single diastereomer or enantiomer.

Certain compounds of the present disclosure possess asymmetric carbon atoms (optical centers) or double bonds; the racemates, diastereomers, geometric isomers, regioisomers and individual isomers (e.g., separate enantiomers) are all intended to be encompassed within the scope of the present disclosure.

The compounds of the present disclosure may also exist in different tautomeric forms, and all such forms are embraced within the scope of the disclosure. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Unless otherwise indicated, the term “a compound of the formula” or “a compound of formula” or “compounds of the formula” or “compounds of formula” refers to any compound selected from the genus of compounds as defined by the formula (including, if not otherwise noted, any embodiment or aspect thereof such as a pharmaceutically acceptable salt or ester of any such compound, a stereoisomer, a geometric isomer, a tautomer, a solvate, a metabolite, an isotope, a pharmaceutically acceptable salt, or a prodrug).

The term “a therapeutically effective amount” of a compound means an amount of compound that is effective to prevent, alleviate or ameliorate symptoms of disease or prolong the survival of the subject being treated. Determination of a therapeutically effective amount is within the skill in the art. The therapeutically effective amount or dosage of a compound according to this disclosure can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 0.1 mg to about 5,000 mg, 1 mg to about 1,000 mg, or 1 mg to 100 mg may be appropriate, although the lower and upper limits may be exceeded when indicated. The daily dosage can be administered as a single dose or in divided doses, or for parenteral administration, it may be given as continuous infusion.

The term “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with a compound of the disclosure, use thereof in the compositions of the disclosure is contemplated. Supplementary active compounds can also be incorporated into the compositions.

Compounds

In some aspects of the present disclosure, the compounds or a pharmaceutically acceptable salt thereof are of the following formula (I):

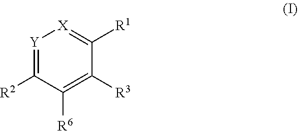

R¹ is selected from —C₁₋₆ alkyl, —C₃₋₈ cycloalkyl, —C₁₋₆ alkyl-C₃₋₈ cycloalkyl, —C₁₋₆ haloalkyl, —O—C₁₋₆ alkyl, —O—C₃₋₈ cycloalkyl, —O—C₁₋₆ alkyl-C₃₋₈ cycloalkyl and —O—C₁₋₆ haloalkyl. In some aspects, R¹ is —O—C₁₋₆ alkyl, such as —O—C₁₋₄ alkyl, —O—C₁₋₂ alkyl or —O—CH₃.

R² is selected from —C(O)—N(R^{a})(R^{b}) and —N(R^{c})—S(O)₂(R^{d}).

Each R^{a}, R^{b}, R^{c} and R^{d} is independently selected from —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₁₋₆ alkyl-C₅₋₂₀ aryl, —C₃₋₈ heterocyclyl, —C₆₋₂₀ aryl and —C₁₋₂₀ heteroaryl, wherein each —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₁₋₆ alkyl-C₅₋₂₀ aryl, —C₃— heterocyclyl, —C₆₋₂₀ aryl, and —C₁₋₂₀ heteroaryl is independently optionally substituted with at least one of oxo, —CN, —C₁₋₁₂ alkyl, —C₁₋₁₂ haloalkyl, halo, —NO₂, —N(R^{e})(R^{f}), —C₁₋₆ alkyl-C(O)—N(R^{e})(R^{f}), and —OR^{e}. Each R^{a}, R^{b} and R^{c} may further optionally independently be H. Each R^{e} and R^{f} is independently selected from hydrogen, —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃₋₈ heterocyclyl, —C₆₋₂₀ aryl and —C₁₋₂₀ heteroaryl, wherein each —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl is independently optionally substituted with at least one of oxo, —CN, —C₁₋₁₂ alkyl, —C₁₋₁₂ haloalkyl, halo, —NO₂, —O—C₁₋₁₂ alkyl and —OH. In some aspects, R^{c} is hydrogen and R^{d} is selected from —C₁₋₁₂ alkyl, —C₂₋₁₂ alkenyl and —C₃₋₈ cycloalkyl, wherein —C₁₋₁₂ alkyl is optionally substituted with —CN. In some aspects, R^{a} and R^{b} are independently selected from hydrogen and —C₁₋₁₂ alkyl, wherein —C₁₋₁₂ alkyl is optionally substituted with at least one —OH. In some aspects, R² is —C(O)—N(R^{a})(R^{b}), R^{a} is hydrogen, and R^{b} is selected from hydrogen, —C₁₋₆ alkyl, —C₁₋₄ alkyl and —C₂₋₄ alkyl, wherein said alkyl is optionally substituted with at least one —OH. In some such aspects, R^{a} is hydrogen and R^{b} is —CH₃. In some aspects, R² is —C(O)—N(R^{a})(R^{b}), R^{a} is hydrogen, and R^{b} is C₁₋₃-alkyl-C₅₋₆ aryl wherein the C₅₋₆ aryl is substituted with —C₁₋₃ alkyl-C(O)—N(R^{e})(R^{f}) wherein R^{e} is H and R^{f} is C₁₋₃ alkyl. In some aspects, R² is —N(R^{c})—S(O)₂(R^{d}), R^{c} is hydrogen, and R^{d} is selected from: (1) —C₁₋₄ alkyl, —C₁₋₂ alkyl, —C₃₋₆ cycloalkyl or —CH₃, (2) —C₂₋₄ alkenyl or —C₂ alkenyl, (3) —C₁₋₆ alkyl-CN or —C₁₋₄ alkyl-CN, and (4) —C₃₋₈ cycloalkyl, —C₃₋₆ cycloalkyl or —C₃ cycloalkyl. In some such aspects, R^{c} is hydrogen and R^{d} is —CH₃.

In some aspects, R² is selected from:

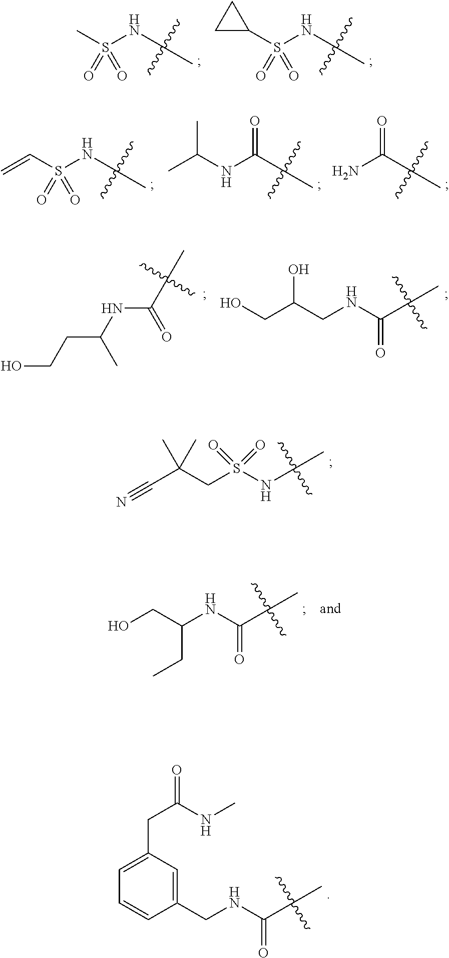

R³ is -(A)ₙ-R⁵. n is 0 or 1.

A is selected from a bond, —C₁₋₁₂ alkyl-, —C₃₋₈ cycloalkyl- and —C₂₋₁₂ alkenyl-, wherein each —C₁₋₁₂ alkyl-, —C₃₋₈ cycloalkyl- and —C₂₋₁₂ alkenyl- is independently optionally substituted with at least one of oxo, —CN, —C₁₋₁₂ alkyl, —C₁₋₁₂ haloalkyl, C-₃₋₈ cycloalkyl, halo, —NO₂, —N(R^{e})(R^{f}), and —OR^{e}. In some aspects, A is selected from (1) —C₁₋₆ alkyl-, —C₁₋₄ alkyl-, —C₁₋₂ alkyl- or —CH₂—, (2) —C₃₋₈ cycloalkyl-, —C₃₋₅ cycloalkyl- or —C₃₋₄ cycloalkyl- and (3) —C₂₋₆ alkenyl-, —C₂₋₄ alkenyl- or —C₂₋₃ alkenyl-. In some aspects, A is selected from (1) —C₃₋₈ cycloalkyl-, —C₃₋₅ cycloalkyl- or —C₃₋₄ cycloalkyl- and (2) —C₂₋₆ alkenyl-, —C₂₋₄ alkenyl- or —C₂₋₃ alkenyl-. In some particular aspects, A is C₂ alkenyl.

R⁵ is selected from hydrogen, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl, and —C₅₋₁₃ spirocycle, wherein each —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl and —C₅₋₁₃ spirocycle is independently optionally substituted with at least one of oxo, —CN, —C₁₋₁₂ alkyl, —C₁₋₁₂ haloalkyl, C-₃₋₈ cycloalkyl, halo, —NO₂, —N(R^{e})(R^{f}), and —OR^{e}. In some aspects, R⁵ is selected from hydrogen, —C₃₋₈ cycloalkyl, —C₆₋₂₀ aryl and —C₅₋₁₃ spirocycle wherein each —C₃₋₈ cycloalkyl, —C₆₋₂₀ aryl and —C₅₋₁₃ spirocycle is independently optionally substituted with at least one of C₁₋₁₂ alkyl, C₁₋₁₂ haloalkyl, halo and C-₃₋₈ cycloalkyl. In some aspects, R⁵ is selected from (1) hydrogen, (2) —C₃₋₈ cycloalkyl, —C₃₋₆ cycloalkyl or —C₄₋₆ cycloalkyl, wherein each said cycloalkyl is optionally substituted with one or more halo, —C₁₋₄ alkyl, —C₁₋₃ alkyl, —CH₃, —C₁₋₄ haloalkyl, —C₁₋₂ haloalkyl, or —C₁ haloalkyl, (3) C₅₋₆ aryl or C₆ aryl, wherein each said aryl is optionally substituted with one or more halo, —C₁₋₄ alkyl, —C₃ alkyl, —CH₃, —C₃₋₆ cycloalkyl, or —C₃ cycloalkyl, and (4) —C₅₋₁₂ spirocycle, —C₅₋₈ spirocycle, or —C₆ spirocycle. In some particular aspects, R⁵ is C₆ cycloalkyl substituted with at least one halo and/or —C₁ haloalkyl.

In some aspects, -(A)ₙ-R⁵ is selected from:

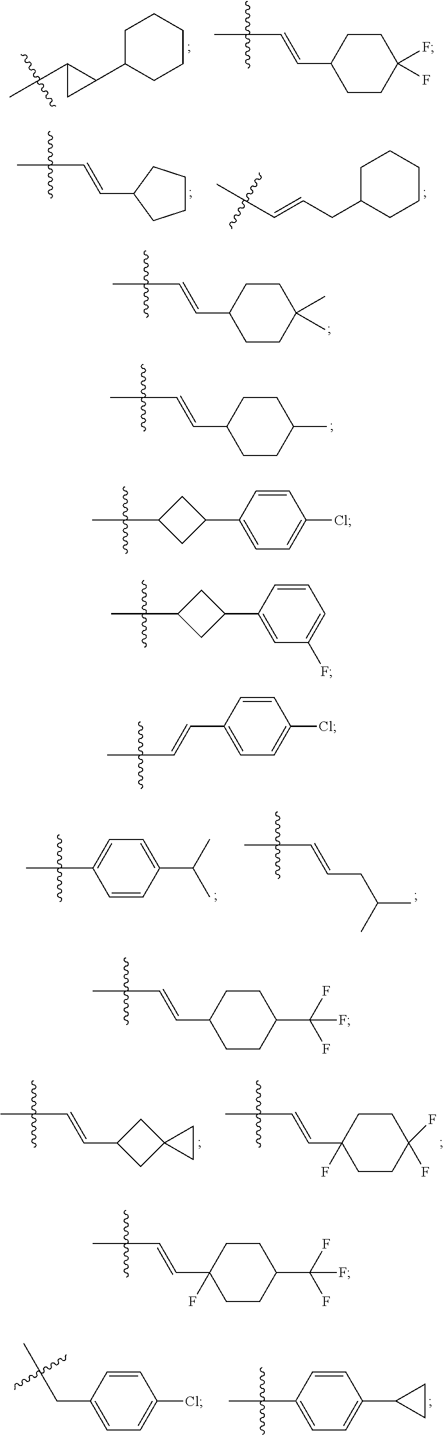

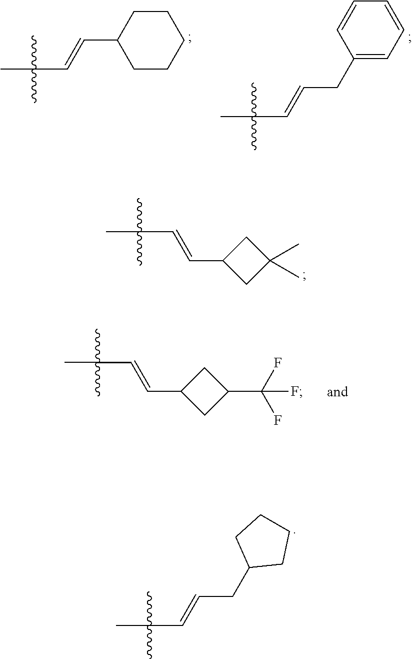

In some such aspects, -(A)ₙ-R⁵ is selected from:

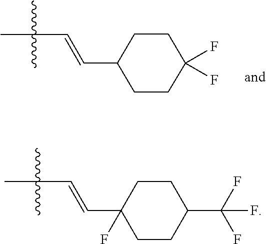 [SMILES: */C=C/C1CCC(F)(F)CC1 ; */C=C/C1(F)CCC(C(F)(F)F)CC1]

Each X and Y is independently selected from CR⁴ and N. Each R⁴ and R⁶ is independently selected from hydrogen, halogen, —C₁₋₆ haloalkyl and CN. In some aspects, each R⁴ is independently selected from hydrogen and halo. In some aspects, R⁶ is hydrogen. In some aspects, X is CH. In some aspects, X is N. In some aspects, Y is CH. In some aspects, Y is CF. In some aspects, Y is N.

In some formula (I) aspects, halo is selected from F and Cl. In some aspects, haloalkyl is selected from —CHF₂ and —CF₃.

In any of the various formula (I) aspects, when X and Y are each CR⁴ and when R² is —C(O)—N(R^{a})(R^{b}), A is selected from optionally substituted —C₁₋₁₂ alkyl-, —C₃₋₈ cycloalkyl- and —C₃₋₁₂ alkenyl- and R⁵ is selected from hydrogen, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl, and —C₅₋₁₃ spirocycle. For A and R⁵, each —C₁₋₁₂ alkyl-, —C₃₋₈ cycloalkyl-, —C₃₋₁₂ alkenyl-, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl and —C₅₋₁₃ spirocycle is independently optionally substituted with at least one of oxo, —CN, —C₁₋₁₂ alkyl, —C₁₋₁₂ haloalkyl, —C-₃₋₈ cycloalkyl, halo, —NO₂, —N(R^{e})(R^{f}), and —OR^{e}.

In some aspects, R¹ is C₁₋₄ alkoxy, C₁₋₄ alkyl or C₃₋₆ cycloalkyl. In some aspects R² is: (1) sulfonamide substituted with —C₁₋₆ alkyl, —C₃₋₆ cycloalkyl, —C₁₋₆ alkenyl or —C₁₋₆ alkyl-CN; or (2) amide substituted with C₁₋₆ alkyl, or amide substituted with C₁₋₆ alkyl that is substituted with one or more —OH. In some aspects, A is a bond (i.e., n=0), —C₃₋₆ cycloalkyl- or —C₂₋₆ alkenyl-. In some aspects, R⁵ is C₄₋₆ cycloalkyl, C₆ aryl, C₁₋₆ alkyl or C₅₋₇ spirocycle, wherein each C₄₋₆ cycloalkyl and C₆ aryl is optionally substituted with one or more halo, C₁₋₄ alkyl, C₁₋₄ haloalkyl or C₃₋₆ cycloalkyl. In some aspects, R⁶ is hydrogen. In some aspects, Y is CH, CF or N. In some aspects, X is CH or N.

The present disclosure is directed to compounds having the structure of formula IA:

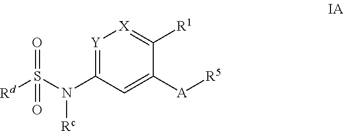

and pharmaceutically acceptable salts thereof.

Each X and Y is independently selected from CR⁴ and N. Each R⁴ is independently selected from hydrogen, halogen, —C₁₋₆ haloalkyl and CN. In some aspects, each R⁴ is independently selected from hydrogen and halo. In some aspects, R⁴ is hydrogen. In some aspects, X is CH. In some aspects, X is N. In some aspects, Y is CH. In some aspects, Y is CF. In some aspects, Y is N.

R¹ is selected from —C₁₋₆ alkyl, —C₃₋₈ cycloalkyl, —C₁₋₆ alkyl-C₃₋₈ cycloalkyl, —C₁₋₆ haloalkyl, —O—C₁₋₆ alkyl, —O—C₃₋₈ cycloalkyl, —O—C₁₋₆ alkyl-C₃₋₈ cycloalkyl and —O—C₁₋₆ haloalkyl. In some aspects, R¹ is —O—C₁₋₆ alkyl, such as —O—C₁₋₄ alkyl, —O—C₁₋₂ alkyl or —O—CH₃.

Each R^{c} and R^{d} is independently selected from hydrogen, —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₁₋₆ alkyl-C₅₋₂₀ aryl, —C₃₋₈ heterocyclyl, —C₆₋₂₀ aryl and —C₁₋₂₀ heteroaryl, wherein each —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₁₋₆ alkyl-C₅₋₂₀ aryl, —C₃— heterocyclyl, —C₆₋₂₀ aryl, and —C₁₋₂₀ heteroaryl is independently optionally substituted with at least one of oxo, —CN, —C₁₋₁₂ alkyl, —C₁₋₁₂ haloalkyl, halo, —NO₂, —N(R^{e})(R^{f}), —C₁₋₆ alkyl-C(O)—N(R^{e})(R^{f}), and —OR^{e}.

Each R^{e} and R^{f} is independently selected from hydrogen, —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃₋₈ heterocyclyl, —C₆₋₂₀ aryl and —C₁₋₂₀ heteroaryl, wherein each —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl is independently optionally substituted with at least one of oxo, —CN, —C₁₋₁₂ alkyl, —C₁₋₁₂ haloalkyl, halo, —NO₂, —O—C₁₋₁₂ alkyl and —OH. In some aspects, R^{c} is hydrogen and R^{d} is selected from —C₁₋₁₂ alkyl, —C₂₋₁₂ alkenyl and —C₃₋₈ cycloalkyl, wherein —C₁₋₁₂ alkyl is optionally substituted with —CN. In some aspects, R^{c} is hydrogen, and R^{d} is selected from: (1) —C₁₋₄ alkyl, —C₁₋₂ alkyl, —C₃₋₆ cycloalkyl or —CH₃, (2) —C₂₋₄ alkenyl or —C₂ alkenyl, (3) —C₁₋₆ alkyl-CN or —C₁₋₄ alkyl-CN, and (4) —C₃₋₈ cycloalkyl, —C₃₋₆ cycloalkyl or —C₃ cycloalkyl. In some such aspects, R^{c} is hydrogen and R^{d} is —CH₃.

A is selected from a bond, —C₁₋₁₂ alkyl-, —C₃₋₈ cycloalkyl- and —C₂₋₁₂ alkenyl-, wherein each —C₁₋₁₂ alkyl-, —C₃₋₈ cycloalkyl- and —C₂₋₁₂ alkenyl- is independently optionally substituted with at least one of oxo, —CN, —C₁₋₁₂ alkyl, —C₁₋₁₂ haloalkyl, C-₃₋₈ cycloalkyl, halo, —NO₂, —N(R^{e})(R^{f}), and —OR^{e}. In some aspects, A is selected from (1) —C₁₋₆ alkyl-, —C₁₋₄ alkyl-, —C₁₋₂ alkyl- or —CH₂—, (2) —C₃₋₈ cycloalkyl-, —C₃₋₅ cycloalkyl- or —C₃₋₄ cycloalkyl- and (3) —C₂₋₆ alkenyl-, —C₂₋₄ alkenyl- or —C₂₋₃ alkenyl-. In some aspects, A is selected from (1) —C₃₋₈ cycloalkyl-, —C₃₋₅ cycloalkyl- or —C₃₋₄ cycloalkyl- and (2) —C₂₋₆ alkenyl-, —C₂₋₄ alkenyl- or —C₂₋₃ alkenyl-. In some particular aspects, A is C₂ alkenyl.

R⁵ is selected from hydrogen, —C₁₋₆ alkyl, —C₃₋₈ cycloalkyl, —C₁₋₆ alkyl-C₃₋₈ cycloalkyl, —C₃₋₈ heterocyclyl, —C₆₋₂₀ aryl, —C₁₋₆ alkyl-C₆₋₂₀ aryl, —C₁₋₂₀ heteroaryl, and —C₅₋₁₃ spirocycle, wherein each —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl and —C₅₋₁₃ spirocycle is independently optionally substituted with at least one of oxo, —CN, —C₁₋₁₂ alkyl, —C₁₋₁₂ haloalkyl, C-₃₋₈ cycloalkyl, halo, —NO₂, —N(R^{e})(R^{f}), and —OR^{e}.

In some aspects, R⁵ is selected from hydrogen, —C₃₋₈ cycloalkyl, —C₆₋₂₀ aryl and —C₅₋₁₃ spirocycle wherein each —C₃₋₈ cycloalkyl, —C₆₋₂₀ aryl and —C₅₋₁₃ spirocycle is independently optionally substituted with at least one of C₁₋₁₂ alkyl, C₁₋₁₂ haloalkyl, halo and C-₃₋₈ cycloalkyl. In some aspects, R⁵ is selected from (1) hydrogen, (2) —C₃₋₈ cycloalkyl, —C₃₋₆ cycloalkyl or —C₄₋₆ cycloalkyl, wherein each said cycloalkyl is optionally substituted with one or more halo, —C₁₋₄ alkyl, —C₁₋₃ alkyl, —CH₃, —C₁₋₄ haloalkyl, —C₁₋₂ haloalkyl, or —C₁ haloalkyl, (3) C₅₋₆ aryl or C₆ aryl, wherein each said aryl is optionally substituted with one or more halo, —C₁₋₄ alkyl, —C₃ alkyl, —CH₃, —C₃₋₆ cycloalkyl, or —C₃ cycloalkyl, and (4) —C₅₋₁₂ spirocycle, —C₅₋₈ spirocycle, or —C₆ spirocycle. In some particular aspects, R⁵ is C₆ cycloalkyl substituted with at least one halo and/or —C₁ haloalkyl.

In embodiments, X and Y are each independently selected from CR⁴ and N, and R⁴ is hydrogen. In embodiments, X and Y are each CR⁴ and R⁴ is hydrogen. In embodiments, X is N and Y is CR⁴ and R⁴ is hydrogen. In embodiments, X is CR⁴ and R⁴ is hydrogen, and Y is N. In embodiments, X and Y are each N.

In embodiments, R¹ is —C₁₋₆ alkyl. In embodiments, R¹ is —O—CH₃. In embodiments, R¹ is —C₃₋₈ cycloalkyl. In embodiments, R¹ is cyclopropyl.

In embodiments, each R^{c} and R^{d} is independently selected from hydrogen, —C₁₋₁₂ alkyl, —C₂₋₁₂ alkenyl and —C₃₋₈ cycloalkyl, wherein each —C₁₋₁₂ alkyl is independently optionally substituted with at least one —CN. In embodiments, R^{c} is hydrogen. In embodiments, R^{d} is —C₁₋₁₂ alkyl, and R^{c} is hydrogen. In embodiments, R^{d} is methyl, and R^{c} is hydrogen. In embodiments, R^{d} is —C₁₋₁₂ alkyl substituted with one CN, and R^{c} is hydrogen. In embodiments, R^{d} is —C₂₋₁₂ alkenyl, and R^{c} is hydrogen. In embodiments, R^{d} is ethylene, and R^{c} is hydrogen. In embodiments, R^{d} is —C₃₋₈ cycloalkyl, and R^{c} is hydrogen. In embodiments, R^{d} is cyclopropyl, and R^{c} is hydrogen.

In embodiments, A is selected from a bond, —C₃₋₈ cycloalkyl- and —C₂₋₁₂ alkenyl-; and R⁵ is selected from —C₁₋₆ alkyl, —C₃₋₈ cycloalkyl, —C₁₋₆ alkyl-C₃₋₈ cycloalkyl, —C₆₋₂₀ aryl, —C₁₋₆ alkyl-C₆₋₂₀ aryl, and —C₅₋₁₃ spirocycle, wherein each —C₃₋₈ cycloalkyl and —C₆₋₂₀ aryl is independently optionally substituted with at least one —C₁₋₁₂ alkyl, —C₁₋₁₂ haloalkyl and halo. In embodiments, halo is chloro or fluoro. In embodiments, A is a bond, and R⁵ is —C₆₋₂₀ aryl substituted with at least one —C₁₋₁₂ alkyl. In embodiments, A is a bond, and R⁵ is phenyl substituted with at least one —C₁₋₁₂ alkyl. In embodiments, A is —C₃₋₈ cycloalkyl-, and R⁵ is —C₃₋₈ cycloalkyl or —C₆₋₂₀ aryl substituted with one halo. In embodiments, A is —C₃₋₄ cycloalkyl-, and R⁵ is —C₄₋₆cycloalkyl or phenyl substituted with one halo. In embodiments, A is —C₃₋₄ cycloalkyl-, and R⁵ is phenyl substituted with one halo. In embodiments, A is —C₂₋₁₂ alkenyl-, and R⁵ is selected from —C₁₋₆ alkyl, —C₃₋₈ cycloalkyl, —C₁₋₆ alkyl-C₃₋₈ cycloalkyl, —C₆₋₂₀ aryl, —C₁₋₆ alkyl-C₆₋₂₀ aryl, and —C₅₋₁₃ spirocycle, wherein each —C₃₋₈ cycloalkyl is independently optionally substituted with at least one —C₁₋₆ alkyl, —C₁₋₁₂ haloalkyl, and halo, and each —C₆₋₂₀ aryl is optionally substituted with at least one halo. In embodiments, A is ethylene. In embodiments, A is ethylene, and R⁵ is —C₁₋₆ alkyl. In embodiments, A is ethylene, and R⁵ is —C₃₋₈ cycloalkyl optionally substituted with at least one of —C₁₋₁₂ alkyl, —C₁₋₁₂ haloalkyl and halo. In embodiments, A is ethylene, and R⁵ is —C₄₋₆ cycloalkyl optionally substituted with at least one —C₁₋₁₂ alkyl, —C₁₋₁₂ haloalkyl and halo. In embodiments, A is ethylene, and R⁵ is —C₁₋₆ alkyl-C₃₋₈ cycloalkyl. In embodiments, A is ethylene, and R⁵ is phenyl substituted with one halo. In embodiments, A is ethylene, and R⁵ is —C₅₋₁₃ spirocycle.

In embodiments, X and Y are each CR⁴, and R⁴ is hydrogen; R¹ is —C₁₋₆ alkyl; and R^{c} is hydrogen.

In embodiments, X and Y are each CR⁴, and R⁴ is hydrogen; R¹ is —C₁₋₆ alkyl; and R^{d} is —C₁₋₁₂ alkyl, and R^{c} is hydrogen.

In embodiments, X and Y are each CR⁴, and R⁴ is hydrogen; R¹ is —O—C₁₋₆ alkyl; and R^{d} is cyclopropyl, and R^{c} is hydrogen.

In embodiments, X and Y are each CR⁴, and R⁴ is hydrogen; R¹ is —C₁₋₆ alkyl; and R^{d} is ethylene, and R^{c} is hydrogen.

In embodiments, X and Y are each CR⁴, and R⁴ is hydrogen; R¹ is —C₁₋₆ alkyl; R^{d} is —C₁₋₁₂ alkyl, and R^{c} is hydrogen; and A is —C₃₋₄ cycloalkyl-.

In embodiments, X and Y are each CR⁴, and R⁴ is hydrogen; R¹ is —O—C₁₋₆ alkyl; R^{d} is —C₁₋₁₂ alkyl, and R^{c} is hydrogen; and A is ethylene.

In embodiments, X and Y are each CR⁴, and R⁴ is hydrogen; R¹ is —O—C₁₋₆ alkyl; R^{d} is —C₁₋₁₂ alkyl, and R^{c} is hydrogen; and A is a bond.

In embodiments, at least one of X or Y is N; R¹ is —O—C₁₋₆ alkyl; R^{d} is —C₁₋₁₂ alkyl, and R^{c} is hydrogen; and A is ethylene.

In embodiments, X is N and Y is CR⁴ and R⁴ is hydrogen; R¹ is —O—C₁₋₆ alkyl; R^{d} is —C₁₋₁₂ alkyl, and R^{c} is hydrogen; and A is ethylene.

In embodiments, X is CR⁴ and R⁴ is hydrogen, and Y is N; R¹ is —O—C₁₋₆ alkyl; R^{d} is —C₁₋₁₂ alkyl, and R^{c} is hydrogen; and A is ethylene.

In embodiments, X and Y are each N; R¹ is —O—C₁₋₆ alkyl; R^{d} is —C₁₋₁₂ alkyl, and R^{c} is hydrogen; and A is ethylene.

In embodiments, X and Y are each independently selected from CR⁴ and N, and R⁴ is hydrogen; R¹ is —O—CH₃; R^{d} is —C₁₋₁₂ alkyl, and R^{c} is hydrogen; A is a bond; and R⁵ is —C₆₋₂₀ aryl substituted with at least one —C₁₋₁₂ alkyl.

In embodiments, X and Y are each independently selected from CR⁴ and N, and R⁴ is hydrogen; R¹ is —O—CH₃; R^{d} is methyl, and R^{c} is hydrogen; A is —C₃₋₄ cycloalkyl-, and R⁵ is —C₄₋₆ cycloalkyl or phenyl substituted with one halo.

In embodiments, X and Y are each independently selected from CR⁴ and N, and R⁴ is hydrogen; R¹ is —O—CH₃; R^{d} is methyl, and R^{c} is hydrogen; A is —C₂₋₁₂ alkenyl-, and R⁵ is selected from —C₁₋₆ alkyl, —C₃₋₈ cycloalkyl, —C₁₋₆ alkyl-C₃₋₈ cycloalkyl, —C₆₋₂₀ aryl, —C₁₋₆ alkyl-C₆₋₂₀ aryl, and —C₅₋₁₃ spirocycle, wherein each —C₃₋₈ cycloalkyl is independently optionally substituted with at least one —C₁₋₆ alkyl, —C₁₋₁₂ haloalkyl, and halo, and each —C₆₋₂₀ aryl is optionally substituted with at least one halo.

In embodiments, X and Y are each independently selected from CR⁴ and N, and R⁴ is hydrogen; R¹ is —O—CH₃; R^{d} is methyl, and R^{c} is hydrogen; A is —C₂₋₁₂ alkenyl-, and R⁵ is —C₄₋₆ cycloalkyl optionally substituted with at least one of —C₁₋₁₂ alkyl, —C₁₋₁₂haloalkyl and halo.

In embodiments, X and Y are each CR⁴, and R⁴ is hydrogen; R¹ is —O—CH₃; R^{d} is methyl, and R^{c} is hydrogen; A is ethylene, and R⁵ is —C₄₋₆ cycloalkyl optionally substituted with at least one halo.

In embodiments, X is N and Y is CR⁴ and R⁴ is hydrogen; R¹ is —O—C₁₋₆ alkyl; R^{d} is —C₁₋₁₂ alkyl, and R^{c} is hydrogen; and A is ethylene; and R⁵ is —C₄₋₆ cycloalkyl optionally substituted with at least one halo.

In embodiments, X is CR⁴ and R⁴ is hydrogen, and Y is N; R¹ is —O—C₁₋₆ alkyl; R^{d} is —C₁₋₁₂ alkyl, and R^{c} is hydrogen; and A is ethylene; and R⁵ is —C₄₋₆ cycloalkyl optionally substituted with at least one halo.

In embodiments, X and Y are each independently selected from CR⁴ and N, and R⁴ is hydrogen; R¹ is —O—CH₃; R^{d} is methyl, and R^{c} is hydrogen; A is —C₂₋₁₂ alkenyl-, and R⁵ is phenyl substituted with one halo.

In embodiments, X and Y are each independently selected from CR⁴ and N, and R⁴ is hydrogen; R¹ is —O—CH₃; R^{d} is methyl, and R^{c} is hydrogen; A is —C₂₋₁₂ alkenyl-, and R⁵ is —C₅₋₁₃ spirocycle.

In embodiments, each X and Y is independently selected from CR⁴ and N; each R⁴ is independently selected from hydrogen and halogen; R¹ is —O—C₁₋₆ alkyl; each R^{c} and R^{d} is independently selected from hydrogen, —C₁₋₁₂ alkyl, —C₂₋₁₂ alkenyl and —C₃₋₈ cycloalkyl, wherein each —C₁₋₁₂ alkyl is independently optionally substituted with at least one —CN; A is selected from a bond, —C₃₋₈ cycloalkyl- and —C₂₋₁₂ alkenyl-; and R⁵ is selected from —C₁₋₆ alkyl, —C₃₋₈ cycloalkyl, —C₁₋₆ alkyl-C₃₋₈ cycloalkyl, —C₆₋₂₀ aryl, —C₁₋₆ alkyl-C₆₋₂₀ aryl and —C₅₋₁₃ spirocycle, wherein —C₃₋₈ cycloalkyl and —C₆₋₂₀ aryl is independently optionally substituted with at least one —C₁₋₁₂ alkyl, —C₁₋₁₂ haloalkyl and halo.

In some aspects, compounds of formula (IA) are selected from the compounds listed in Table 1 below, including racemic mixtures and resolved isomers:

TABLE 1

| Cmpd | Structure | Name |
|---|---|---|
| 1 | 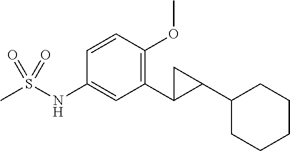 [SMILES: COc1ccc(NS(C)(=O)=O)cc1C1CC1C1CCCCC1] | N-[3-(2-cyclohexylcyclopropyl)-4- methoxy-phenyl]methanesulfonamide |
| 2 | 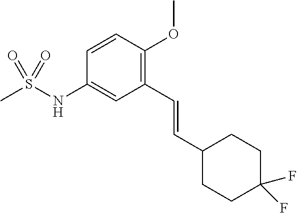 [SMILES: COc1ccc(NS(C)(=O)=O)cc1/C=C/C1CCC(F)(F)CC1] | N-[3-[(E)-2-(4,4- difluorocyclohexyl)vinyl]-4-methoxy- phenyl]methanesulfonamide |
| 3 | 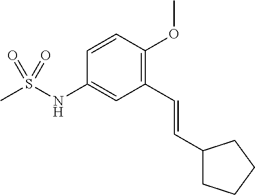 [SMILES: COc1ccc(NS(C)(=O)=O)cc1/C=C/C1CCCC1] | N-[3-[(E)-2-cyclopentylvinyl]-4- methoxy-phenyl]methanesulfonamide |
| 4 | 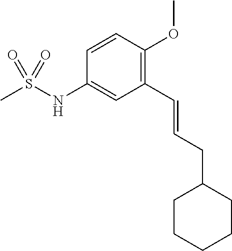 [SMILES: COc1ccc(NS(C)(=O)=O)cc1/C=C/CC1CCCCC1] | N-[3-[(E)-3-cyclohexylprop-1-enyl]-4- methoxy-phenyl]methanesulfonamide |
| 5 | 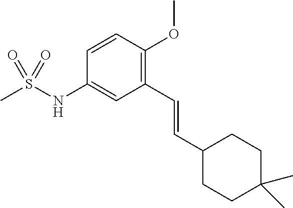 | N-[3-[(E)-2-(4,4- dimethylcyclohexyl)vinyl]-4-methoxy- phenyl]methanesulfonamide |
| 6 | 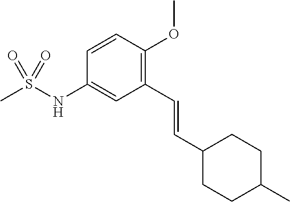 | N-[4-methoxy-3-[(E)-2-(4- methylcyclohexyl)vinyl]phenyl] methanesulfonamide |
| 7 | 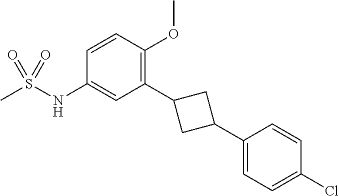 [SMILES: COc1ccc(NS(C)(=O)=O)cc1C1CC(c2ccc(Cl)cc2)C1] | N-[3-[3-(4-chlorophenyl)cyclobutyl]-4- methoxy-phenyl]methanesulfonamide |
| 7A | 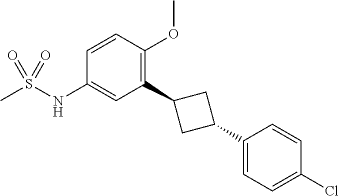 [SMILES: COc1ccc(NS(C)(=O)=O)cc1[C@H]1C[C@H](c2ccc(Cl)cc2)C1] | N-(3-((1R,3R)-3-(4- chlorophenyl)cyclobutyl)-4- methoxyphenyl)methanesulfonamide |
| 7B | 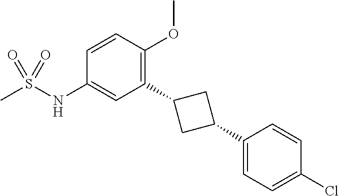 [SMILES: COc1ccc(NS(C)(=O)=O)cc1[C@H]1C[C@@H](c2ccc(Cl)cc2)C1] | N-(3-((1S,3S)-3-(4- chlorophenyl)cyclobutyl)-4- methoxyphenyl)methanesulfonamide |
| 8 | 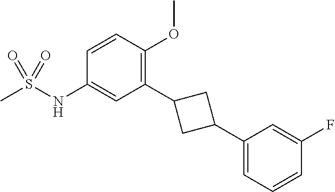 [SMILES: COc1ccc(NS(C)(=O)=O)cc1C1CC(c2cccc(F)c2)C1] | N-[3-[3-(3-fluorophenyl)cyclobutyl]-4- methoxy-phenyl]methanesulfonamide |
| 8A | 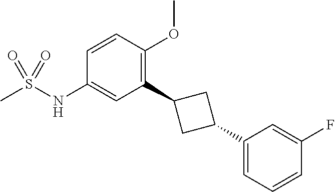 [SMILES: COc1ccc(NS(C)(=O)=O)cc1[C@H]1C[C@H](c2cccc(F)c2)C1] | N-(3-((1R,3R)-3-(3- fluorophenyl)cyclobutyl)-4- methoxyphenyl)methanesulfonamide |
| 8B | 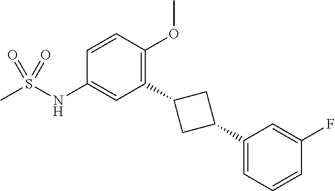 [SMILES: COc1ccc(NS(C)(=O)=O)cc1[C@H]1C[C@@H](c2cccc(F)c2)C1] | N-(3-((1S,3S)-3-(3- fluorophenyl)cyclobutyl)-4- methoxyphenyl)methanesulfonamide |
| 10 | 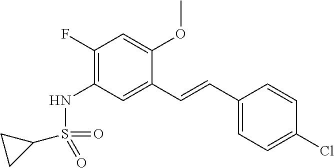 [SMILES: COc1cc(F)c(NS(=O)(=O)C2CC2)cc1/C=C/c1ccc(Cl)cc1] | N-[5-[(E)-2-(4-chlorophenyl)vinyl]-2- fluoro-4-methoxy- phenyl]cyclopropanesulfonamide |
| 11 | 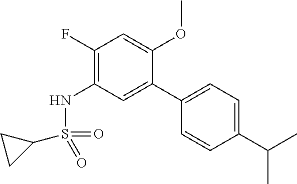 [SMILES: COc1cc(F)c(NS(=O)(=O)C2CC2)cc1-c1ccc(C(C)C)cc1] | N-[2-fluoro-5-(4-isopropylphenyl)-4- methoxy- phenyl]cyclopropanesulfonamide |
| 12 | 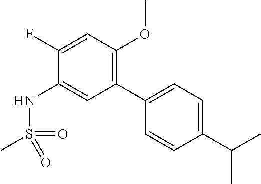 [SMILES: COc1cc(F)c(NS(C)(=O)=O)cc1-c1ccc(C(C)C)cc1] | N-[2-fluoro-5-(4-isopropylphenyl)-4- methoxy-phenyl]methanesulfonamide |
| 13 | 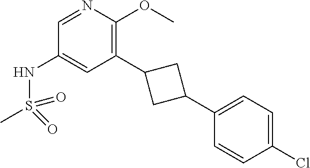 [SMILES: COc1ncc(NS(C)(=O)=O)cc1C1CC(c2ccc(Cl)cc2)C1] | N-[5-[3-(4-chlorophenyl)cyclobutyl]-6- methoxy-3-pyridyl]methanesulfonamide |
| 13A | 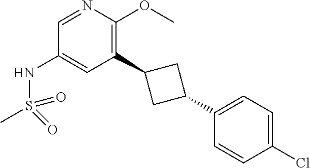 [SMILES: COc1ncc(NS(C)(=O)=O)cc1[C@H]1C[C@H](c2ccc(Cl)cc2)C1] | N-(5-((1R,3R)-3-(4- chlorophenyl)cyclobutyl)-6- methoxypyridin-3- yl)methanesulfonamide |
| 14 | 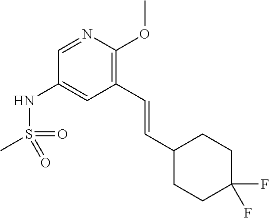 [SMILES: COc1ncc(NS(C)(=O)=O)cc1/C=C/C1CCC(F)(F)CC1] | N-[5-[(E)-2-(4,4- difluorocyclohexyl)vinyl]-6-methoxy-3- pyridyl]methanesulfonamide |
| 15 | 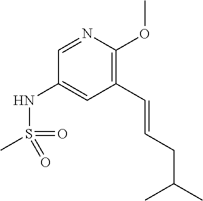 [SMILES: COc1ncc(NS(C)(=O)=O)cc1/C=C/CC(C)C] | N-[6-methoxy-5-[(E)-4-methylpent-1- enyl]-3-pyridyl]methanesulfonamide |
| 16 | 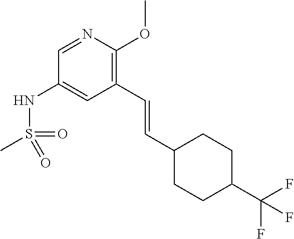 [SMILES: COc1ncc(NS(C)(=O)=O)cc1/C=C/C1CCC(C(F)(F)F)CC1] | N-[6-methoxy-5-[(E)-2-[4- (trifluoromethyl)cyclohexyl]vinyl]-3- pyridyl]methanesulfonamide |
| 16A | 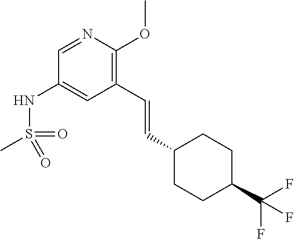 [SMILES: COc1ncc(NS(C)(=O)=O)cc1/C=C/[C@H]1CC[C@H](C(F)(F)F)CC1] | N-(6-methoxy-5-((E)-2-((1R,4R)-4- (trifluoromethyl)cyclohexyl)vinyl) pyridin-3-yl)methanesulfonamide |
| 17 | 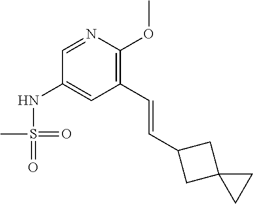 [SMILES: COc1ncc(NS(C)(=O)=O)cc1/C=C/C1CC2(CC2)C1] | N-[6-methoxy-5-[(E)-2-spiro[2.3]hexan- 5-ylvinyl]-3-pyridyl]methanesulfonamide |
| 18 | 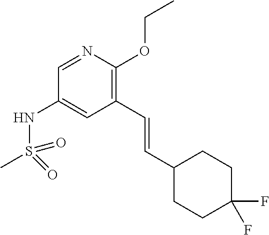 [SMILES: CCOc1ncc(NS(C)(=O)=O)cc1/C=C/C1CCC(F)(F)CC1] | N-[5-[(E)-2-(4,4- difluorocyclohexyl)vinyl]-6-ethoxy-3- pyridyl]methanesulfonamide |
| 20 | 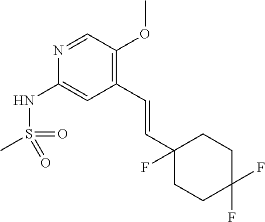 [SMILES: COc1cnc(NS(C)(=O)=O)cc1/C=C/C1(F)CCC(F)(F)CC1] | N-[5-methoxy-4-[(E)-2-(1,4,4- trifluorocyclohexyl)vinyl]-2- pyridyl]methanesulfonamide |
| 21 | 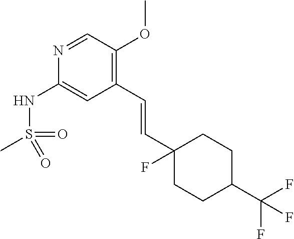 [SMILES: COc1cnc(NS(C)(=O)=O)cc1/C=C/C1(F)CCC(C(F)(F)F)CC1] | N-[4-[(E)-2-[1-fluoro-4- (trifluoromethyl)cyclohexyl]vinyl]-5- methoxy-2-pyridyl]methanesulfonamide |
| 21A | 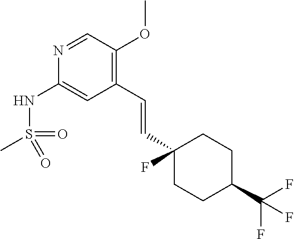 [SMILES: COc1cnc(NS(C)(=O)=O)cc1/C=C/[C@]1(F)CC[C@@H](C(F)(F)F)CC1] | N-(4-((E)-2-((1S,4S)-1-fluoro-4- (trifluoromethyl)cyclohexyl)vinyl)-5- methoxypyridin-2- yl)methanesulfonamide |
| 25 | 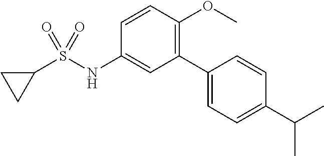 [SMILES: COc1ccc(NS(=O)(=O)C2CC2)cc1-c1ccc(C(C)C)cc1] | N-[3-(4-isopropylphenyl)-4-methoxy- phenyl]cyclopropanesulfonamide |
| 27 | 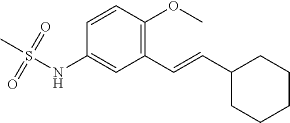 [SMILES: COc1ccc(NS(C)(=O)=O)cc1/C=C/C1CCCCC1] | N-[3-[(E)-2-cyclohexylvinyl]-4-methoxy- phenyl]methanesulfonamide |
| 28 | 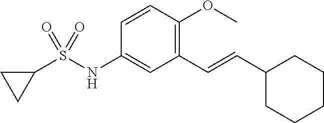 [SMILES: COc1ccc(NS(=O)(=O)C2CC2)cc1/C=C/C1CCCCC1] | N-[3-[(E)-2-cyclohexylvinyl]-4-methoxy- phenyl]cyclopropanesulfonamide |
| 33 | 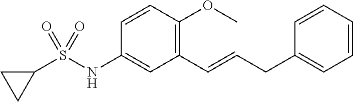 [SMILES: COc1ccc(NS(=O)(=O)C2CC2)cc1/C=C/Cc1ccccc1] | N-[4-methoxy-3-[(E)-3-phenylprop-1- enyl]phenyl]cyclopropanesulfonamide |
| 34 | 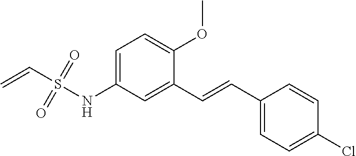 [SMILES: C=CS(=O)(=O)Nc1ccc(OC)c(/C=C/c2ccc(Cl)cc2)c1] | N-[3-[(E)-2-(4-chlorophenyl)vinyl]-4- methoxy-phenyl]ethenesulfonamide |
| 36 | 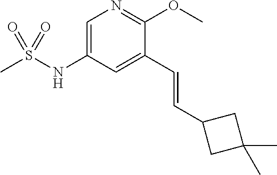 | N-[5-[(E)-2-(3,3- dimethylcyclobutyl)vinyl]-6-methoxy-3- pyridyl]methanesulfonamide |
| 37 | 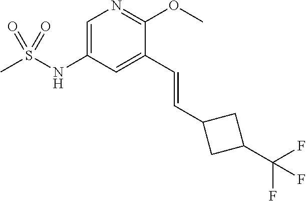 [SMILES: COc1ncc(NS(C)(=O)=O)cc1/C=C/C1CC(C(F)(F)F)C1] | N-[6-methoxy-5-[rac-(E)-2-[3- (trifluoromethyl)cyclobutyl]vinyl]-3- pyridyl]methanesulfonamide |
| 37A | 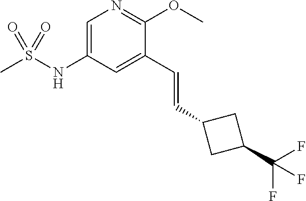 [SMILES: COc1ncc(NS(C)(=O)=O)cc1/C=C/[C@H]1C[C@H](C(F)(F)F)C1] | N-(6-methoxy-5-((E)-2-((1R,3R)-3- (trifluoromethyl)cyclobutyl)vinyl) pyridin-3-yl)methanesulfonamide |
| 38 | 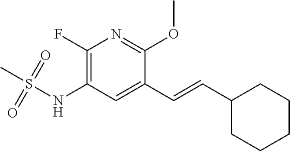 [SMILES: COc1nc(F)c(NS(C)(=O)=O)cc1/C=C/C1CCCCC1] | N-[5-[(E)-2-cyclohexylvinyl]-2-fluoro-6- methoxy-3-pyridyl]methanesulfonamide |
| 39 | 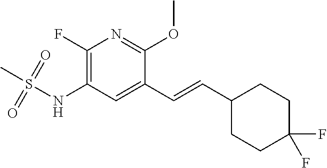 [SMILES: COc1nc(F)c(NS(C)(=O)=O)cc1/C=C/C1CCC(F)(F)CC1] | N-[5-[(E)-2-(4,4- difluorocyclohexyl)vinyl]-2-fluoro-6- methoxy-3-pyridyl]methanesulfonamide |
| 40 | 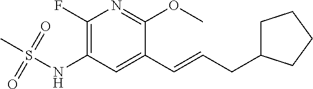 [SMILES: COc1nc(F)c(NS(C)(=O)=O)cc1/C=C/CC1CCCC1] | N-[5-[(E)-3-cyclopentylprop-1-enyl]-2- fluoro-6-methoxy-3- pyridyl]methanesulfonamide |
| 45 | 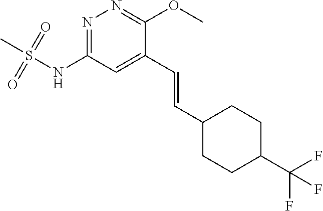 [SMILES: COc1nnc(NS(C)(=O)=O)cc1/C=C/C1CCC(C(F)(F)F)CC1] | (E)-N-(6-methoxy-5-(2-(4- (trifluoromethyl)cyclohexyl)vinyl) pyridazin-3-yl)methanesulfonamide |
| 45A | 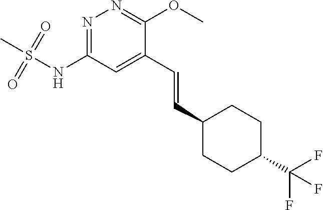 [SMILES: COc1nnc(NS(C)(=O)=O)cc1/C=C/[C@H]1CC[C@H](C(F)(F)F)CC1] | N-[6-methoxy-5-[rac-(E)-2-[4- (trifluoromethyl)cyclohexyl]vinyl] pyridazin-3-yl]methanesulfonamide |
| 48 | 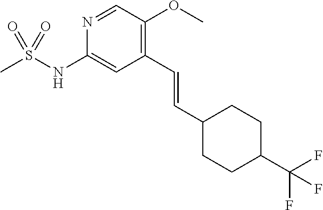 [SMILES: COc1cnc(NS(C)(=O)=O)cc1/C=C/C1CCC(C(F)(F)F)CC1] | (E)-N-(5-methoxy-4-(2-(4- (trifluoromethyl)cyclohexyl)vinyl) pyridin-2-yl)methanesulfonamide |
| 48A | 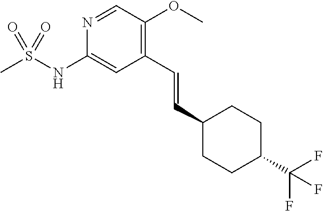 [SMILES: COc1cnc(NS(C)(=O)=O)cc1/C=C/[C@H]1CC[C@H](C(F)(F)F)CC1] | N-[5-methoxy-4-[rac-(E)-2-[4- (trifluoromethyl)cyclohexyl]vinyl]-2- pyridyl]methanesulfonamide |
| 49 | 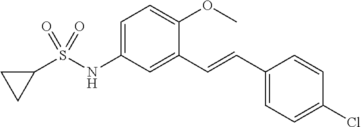 [SMILES: COc1ccc(NS(=O)(=O)C2CC2)cc1/C=C/c1ccc(Cl)cc1] | N-[3-[(E)-2-(4-chlorophenyl)vinyl]-4- methoxy- phenyl]cyclopropanesulfonamide |
| 50 | 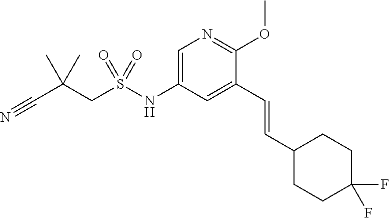 [SMILES: COc1ncc(NS(=O)(=O)CC(C)(C)C#N)cc1/C=C/C1CCC(F)(F)CC1] | 2-cyano-N-[5-[(E)-2-(4,4- difluorocyclohexyl)vinyl]-6-methoxy-3- pyridyl]-2-methyl-propane-1-sulfonamide |
| 52 | 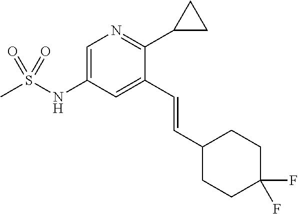 [SMILES: CS(=O)(=O)Nc1cnc(C2CC2)c(/C=C/C2CCC(F)(F)CC2)c1] | N-[6-cyclopropyl-5-[(E)-2-(4,4- difluorocyclohexyl)vinyl]-3- pyridyl]methanesulfonamide |
| 58 | 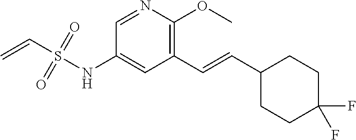 [SMILES: C=CS(=O)(=O)Nc1cnc(OC)c(/C=C/C2CCC(F)(F)CC2)c1] | N-[5-[(E)-2- (4,4-difluorocyclohexyl)vinyl]-6-methoxy-3- pyridyl]ethenesulfonamide |

The present disclosure is directed to compounds having the structure of formula IB:

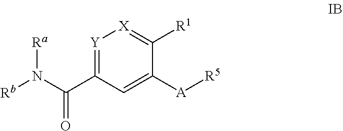

and pharmaceutically acceptable salts thereof.

Each X and Y is independently selected from CR⁴ and N. Each R⁴ is independently selected from hydrogen, halogen, —C₁₋₆ haloalkyl and CN. In some aspects, each R⁴ is independently selected from hydrogen and halo. In some aspects, R⁴ is hydrogen. In some aspects, X is CH. In some aspects, X is N. In some aspects, Y is CH. In some aspects, Y is CF. In some aspects, Y is N.

R¹ is selected from —C₁₋₆ alkyl, —C₃₋₈ cycloalkyl, —C₁₋₆ alkyl-C₃₋₈ cycloalkyl, —C₁₋₆ haloalkyl, —O—C₁₋₆ alkyl, —O—C₃₋₈ cycloalkyl, —O—C₁₋₆ alkyl-C₃₋₈ cycloalkyl and —O—C₁₋₆ haloalkyl. In some aspects, R¹ is —O—C₁₋₆ alkyl, such as —O—C₁₋₄ alkyl, —O—C₁₋₂ alkyl or —O—CH₃.

Each R^{a} and R^{b} is independently selected from hydrogen, —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₁₋₆ alkyl-C₅₋₂₀ aryl, —C₃₋₈ heterocyclyl, —C₆₋₂₀ aryl and —C₁₋₂₀ heteroaryl, wherein each —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₁₋₆ alkyl-C₅₋₂₀ aryl, —C₃₋₈ heterocyclyl, —C₆₋₂₀ aryl, and —C₁₋₂₀ heteroaryl is independently optionally substituted with at least one of oxo, —CN, —C₁₋₁₂ alkyl, —C₁₋₁₂ haloalkyl, halo, —NO₂, —N(R^{e})(R^{f}), —C₁₋₆ alkyl-C(O)—N(R^{e})(R^{f}), and —OR^{e}.

Each R^{e} and R^{f} is independently selected from hydrogen, —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃₋₈ heterocyclyl, —C₆₋₂₀ aryl and —C₁₋₂₀ heteroaryl, wherein each —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl is independently optionally substituted with at least one of oxo, —CN, —C₁₋₁₂ alkyl, —C₁₋₁₂ haloalkyl, halo, —NO₂, —O—C₁₋₁₂ alkyl and —OH.

In some aspects, R^{a} and R^{b} are independently selected from hydrogen and —C₁₋₁₂ alkyl, wherein —C₁₋₁₂ alkyl is optionally substituted with at least one —OH. In some aspects, R^{a} is hydrogen, and R^{b} is selected from hydrogen, —C₁₋₆ alkyl, —C₁₋₄ alkyl and —C₂₋₄ alkyl, wherein said alkyl is optionally substituted with at least one —OH. In some such aspects, R^{a} is hydrogen and R^{b} is —CH₃. In some aspects, R^{a} is hydrogen, and R^{b} is C₁₋₃-alkyl-C₅₋₆ aryl wherein the C₅₋₆ aryl is substituted with —C₁₋₃ alkyl-C(O)—N(R^{e})(R^{f}) wherein R^{e} is H and R^{f} is C₁₋₃ alkyl.

A is selected from a bond, —C₁₋₁₂ alkyl-, —C₃₋₈ cycloalkyl- and —C₂₋₁₂ alkenyl-, wherein each —C₁₋₁₂ alkyl-, —C₃₋₈ cycloalkyl- and —C₂₋₁₂ alkenyl- is independently optionally substituted with at least one of oxo, —CN, —C₁₋₁₂ alkyl, —C₁₋₁₂ haloalkyl, C-₃₋₈ cycloalkyl, halo, —NO₂, —N(R^{e})(R^{f}), and —OR^{e}. In some aspects, A is selected from (1) —C₁₋₆ alkyl-, —C₁₋₄ alkyl-, —C₁₋₂ alkyl- or —CH₂—, (2) —C₃₋₈ cycloalkyl-, —C₃₋₅ cycloalkyl- or —C₃₋₄ cycloalkyl- and (3) —C₂₋₆ alkenyl-, —C₂₋₄ alkenyl- or —C₂₋₃ alkenyl-. In some aspects, A is selected from (1) —C₃₋₈ cycloalkyl-, —C₃₋₅ cycloalkyl- or —C₃₋₄ cycloalkyl- and (2) —C₂₋₆ alkenyl-, —C₂₋₄ alkenyl- or —C₂₋₃ alkenyl-. In some particular aspects, A is C₂ alkenyl.

R⁵ is selected from hydrogen, —C₁₋₆ alkyl, —C₃₋₈ cycloalkyl, —C₁₋₆ alkyl-C₃₋₈ cycloalkyl, —C₃₋₈ heterocyclyl, —C₆₋₂₀ aryl, —C₁₋₆ alkyl-C₆₋₂₀ aryl, —C₁₋₂₀ heteroaryl, and —C₅₋₁₃ spirocycle, wherein each —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl and —C₅₋₁₃ spirocycle is independently optionally substituted with at least one of oxo, —CN, —C₁₋₁₂ alkyl, —C₁₋₁₂ haloalkyl, C-₃₋₈ cycloalkyl, halo, —NO₂, —N(R^{e})(R^{f}), and —OR^{e}.

In some aspects, R⁵ is selected from hydrogen, —C₃₋₈ cycloalkyl, —C₆₋₂₀ aryl and —C₅₋₁₃ spirocycle wherein each —C₃₋₈ cycloalkyl, —C₆₋₂₀ aryl and —C₅₋₁₃ spirocycle is independently optionally substituted with at least one of C₁₋₁₂ alkyl, C₁₋₁₂ haloalkyl, halo and C-₃₋₈ cycloalkyl. In some aspects, R⁵ is selected from (1) hydrogen, (2) —C₃₋₈ cycloalkyl, —C₃₋₆ cycloalkyl or —C₄₋₆ cycloalkyl, wherein each said cycloalkyl is optionally substituted with one or more halo, —C₁₋₄ alkyl, —C₁₋₃ alkyl, —CH₃, —C₁₋₄ haloalkyl, —C₁₋₂ haloalkyl, or —C₁ haloalkyl, (3) C₅₋₆ aryl or C₆ aryl, wherein each said aryl is optionally substituted with one or more halo, —C₁₋₄ alkyl, —C₃ alkyl, —CH₃, —C₃₋₆ cycloalkyl, or —C₃ cycloalkyl, and (4) —C₅₋₁₂ spirocycle, —C₅₋₈ spirocycle, or —C₆ spirocycle. In some particular aspects, R⁵ is C₆ cycloalkyl substituted with at least one halo and/or —C₁ haloalkyl.

In embodiments, X and Y are each independently selected from CR⁴ and N, and R⁴ is hydrogen. In embodiments, X and Y are each CR⁴ and R⁴ is hydrogen. In embodiments, X is N and Y is CR⁴ and R⁴ is hydrogen. In embodiments, X is CR⁴ and R⁴ is hydrogen, and Y is N. In embodiments, X and Y are each N.

In embodiments, R¹ is —O—C₁₋₆ alkyl. In embodiments, R¹ is —O—CH₃.

In embodiments, each R^{a} and R^{b} is independently selected from hydrogen, —C₁₋₁₂ alkyl, and —C₁₋₆ alkyl-C₅₋₂₀ aryl, wherein each —C₁₋₁₂ alkyl and —C₁₋₆ alkyl-C₅₋₂₀ aryl is independently optionally substituted with at least one of hydroxyl and —C₁₋₆ alkyl-C(O)—N(R^{e})(R^{f}) and each R^{e} and R^{f} is independently selected from hydrogen and —C₁₋₁₂ alkyl. In embodiments, R^{b} is hydrogen. In embodiments, R^{a} is —C₁₋₁₂ alkyl, and R^{b} is hydrogen. In embodiments, R^{a} is —C₁₋₁₂ alkyl substituted with one —OR^{e} where R^{e} is hydrogen, and R^{b} is hydrogen. In embodiments, R^{a} is —C₁₋₁₂ alkyl substituted with two —OR^{e} where each R^{e} is hydrogen, and R^{b} is hydrogen. In embodiments, R^{a} is —C₁₋₆ alkyl-C₅₋₂₀ aryl substituted with —C₁₋₆ alkyl-C(O)—N(R^{e})(R^{f}) and each R^{e} and R^{f} is independently selected from hydrogen and —C₁₋₁₂ alkyl, and R^{b} is hydrogen.

In embodiments, A is selected from a bond, —C₃₋₈ cycloalkyl- and —C₂₋₁₂ alkenyl-; and R⁵ is selected from —C₃₋₈ cycloalkyl, —C₆₋₂₀ aryl, —C₁₋₆ alkyl-C₆₋₂₀ aryl, and —C₅₋₁₃ spirocycle, wherein each —C₃₋₈ cycloalkyl and —C₆₋₂₀ aryl is independently optionally substituted with at least one of —C₁₋₁₂ haloalkyl, C-₃₋₈ cycloalkyl and halo. In embodiments, A is a bond, and R⁵ is —C₆₋₂₀ aryl, substituted with at least one C-₃₋₈ cycloalkyl. In embodiments, A is —C₃₋₈ cycloalkyl-; and R⁵ is —C₆₋₂₀ aryl substituted with one halo. In embodiments, A is —C₂₋₁₂ alkenyl-; and R⁵ is selected from —C₃₋₈ cycloalkyl, —C₆₋₂₀ aryl, —C₁₋₆ alkyl-C₆₋₂₀ aryl, and —C₅₋₁₃ spirocycle, wherein each —C₃₋₈ cycloalkyl is independently optionally substituted with at least one of —C₁₋₁₂haloalkyl, C-₃₋₈ cycloalkyl and halo. In embodiments, A is —C₂₋₁₂ alkenyl-; and R⁵ is —C₃₋₈ cycloalkyl optionally substituted with at least one of —C₁₋₁₂ haloalkyl and halo. In embodiments, A is —C₂₋₁₂ alkenyl-; and R⁵ is —C₁₋₆ alkyl-C₆₋₂₀ aryl.

In embodiments, X and Y are each CR⁴, and R⁴ is hydrogen; R¹ is —O—C₁₋₆ alkyl; R^{a} is —C₁₋₁₂ alkyl, and R^{b} is hydrogen; A is —C₃₋₈ cycloalkyl-; and R⁵ is —C₆₋₂₀ aryl substituted with one halo.

In embodiments, X and Y are each CR⁴, and R⁴ is hydrogen; R¹ is —C₁₋₆ alkyl; each R^{a} and R^{b} is independently selected from hydrogen and —C₁₋₁₂ alkyl; A is a bond or —C₂₋₁₂ alkenyl-; and R⁵ is selected from —C₃₋₈ cycloalkyl, —C₆₋₂₀ aryl, —C₁₋₆ alkyl-C₆₋₂₀ aryl, wherein each —C₃₋₈ cycloalkyl and —C₆₋₂₀ aryl is independently optionally substituted with at least one of —C₁₋₁₂haloalkyl and halo.

In embodiments, at least one of X and Y is N, and at least one of X and Y is CR⁴, and R⁴ is hydrogen, or each of X and Y is N; R¹ is —O—C₁₋₆ alkyl; each R^{a} is —C₁₋₁₂ alkyl substituted with at least one —OR^{e} where each R^{e} is hydrogen, and R^{b} is hydrogen; A is —C₂₋₁₂ alkenyl-; and R⁵ is —C₃₋₈ cycloalkyl, - or —C₅₋₁₃ spirocycle, wherein —C₃₋₈ cycloalkyl is substituted with at least one of —C₁₋₁₂ haloalkyl and halo.

In embodiments, at least one of X and Y is N, and at least one of X and Y is CR⁴, and R⁴ is hydrogen; R¹ is —O—C₁₋₆ alkyl; each R^{a} and R^{b} is independently selected from hydrogen and —C₁₋₆ alkyl-C₅₋₂₀ aryl substituted with at —C₁₋₆alkyl-C(O)—N(R^{e})(R^{f}) and each R^{e} and R^{f} is independently selected from hydrogen and —C₁₋₁₂ alkyl; A is —C₂₋₁₂ alkenyl-; and R⁵ is —C₃₋₈ cycloalkyl substituted with one —C₁₋₁₂ haloalkyl or two halo.

In embodiments, X and Y are each independently selected from CR⁴ and N, and R⁴ is hydrogen; R¹ is —O—C₁₋₆ alkyl; each R^{a} and R^{b} is independently selected from hydrogen, —C₁₋₁₂ alkyl, and —C₁₋₆ alkyl-C₅₋₂₀ aryl, wherein each —C₁₋₁₂ alkyl and —C₁₋₆ alkyl-C₅₋₂₀ aryl is independently optionally substituted with at least one —OR^{e} where R^{e} is hydrogen, and —C₁₋₆ alkyl-C(O)—N(R^{e})(R^{f}) and each R^{e} and R^{f} is independently selected from hydrogen and —C₁₋₁₂ alkyl; A is selected from a bond, —C₃₋₈ cycloalkyl- and —C₂₋₁₂ alkenyl-; and R⁵ is selected from —C₃₋₈ cycloalkyl, —C₆₋₂₀ aryl, —C₁₋₆ alkyl-C₆₋₂₀ aryl, and —C₅₋₁₃ spirocycle, wherein each —C₃₋₈ cycloalkyl and —C₆₋₂₀ aryl is independently optionally substituted with at least one of —C₁₋₁₂ haloalkyl, C-₃₋₈ cycloalkyl and halo.

In some aspects, compounds of formula (IB) are selected from the compounds listed in Table 2 below, including racemic mixtures and resolved isomers:

TABLE 2

| Cmpd | Structure | Name |
|---|---|---|
| 9 | 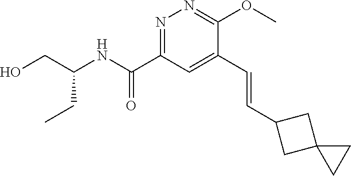 [SMILES: CC[C@H](CO)NC(=O)c1cc(/C=C/C2CC3(CC3)C2)c(OC)nn1] | 3-[3-(4-chlorophenyl)cyclobutyl]-N- isopropyl-4-methoxy-benzamide |
| 9A | 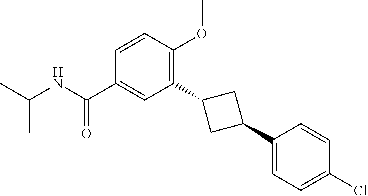 [SMILES: COc1ccc(C(=O)NC(C)C)cc1[C@H]1C[C@H](c2ccc(Cl)cc2)C1] | 3-((1R,3R)-3-(4- chlorophenyl)cyclobutyl)-N-isopropyl- 4-methoxybenzamide |
| 9B | 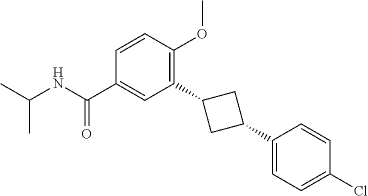 [SMILES: COc1ccc(C(=O)NC(C)C)cc1[C@H]1C[C@@H](c2ccc(Cl)cc2)C1] | 3-((1S,3S)-3-(4- chlorophenyl)cyclobutyl)-N-isopropyl- 4-methoxybenzamide |
| 19 | 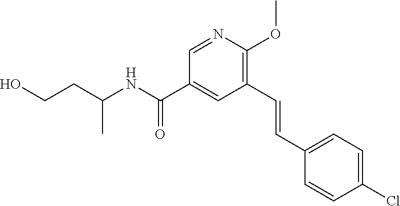 [SMILES: COc1ncc(C(=O)NC(C)CCO)cc1/C=C/c1ccc(Cl)cc1] | 5-[(E)-2-(4-chlorophenyl)vinyl]-N-(3- hydroxy-1-methyl-propyl)-6-methoxy- pyridine-3-carboxamide |
| 26 | 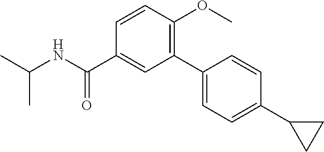 [SMILES: COc1ccc(C(=O)NC(C)C)cc1-c1ccc(C2CC2)cc1] | 3-(4-cyclopropylphenyl)-N-isopropyl-4- methoxy-benzamide |
| 29 | 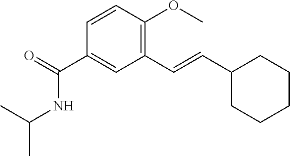 [SMILES: COc1ccc(C(=O)NC(C)C)cc1/C=C/C1CCCCC1] | 3-(E)-2-cyclohexylvinyl]-N-isopropyl-4- methoxy-benzamide |
| 30 | 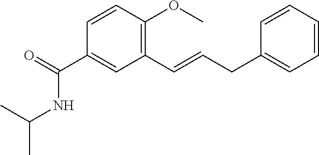 [SMILES: COc1ccc(C(=O)NC(C)C)cc1/C=C/Cc1ccccc1] | N-isopropyl-4-methoxy-3-[(E)-3- phenylprop-1-enyl]benzamide |
| 31 | 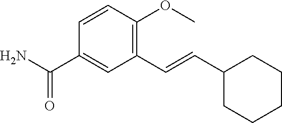 [SMILES: COc1ccc(C(N)=O)cc1/C=C/C1CCCCC1] | 3-[(E)-2-cyclohexylvinyl]-4-methoxy- benzamide |
| 32 | 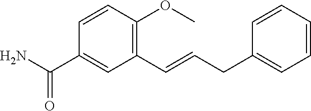 [SMILES: COc1ccc(C(N)=O)cc1/C=C/Cc1ccccc1] | 4-methoxy-3-[(E)-3-phenylprop-1- enyl]benzamide |
| 35 | 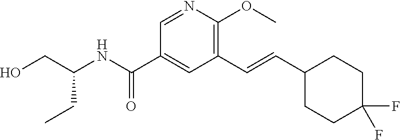 [SMILES: CC[C@H](CO)NC(=O)c1cnc(OC)c(/C=C/C2CCC(F)(F)CC2)c1] | 6-methoxy-5-[rac-(E)-2-(4,4- difluorocyclohexyl)vinyl]-N-[rac-(1R)-1- (hydroxymethyl)propyl]pyridine-3- carboxamide |
| 41 | 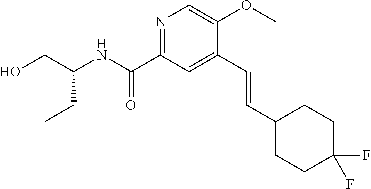 [SMILES: CC[C@H](CO)NC(=O)c1cc(/C=C/C2CCC(F)(F)CC2)c(OC)cn1] | 5-methoxy-4-[rac-(E)-2-(4,4- difluorocyclohexyl)vinyl]-N-[rac-(1R)-1- (hydroxymethyl)propyl]pyridine-2- carboxamide |
| 42 | 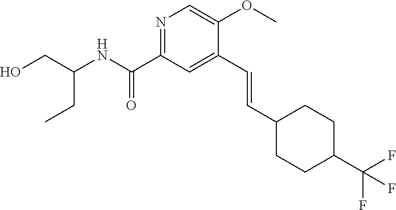 [SMILES: CCC(CO)NC(=O)c1cc(/C=C/C2CCC(C(F)(F)F)CC2)c(OC)cn1] | (E)-N-(1-hydroxybutan-2-yl)-5- methoxy-4-(2-(4- (trifluoromethyl)cyclohexyl)vinyl) picolinamide |
| 42A | 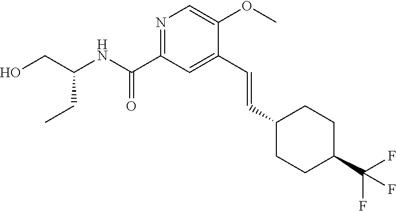 [SMILES: CC[C@H](CO)NC(=O)c1cc(/C=C/[C@H]2CC[C@H](C(F)(F)F)CC2)c(OC)cn1] | 5-methoxy-N-[rac-(1R)-1- (hydroxymethyl)propyl]-4-[rac-(E)-2-[4- (trifluoromethyl)cyclohexyl]vinyl]pyridine- 2-carboxamide |
| 43 | 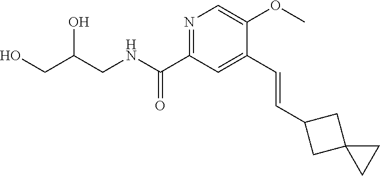 [SMILES: COc1cnc(C(=O)NCC(O)CO)cc1/C=C/C1CC2(CC2)C1] | (E)-N-(2,3-dihydroxypropyl)-5- methoxy-4-(2-(spiro[2.3]hexan-5- yl)vinyl)picolinamide |
| 43A | 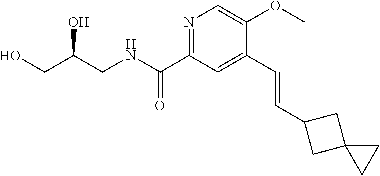 [SMILES: COc1cnc(C(=O)NC[C@H](O)CO)cc1/C=C/C1CC2(CC2)C1] | 5-methoxy-N-[rac-(2S)-2,3- dihydroxypropyl]-4-[rac-(E)-2- spiro[2.3]hexan-5-ylvinyl]pyridine-2- carboxamide |
| 44 | 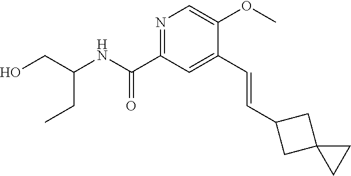 [SMILES: CCC(CO)NC(=O)c1cc(/C=C/C2CC3(CC3)C2)c(OC)cn1] | (E)-N-(1-hydroxybutan-2-yl)-5- methoxy-4-(2-(spiro[2.3]hexan-5- yl)vinyl)picolinamide |
| 44A | 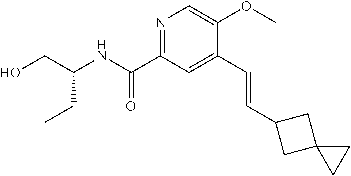 [SMILES: CC[C@H](CO)NC(=O)c1cc(/C=C/C2CC3(CC3)C2)c(OC)cn1] | 5-methoxy-N-[rac-(1R)-1- (hydroxymethyl)propyl]-4-[rac-(E)-2- spiro[2.3]hexan-5-ylvinyl]pyridine-2- carboxamide |
| 46 | 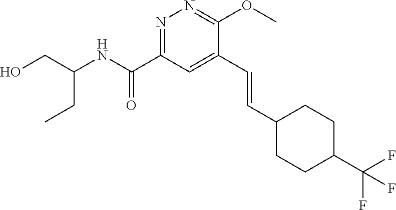 [SMILES: CCC(CO)NC(=O)c1cc(/C=C/C2CCC(C(F)(F)F)CC2)c(OC)nn1] | (E)-N-(1-hydroxybutan-2-yl)-6- methoxy-5-(2-(4- (trifluoromethyl)cyclohexyl)vinyl) pyridazine-3-carboxamide |
| 46A | 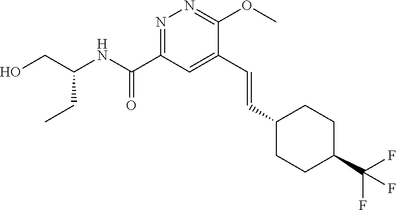 [SMILES: CC[C@H](CO)NC(=O)c1cc(/C=C/[C@H]2CC[C@H](C(F)(F)F)CC2)c(OC)nn1] | 6-methoxy-N-[rac-(1R)-1- (hydroxymethyl)propyl]-5-[rac-(E)-2-[4- (trifluoromethyl)cyclohexyl]vinyl] pyridazine-3-carboxamide |
| 47 | 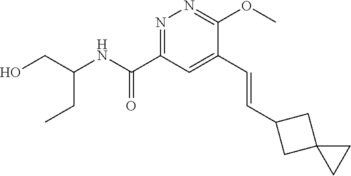 [SMILES: CCC(CO)NC(=O)c1cc(/C=C/C2CC3(CC3)C2)c(OC)nn1] | (E)-N-(1-hydroxybutan-2-yl)-6- methoxy-5-(2-(spiro[2.3]hexan-5- yl)vinyl)pyridazine-3-carboxamide |
| 47A | 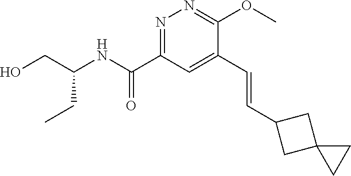 [SMILES: CC[C@H](CO)NC(=O)c1cc(/C=C/C2CC3(CC3)C2)c(OC)nn1] | 6-methoxy-N-[rac-(1R)-1- (hydroxymethyl)propyl]-5-[rac-(E)-2- spiro[2.3]hexan-5-ylvinyl]pyridazine-3- carboxamide |
| 51 | 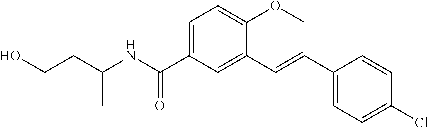 [SMILES: COc1ccc(C(=O)NC(C)CCO)cc1/C=C/c1ccc(Cl)cc1] | 3-[(E)-2-(4-chlorophenyl)vinyl]-N-(3- hydroxy-1-methyl-propyl)-4-methoxy- benzamide |
| 54 | 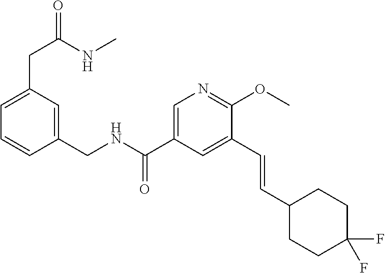 [SMILES: CNC(=O)Cc1cccc(CNC(=O)c2cnc(OC)c(/C=C/C3CCC(F)(F)CC3)c2)c1] | (E)-5-(2-(4,4-difluorocyclohexyl)vinyl)-6- methoxy-N-(3-(2-(methylamino)-2- oxoethyl)benzyl)nicotinamide |
| 55 | 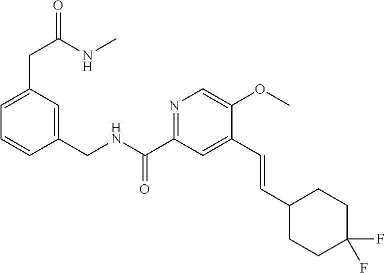 [SMILES: CNC(=O)Cc1cccc(CNC(=O)c2cc(/C=C/C3CCC(F)(F)CC3)c(OC)cn2)c1] | (E)-4-(2-(4,4-Difluorocyclohexyl)vinyl)-5- methoxy-N-(3-(2-(methylamino)-2- oxoethyl)benzyl)picolinamide |
| 56 | 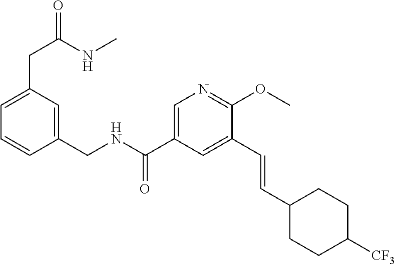 [SMILES: CNC(=O)Cc1cccc(CNC(=O)c2cnc(OC)c(/C=C/C3CCC(C(F)(F)F)CC3)c2)c1] | (E)-6-methoxy-N-(3-(2-(methylamino)-2- oxoethyl)benzyl)-5-(2-(4-(trifluoromethyl) cyclohexyl)vinyl)nicotinamide |
| 56A | 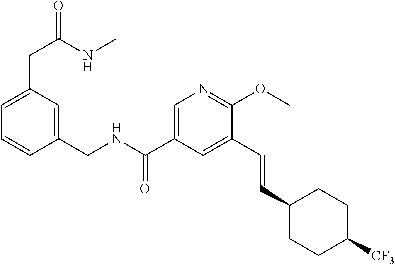 [SMILES: CNC(=O)Cc1cccc(CNC(=O)c2cnc(OC)c(/C=C/[C@H]3CC[C@@H](C(F)(F)F)CC3)c2)c1] | 6-Methoxy-N-(3-(2-(methylamino)-2- oxoethyl)benzyl)-5-((E)-2-(cis-4-(trifluoro methyl)cyclohexyl)vinyl)nicotinamide |
| 56B | 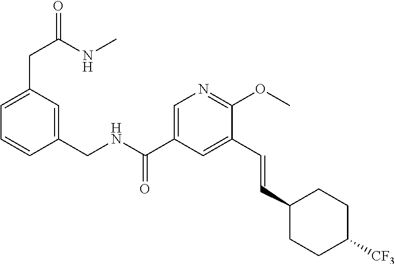 [SMILES: CNC(=O)Cc1cccc(CNC(=O)c2cnc(OC)c(/C=C/[C@H]3CC[C@H](C(F)(F)F)CC3)c2)c1] | 6-methoxy-N-(3-(2-(methyl amino)-2- oxoethyl)benzyl)-5-((E)-2-(trans-4- (trifluoromethyl)cyclohexyl)vinyl) nicotinamide |
| 57 | 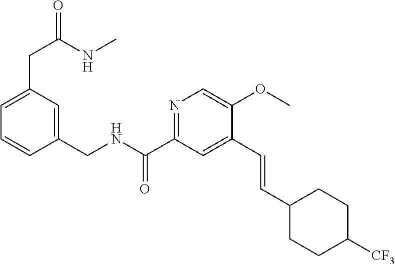 [SMILES: CNC(=O)Cc1cccc(CNC(=O)c2cc(/C=C/C3CCC(C(F)(F)F)CC3)c(OC)cn2)c1] | (E)-5-methoxy-N-(3-(2-(methylamino)-2- oxoethyl)benzyl)-4-(2-(4-(trifluoromethyl) cyclohexyl)vinyl)picolinamide |
| 57A | 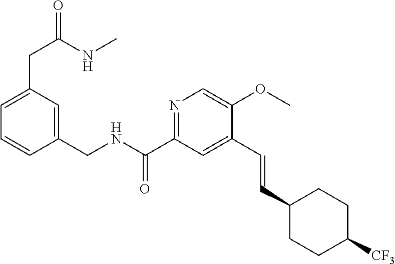 [SMILES: CNC(=O)Cc1cccc(CNC(=O)c2cc(/C=C/[C@H]3CC[C@@H](C(F)(F)F)CC3)c(OC)cn2)c1] | 5-Methoxy-N-(3-(2-(methylamino)-2- oxoethyl)benzyl)-4-((E)-2-(cis-4- (trifluoromethyl)cyclohexyl)vinyl) picolinamide |
| 57B | 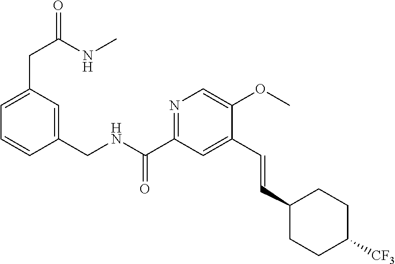 [SMILES: CNC(=O)Cc1cccc(CNC(=O)c2cc(/C=C/[C@H]3CC[C@H](C(F)(F)F)CC3)c(OC)cn2)c1] | 5-methoxy-N-(3-(2-(methylamino)-2- oxoethyl)benzyl)-4-((E)-2-(trans-4- (trifluoromethyl)cyclohexyl)vinyl) picolinamide |

In some aspects of the present disclosure, the compounds or a pharmaceutically acceptable salt thereof are of the following formula (II):

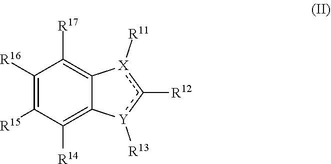

R¹¹ is selected from hydrogen, —C₁₋₆ alkyl, —C₃₋₈cycloalkyl, —C₁₋₆ alkyl-C₃₋₈ cycloalkyl, and —C₁₋₆ haloalkyl. In some aspects, R¹¹ is —C₁₋₆ alkyl. In some aspects, R¹¹ is selected from —C₁₋₄ alkyl, —C₁₋₂ alkyl and —CH₃.

R¹⁵ is —C(O)—N(R^{g})(R^{h}) or —N(Rⁱ)—S(O)₂(R^{j}).

Each R^{g}, R^{h}, Rⁱ, R^{j}, R^{k} and R^{l} is independently selected from —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃— heterocyclyl, —C₆₋₂₀ aryl and —C₁₋₂₀ heteroaryl, and wherein each —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃— heterocyclyl, —C₆₋₂₀ aryl and —C₁₋₂₀ heteroaryl is independently optionally substituted with at least one of oxo, —CN, —C₁₋₁₂ alkyl, —C₁₋₁₂ haloalkyl, halo, —NO₂, —N(R^{k})(R^{l}), and —OR^{k}. Each R^{g}, R^{h}, Rⁱ, R^{k} and R^{l} is may further optionally be H. In some aspects, R^{g} and R^{h} are independently selected from hydrogen, —C₁₋₁₂ alkyl and —C₃₋₈ cycloalkyl, wherein said —C₁₋₁₂ alkyl and —C₃₋₈ cycloalkyl are independently optionally substituted with at least one —OH. In some aspects, Rⁱ is hydrogen and R^{j} is selected from —C₁₋₁₂ alkyl, —C₂₋₁₂ alkenyl and —C₃₋₈ cycloalkyl, wherein —C₁₋₁₂ alkyl is optionally substituted with —CN.

In some aspects, R¹⁵ is —N(Rⁱ)—S(O)₂(R^{j}), Rⁱ is hydrogen, and R^{j} is selected from —C₁₋₄ alkyl, —C₁₋₂ alkyl and —CH₃. In some aspects, R¹⁵ is selected from:

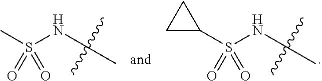

R¹³ is -(A)ₙ-R¹⁸. n is 0 or 1. In some aspects, R¹³ is selected from hydrogen and C₁₋₆ alkyl.

A is selected from —C₁₋₁₂ alkyl-, —C₃₋₈ cycloalkyl- and —C₂₋₁₂ alkenyl-. In some aspects, A is selected from (1) —C₁₋₆ alkyl-, —C₁₋₄ alkyl-, —C₁₋₂ alkyl- or —CH₂—, (2) —C₃₋₈ cycloalkyl-, —C₃₋₅ cycloalkyl- or —C₃₋₄ cycloalkyl- and (3) —C₂₋₆ alkenyl-, —C₂₋₄ alkenyl- or —C₂₋₃ alkenyl-. In some aspects, A is selected from (1) —C₁₋₆ alkyl-, —C₁₋₄ alkyl-, —C₁₋₂ alkyl- and —CH₂—. In some particular aspects, A is —CH₂—.

R¹⁸ is selected from hydrogen, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl and —C₅₋₁₃ spirocycle. For A and R¹⁸, each —C₁₋₁₂ alkyl-, —C₃₋₈ cycloalkyl-, —C₂₋₁₂ alkenyl-, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl and —C₅₋₁₃ spirocycle is independently optionally substituted with at least one of oxo, —CN, —C₁₋₁₂ alkyl, —C₁₋₁₂ haloalkyl, —C₃₋₈ cycloalkyl, halo, —NO₂, —N(R^{k})(R^{l}), and —OR^{k}. In some aspects, R¹⁸ is selected from hydrogen, —C₃₋₈ cycloalkyl, —C₆₋₂₀ aryl and —C₅₋₁₃ spirocycle wherein each —C₃₋₈ cycloalkyl, —C₆₋₂₀ aryl and —C₅₋₁₃ spirocycle is independently optionally substituted with at least one of —C₁₋₁₂ alkyl, —C₁₋₁₂ haloalkyl, halo and —C₃₋₈ cycloalkyl. In some aspects, R¹⁸ is —C₅₋₆ aryl or —C₆ aryl, wherein said aryl is optionally substituted with one or more halo.

In some aspects, -(A)ₙ-R¹⁸ is

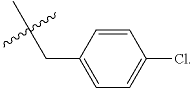 [SMILES: *Cc1ccc(Cl)cc1]

The dashed lines represent optional double bonds. In some aspects, X is C, Y is N, the bond between X and the ring carbon atom bearing R¹² is a double bond, and the bond between Y and the ring carbon atom bearing R¹² is a single bond. In some aspects, X is N, Y is C, the bond between X and the ring carbon atom bearing R¹² is a single bond, and the bond between Y and the ring carbon atom bearing R¹² is a double bond.

Each R¹², R¹⁴, R¹⁶ and R¹⁷ is independently selected from hydrogen, halogen, —C₁₋₆ alkyl and —C₁₋₆ haloalkyl. In some aspects, each of R¹², R¹⁴, R¹⁶ and R¹⁷ is hydrogen.

In some formula (II) aspects, halo is Cl.

In some aspects, R¹¹ is C₁₋₄ alkyl. In some aspects, R¹², R¹⁴, R¹⁶ and R¹⁷ are hydrogen. In some aspects, R¹⁵ is sulfonamide substituted with C₁₋₄ alkyl or C₃₋₆ cycloalkyl. In some aspects, A is —C₁₋₄ alkyl- and n is 1. In some aspects, R¹⁵ is C₆ aryl or C₄₋₆ cycloalkyl, and each C₆ aryl and C₄₋₆ cycloalkyl optionally substituted with one or more halo or C₁₋₄ haloalkyl. In some aspects: (a) X is C, Y is N, the bond between X and the ring carbon atom bearing R¹² is a double bond, and the bond between Y and the ring carbon atom bearing R¹² is a single bond, or (b) X is N, and Y is CH, the bond between X and the ring carbon atom bearing R¹² is a single bond, and the bond between Y and the ring carbon atom bearing R¹² is a double bond.

In some aspects, compounds of formula (II) are selected from the compounds listed in Table 3 below, including racemic mixtures and resolved isomers:

TABLE 3

| Cmpd | Structure | Name |
|---|---|---|
| 22 | 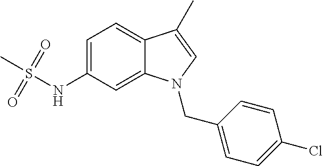 [SMILES: Cc1cn(Cc2ccc(Cl)cc2)c2cc(NS(C)(=O)=O)ccc12] | N-[1-[(4-chlorophenyl)methyl]-3-methyl- indol-6-yl]methanesulfonamide |
| 23 | 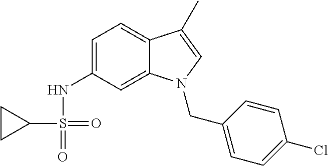 [SMILES: Cc1cn(Cc2ccc(Cl)cc2)c2cc(NS(=O)(=O)C3CC3)ccc12] | N-[1-[(4-chlorophenyl)methyl]-3-methyl- indol-6-yl]cyclopropanesulfonamide |
| 24 | 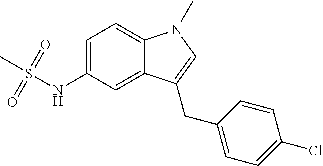 [SMILES: Cn1cc(Cc2ccc(Cl)cc2)c2cc(NS(C)(=O)=O)ccc21] | N-[3-[(4-chlorophenyl)methyl]-1-methyl- indol-5-yl]methanesulfonamide |

In some aspects, the compounds of the disclosure are isotopically-labeled by having one or more atoms therein replaced by an atom having a different atomic mass or mass number. Such isotopically-labeled (i.e., radiolabeled) compounds of formula (I) and/or formula (II) are considered to be within the scope of this disclosure. Examples of isotopes that can be incorporated into the compounds of formula (I) and/or formula (II) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These isotopically-labeled compounds would be useful to help determine or measure the effectiveness of the compounds, by characterizing, for example, the site or mode of action, or binding affinity to TEAD. Certain isotopically-labeled compounds of formula (I) and/or formula (II), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) and/or (II) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) and/or (II) can generally be prepared by conventional techniques known to those skilled in the art or by processes analogous to those described in the Examples as set out below using an appropriate isotopically-labeled reagent in place of the non-labeled reagent previously employed.

Pharmaceutical Compositions and Administration

In addition to one or more of the compounds provided above (including stereoisomers, geometric isomers, tautomers, solvates, metabolites, isotopes, pharmaceutically acceptable salts, or prodrugs thereof), the disclosure also provides for compositions and medicaments comprising a compound of the present disclosure or an embodiment or aspect thereof and at least one pharmaceutically acceptable carrier. The compositions of the disclosure can be used to selectively inhibit TEAD in patients (e.g., humans).

In one aspect, the disclosure provides for pharmaceutical compositions or medicaments comprising a compound of the disclosure (or embodiments and aspects thereof including stereoisomers, geometric isomers, tautomers, solvates, metabolites, isotopes, pharmaceutically acceptable salts, and prodrugs) and a pharmaceutically acceptable carrier, diluent or excipient. In another aspect, the disclosure provides for preparing compositions (or medicaments) comprising compounds of the disclosure. In another aspect, the disclosure provides for administering compounds of the disclosure and compositions comprising compounds of the disclosure to a patient (e.g., a human patient) in need thereof.

The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of a compound of the disclosure which are prepared by dissolving solid compounds of the disclosure in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of a compound of the disclosure together with a suitable carrier so as to prepare the proper dosage form for proper administration to the recipient.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The effective amount of the compound to be administered will be governed by such considerations, and is the minimum amount necessary to inhibit TEAD activity as required to prevent or treat the undesired disease or disorder, such as for example, pain. For example, such amount may be below the amount that is toxic to normal cells, or the mammal as a whole.

In one example, the therapeutically effective amount of the compound of the disclosure administered parenterally per dose will be in the range of about 0.01-100 mg/kg, alternatively about e.g., 0.1 to 20 mg/kg of patient body weight per day, with the typical initial range of compound used being 0.3 to 15 mg/kg/day. The daily does is, in certain aspects, given as a single daily dose or in divided doses two to six times a day, or in sustained release form. In the case of a 70 kg adult human, the total daily dose will generally be from about 7 mg to about 1,400 mg. This dosage regimen may be adjusted to provide the optimal therapeutic response. The compounds may be administered on a regimen of 1 to 4 times per day, preferably once or twice per day.

The compounds of the present disclosure may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

The compositions comprising compounds of the disclosure (or embodiments or aspects thereof including stereoisomers, geometric isomers, tautomers, solvates, metabolites, isotopes, pharmaceutically acceptable salts, and prodrugs thereof) are normally formulated in accordance with standard pharmaceutical practice as a pharmaceutical composition. A typical formulation is prepared by mixing a compound of the present disclosure and a diluent, carrier or excipient. Suitable diluents, carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present disclosure or pharmaceutical composition thereof) or aid in the manufacturing of the pharmaceutical product (i.e., medicament). Suitable carriers, diluents and excipients are well known to those skilled in the art and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG). An active pharmaceutical ingredient of the disclosure (e.g., a compound of formula (I) or formula (II) or an embodiment or aspect thereof) can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington: The Science and Practice of Pharmacy: Remington the Science and Practice of Pharmacy (2005) 21^{st} Edition, Lippincott Williams & Wilkins, Philadelphia, Pa. The particular carrier, diluent or excipient used will depend upon the means and purpose for which a compound of the present disclosure is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof.

Sustained-release preparations of a compound of the disclosure (e.g., compound of formula (I) or formula (II) or an embodiment or aspect thereof) can be prepared. Suitable examples of sustained-release preparations include semipermeable matrices of solid hydrophobic polymers containing a compound of formula (I) or formula (II) or an embodiment or aspect thereof, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma-ethyl-L-glutamate (Sidman et al., Biopolymers 22:547, 1983), non-degradable ethylene-vinyl acetate (Langer et al., J. Biomed. Mater. Res. 15:167, 1981), degradable lactic acid-glycolic acid copolymers such as the LUPRON DEPOT™ (injectable microspheres composed of lactic acid-glycolic acid copolymer and leuprolide acetate) and poly-D-(−)-3-hydroxybutyric acid (EP 133,988A). Sustained release compositions also include liposomally entrapped compounds, which can be prepared by methods known per se (Epstein et al., Proc. Natl. Acad. Sci. U.S.A. 82:3688, 1985; Hwang et al., Proc. Natl. Acad. Sci. U.S.A. 77:4030, 1980; U.S. Pat. Nos. 4,485,045 and 4,544,545; and EP 102,324A). Ordinarily, the liposomes are of the small (about 200-800 Angstroms) unilamelar type in which the lipid content is greater than about 30 mol % cholesterol, the selected proportion being adjusted for the optimal therapy.

In one example, compounds of the disclosure or an embodiment or aspect thereof may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of the disclosure (or an embodiment or aspect thereof) is formulated in an acetate buffer, at pH 5. In another aspect, the compounds of the disclosure or an embodiment thereof are sterile. The compound may be stored, for example, as a solid or amorphous composition, as a lyophilized formulation or as an aqueous solution

Formulations of a compound of the disclosure suitable for oral administration can be prepared as discrete units such as pills, capsules, cachets or tablets each containing a predetermined amount of a compound of the disclosure.

Compressed tablets can be prepared by compressing in a suitable machine a compound of the disclosure in a free-flowing form such as a powder or granules, optionally mixed with a binder, lubricant, inert diluent, preservative, surface active or dispersing agent. Molded tablets can be made by molding in a suitable machine a mixture of a powdered compound of the disclosure moistened with an inert liquid diluent. The tablets can optionally be coated or scored and optionally are formulated so as to provide slow or controlled release of a compound of the disclosure therefrom.

Tablets, troches, lozenges, aqueous or oil suspensions, dispersible powders or granules, emulsions, hard or soft capsules, e.g., gelatin capsules, syrups or elixirs can be prepared for oral use. Formulations of a compound of the disclosure intended for oral use can be prepared according to any method known to the art for the manufacture of pharmaceutical compositions and such compositions can contain one or more agents including sweetening agents, flavoring agents, coloring agents and preserving agents, in order to provide a palatable preparation. Tablets containing a compound of the disclosure in admixture with non-toxic pharmaceutically acceptable excipient which are suitable for manufacture of tablets are acceptable. These excipients can be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets can be uncoated or can be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax can be employed.

An example of a suitable oral administration form is a tablet containing about 0.1 mg, about 1 mg, about 5 mg, about 10 mg, about 25 mg, about 30 mg, about 50 mg, about 80 mg, about 100 mg, about 150 mg, about 250 mg, about 300 mg and about 500 mg of the compounds (or an embodiment or aspect thereof) of the disclosure compounded with a filler (e.g., lactose, such as about 90-30 mg anhydrous lactose), a disintegrant (e.g, croscarmellose, such as about 5-40 mg sodium croscarmellose), a polymer (e.g. polyvinylpyrrolidone (PVP), a cellulose (e.g., hydroxypropylmethyl cellulose (HPMC), and/or copovidone, such as about 5-30 mg PVP, HPMC or copovidone), and a lubricant (e.g., magnesium stearate, such as about 1-10 mg). Wet granulation, dry granulation or dry blending may be used. In one wet granulation aspect, powdered ingredients are first mixed together and then mixed with a solution or suspension of the polymer (e.g., PVP). The resulting composition can be dried, granulated, mixed with lubricant and compressed to tablet form using conventional equipment. An example of an aerosol formulation can be prepared by dissolving the compound, for example 5-400 mg, of the disclosure in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

For treatment of the eye or other external tissues, e.g., mouth and skin, the formulations are preferably applied as a topical ointment or cream containing the compounds of the disclosure in an amount of, for example, 0.075 to 20% w/w. When formulated in an ointment, the compounds of the disclosure can be employed with either a paraffinic or a water-miscible ointment base. Alternatively, the compounds of the disclosure can be formulated in a cream with an oil-in-water cream base. If desired, the aqueous phase of the cream base can include a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations can desirably include a compound which enhances absorption or penetration of a compound of the disclosure through the skin or other affected areas. Examples of such dermal penetration enhancers include dimethyl sulfoxide and related analogs.

For topical formulations, it is desired to administer an effective amount of a pharmaceutical composition according to the disclosure to target area, e.g., skin surfaces, mucous membranes, and the like, which are adjacent to peripheral neurons which are to be treated. This amount will generally range from about 0.0001 mg to about 1 g of a compound of the disclosure (or an embodiment or aspect thereof) per application, depending upon the area to be treated, whether the use is diagnostic, prophylactic or therapeutic, the severity of the symptoms, and the nature of the topical vehicle employed. A preferred topical preparation is an ointment, wherein about 0.001 to about 50 mg of a compound of the disclosure is used per cc of ointment base. The pharmaceutical composition can be formulated as transdermal compositions or transdermal delivery devices (“patches”). Such compositions include, for example, a backing, compound of the disclosure reservoir, a control membrane, liner and contact adhesive. Such transdermal patches may be used to provide continuous pulsatile, or on demand delivery of the compounds of the present disclosure as desired.

The formulations can be packaged in unit-dose or multi-dose containers, for example sealed ampoules and vials, and can be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of a compound of the disclosure.

When the binding target is located in the brain, certain aspects of the disclosure provide for a compound of the disclosure (or an embodiment or aspect thereof) to traverse the blood-brain barrier. Certain neurodegenerative diseases are associated with an increase in permeability of the blood-brain barrier, such that a compound of the disclosure (or an embodiment or aspect thereof) can be readily introduced to the brain. When the blood-brain barrier remains intact, several art-known approaches exist for transporting molecules across it, including, but not limited to, physical methods, lipid-based methods, and receptor and channel-based methods.

Physical methods of transporting a compound of the disclosure (or an embodiment or aspect thereof) across the blood-brain barrier include, but are not limited to, circumventing the blood-brain barrier entirely, or by creating openings in the blood-brain barrier.

Circumvention methods include, but are not limited to, direct injection into the brain (see, e.g., Papanastassiou et al., Gene Therapy 9:398-406, 2002), interstitial infusion/convection-enhanced delivery (see, e.g., Bobo et al., Proc. Natl. Acad. Sci. U.S.A. 91:2076-2080, 1994), and implanting a delivery device in the brain (see, e.g., Gill et al., Nature Med. 9:589-595, 2003; and Gliadel Wafers™, Guildford Pharmaceutical).

Methods of creating openings in the barrier include, but are not limited to, ultrasound (see, e.g., U.S. Patent Publication No. 2002/0038086), osmotic pressure (e.g., by administration of hypertonic mannitol (Neuwelt, E. A., Implication of the Blood-Brain Barrier and its Manipulation, Volumes 1 and 2, Plenum Press, N.Y., 1989)), and permeabilization by, e.g., bradykinin or permeabilizer A-7 (see, e.g., U.S. Pat. Nos. 5,112,596, 5,268,164, 5,506,206, and 5,686,416).

Lipid-based methods of transporting a compound of formula of the disclosure (or an embodiment or aspect thereof) across the blood-brain barrier include, but are not limited to, encapsulating the a compound of the disclosure (or an embodiment or aspect thereof) in liposomes that are coupled to antibody binding fragments that bind to receptors on the vascular endothelium of the blood-brain barrier (see, e.g., U.S. Patent Application Publication No. 2002/0025313), and coating a compound of the disclosure (or an embodiment or aspect thereof) in low-density lipoprotein particles (see, e.g., U.S. Patent Application Publication No. 2004/0204354) or apolipoprotein E (see, e.g., U.S. Patent Application Publication No. 2004/0131692).

Receptor and channel-based methods of transporting a compound of the disclosure (or an embodiment or aspect thereof) across the blood-brain barrier include, but are not limited to, using glucocorticoid blockers to increase permeability of the blood-brain barrier (see, e.g., U.S. Patent Application Publication Nos. 2002/0065259, 2003/0162695, and 2005/0124533); activating potassium channels (see, e.g., U.S. Patent Application Publication No. 2005/0089473), inhibiting ABC drug transporters (see, e.g., U.S. Patent Application Publication No. 2003/0073713); coating a compound of the disclosure (or an embodiment or aspect thereof) with a transferrin and modulating activity of the one or more transferrin receptors (see, e.g., U.S. Patent Application Publication No. 2003/0129186), and cationizing the antibodies (see, e.g., U.S. Pat. No. 5,004,697).

For intracerebral use, in certain aspects, the compounds can be administered continuously by infusion into the fluid reservoirs of the CNS, although bolus injection may be acceptable. The inhibitors can be administered into the ventricles of the brain or otherwise introduced into the CNS or spinal fluid. Administration can be performed by use of an indwelling catheter and a continuous administration means such as a pump, or it can be administered by implantation, e.g., intracerebral implantation of a sustained-release vehicle. More specifically, the inhibitors can be injected through chronically implanted cannulas or chronically infused with the help of osmotic mini pumps. Subcutaneous pumps are available that deliver proteins through a small tubing to the cerebral ventricles. Highly sophisticated pumps can be refilled through the skin and their delivery rate can be set without surgical intervention. Examples of suitable administration protocols and delivery systems involving a subcutaneous pump device or continuous intracerebroventricular infusion through a totally implanted drug delivery system are those used for the administration of dopamine, dopamine agonists, and cholinergic agonists to Alzheimer's disease patients and animal models for Parkinson's disease, as described by Harbaugh, J. Neural Transm. Suppl. 24:271, 1987; and DeYebenes et al., Mov. Disord. 2: 143, 1987.

Indications and Methods of Treatment

Representative compounds of the disclosure have been shown to modulate TEAD activity. Accordingly, the compounds of the disclosure (or an embodiment or aspect thereof) are useful as a medical therapy for treating diseases and conditions mediated by TEAD activity. Such diseases and conditions include but are not limited to cancers including acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

In a specific embodiment, compounds of the disclosure (or an embodiment or aspect thereof) can be administered as a medical therapy to treat proliferative disorders including acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldensto6m's macroglobulinemia, testicular tumors, uterine cancer and Wilms' tumor.

In one specific aspect, compounds of the disclosure (or an embodiment or aspect thereof) are administered as a medical therapy to treat acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

In another aspect, the disclosure provides for a method for treating acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor, comprising the step of administering a therapeutically effective amount of a compound according to formula (I) or formula (II) (or an embodiment or aspect thereof) as described elsewhere herein to a subject in need thereof.

In another aspect, the disclosure provides for a compound of formula (I) or formula (II) as described elsewhere herein or (or an embodiment or aspect thereof) for modulating TEAD activity. In some embodiments, the disclosure provides for a pharmaceutically acceptable salt of compound of formula (I) or formula (II) for modulating TEAD activity.

In another aspect, the disclosure provides for a compound of formula (I) or formula (II) as described elsewhere herein, or an embodiment or aspect thereof such as a pharmaceutically acceptable salt thereof for use in medical therapy.

In another aspect, the disclosure provides for a method for treatment or prophylaxis of acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor, comprising the step of administering a therapeutically effective amount of a compound according to formula (I) or formula (II) (or an embodiment or aspect thereof) as described elsewhere herein to a subject in need thereof.

In another aspect, the disclosure provides for a compound of formula (I) or formula (II) as described elsewhere herein or an embodiment or aspect thereof such as a pharmaceutically acceptable salt thereof for the treatment or prophylaxis of acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

In another aspect, the disclosure provides for the use of a compound of formula (I) or formula (II) as described elsewhere herein or an embodiment or aspect thereof such as a pharmaceutically acceptable salt thereof for the preparation of a medicament for the treatment or prophylaxis of acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

In another aspect, the disclosure provides for a method for treating acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor in a mammal (e.g., a human) comprising administering a compound of formula (I) or formula (II) as described elsewhere herein or an embodiment or aspect thereof such as a pharmaceutically acceptable salt thereof to the mammal.

In another aspect, the disclosure provides for a method for modulating TEAD activity, comprising contacting TEAD with a compound of formula (I) or formula (II) as described elsewhere herein or an embodiment or aspect thereof such as a pharmaceutically acceptable salt thereof.

In another aspect, the disclosure provides for a compound of formula (I) or formula (II) as described elsewhere herein or an embodiment or aspect thereof such as a pharmaceutically acceptable salt thereof for the treatment or prophylaxis of a disease or condition mediated by TEAD activity. Within aspects of this embodiment, the disease or condition is acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

In another aspect, the disclosure provides for the use of a compound of formula (I) or formula (II) as described elsewhere herein or an embodiment or aspect thereof such as a pharmaceutically acceptable salt thereof for the preparation of a medicament for the treatment or prophylaxis of a disease or condition that is mediated by TEAD activity. Within aspects of this embodiment, the disease or condition is acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

In one aspect, compounds of the disclosure demonstrate higher potency as compared to other analogues. Such representative compounds, commensurate in scope of the present invention, are shown in Table 4.

Combination Therapy

The compounds of formula (I) or formula (II) or salts thereof may be employed alone or in combination with other agents for treatment. For example, the second agent of the pharmaceutical combination formulation or dosing regimen may have complementary activities to the compound of formula (I) or formula (II) such that they do not adversely affect each other. The compounds may be administered together in a unitary pharmaceutical composition or separately. In one embodiment a compound or a pharmaceutically acceptable salt can be co-administered with a cytotoxic agent to treat proliferative diseases and cancer.

The term “co-administering” refers to either simultaneous administration, or any manner of separate sequential administration, of a compound of formula (I) or formula (II) or a salt thereof, and a further active pharmaceutical ingredient or ingredients, including cytotoxic agents and radiation treatment. If the administration is not simultaneous, the compounds are administered in a close time proximity to each other. Furthermore, it does not matter if the compounds are administered in the same dosage form, e.g. one compound may be administered topically and another compound may be administered orally.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of formula I or formula II, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. In certain embodiments, compositions of this invention are formulated such that a dosage of between 0.01-100 mg/kg body weight/day of an inventive can be administered.

Typically, any agent that has activity against a disease or condition being treated may be co-administered. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the disease involved.

In one embodiment, the treatment method includes the co-administration of a compound of formula (I) or formula (II) or a pharmaceutically acceptable salt thereof and at least one cytotoxic agent. The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof.

Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signaling inhibitors; HDAC inhibitors, proteasome inhibitors; and inhibitors of cancer metabolism.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG(geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYM®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN, Genentech); cetuximab (ERBITUX®, Imclone); panitumumab (VECTIBIX, Amgen), rituximab (RITUXAN®, Genentech/Biogen Idec), pertuzumab (OMNITARG, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG₁λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see W98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc.); and mAb 806 or humanized mAb 806 (Johns et al., J Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/ER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc.); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof, autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

In certain embodiments, chemotherapeutic agents include, but are not limited to, doxorubicin, dexamethasone, vincristine, cyclophosphamide, fluorouracil, topotecan, interferons, platinum derivatives, taxanes (e.g., paclitaxel, docetaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, trimetrexate, metoprine, cyclosporine, daunorubicin, teniposide, amphotericin, alkylating agents (e.g., chlorambucil), 5-fluorouracil, camptothecin, cisplatin, metronidazole, and imatinib mesylate, among others. In other embodiments, a compound of the present invention is administered in combination with a biologic agent, such as bevacizumab or panitumumab.

In certain embodiments, compounds of the present invention, or a pharmaceutically acceptable composition thereof, are administered in combination with an antiproliferative or chemotherapeutic agent selected from any one or more of abarelix, aldesleukin, alemtuzumab, alitretinoin, allopurinol, altretamine, amifostine, anastrozole, arsenic trioxide, asparaginase, azacitidine, BCG live, bevacuzimab, fluorouracil, bexarotene, bleomycin, bortezomib, busulfan, calusterone, capecitabine, camptothecin, carboplatin, carmustine, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, elotinib, estramustine, etoposide phosphate, etoposide, exemestane, filgrastim, floxuridine, fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

Chemotherapeutic agents also include treatments for Alzheimer's Disease such as donepezil hydrochloride and rivastigmine; treatments for Parkinson's Disease such as L-DOPA/carbidopa, entacapone, ropinrole, pramipexole, bromocriptine, pergolide, trihexephendyl, and amantadine; agents for treating multiple sclerosis (MS) such as beta interferon (e.g., Avonex® and Rebif®), glatiramer acetate, and mitoxantrone; treatments for asthma such as albuterol and montelukast sodium; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

Additionally, chemotherapeutic agents include pharmaceutically acceptable salts, acids or derivatives of any of chemotherapeutic agents, described herein, as well as combinations of two or more of them.

In another embodiment, provided are methods of using a compound of formula (I) or formula (II) or a pharmaceutically acceptable salt thereof as described elsewhere herein, or an embodiment or aspect thereof, to treat cancer in combination with a PD-1 axis binding antagonist.

The term “PD-1 axis binding antagonist” refers to a molecule that inhibits the interaction of a PD-1 axis binding partner with either one or more of its binding partner, so as to remove T-cell dysfunction resulting from signaling on the PD-1 signaling axis—with a result being to restore or enhance T-cell function (e.g., proliferation, cytokine production, target cell killing). As used herein, a PD-1 axis binding antagonist includes a PD-1 binding antagonist, a PD-L1 binding antagonist and a PD-L2 binding antagonist.

The term “PD-1 binding antagonist” refers to a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody. Specific examples of PD-1 binding antagonists are provided infra.

The term “PD-L1 binding antagonist” refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody. Specific examples of PD-L1 binding antagonists are provided infra.

The term “PD-L2 binding antagonist” refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to one or more of its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

PD-1 Axis Binding Antagonists

Provided herein are methods for treating cancer in an individual comprising administering to the individual an effective amount of a PD-1 axis binding antagonist and a compound of formula (I) or formula (II) or a pharmaceutically acceptable salt thereof as described elsewhere herein. Also provided herein are methods of enhancing immune function or response in an individual (e.g., an individual having cancer) comprising administering to the individual an effective amount of a PD-1 axis binding antagonist and a compound of formula (I) or formula (II) or a pharmaceutically acceptable salt thereof as described elsewhere herein.

In such methods, the PD-1 axis binding antagonist includes a PD-1 binding antagonist, a PDL1 binding antagonist, and/or a PDL2 binding antagonist. Alternative names for “PD-” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 binding antagonist is a molecule that inhibits the binding of PD-1 to its ligand binding partner(s). In a specific aspect the PD-1 ligand binding partners are PDL1 and/or PDL2. In another embodiment, a PDL1 binding antagonist is a molecule that inhibits the binding of PDL1 to its binding partner(s). In a specific aspect, PDL1 binding partner(s) are PD-1 and/or B7-1. In another embodiment, the PDL2 binding antagonist is a molecule that inhibits the binding of PDL2 to its binding partner(s). In a specific aspect, a PDL2 binding partner is PD-1. The antagonist may be an antibody, an antigen binding fragment thereof, an immunoadhesin, a fusion protein, an oligopeptide or a small molecule. If the antagonist is an antibody, in some embodiments the antibody comprises a human constant region selected from the group consisting of IgG1, IgG2, IgG3 and IgG4

Anti-PD-1 Antibodies

In some embodiments, the PD-1 binding antagonist is an anti-PD-1 antibody. A variety of anti-PDL1 antibodies can be utilized in the methods disclosed herein. In any of the embodiments herein, the PD-1 antibody can bind to a human PD-1 or a variant thereof. In some embodiments the anti-PD-1 antibody is a monoclonal antibody. In some embodiments, the anti-PD-1 antibody is an antibody fragment selected from the group consisting of Fab, Fab′, Fab′-SH, Fv, scFv, and (Fab′)₂ fragments. In some embodiments, the anti-PD-1 antibody is a chimeric or humanized antibody. In other embodiments, the anti-PD-1 antibody is a human antibody.

In some embodiments, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO2006/121168. Nivolumab comprises a heavy chain and a light chain sequence, wherein:

(a) the heavy chain comprises the amino acid
sequence.
(SEQ ID NO: 1)
QVQLVESGGGVVQPGRSLRLDCKASGITFSNSGMHWVRQAPGKGLEWVAV
IWY DGSKRYYADSVKGRFTISRDNSKNTLFLQMNSLRAEDTAVYYCATN
DDWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGK,
and
(b) the light chain comprises the amino acid
sequence:
(SEQ ID NO: 2)
EIVLTQSPATLSLSPGERATLSCRASQSVSSYLAWYQQKPGQAPRLLIYD
ASNRAT GIPARFSGSGSGTDFTLTISSLEPEDFAVYYCQQSSNWPRTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

In some embodiments, the anti-PD-1 antibody comprises the six HVR sequences from SEQ ID NO:1 and SEQ ID NO:2 (e.g., the three heavy chain HVRs from SEQ ID NO:1 and the three light chain HVRs from SEQ ID NO:2). In some embodiments, the anti-PD-1 antibody comprises the heavy chain variable domain from SEQ ID NO:1 and the light chain variable domain from SEQ ID NO:2.

In some embodiments, the anti-PD-1 antibody is pembrolizumab (CAS Registry Number: 1374853-91-4). Pembrolizumab (Merck), also known as MK-3475, Merck 3475, lambrolizumab, SCH-900475, and KEYTRUDA® is an anti-PD-1 antibody described in WO2009/114335. Pembrolizumab comprises a heavy chain and a light chain sequence, wherein:

(a) the heavy chain comprises the amino acid
sequence:
(SEQ ID NO: 3)
QVQLVQSGVEVKKPGASVKVSCKASGYTFTNYYMYWVRQAPGQGLEWMG
G INPSNGGTNFNEKFKNRVTLTTDSSTTTAYMELKSLQFDDTAVYYCAR
RDYRFDMGFDYW GQGTTVTVSSASTKGPSVFPLAPCSRSTSESTAALGC
LVKDYFPEPVTVSWNSGALTSGV HTFPAVLQSSGLYSLSSVVTVPSSSL
GTKTYTCNVDEEKPSNTKVDKRVESKYGPPCPPCP APEFLGGPSVFLFP
PKPKDTLMISRTPEVTCVVVDVSQEDPEVQFNWYVDGVEVHNAKTK PRE
EQFNSTYRVVSVLTVLHQDWLNGKEYKCKVSNKGLPSSIEKTISKAK GQ
PREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLS
LSLGK,
and
(b) the light chain comprises the amino acid
sequence:
(SEQ ID NO: 4)
EIVLTQSPAT LSLSPGERATLSCRASKGVSTSGYSYLHWYQQKPGQAPR
LLIYLASYLES GVPARFSGSGSGTDFTLTISSLEPEDFAVYYCQHSRDL
PLTFGGGTKVEIKRTVAAPSVF IFPPSDEQLKSGTASVVCLLNNFYPRE
AKVQWKVDNALQSGNSQESVTEQ DSKDSTYSLSSTLTLSKADYEKHKVY
ACEVTHQGLSSPVTKSFNRGEC.

In some embodiments, the anti-PD-1 antibody comprises the six HVR sequences from SEQ ID NO:3 and SEQ ID NO:4 (e.g., the three heavy chain HVRs from SEQ ID NO:3 and the three light chain HVRs from SEQ ID NO:4). In some embodiments, the anti-PD-1 antibody comprises the heavy chain variable domain from SEQ ID NO:3 and the light chain variable domain from SEQ ID NO:4.

In some embodiments, the anti-PD-1 antibody is MEDI-0680 (AMP-514; AstraZeneca). MEDI-0680 is a humanized IgG4 anti-PD-1 antibody.

In some embodiments, the anti-PD-1 antibody is PDR001 (CAS Registry No. 1859072-53-9; Novartis). PDR001 is a humanized IgG4 anti-PD1 antibody that blocks the binding of PDL1 and PDL2 to PD-1.

In some embodiments, the anti-PD-1 antibody is REGN2810 (Regeneron). REGN2810 is a human anti-PD1 antibody.

In some embodiments, the anti-PD-1 antibody is BGB-108 (BeiGene). In some embodiments, the anti-PD-1 antibody is BGB-A317 (BeiGene).

In some embodiments, the anti-PD-1 antibody is JS-001 (Shanghai Junshi). JS-001 is a humanized anti-PD1 antibody.

In some embodiments, the anti-PD-1 antibody is STI-A1110 (Sorrento). STI-A1110 is a human anti-PD1 antibody.

In some embodiments, the anti-PD-1 antibody is INCSHR-1210 (Incyte). INCSHR-1210 is a human IgG4 anti-PD1 antibody.

In some embodiments, the anti-PD-1 antibody is PF-06801591 (Pfizer).

In some embodiments, the anti-PD-1 antibody is TSR-042 (also known as ANB011; Tesaro/AnaptysBio).

In some embodiments, the anti-PD-1 antibody is AM0001 (ARMO Biosciences).

In some embodiments, the anti-PD-1 antibody is ENUM 244C8 (Enumeral Biomedical Holdings). ENUM 244C8 is an anti-PD1 antibody that inhibits PD-1 function without blocking binding of PDL1 to PD-1.

In some embodiments, the anti-PD-1 antibody is ENUM 388D4 (Enumeral Biomedical Holdings). ENUM 388D4 is an anti-PD1 antibody that competitively inhibits binding of PDL1 to PD-1.

In some embodiments, the PD-1 antibody comprises the six HVR sequences (e.g., the three heavy chain HVRs and the three light chain HVRs) and/or the heavy chain variable domain and light chain variable domain from a PD-1 antibody described in WO2015/112800 (Applicant: Regeneron), WO2015/112805 (Applicant: Regeneron), WO2015/112900 (Applicant: Novartis), US20150210769 (Assigned to Novartis), WO2016/089873 (Applicant: Celgene), WO2015/035606 (Applicant: Beigene), WO2015/085847 (Applicants: Shanghai Hengrui Pharmaceutical/Jiangsu Hengrui Medicine), WO2014/206107 (Applicants: Shanghai Junshi Biosciences/Junmeng Biosciences), WO2012/145493 (Applicant: Amplimmune), U.S. Pat. No. 9,205,148 (Assigned to MedImmune), WO2015/119930 (Applicants: Pfizer/Merck), WO2015/119923 (Applicants: Pfizer/Merck), WO2016/032927 (Applicants: Pfizer/Merck), WO2014/179664 (Applicant: AnaptysBio), WO2016/106160 (Applicant: Enumeral), and WO2014/194302 (Applicant: Sorrento).

Anti-PDL1 Antibodies

In some embodiments, the PD-1 axis binding antagonist is an anti-PDL1 antibody. A variety of anti-PDL1 antibodies are contemplated and described herein. In any of the embodiments herein, the isolated anti-PDL1 antibody can bind to a human PDL1, for example a human PDL1 as shown in UniProtKB/Swiss-Prot Accession No. Q9NZQ7.1, or a variant thereof. In some embodiments, the anti-PDL1 antibody is capable of inhibiting binding between PDL1 and PD-1 and/or between PDL1 and B7-1. In some embodiments, the anti-PDL1 antibody is a monoclonal antibody. In some embodiments, the anti-PDL1 antibody is an antibody fragment selected from the group consisting of Fab, Fab′-SH, Fv, scFv, and (Fab′)₂ fragments. In some embodiments, the anti-PDL1 antibody is a chimeric or humanized antibody. In some embodiments, the anti-PDL1 antibody is a human antibody. Examples of anti-PDL1 antibodies useful in the methods of this invention and methods of making them are described in PCT patent application WO 2010/077634 and U.S. Pat. No. 8,217,149, both of which are incorporated herein.

In some embodiments, the anti-PDL1 antibody is atezolizumab (CAS Registry Number: 1422185-06-5). Atezolizumab (Genentech), also known as MPDL3280A, is an anti-PDL1 antibody.

Atezolizumab comprises:

(a) an HVR-H1, HVR-H2, and HVR-H3 sequence of GFTFSDSWIH (SEQ ID NO:5), AWISPYGGSTYYADSVKG (SEQ ID NO:6) and RHWPGGFDY (SEQ ID NO:7), respectively, and

(b) an HVR-L1, HVR-L2, and HVR-L3 sequence of RASQDVSTAVA (SEQ ID NO:8), SASFLYS (SEQ ID NO:9) and QQYLYHPAT (SEQ ID NO:10), respectively.

Atezolizumab comprises a heavy chain and a light chain sequence, wherein:

(a) the heavy chain variable region sequence comprises the amino acid sequence: EVQLVESGGGLVQPGGSLRLSCAASGFTFSDSWIHWVRQAPGKGLEWVAWIS PYGGSTYYADSVKGRFTISADTSKNTAYLQMNSLRAEDTAVYYCARRHWPGGFDY WGQGTLVTVSS (SEQ ID NO:11), and

(b) the light chain variable region sequence comprises the amino acid sequence: DIQMTQSPSSLSASVGDRVTITCRASQDVSTAVAWYQQKPGKAPKLLIY SASF LYSGVPSRFSGSGSGTDFTLTISSLQPEDFATYYCQQYLYHPATFGQGTKVEIKR (SEQ ID NO: 12).

Atezolizumab comprises a heavy chain and a light chain sequence, wherein:

(a) the heavy chain comprises the amino acid
sequence:
(SEQ ID NO: 13)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG,
and
(b) the light chain comprises the amino acid
sequence:
(SEQ ID NO: 14)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPSVFIFPPSDEQLKSGTASVVCLLNNFYPREAKVQWKV
DNALQSGNSQESVTEQDSKDSTYSLSSTLTLSKADYEKEIKVYACEVTHQ
GLSSPVTKSFNRGEC.

In some embodiments, the anti-PDL1 antibody is avelumab (CAS Registry Number: 1537032-82-8). Avelumab, also known as MSB0010718C, is a human monoclonal IgG1 anti-PDL1 antibody (Merck KGaA, Pfizer). Avelumab comprises a heavy chain and a light chain sequence, wherein:

(a) the heavy chain comprises the amino acid
sequence:
(SEQ ID NO: 15)
EVQLLESGGGLVQPGGSLRLSCAASGFTFSSYIMMWVRQAPGKGLEWVSS
IYPSGGITFYADTVKGRFTISRDNSKNTLYLQMNSLRAEDTAVYYCARIK
LGTVTTVDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG,
and
(b) the light chain comprises the amino acid
sequence:
(SEQ ID NO: 16)
QSALTQPASVSGSPGQSITISCTGTSSDVGGYNYVSWYQQHPGKAPKLMI
YDVSNRPSGVSNRFSGSKSGNTASLTISGLQAEDEADYYCSSYTSSSTRV
FGTGTKVTVLGQPKANPTVTLFPPSSEELQANKATLVCLISDFYPGAVTV
AWKADGSPVKAGVETTKPSKQSNNKYAASSYLSLTPEQWKSHRSYSCQVT
HEGSTVEKTVAPTECS.

In some embodiments, the anti-PDL1 antibody comprises the six HVR sequences from SEQ ID NO:15 and SEQ ID NO:16 (e.g., the three heavy chain HVRs from SEQ ID NO:15 and the three light chain HVRs from SEQ ID NO:16). In some embodiments, the anti-PDL1 antibody comprises the heavy chain variable domain from SEQ ID NO:15 and the light chain variable domain from SEQ ID NO:16.

In some embodiments, the anti-PDL1 antibody is durvalumab (CAS Registry Number: 1428935-60-7). Durvalumab, also known as MEDI4736, is an Fc-optimized human monoclonal IgG1 kappa anti-PDL1 antibody (MedImmune, AstraZeneca) described in WO2011/066389 and US2013/034559. Durvalumab comprises a heavy chain and a light chain sequence, wherein:

(a) the heavy chain comprises the amino acid
sequence:
(SEQ ID NO: 17)
EVQLVESGGGLVQPGGSLRLSCAASGFTFSRYWMSWVRQAPGKGLEWVAN
IKQDGSEKYYVDSVKGRFTISRDNAKNSLYLQMNSLRAEDTAVYYCAREG
GWFGELAFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPEFEGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPASIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSP
G,
and
(b) the light chain comprises the amino acid
sequence:
(SEQ ID NO: 18)
EIVLTQSPGTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

In some embodiments, the anti-PDL1 antibody comprises the six HVR sequences from SEQ ID NO:17 and SEQ ID NO:18 (e.g., the three heavy chain HVRs from SEQ ID NO:17 and the three light chain HVRs from SEQ ID NO:18). In some embodiments, the anti-PDL1 antibody comprises the heavy chain variable domain from SEQ ID NO:17 and the light chain variable domain from SEQ ID NO:18.

In some embodiments, the anti-PDL1 antibody is MDX-1105 (Bristol Myers Squibb). MDX-1105, also known as BMS-936559, is an anti-PDL1 antibody described in WO2007/005874.

In some embodiments, the anti-PDL1 antibody is LY3300054 (Eli Lilly).

In some embodiments, the anti-PDL1 antibody is STI-A1014 (Sorrento). STI-A1014 is a human anti-PDL1 antibody.

In some embodiments, the anti-PDL1 antibody is KN035 (Suzhou Alphamab). KN035 is single-domain antibody (dAB) generated from a camel phage display library.

In some embodiments, the anti-PDL1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some embodiments, the anti-PDL1 antibody is CX-072 (CytomX Therapeutics).

In some embodiments, the PDL1 antibody comprises the six HVR sequences (e.g., the three heavy chain HVRs and the three light chain HVRs) and/or the heavy chain variable domain and light chain variable domain from a PDL1 antibody described in US20160108123 (Assigned to Novartis), WO2016/000619 (Applicant: Beigene), WO2012/145493 (Applicant: Amplimmune), U.S. Pat. No. 9,205,148 (Assigned to MedImmune), WO2013/181634 (Applicant: Sorrento), and WO2016/061142 (Applicant: Novartis).

In a still further specific aspect, the PD-1 or PDL1 antibody has reduced or minimal effector function. In a still further specific aspect the minimal effector function results from an “effector-less Fc mutation” or aglycosylation mutation. In still a further embodiment, the effector-less Fc mutation is an N297A or D265A/N297A substitution in the constant region. In some embodiments, the isolated anti-PDL1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

Other PD-1 Antagonists

In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. AMP-224 (CAS Registry No. 1422184-00-6; GlaxoSmithKline/MedImmune), also known as B7-DCIg, is a PDL2-Fc fusion soluble receptor described in WO2010/027827 and WO2011/066342.

In some embodiments, the PD-1 binding antagonist is a peptide or small molecule compound. In some embodiments, the PD-1 binding antagonist is AUNP-12 (PierreFabre/Aurigene). See, e.g., WO2012/168944, W2015/036927, W2015/044900, WO2015/033303, WO2013/144704, WO2013/132317, and WO2011/161699.

In some embodiments, the PDL1 binding antagonist is a small molecule that inhibits PD-1. In some embodiments, the PDL1 binding antagonist is a small molecule that inhibits PDL1. In some embodiments, the PDL1 binding antagonist is a small molecule that inhibits PDL1 and VISTA. In some embodiments, the PDL1 binding antagonist is CA-170 (also known as AUPM-170). In some embodiments, the PDL1 binding antagonist is a small molecule that inhibits PDL1 and TIM3. In some embodiments, the small molecule is a compound described in WO2015/033301 and WO2015/033299.

As used herein “combination” refers to any mixture or permutation of one or more compounds of the disclosure (or an embodiment or aspect thereof) and one or more other compounds of the disclosure or one or more additional therapeutic agent. Unless the context makes clear otherwise, “combination” may include simultaneous or sequentially delivery of a compound of the invention with one or more therapeutic agents. Unless the context makes clear otherwise, “combination” may include dosage forms of a compound of the disclosure with another therapeutic agent. Unless the context makes clear otherwise, “combination” may include routes of administration of a compound of the disclosure with another therapeutic agent. Unless the context makes clear otherwise, “combination” may include formulations of a compound of the disclosure with another therapeutic agent. Dosage forms, routes of administration and pharmaceutical compositions include, but are not limited to, those described herein.

Bifunctional Degrader Compounds

In some aspects, the present disclosure relates to bifunctional degrader compounds which can be used for degradation of target proteins, the bifunctional compounds comprising a compound of the present disclosure as a protein binding moiety (“PB”) in combination with a ligand moiety (“ligand”) comprising a ligase or a protease. In some such aspects, the present disclosure is directed to bifunctional degrader compounds which contain on one end a von Hippel-Lindau (VHL) tumor suppressor ligand moiety, which binds to the VHL E3 ubiquitin ligase, and on the other end a compound of the present disclosure that is a protein binding moiety such that degradation of the target protein/polypeptide is effectuated.

In some such aspects, the bifunctional degrader compounds may be of the structure PB-Ligand, PB-L-Ligand or PB-L-Y-Ligand where PB refers to the compositions of the present disclosure that are protein binders, “L” refers to a linker (or a pharmaceutically acceptable salt, enantiomer, stereoisomer, solvate or polymorph thereof), “Ligand” refers to a moiety comprising a ligase or protease, and “Y” refers to an optional moiety. In aspects directed to PB-linker, the PB and linker are connected via a bond.

As used herein, PB refers to a protein binding moiety and is used to describe compounds of the present disclosure which bind to a target protein or other protein or polypeptide of interest and places/presents that protein or polypeptide in proximity to the protease or ligase end of the bifunctional degrader such that degradation of the protein or polypeptide may occur. Generally, when used as a PB moiety in a degrader, the compounds of the present disclosure exhibit binding affinity to TEAD. In some aspects, the ligase is an ubiquitin ligase. By coupling the VHL ligand to a protein binding moiety (PB), the target protein or polypeptide is ubiquitinated and/or degraded by the proteasome.

The PB moiety may be coupled to L or to the ligand at any site on the PB, or a substitutent thereon, within the scope of the disclosure that does not materially affect the binding of the PB to a target protein or other protein or polypeptide of interest. In some aspects, coupling may be at a carbon atom, nitrogen atom or oxygen atom on the PB or on a substituent thereon.

The crystal structure of VHL with ligands has been obtained, confirming that a small compound can mimic the binding mode of the transcription factor HIF-1α, the major substrate of VHL. Using rational design, the first small molecule ligands of Von Hippel Lindau (VHL) the substrate recognition subunit of the E3 ligase VCB (a target in cancer, chronic anemia and ischemia) were generated.

E3 ubiquitin ligases (of which over 600 are known in humans) confer substrate specificity for ubiquitination. There are known ligands which bind to these ligases. An E3 ubiquitin ligase binding group (E3LB) is a peptide or small molecule that can bind an E3 ubiquitin ligase.

One E3 ligase with therapeutic potential is the von Hippel-Lindau (VHL) tumor suppressor, the substrate recognition subunit of the E3 ligase complex VCB, which also consists of elongins B and C, Cul2 and Rbx1. The primary substrate of VHL is Hypoxia Inducible Factor 1α (HIF-1α), a transcription factor that upregulates genes such as the pro-angiogenic growth factor VEGF and the red blood cell inducing cytokine erythropoietin in response to low oxygen levels. While HIF-1α is constitutively expressed, its intracellular levels are kept very low under normoxic conditions via its hydroxylation by prolyl hydroxylase domain (PHD) proteins and subsequent VIAL-mediated ubiquitination.

The terms “VCB E3 Ubiquitin Ligase,” “Von Hippel-Lindau (or VHL) E3 Ubiquitin Ligase,” “VHL,” or “Ubiquitin Ligase,” which may generally be used interchangeably unless the context indicates otherwise, are used to describe a target enzyme(s) binding site of ubiquitin ligase moieties as described herein, e.g., in the bifunctional (chimeric) compounds as described herein. “VCB” refers to the E3 ubiquitin ligase family VHL-Elongin C/Elongin B. VCB E3 is a protein that in combination with an E2 ubiquitin-conjugating enzyme causes the attachment of ubiquitin to a lysine on a target protein; the E3 ubiquitin ligase targets specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation, but may instead be altered in their cellular location or function, for example, via binding other proteins that have domains capable of binding ubiquitin. Further complicating matters, different lysines on ubiquitin can be targeted by an E3 to make chains. The most common lysine is Lys48 on the ubiquitin chain. This is the lysine used to make polyubiquitin, which is recognized by the proteasome.

In some aspects, the VHL ligand moiety is a small molecule (i.e., not peptide based). As used herein, A “small molecule” generally refers to an organic molecule that is less than 5 kilodaltons (Kd) in size, such as less than 4 Kd, less than 3 Kd, less than 2 Kd, less than 1 Kd, less than 800 daltons (D), less than 600 D, less than 500 D, less than 400 D, less than 300 D, less than 200 D, less than 100 D, less than 2000 g/mol, less than 1500 g/mol, less than 1000 g/mol, less than 800 g/mol, or less than 500 g/mol. In some aspects, small molecules are non-polymeric. Small molecules are not proteins, polypeptides, oligopeptides, peptides, polynucleotides, oligonucleotides, polysaccharides, glycoproteins, proteoglycans, etc. A derivative of a small molecule refers to a molecule that shares the same structural core as the original small molecule, but which can be prepared by a series of chemical reactions from the original small molecule.

The VHL ligand moiety and PB moiety of bifunctional degrader compounds as described herein can be connected with L. In certain embodiments, L is a group comprising one or more covalently connected structural units of A, wherein each A unit is a group coupled to at least one of a VHL ligand moiety, a PB moiety, another A unit, or a combination thereof. In certain embodiments, an A unit links a VHL ligand moiety, a PB moiety, or a combination thereof directly to another VHL ligand, PB moiety, or combination thereof. In other embodiments, an A unit links a VHL ligand moiety, a PB moiety, or a combination thereof indirectly to another VHL ligand moiety, PB moiety, or combination thereof through one or more different A unit(s). In any of the embodiments disclosed herein, one or more covalently connected structural units of A may be coupled to the VHL ligand moiety of the bifunctional degrader compounds of the present disclosure at substituent Y. Thus, in certain embodiments, L may be coupled to Y, PB, or combinations thereof.

In certain embodiments, L is (A)_{q}, and each A is independently selected from the group consisting of a bond, CR^{La}R^{Lb}, O, S, SO, SO₂, NRLc, SO₂NR^{Lc}, SONR^{Lc}, CONR^{Lc}, NR^{Lc}CONR^{Ld}, NR^{Lc}SO₂NR^{Ld}, CO, CR^{La}═CR^{Lb}, C≡C, SiR^{La}R^{Lb}, P(O)R^{La}, P(O)OR^{La}, NR^{Lc}C(═NCN)NR^{Ld}, NR^{Lc}C(═NCN), NR^{Lc}C(═CNO₂)NR^{Ld}, C₃₋₁₁ cycloalkylene, C₃₋₁₁ heterocyclylene, arylene, and heteroarylene, wherein the C₃₋₁₁ cycloalkylene, C₃₋₁₁ heteocyclylene, arylene, and heteroarylene are independently either unsubstituted or substituted with 1, 2, 3, 4, 5, or 6 substituents selected from the group consisting of R^{La}, R^{Lb} and combinations thereof, where R^{La} or R^{Lb}, each independently, can be linked to other A groups to form cycloalkylene and/or heterocyclylene moiety, wherein the cycloalkylene and heterocyclylene moieties are independently unsubstituted or substituted with 1, 2, 3, or 4 R^{Le} groups; wherein R^{La}, R^{Lb}, R^{Lc}, R^{Ld} and R^{Le} are, each independently, selected from the group consisting of H, halogen, R^{Lf}, —OR^{Lh}, —SR^{Lh}, —NHR^{Lh}, —N(R^{Lh})₂, C₃₋₁₁ cycloalkyl, aryl, heteroaryl, C₃₋₁₁ heterocyclyl, —N(R^{Lg})(R^{Lf}), —OH, —NH₂, —SH, —SO₂R^{Lf}, —P(O)(OR^{Lf})(R^{Lf}), —P(O)(OR^{Lf})₂, —C≡C—R^{Lf}, —C≡CH, —CH═CH(R^{Lf}), —C(R^{Lf})═CH(R^{Lf}), —C(R^{Lf})≡C(R^{Lf})₂, —Si(OH)₃, —Si(R^{Lf})₃, —Si(OH)(R^{Lf})₂, —COR^{Lf}, —C₂H, —CN, —CF₃, —CHF₂, —CH₂F, —NO₂, —SF₅, —SO₂NHR^{Lf}, —SO₂N(R^{Lf})₂, —SONHR^{Lf}, —SON(R^{Lf})₂, —CONHR^{Lf}, —CON(R^{Lf})₂, —N(R^{Lf})CONH(R^{Lf}), —N(R^{Lf})CON(R^{Lf})₂, —NHCONH(R^{Lf}), —NHCON(R^{Lf})₂, —NHCONH₂, —N(R^{Lf})SO₂NH(R^{Lf}), —N(R^{Lf})SO₂N(R^{Lf})₂, —NHSO₂NH(R^{Lf}), —NHSO₂N(R^{L})₂, and —NHSO₂NH₂, wherein R^{Lf} is a substituted or unsubstituted C₁₋₈ alkyl; R^{Lg} is a substituted or unsubstituted C₁₋₈-cycloalkyl; and R^{Lh} is R^{Lf} or R^{Lg}.

When present, Y may suitably be selected from the group consisting of substituted or unsubstituted heteroarylene, substituted or unsubstituted heterocyclylene, O, S, —N(R¹¹)—, —N(R¹¹)—C(O)—, and —N(R¹¹)—SO₂—. R¹¹ may be selected from the group consisting of H and substituted or unsubstituted alkyl.

Although the VHL ligand moiety and PB moiety may be covalently linked to the linker group through any group which is appropriate and stable to the chemistry of the linker, in some aspects, the linker may be independently covalently bonded to the VHL ligand moiety and the PB moiety through an amide, ester, thioester, keto group, carbamate (urethane), carbon or ether, each of which groups may be inserted anywhere on the VHL ligand moiety and PB moiety to provide maximum binding of the VHL ligand moiety on the VHL ubiquitin ligase and the PB moiety on the target protein to be degraded. (It is noted that in certain aspects where the PB group is a VHL ligand moiety, the target protein for degradation may be the ubiquitin ligase itself). In certain aspects, the linker may be linked to an optionally substituted alkyl, alkylene, alkene or alkyne group, an aryl group or a heterocyclic group on the VHL ligand moiety and/or PB moiety.

In other degrader aspects, the disclosure provides a method of degrading (ubiquitinating) a target protein in a cell. The method comprises administering a bifunctional compound or a pharmaceutical composition comprising a bifunctional compound of the present disclosure, such as comprising a VHL ligand moiety and a protein binding moiety composition of the present disclosure, optionally linked through a linker moiety, as otherwise described herein, wherein the VHL ligand moiety is coupled to the protein binding moiety and wherein the VHL ligand moiety recognizes a ubiquitin pathway protein (e.g., an ubiquitin ligase, preferably a VHL ubiquitin ligase (E3)) and the protein binding moiety recognizes the target protein such that degradation of the target protein will occur when the target protein is placed in proximity to the ubiquitin ligase, thus resulting in degradation/inhibition of the effects of the target protein and the control of protein levels. The control of protein levels afforded by the present disclosure provides treatment of a disease state or condition, which is modulated through the target protein by lowering the level of that protein in the cells of a patient.

General Preparation of Compounds of Formula (I) and Formula (II)

The following synthetic reaction schemes detailed in the General Schemes and Examples and certain disclosed intermediates are merely illustrative of some of the methods by which the compounds of the present disclosure (or an embodiment or aspect thereof) can be synthesized. Various modifications to these synthetic reaction schemes can be made and will be suggested to one skilled in the art having referred to the disclosure contained in this Application.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

Although certain exemplary embodiments are depicted and described herein, the compounds of the present disclosure (or an embodiment or aspect thereof) can be prepared using appropriate starting materials according to the methods described generally herein and/or by methods available to one of ordinary skill in the art.

Intermediates and final compounds were purified by either flash chromatography, and/or by reverse-phase preparative HPLC (high performance liquid chromatography), and/or by supercritical fluid chromatography. Unless otherwise noted, flash chromatography was carried out using pre-packed silica gel cartridges from either ISCO or SiliCycle on an ISCO CombiFlash® chromatography instrument (from Teledyne Isco, Inc.).

Mass spectrometry (MS) was performed using a (1) Sciex 15 mass spectrometer in ES+ mode, or (2) Shimadzu liquid chromatography-mass spectrometry (LCMS) 2020 mass spectrometer in ESI+ mode. Mass spectra data generally only indicates the parent ions unless otherwise stated. MS or HRMS data is provided for a particular intermediate or compound where indicated.

Nuclear magnetic resonance spectroscopy (NMR) was performed using a (1) Bruker AV III300 NMR spectrometer, (2) Bruker AV III400 NMR spectrometer, or (3) Bruker AV III 500 NMR spectrometer, and referenced to tetramethylsilane. NMR data is provided for a particular intermediate or compound where indicated.

All reactions involving air-sensitive reagents were performed under an inert atmosphere. Reagents were used as received from commercial suppliers unless otherwise noted.

General Schemes

The following generalized schemes are used to prepare the disclosed compounds, intermediates, and pharmaceutically acceptable salts thereof. Disclosed compounds and intermediates may be prepared using standard organic synthetic techniques and from commerically available starting materials and reagents. It will be appreciated that synthetic procedures employed in the preparation of disclosed compounds and intermediates will depend on the particular substituents present in the compound or intermediate and that various protection, deprotection, and conversion steps that are standard in organic synthesis may be required, but may not be illustrated in the following general schemes. It is also to be understood that any of the steps shown in any of the following general schemes may be used in any combination and in any order that is chemically feasible to achieve a desired intermediate or disclosed compound.

Schemes 1-12 below describe the synthesis of intermediates and disclosed compounds, and pharmaceutically acceptable salts thereof, having the structure of Formula IA.

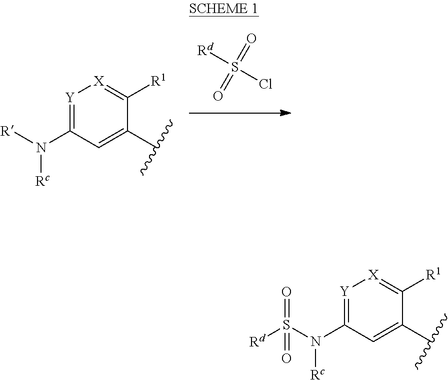

Scheme 1 describes a general synthetic route for converting an amino group to a sulfonic amide group using a sulfonic chloride compound. R^{l}, R^{c}, R^{d}, X, and Y are as defined above for Formula IA. R′ may be any suitable atom or group, including, for example, hydrogen. The

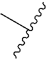

moiety may be any suitable atom or group, including, for example: a halogen; or the -A-R⁵ moiety as defined above for Formula IA. In some embodiments, the halogen is chlorine, iodine, or bromine.

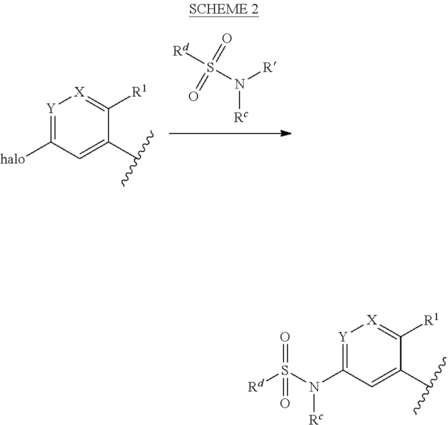

Scheme 2 describes a general synthetic route for converting a halogen (halo) group to a sulfonic amide group using a sulfonic amide compound. Halo refers to any halogen. In some embodiments, the halogen is chlorine, bromine or iodine. R^{l}, R^{c}, R^{d}, X, and Y are as defined above for Formula IA. R′ may be any suitable atom or group, including, for example: hydrogen or —C(O)OC(CH₃)₃. The

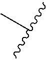

moiety may be any suitable atom or group, including, for example: a halogen, such as chlorine, bromine or iodine; the -A-R⁵ moiety as defined above for Formula IA; —CH₂P(O)(OR^{y})₂, wherein R^{y} is any suitable atom or group, including, for example, C₁₋₈ alkyl; or —CH₂OR^{x}, wherein R^{x} is any suitable protecting group, including, for example, TBDPS (tert-butyldiphenylsilyl).

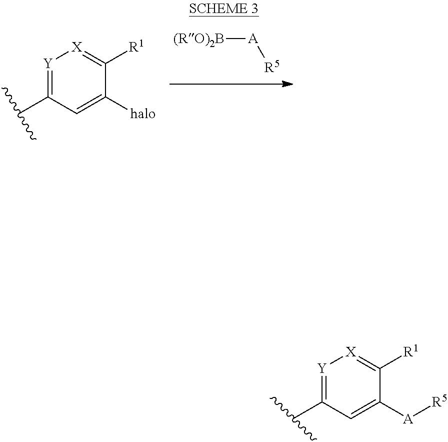

Scheme 3 describes a general synthetic route for converting a halogen (halo) group to the -A-R⁵ moiety defined above for Formula IA, using a boronic acid or a boronic ester compound. Halo refers to any halogen. In some embodiments, the halogen group is chlorine, bromine or iodine. R¹, R⁵, A, X, and Y are as defined above for Formula IA. R″ may be any suitable atom or group, including, for example, hydrogen. In some embodiments, the compound of formula

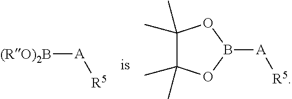

### The

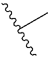

moiety may be any suitable atom or group, including, for example, a halogen such as chlorine, bromine or iodine.

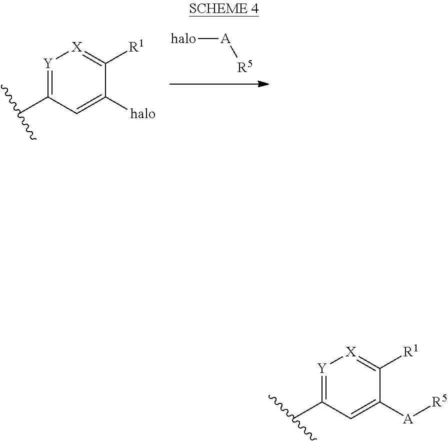

Scheme 4 describes a general synthetic route for converting a halogen (halo) group to the -A-R⁵ moiety defined above for Formula IA, using a halo compound. Halo refers to any halogen. In some embodiments, the halogen is chlorine, bromine, or iodine. R¹, R⁵, A, X, and Y are as defined above for Formula IA. The

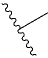

moiety may be any suitable atom or group, including, for example, the —NR^{c}SO₂R^{d} moiety defined above for Formula IA.

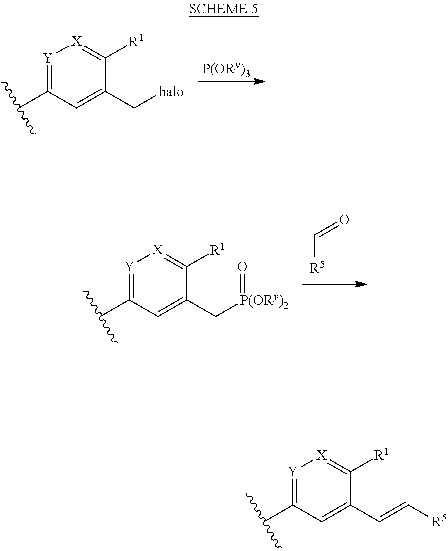

Scheme 5 describes a general synthetic route for converting a —CH₂-halo group to a —CH═CHR⁵ moiety using a phosphate compound and an aldehyde compound. R¹, R⁵, X, and Y are as defined above for Formula IA. Halo refers to any halogen. In some embodiments, the halogen is chlorine, bromine, or iodine. In some embodiments, the phosphate compound is P(OR^{y})₃, wherein R^{y} is any suitable atom or group, including, for example, C₁₋₈ alkyl. In certain variations, the phosphate compound is P(OEt)₃. The

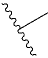

moiety may be any suitable atom or group, including, for example: a halogen, such a chlorine, bromine, or iodine; or —NR^{s}R^{t}, wherein R^{s} and R^{t} are each independently any suitable atom or group, including, for example, a protecting group. In some variations, R^{s} and R^{t} are different. In other variations, R^{s} and R^{t} are the same. In one embodiment, —NR^{s}R^{t} is —NO₂.

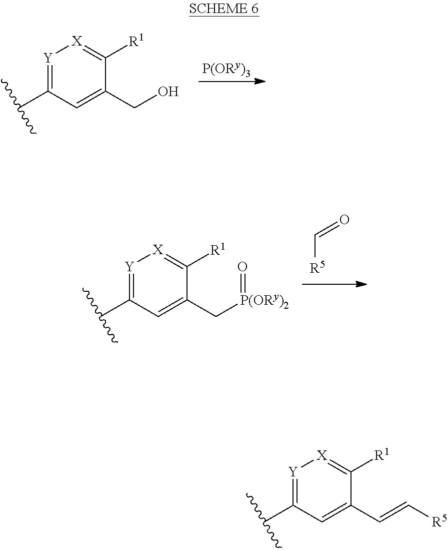

Scheme 6 describes a general synthetic route for converting a —CH₂—OH group to a —CH═CHR⁵ moiety using a phosphate compound and an aldehyde compound. R¹, R⁵, X, and Y are as defined above for Formula IA. In some embodiments, the phosphate compound is P(OR^{y})₃, wherein R^{y} is any suitable atom or group, including, for example, C₁₋₈ alkyl. In certain variations, the phosphate compound is P(OEt)₃. The

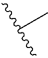

moiety may be any suitable atom or group, including, for example: a halogen, such as chlorine, bromine, or iodine; or —NR^{s}R^{t}, wherein R^{s} and R^{t} are each independently any suitable atom or group, including, for example, a protecting group. In some variations, R^{s} and R^{t} are different. In other variations, R^{s} and R^{t} are the same. In one embodiment, —NR^{s}R^{t} is —NO₂.

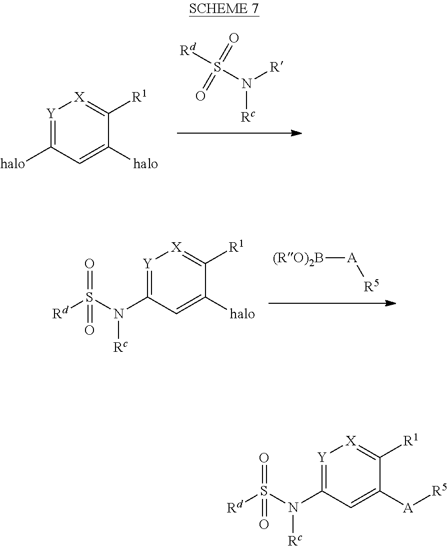

Scheme 7 describes a general synthetic route that sequentially combines the general synthetic routes outlined in Scheme 2 and Scheme 3 above.

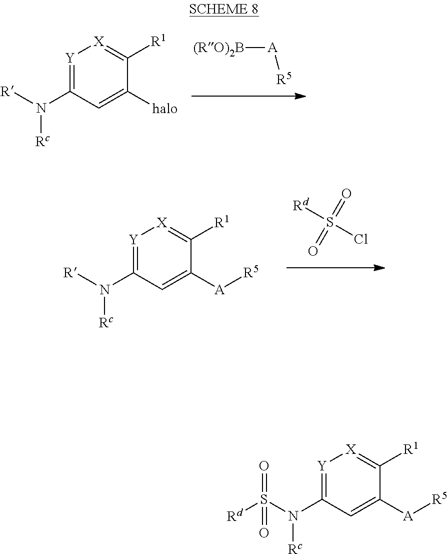

Scheme 8 describes a general synthetic route that sequentially combines the general synthetic routes outlined in Scheme 3 and Scheme 1.

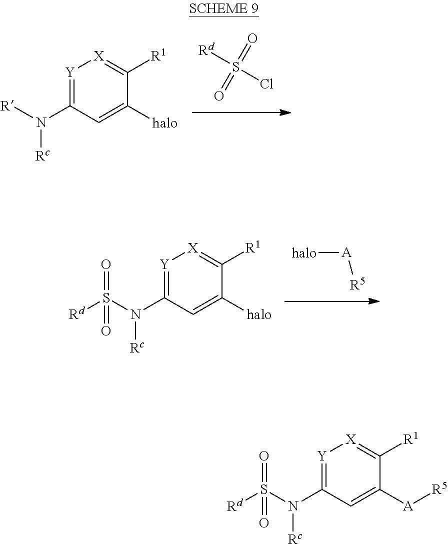

Scheme 9 describes a general synthetic route that sequentially combines the general synthetic routes outlined in Scheme 1 and Scheme 4.

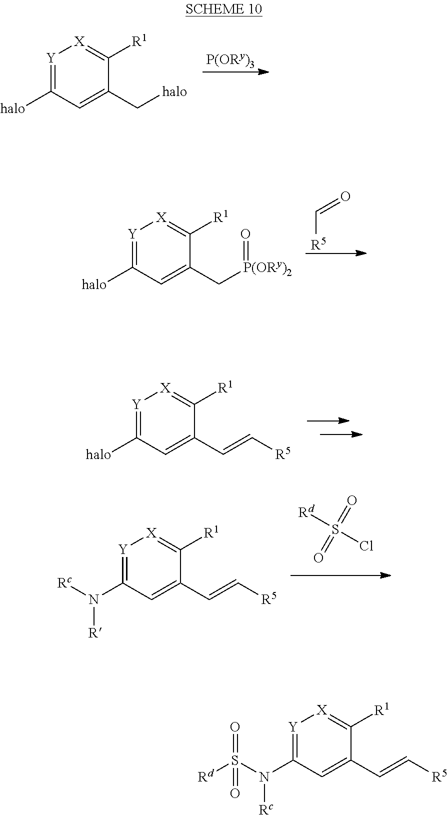

Scheme 10 describes a general synthetic route that sequentially combines the general synthetic routes outlined in Scheme 5 and Scheme 1. It is to be understood that the conversion of the halogen (halo) group to the amino (—NR^{c}R′) group between Scheme 5 and Scheme 1 may be achieved using any standard synthetic techniques and any commercially available reagents.

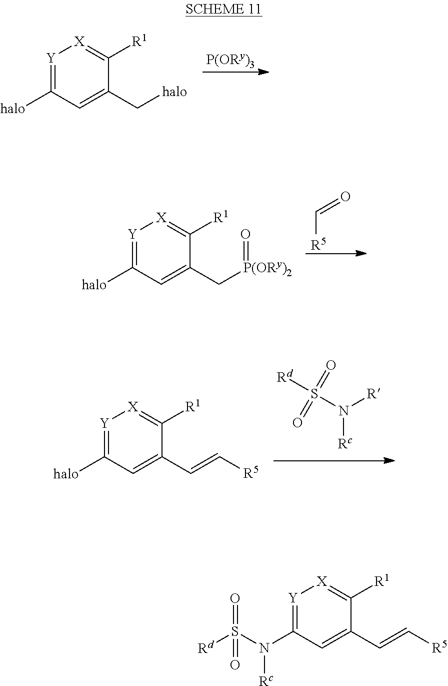

Scheme 11 describes a general synthetic route that sequentially combines the general synthetic routes outlined in Scheme 6 and Scheme 2.

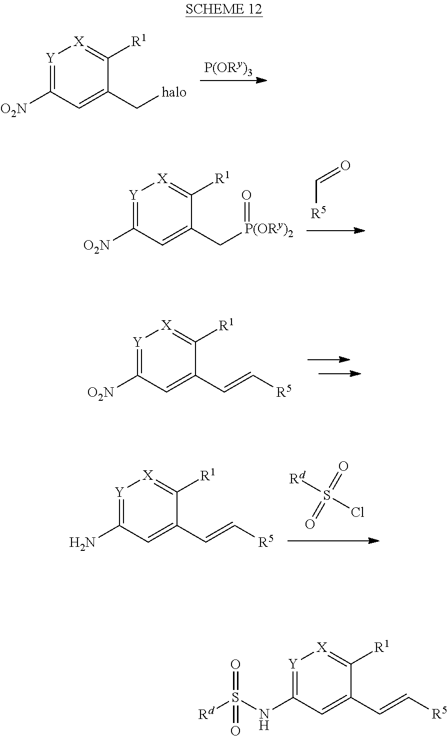

Scheme 12 describes a general synthetic route that sequentially combines the general synthetic routes outlined in Scheme 5 and Scheme 1. It is to be understood that the conversion of the nitro group (—NO₂) to the amino group (—NH₂) between Scheme 5 and Scheme 1 may be achieved using any standard synthetic techniques and any commercially available reagents.

Schemes 13-19 below describe the synthesis of intermediate and disclosed compounds, and pharmaceutically acceptable salts thereof, having the structure of Formula IB.

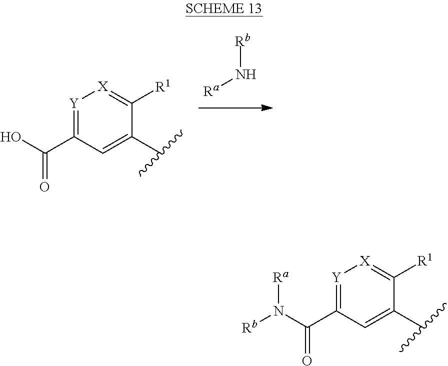

Scheme 13 describes a general synthetic route for converting a —COOH group to an amide group using an amine. R¹, R^{a}, R^{b}, X, and Y are as defined above for Formula IB. The

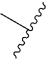

moiety may be any suitable atom or group, including, for example, the -A-R⁵ moiety as defined above for Formula IB.

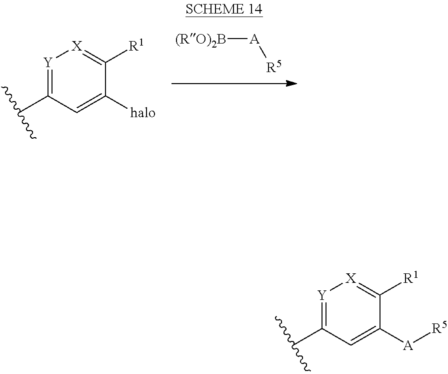

Scheme 14 describes a general synthetic route for converting a halogen (halo) group to the -A-R⁵ moiety defined above for Formula IB, using a boronic acid or a boronic ester compound. Halo refers to any halogen. In some embodiments, the halogen group is chlorine, bromine, or iodine. R¹, R⁵, A, X, and Y are as defined above for Formula IB. R″ may be any suitable atom or group, including, for example, hydrogen. In some embodiments, the compound of formula

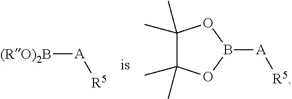

### The

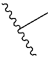

moiety may be any suitable atom or group, including, for example, a halogen such as chlorine, bromine, or iodine.

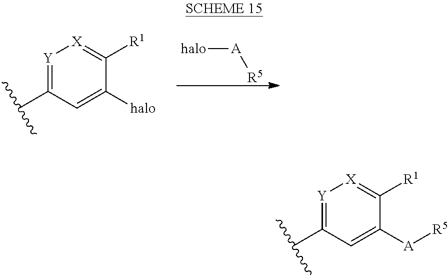

Scheme 15 describes a general synthetic route for converting a halogen (halo) group to the -A-R⁵ moiety defined above for Formula IB, using a halo compound. Halo refers to any halogen. In some embodiments, the halogen is chlorine, bromine, or iodine. R¹, R⁵, A, X, and Y are as defined above for Formula IA. The

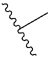

moiety may be any suitable atom or group, including, for example, the —NR^{c}SO₂R^{d} moiety defined above for Formula IB.

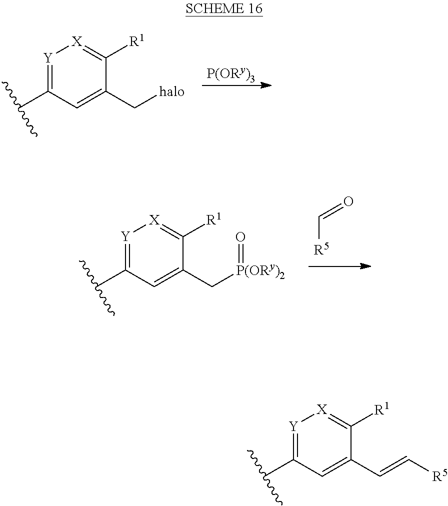

Scheme 16 describes a general synthetic route for converting a —CH₂-halo group to a —CH═CHR⁵ moiety using a phosphate compound and an aldehyde compound. R¹, R⁵, X, and Y are as defined above for Formula IB. Halo refers to any halogen. In some embodiments, the halogen is chlorine, bromine, or iodine. In some embodiments, the phosphate compound is P(OR^{y})₃, wherein R^{y} is any suitable atom or group, including, for example, C₁₋₈ alkyl. In certain variations, the phosphate compound is P(OEt)₃. The

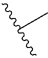

moiety may be any suitable atom or group, including, for example: a halogen, such as chlorine, bromine, or iodine; or —NR^{s}R^{t}, wherein R^{s} and R^{t} are each independently any suitable atom or group, including, for example, a protecting group. In some variations, R^{s} and R^{t} are different. In other variations, R^{s} and R^{t} are the same. In one embodiment, —NR^{s}R^{t} is —NO₂.

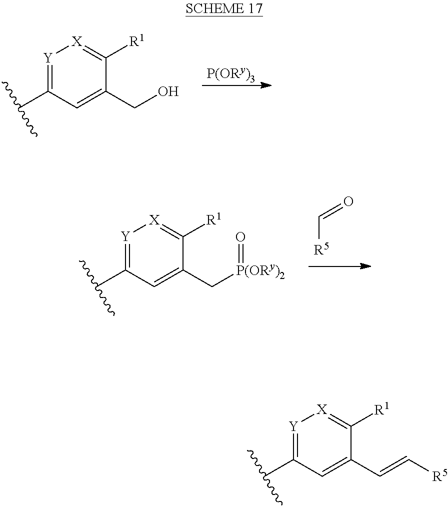

Scheme 17 describes a general synthetic route for converting a —CH₂—OH group to a —CH═CHR⁵ moiety using a phosphate compound and an aldehyde compound. R¹, R⁵, X, and Y are as defined above for Formula IB. In some embodiments, the phosphate compound is P(OR^{y})₃, wherein R^{y} is any suitable atom or group, including, for example, C₁₋₈ alkyl. In certain variations, the phosphate compound is P(OEt)₃. The

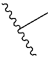

moiety may be any suitable atom or group, including, for example: a halogen; or —NR^{s}R^{t}, wherein R^{s} and R^{t} are each independently any suitable atom or group, including, for example, a protecting group. In some variations, R^{s} and R^{t} are different. In other variations, R^{s} and R^{t} are the same. In one embodiment, —NR^{s}R^{t} is —NO₂. In some embodiments, the halogen is iodine.

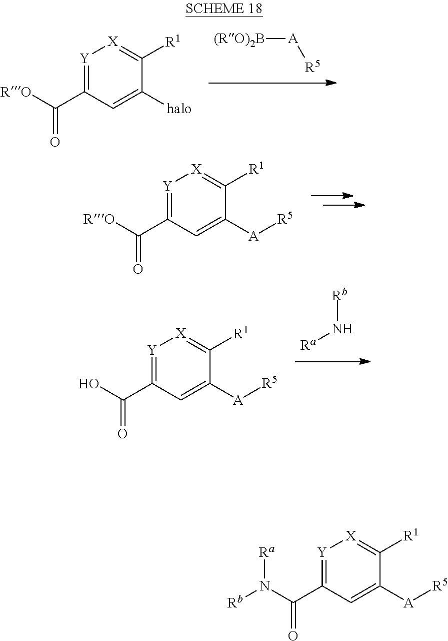

Scheme 18 describes a general synthetic route that sequentially combines the general synthetic routes outlined in Scheme 14 and Scheme 13. R′″ can be any suitable atom or group, for example, C₁₋₆alkyl. In some embodiments, R′″ is methyl. It is to be understood that the conversion of the —COOR′″ group to the —COOH group between Scheme 14 and Scheme 13 may be achieved using any standard synthetic techniques and any commercially available reagents.

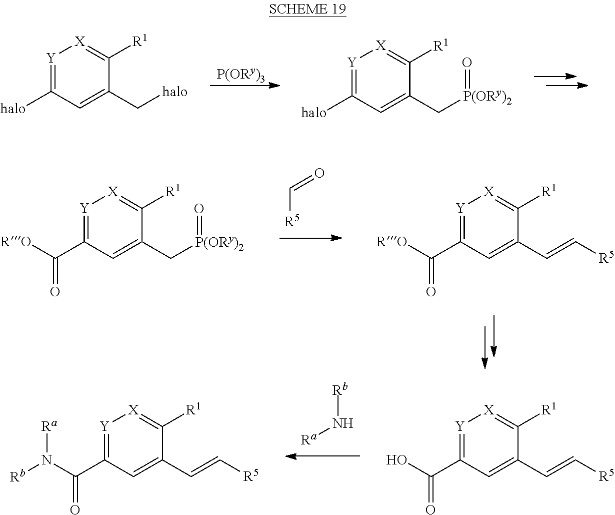

Scheme 19 describes a general synthetic route that combines the general synthetic routes outlined in Scheme 16 and Scheme 13. R′″ can be any suitable atom or group including, for example, C₁₋₆ alkyl or C₆₋₂₀ aryl. In some embodiments, R′″ is methyl. It is to be understood that the conversion of the -halo group to the —COOR′″ group and the conversion of the —COOR′″ group to the —COOH group may be achieved using any standard synthetic techniques and any commercially available reagents.

Scheme 20 below describes the synthesis of intermediates and disclosed compounds, and pharmaceutically acceptable salts thereof, having the structure of formula (II).

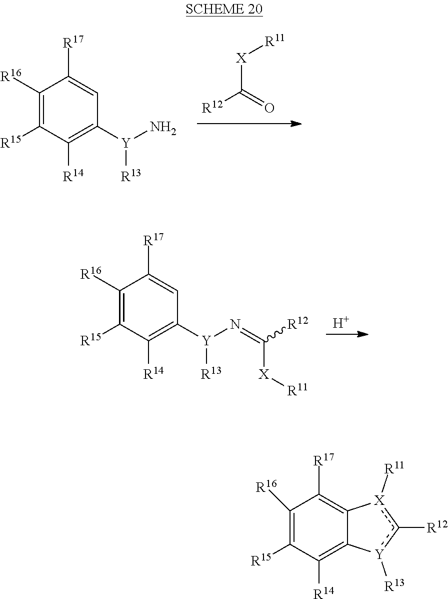

Scheme 20 describes a general synthetic route for preparing a compound of formula (II) from an amine and a carbonyl compound. R¹¹, R¹², R¹³, R¹⁴, R¹⁵, R¹⁶, R¹⁷, X, and Y are as defined above for the compound of formula (II). In some embodiments, Y is nitrogen, such that the amine is a hydrazine. In certain variations, the acid in the second step of Scheme 20 is phosphoric(V) acid, H₃PO₄.

Disclosed herein are certain intermediates including compounds having a structure of formula (III):

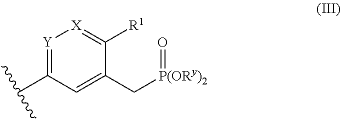

or a pharmaceutically acceptable salt thereof. X, Y, and R¹ are as defined above in Formula IA or Formula IB. R^{y} is any suitable atom or group, including, for example, C₁₋₈ alkyl. The

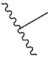

may be any suitable atom or group, including, for example, a halogen, or —NR^{s}R^{t}, wherein R^{s} and R^{t} are each independently any suitable atom or group, including, for example, a protecting group. In some variations, R^{s} and R^{t} are different. In other variations, R^{s} and R^{t} are the same. In one embodiment, —NR^{s}R^{t} is —NO₂. In some embodiments, the halogen is chlorine, bromine, or iodine.

Methods of making a compound described herein, or a pharmaceutically acceptable salt thereof, are provided. For illustrative purposes only, it is appreciated that, in one embodiment, is provided a method of preparing a compound of formula IA,

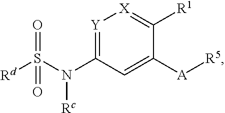

or a pharmaceutically acceptable salt thereof, wherein the various substituents are as defined above. The method comprises the steps of combining a compound of formula

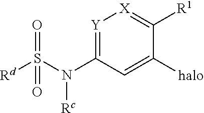

with a compound of formula

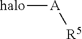

to produce the compound of formula IA. In one such embodiment, the method further comprises combining a compound of formula

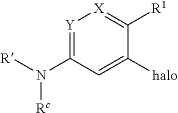

with a compound of formula

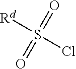

to produce the compound of formula

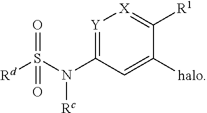

Note that, for all the compounds disclosed herein, A, X, Y, R¹, R⁵, R^{c}, R^{d}, and halo are as defined above in the General Schemes.

A further illustrative embodiment is a method of preparing a compound of formula IA, or a pharmaceutically acceptable salt thereof, wherein the -A-R⁵ moiety is —CH═CHR⁵, such that the compound has a formula

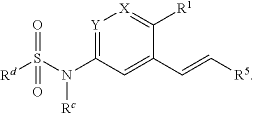

The method comprises the steps of combining a compound of formula

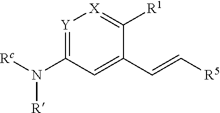

with a compound of formula

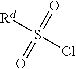

to produce the compound of formula

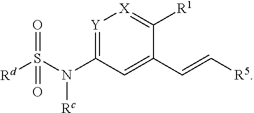

In one such embodiment, the method further comprises combining a compound of formula

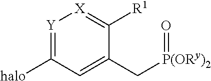

with a compound of formula

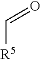

to produce a compound of formula

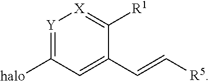

In said embodiment, the compound of

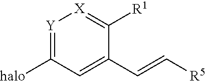

is then converted to the compound of formula

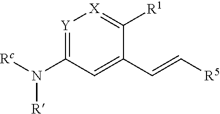

using standard synthetic techniques and commercially available reagents. In such an embodiment, the method further comprises combining a compound of formula

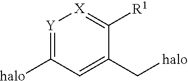

with a compound of formula P(OR^{y})₃ to produce the compound of formula

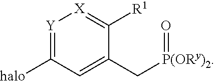

Note that, for all the compounds disclosed herein, A, X, Y, R¹, R⁵, R^{c}, R^{d}, R^{y}, R′, and halo are as defined above in the General Schemes.

Other such methods are included and described herein, and find basis in the general schemes and specific examples, the same as if each and every method were specifically and individually listed for each and every general scheme and example.

### EXAMPLES

### Example 1

### Preparation of N-(3-(2-Cyclohexylcyclopropyl)-4-methoxyphenyl) methanesulfonamide (Enantiomer A and Enantiomer B)

The reaction scheme was as follows:

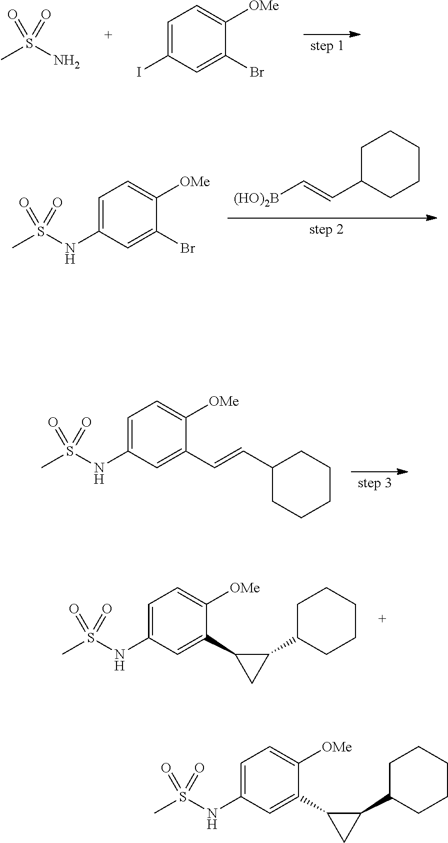

### Step 1: N-(3-Bromo-4-methoxyphenyl)methanesulfonamide

A flask was charged with 2-bromo-4-iodoanisole (1.0 g, 3.0 mmol), methanesulfonamide (1.4 g, 15 mmol), cuprous iodide (590 mg, 3.03 mmol), N,N-dimethylglycine (319 mg, 3.03 mmol) and potassium phosphate tribasic (1.3 g, 6.1 mmol) and the flask was purged with nitrogen. N,N-Dimethylacetamide (10 mL) was then added and the reaction was stirred at 100° C. for 2 hours. The reaction mixture was diluted with water (10 mL) and 10% aqueous glycine (10 mL) and acidified to pH=1 with 1N HCl, extracted with i-PrOAc (3×10 mL), dried with anhydrous MgSO₄, concentrated and purified by silica gel column chromatography (0% to 100% i-PrOAc in heptane) to give the title compound as a white solid (598 mg, 70% yield). LCMS (ESI+) m/z 280 (M+H)⁺.

### Step 2: (E)-N-(3-(2-Cyclohexylvinyl)-4-methoxyphenyl) methanesulfonamide

A vial was charged with N-(3-bromo-4-methoxyphenyl)methanesulfonamide (150 mg, 0.508 mmol), 2-cyclohexylethenylboronic acid (206 mg, 1.27 mmol), chloro(2-dicyclohexylphosphino-2′,6′-dimethoxy-1,1′-biphenyl)(2′-amino-1,1′-biphenyl-2-yl) palladium(II) (19 mg, 0.025 mmol), 2-dicyclohexylphosphino-2′,6′-dimethoxybiphenyl (11 mg, 0.025 mmol) and potassium phosphate tribasic (551 mg, 2.54 mmol), and the vial was purged with nitrogen. Toluene (1 ml) and water (0.1 ml) were added and the reaction was stirred at 100° C. for 2 hours. The reaction was then partitioned between 1N HCl (5 mL) and dichloromethane (5 mL), the product was extracted with dichloromethane and purified by silica gel column chromatography (0% to 100% i-PrOAc in heptane) to give the title compound as a white solid (157 mg, >99% yield). LCMS (ESI+) m/z 308 (M−H)⁻.

### Step 3: N-(3-(2-Cyclohexylcyclopropyl)-4-methoxyphenyl) methanesulfonamide

Diethylzinc (1.0 mol/L in hexanes, 2.7 mL, 2.7 mmol) was added to anhydrous dichloromethane (3 ml) and the solution was cooled to 0° C. A solution of trifluoroacetic acid (0.2 mL, 2.7 mmol) in dichloromethane (1 ml) was added to the diethylzinc solution dropwise and the resulting mixture was stirred at 0° C. for 20 minutes. A solution of diiodomethane (0.2 mL, 2.7 mmol) in dichloromethane (1 ml) was then added and the reaction was stirred at 0° C. for an additional 20 minutes. (E)-N-(3-(2-Cyclohexylvinyl)-4-methoxyphenyl)methanesulfonamide (168 mg, 0.543 mmol) was then dissolved in dichloromethane (1 ml) and added to the CF₃CO₂ZnCH₂I solution. The resulting solution was stirred at room temperature for 30 minutes, then quenched with saturated aqueous ammonium chloride (25 ml), acidified to pH=1 with 1N HCl, extracted with dichloromethane (3×10 ml), dried with anhydrous MgSO₄, concentrated under reduced pressure and purified by reverse-phase preparative HPLC. The enantiomers were then separated by chiral supercritical fluid chromatography (Chiralpak AS, isocratic 15% MeOH w/ 0.1% NH₄OH, 40° C., 2.5 min) to give the title compounds enantiomer A (2.8 mg) and enantiomer B (2.6 mg) as white solids.

N-(3-(2-Cyclohexylcyclopropyl)-4-methoxyphenyl)methanesulfonamide (enantiomer A): Chiral SFC Peak 1 (RT=0.463 min); ¹H NMR (400 MHz, DMSO-d₆) δ 9.18 (s, 1H), 6.96 (dd, J=8.7, 2.6 Hz, 1H), 6.88 (d, J=8.7 Hz, 1H), 6.64 (d, J=2.6 Hz, 1H), 3.78 (s, 3H), 2.84 (s, 3H), 1.90-1.53 (m, 6H), 1.29-0.98 (m, 5H), 0.82-0.63 (m, 4H); LCMS (ESI+) m/z 324.1 (M+H)⁺.

N-(3-(2-Cyclohexylcyclopropyl)-4-methoxyphenyl)methanesulfonamide (enantiomer B): Chiral SFC Peak 2 (RT=0.510 min); ¹H NMR (400 MHz, DMSO-d₆) δ 9.18 (s, 1H), 6.96 (dd, J=8.6, 2.6 Hz, 1H), 6.88 (d, J=8.7 Hz, 1H), 6.64 (d, J=2.6 Hz, 1H), 3.78 (s, 3H), 2.84 (s, 3H), 1.91-1.53 (m, 6H), 1.26-0.99 (m, 5H), 0.84-0.61 (m, 4H); LCMS (ESI+) m/z 324.1 (M+H)⁺.

### Example 2

### (E)-N-(3-(2-(4,4-difluorocyclohexyl)vinyl)-4-methoxyphenyl) methanesulfonamide)

The reaction scheme was as follows:

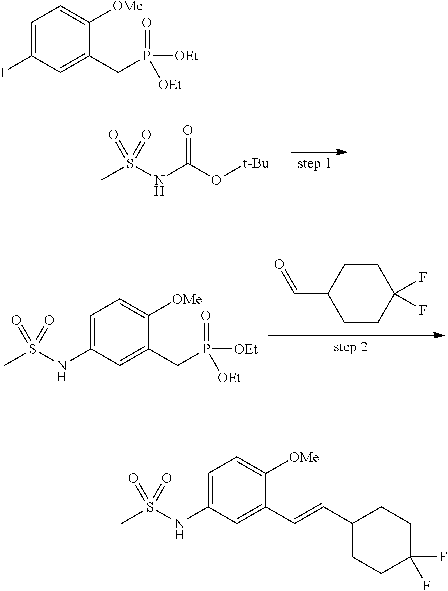 [SMILES: CCOP(=O)(Cc1cc(I)ccc1OC)OCC ; CC(C)(C)OC(=O)NS(C)(=O)=O ; CCOP(=O)(Cc1cc(NS(C)(=O)=O)ccc1OC)OCC ; O=CC1CCC(F)(F)CC1 ; COc1ccc(NS(C)(=O)=O)cc1/C=C/C1CCC(F)(F)CC1]

### Step 1: Diethyl (2-methoxy-5-(methylsulfonamido)benzyl)phosphonate

A 1-L flask was charged with 2-(diethoxyphosphorylmethyl)-4-iodo-1-methoxy-benzene (5.0 g, 13 mmol), tert-butyl n-methylsulfonylcarbamate (7.1 g, 36 mmol), cuprous iodide (2.5 g, 13 mmol), N,N-dimethylglycine (1.4 g, 13 mmol) and potassium phosphate tribasic (11.4 g, 52.1 mmol) and the flask was purged with nitrogen. N,N-Dimethylacetamide (43 mL) was then added and the flask was purged with nitrogen again, and an empty balloon was placed on top of the reaction vessel to allow room for gas generation. The reaction was stirred at 110° C. for 16 hours. The resulting mixture was diluted with 10% glycine in water and acidified to pH 1 with 1N HCl, extracted with dichloromethane (3×50 mL), dried with anhydrous MgSO₄, concentrated under reduced pressure and purified by silica gel column chromatography (0% to 10% methanol in dichloromethane) to give the title compound as a white solid (922 mg, 20% yield). LCMS (ESI+) m/z 352 (M+H)⁺.

### Step 2: (E)-N-(3-(2-(4,4-difluorocyclohexyl)vinyl)-4-methoxyphenyl) methanesulfonamide)

To a mixture of diethyl (2-methoxy-5-(methylsulfonamido)benzyl)phosphonate (70 mg, 0.20 mmol) and 4,4-difluorocyclohexane-1-carbaldehyde (44 mg, 0.30 mmol) in anhydrous tetrahydrofuran (1 mL) was added potassium tert-butoxide (56 mg, 0.50 mmol), and the reaction mixture was stirred under nitrogen for 16 hours. The resulting mixture was then partitioned between 1N HCl (5 mL) and dichloromethane (5 mL), the product was extracted with dichloromethane (5 mL), concentrated under reduced pressure and purified by reverse-phase preparative HPLC (0.1% Formic Acid in water/Acetonitrile 30-70, Gemini-NX C18 5 um, 110 A) to give the title compound as a white solid (27 mg, 41% yield). ¹H NMR (400 MHz, DMSO-d₆) δ 9.28 (s, 1H), 7.26 (d, J=2.7 Hz, 1H), 7.07 (dd, J=8.7, 2.7 Hz, 1H), 6.95 (d, J=8.8 Hz, 1H), 6.68-6.61 (m, 1H), 6.13 (dd, J=16.1, 7.0 Hz, 1H), 3.77 (s, 3H), 2.88 (s, 3H), 2.35-2.28 (m, 1H), 2.12-1.96 (m, 2H), 1.98-1.88 (m, 1H), 1.88-1.73 (m, 3H), 1.51-1.34 (m, 2H); LCMS (ESI+) m/z 346.1 (M+H)⁺.

### Example 3

### (E)-N-(3-(2-Cyclopentylvinyl)-4-methoxyphenyl)methanesulfonamide

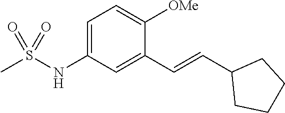 [SMILES: COc1ccc(NS(C)(=O)=O)cc1/C=C/C1CCCC1]

The title compound was prepared according to the procedure of Example 2 using cyclopentanecarbaldehyde (14 mg, 38% yield). ¹H NMR (400 MHz, DMSO-d₆) δ 9.27 (s, 1H), 7.26 (d, J=2.6 Hz, 1H), 7.06 (dd, J=8.8, 2.6 Hz, 1H), 6.95 (d, J=8.8 Hz, 1H), 6.57 (dd, J=16.0, 1.1 Hz, 1H), 6.14 (dd, J=16.0, 7.9 Hz, 1H), 3.77 (s, 3H), 2.88 (s, 3H), 2.64-2.54 (m, 1H), 1.89-1.75 (m, 2H), 1.75-1.50 (m, 4H), 1.43-1.27 (m, 2H); LCMS (ESI+) m/z 296.1 (M+H)⁺.

### Example 4

### (E)-N-(3-(3-Cyclohexylprop-1-en-1-yl)-4-methoxyphenyl)methanesulfonamide

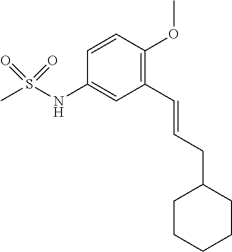 [SMILES: COc1ccc(NS(C)(=O)=O)cc1/C=C/CC1CCCCC1]

The title compound was prepared according to the procedure of Example 2 using 2-cyclohexylacetaldehyde (2.6 mg, 7% yield). LCMS (ESI+) m/z 324.1 (M+H)⁺.

### Example 5

### (E)-N-(3-(2-(4,4-Dimethylcyclohexyl)vinyl)-4-methoxyphenyl) methanesulfonamide

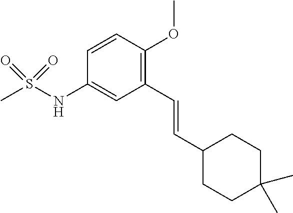

The title compound was prepared according to the procedure of Example 2 using 4,4-dimethylcyclohexane-1-carbaldehyde (4.5 mg, 11% yield). LCMS (ESI+) m/z 338.1 (M+H)⁺.

### Example 6

### (E)-N-(4-Methoxy-3-(2-(4-methylcyclohexyl)vinyl)phenyl) methanesulfonamide (Diastereomer A and Diastereomer B)

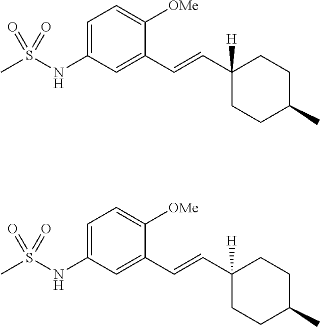

The title compounds were prepared according to the procedure of Example 2 using 4-methylcyclohexane-1-carbaldehyde. The diastereomers were separated using chiral supercritical fluid chromatography (Chiralpak AD, isocratic 20% MeOH, 40° C., 2.5 min) to give diastereomer A (4.0 mg) and diastereomer B (0.4 mg).

(E)-N-(4-Methoxy-3-(2-(4-methylcyclohexyl)vinyl)phenyl) methanesulfonamide (Diastereomer A): Chiral SFC Peak 2 (RT=0.846 min); ¹H NMR (400 MHz, DMSO-d₆) δ 9.28 (s, 1H), 7.25 (d, J=2.6 Hz, 1H), 7.06 (dd, J=8.8, 2.6 Hz, 1H), 6.95 (d, J=8.8 Hz, 1H), 6.55 (dd, J=16.3, 1.3 Hz, 1H), 6.10 (dd, J=16.1, 7.0 Hz, 1H), 3.76 (s, 3H), 2.88 (s, 3H), 2.17-1.97 (m, 1H), 1.84-1.63 (m, 4H), 1.47-1.23 (m, 1H), 1.23-1.07 (m, 2H), 1.06-0.89 (m, 2H), 0.88 (d, J=6.5 Hz, 3H); LCMS (ESI+) m/z 324.1 (M+H)⁺.

(E)-N-(4-Methoxy-3-(2-(4-methylcyclohexyl)vinyl)phenyl) methanesulfonamide (Diastereomer B): Chiral SFC Peak 1 (RT=0.737 min); LCMS (ESI+) m/z 324.1 (M+H)⁺.

### Example 7

### N-(3-(3-(4-Chlorophenyl)cyclobutyl)-4-methoxyphenyl) methanesulfonamide (Diastereomer A and Diastereomer B)

The reaction scheme was as follows:

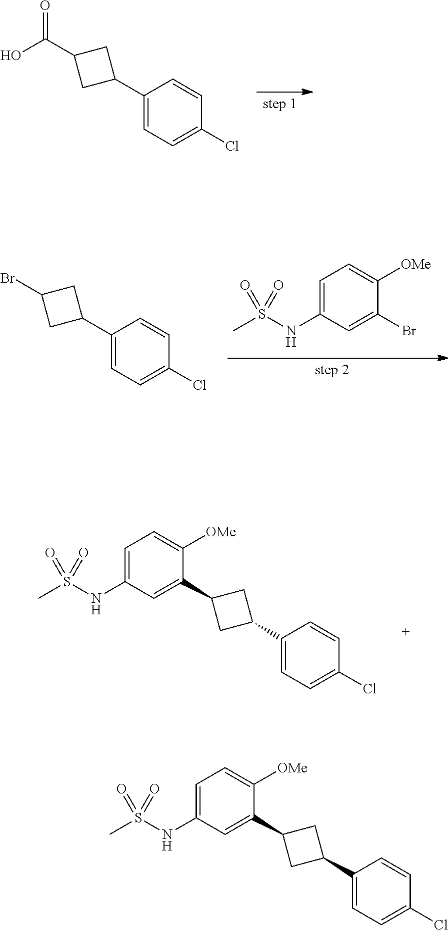 [SMILES: O=C(O)C1CC(c2ccc(Cl)cc2)C1 ; Clc1ccc(C2CC(Br)C2)cc1 ; COc1ccc(NS(C)(=O)=O)cc1Br ; COc1ccc(NS(C)(=O)=O)cc1[C@H]1C[C@H](c2ccc(Cl)cc2)C1 ; COc1ccc(NS(C)(=O)=O)cc1[C@H]1C[C@@H](c2ccc(Cl)cc2)C1]

### Step 1: 1-(3-Bromocyclobutyl)-4-chlorobenzene

A flame-dried flask was charged with [4,4′-bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate (256 mg, 0.228 mmol) and cesium carbonate (1.5 g, 4.6 mmol) and 3-(4-chlorophenyl)cyclobutane-1-carboxylic acid (1.0 g, 4.6 mmol) and the flask was purged with argon. Chlorobenzene (100 mL) and diethyl bromomalonate (8.5 mL, 46 mmol) were then added and argon was bubbled through the reaction mixture under sonication for 5 minutes. The flask was then sealed with parafilm and the mixture was irradiated with a 34W blue LED and a cooling fan for 4 hours. The crude mixture was then filtered through a short pad of silica gel, rinsing with dichloromethane, concentrated under reduced pressure and purified by silica gel column chromatography (100% heptane) to give the title compound (240 mg, 21% yield). ¹H NMR (400 MHz, Chloroform-d) δ 7.42-7.24 (m, 2H), 7.24-7.09 (m, 2H), 4.74-4.40 (m, 1H), 4.13-3.25 (m, 1H), 3.24-2.99 (m, 1H), 2.99-2.73 (m, 2H), 2.73-2.47 (m, 1H).

### Step 2: N-(3-(3-(4-Chlorophenyl)cyclobutyl)-4-methoxyphenyl) methanesulfonamide

A vial was charged with N-(3-bromo-4-methoxyphenyl)methanesulfonamide (100 mg, 0.34 mmol), [4,4′-bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate (19 mg, 0.017 mmol) and anhydrous sodium carbonate (72 mg, 0.68 mmol) and the vial was purged with nitrogen for 2 minutes. A solution of 1-(3-bromocyclobutyl)-4-chloro-benzene (92 mg, 0.37 mmol) in anhydrous 1,2-dimethoxyethane (2 mL) was then added, followed by tris(trimethylsilyl)silane (0.11 mL, 0.34 mmol) and nitrogen was bubbled through the resulting mixture for 5 minutes. A separate vial was charged with nickel(ii) chloride ethylene glycol dimethyl ether complex (3.8 mg, 0.017 mmol) and 4,4′-di-tert-butyl-2,2′-bipyridine (4.6 mg, 0.017 mmol) and the vial was purged with nitrogen for 5 minutes. 1,2-Dimethoxyethane (2 mL) was then added and nitrogen was bubbled through the mixture under sonication for 5 minutes. This resulted in the formation of a green solution. The green solution was transferred to the first vial using a syringe and the resulting mixture was further sonicated under nitrogen for 1 minute and sealed with parafilm. The reaction mixture was then stirred at room temperature and irradiated with a 34W blue LED and a cooling fan for 16 hours. The reaction was quenched by exposure to air and concentrated on silica gel. It was then purified by silica gel column chromatography and the two diastereomers were separated using chiral supercritical fluid chromatography (Chiralpak ID, isocratic 15% MeOH w/ 0.1% NH₄OH, 40° C., 2.5 min) to give diastereomer A (9.3 mg) and diastereomer B (16.8 mg).

N-(3-(3-(4-Chlorophenyl)cyclobutyl)-4-methoxyphenyl) methanesulfonamide (Diastereomer A): Chiral SFC Peak 2 (RT=1.166 min); ¹H NMR (400 MHz, DMSO-d₆) δ 9.30 (s, 1H), 7.39 (s, 4H), 7.25 (dd, J=2.7, 0.8 Hz, 1H), 7.07 (dd, J=8.6, 2.6 Hz, 1H), 6.92 (d, J=8.8 Hz, 1H), 3.74 (s, 3H), 3.73-3.69 (m, 1H), 3.60-3.50 (m, 1H), 2.90 (s, 3H), 2.49-2.43 (m, 4H); LCMS (ESI+) m/z 365.1 (M+H)⁺.

N-(3-(3-(4-Chlorophenyl)cyclobutyl)-4-methoxyphenyl) methanesulfonamide (Diastereomer B): Chiral SFC Peak 1 (RT=0.946 min); ¹H NMR (400 MHz, DMSO-d₆) δ 9.27 (s, 1H), 7.38-7.31 (m, 2H), 7.31-7.22 (m, 2H), 7.13-7.02 (m, 2H), 6.91 (d, J=8.6 Hz, 1H), 3.76 (s, 3H), 3.66-3.53 (m, 1H), 3.53-3.43 (m, 1H), 2.88 (s, 3H), 2.77-2.62 (m, 2H), 2.11-1.94 (m, 2H); LCMS (ESI+) m/z 365.1 (M+H)⁺.

### Example 8

### N-(3-(3-(3-Fluorophenyl)cyclobutyl)-4-methoxyphenyl) methanesulfonamide (Diastereomer A and Diastereomer B)

The reaction scheme was as follows:

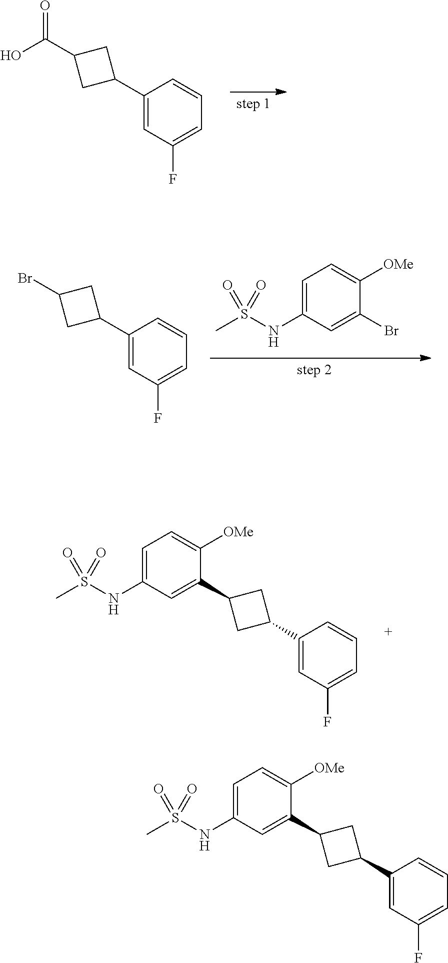

### Step 1: 1-(3-Bromocyclobutyl)-3-fluorobenzene

The title compound was prepared according to the procedure of Example 7 using 3-(3-fluorophenyl)cyclobutane-1-carboxylic acid (16% yield, volatile compound). ¹H NMR (400 MHz, Chloroform-d) δ 7.31-7.23 (m, 1H), 7.01-6.95 (m, 1H), 6.95-6.87 (m, 2H), 4.66-4.40 (m, 1H), 4.12-3.23 (m, 1H), 3.12-3.00 (m, 1H), 2.92-2.75 (m, 2H), 2.70-2.53 (m, 1H).

### Step 2: N-(3-(3-(3-fluorophenyl)cyclobutyl)-4-methoxyphenyl) methanesulfonamide

The title compound was prepared according to the procedure of Example 7 using 1-(3-bromocyclobutyl)-3-fluorobenzene to give diastereomer A (6.7 mg) and diastereomer B (7.2 mg).

N-(3-(3-(3-Fluorophenyl)cyclobutyl)-4-methoxyphenyl) methanesulfonamide (Diastereomer A): Chiral SFC Peak 2 (RT=1.373 min); LCMS (ESI+) m/z 350.1 (M+H)⁺.

N-(3-(3-(4-Chlorophenyl)cyclobutyl)-4-methoxyphenyl) methanesulfonamide (Diastereomer B): Chiral SFC Peak 1 (RT=1.116 min); ¹H NMR (400 MHz, DMSO-d₆) δ 9.27 (s, 1H), 7.35 (m, J=7.9, 6.2 Hz, 1H), 7.13-6.97 (m, 5H), 6.91 (d, J=8.6 Hz, 1H), 3.76 (s, 3H), 3.66-3.45 (m, 2H), 2.88 (s, 3H), 2.70 (qd, J=7.8, 2.7 Hz, 2H), 2.12-2.01 (m, 2H); LCMS (ESI+) m/z 350.1 (M+H)⁺.

### Example 9

### 3-(3-(4-Chlorophenyl)cyclobutyl)-N-isopropyl-4-methoxybenzamide (Diastereomer A and Diastereomer B)

The overall reaction scheme was as follows:

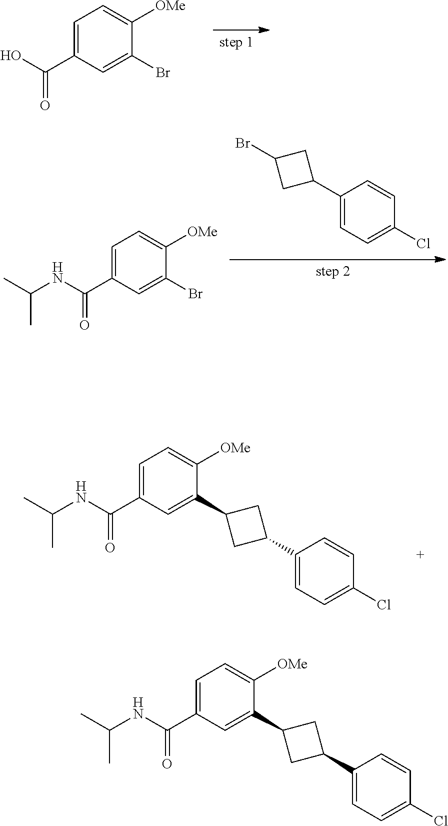 [SMILES: COc1ccc(C(=O)O)cc1Br ; COc1ccc(C(=O)NC(C)C)cc1Br ; Clc1ccc(C2CC(Br)C2)cc1 ; COc1ccc(C(=O)NC(C)C)cc1[C@H]1C[C@H](c2ccc(Cl)cc2)C1 ; COc1ccc(C(=O)NC(C)C)cc1[C@H]1C[C@@H](c2ccc(Cl)cc2)C1]

### Step 1: 3-Bromo-N-isopropyl-4-methoxybenzamide

A flask was charged with 3-bromo-4-methoxybenzoic acid (250 mg, 1.1 mmol), N,N-dimethylformamide (4 mL) and N,N-diisopropylethylamine (0.57 mL, 3.24 mmol), followed by 1-[bis(dimethylamino)methylene]-1H-1,2,3-triazolo[4,5-b]pyridinium 3-oxide hexafluorophosphate (636 mg, 1.62 mmol) and the solution was stirred for 1 minute until dissolution occurred. Isopropylamine (0.23 mL, 2.7 mmol) was then added and the reaction was stirred at room temperature for 1 hour. The reaction was partitioned between saturated aqueous NaHCO₃ (10 mL) and i-PrOAc (10 mL), extracted with i-PrOAc (10 mL), washed with water and brine, dried with anhydrous MgSO₄, concentrated under reduced pressure and purified by silica gel column chromatography (0% to 100% i-PrOAc in heptane) to give the title compound as a white solid (253 mg, 86% yield). LCMS (ESI+) m/z 272 (M+H)⁺.

### Step 2: 3-(3-(4-Chlorophenyl)cyclobutyl)-N-isopropyl-4-methoxybenzamide

The title compound was prepared according to the procedure of Example 7 using 3-Bromo-N-isopropyl-4-methoxybenzamide to give diastereomer A (23.4 mg) and diastereomer B (14.4 mg). 3-(3-(4-Chlorophenyl)cyclobutyl)-N-isopropyl-4-methoxybenzamide (Diastereomer A): Chiral SFC Peak 2 (RT=1.134 min); ¹H NMR (400 MHz, DMSO-d₆) δ 8.04 (d, J=7.8 Hz, 1H), 7.73 (dd, J=8.5, 2.2 Hz, 1H), 7.66 (dd, J=2.3, 0.8 Hz, 1H), 7.40-7.33 (m, 2H), 7.33-7.24 (m, 2H), 6.98 (d, J=8.5 Hz, 1H), 4.16-4.02 (m, 1H), 3.83 (s, 3H), 3.67-3.54 (m, 1H), 3.54-3.43 (m, 1H), 2.78-2.62 (m, 2H), 2.20-2.04 (m, 2H), 1.15 (d, J=6.6 Hz, 6H); LCMS (ESI+) m/z 358.1 (M+H)⁺.

### 3-(3-(4-Chlorophenyl)cyclobutyl)-N-isopropyl-4-methoxybenzamide

(Diastereomer B): SFC Peak 1 (RT=0.889 min); ¹H NMR (400 MHz, DMSO-d₆) δ 8.09 (d, J=7.8 Hz, 1H), 7.87 (dd, J=2.3, 0.8 Hz, 1H), 7.76 (dd, J=8.5, 2.3 Hz, 1H), 7.40 (s, 4H), 7.00 (d, J=8.6 Hz, 1H), 4.18-4.07 (m, 1H), 3.81 (s, 3H), 3.80-3.72 (m, 1H), 3.67-3.51 (m, 1H), 3.29-3.24 (m, 2H), 2.63-2.53 (m, 2H), 1.18 (d, J=6.6 Hz, 6H); LCMS (ESI+) m/z 358.1 (M+H)⁺.

### Examples 10 to 12

The overall reaction scheme for Examples 10 to 12 was as follows: Examples 10-12:

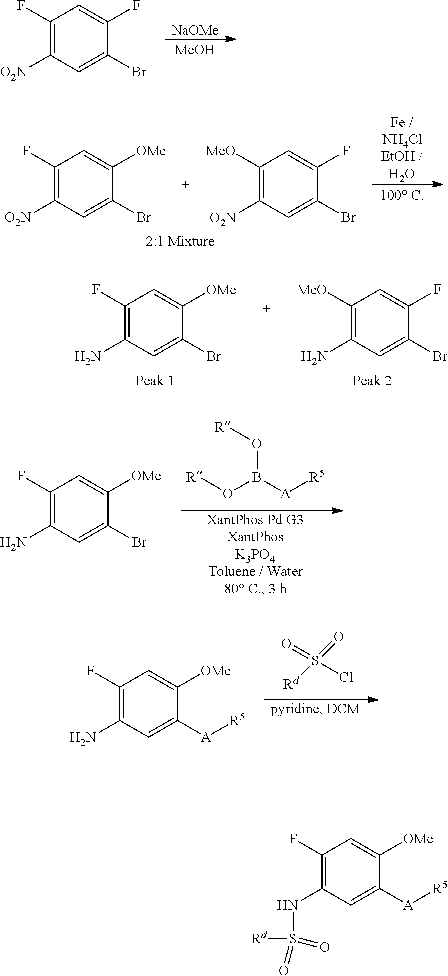

### Example 10

### (E)-N-(5-(4-chlorostyryl)-2-fluoro-4-methoxyphenyl) cyclopropanesulfonamide

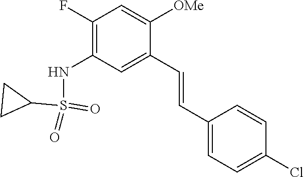 [SMILES: COc1cc(F)c(NS(=O)(=O)C2CC2)cc1/C=C/c1ccc(Cl)cc1]

### Step 1: 1-Bromo-4-fluoro-2-methoxy-5-nitrobenzene and 1-bromo-2-fluoro-4-methoxy-5-nitro-benzene

To a stirred solution of 1-bromo-2,4-difluoro-5-nitro-benzene (12.1 g, 50.8 mmol) in MeOH (100 mL) was added 25% sodium methoxide in MeOH (12 mL, 53.4 mmol, 12 mL) at 0° C., and the reaction mixture was stirred at 0° C. for 2 h and then at RT for 20 h. Volatile solvent was removed under reduced pressure, and the resultant residue was partitioned between ⁱPrOAc and water. The organic layer was washed with water and brine, dried over Na₂SO₄, filtered and concentrated in vacuo. The crude product was purified by column chromatography (SiO₂: ⁱPrOAc/heptane) to afford 10.9 g (86% yield) of a mixture of 1-bromo-4-fluoro-2-methoxy-5-nitro-benzene and 1-bromo-2-fluoro-4-methoxy-5-nitro-benzene (˜2:1 ratio). 1-Bromo-4-fluoro-2-methoxy-5-nitrobenzene: ¹H NMR (400 MHz, CDCl₃) δ 8.36 (d, J=8.0 Hz, 1H), 6.77 (d, J=12.3 Hz, 1H), 4.00 (s, 3H); 1-bromo-2-fluoro-4-methoxy-5-nitro-benzene: ¹H NMR (400 MHz, CDCl₃) δ 8.16 (d, J=7.1 Hz, 1H), 6.89 (d, J=9.8 Hz, 1H), 3.97 (s, 3H).

### Step 2: 5-Bromo-2-fluoro-4-methoxyaniline and 5-bromo-4-fluoro-2-methoxyaniline

To a mixture of 1-bromo-4-fluoro-2-methoxy-5-nitro-benzene and 1-bromo-2-fluoro-4-methoxy-5-nitro-benzene (˜2:1 ratio) (6.1 g, 24.3 mmol) dissolved in EtOH (162 mL) was added ammonium chloride (13.0 g, 243.2 mmol) in water (49 mL), followed by iron powder (6.8 g, 121.6 mmol). The reaction mixture was stirred at reflux for 20 h. The reaction was cooled to RT and the reaction was filtered through a pad of Celite®. The pad of rinsed well with DCM and EtOH, and the filtrate was basified with sat. aq. NaHCO₃ solution until pH 7 and extracted with ⁱPrOAc (3×). The combined organic layers were washed with water, brine, dried over Na₂SO₄, filtered and concentrated in vacuo. The crude products were purified by column chromatography (SiO₂: ⁱPrOAc/hexane) to retrieve 3.3 g (61% yield) of 5-bromo-2-fluoro-4-methoxyaniline followed by 2.0 g (36% yield) of 5-bromo-4-fluoro-2-methoxyaniline. 5-bromo-2-fluoro-4-methoxyaniline: ¹H NMR (400 MHz, CDCl₃) δ 7.00 (d, J=9.3 Hz, 1H), 6.66 (d, J=12.1 Hz, 1H), 3.80 (s, 3H), 3.47 (s, 2H); MS (ESI+) m/z 220/222 (M+H)⁺. 5-bromo-4-fluoro-2-methoxyaniline: ¹H NMR (400 MHz, CDCl₃) δ 6.82 (d, J=6.9 Hz, 1H), 6.61 (d, J=10.0 Hz, 1H), 3.83 (s, 3H), 3.68 (s, 2H); MS (ESI+) m/z 220/222 (M+H)⁺.

### Step 3: (E)-5-(4-chlorostyryl)-2-fluoro-4-methoxyaniline

A screwed top flask was charged with 5-bromo-2-fluoro-4-methoxy-aniline (1.02 g, 4.6 mmol), 2-[(E)-2-(4-chlorophenyl)vinyl]-4,4,5,5-tetramethyl-1,3,2-dioxaborolane (1.6 g, 6.0 mmol), potassium phosphate (2.0 g, 9.2 mmol, 2022.4 mg), SPhos pre-catalyst G3 (0.36 g, 0.46 mmol), SPhos (0.34 g, 0.79 mmol), toluene (15 mL), and water (1.5 mL). The reaction mixture was vacuum purged/back-filled with N₂ (3×). The flask was screwed tightly with a cap, and the reaction mixture was stirred at 95° C. for 18 h. The cooled reaction mixture was diluted with ⁱPrOAc and filtered through a pad of Celite®. The pad was rinsed with additional ⁱPrOAc. The filtrate was washed with water and brine, dried over Na₂SO₄, filtered and concentrated in vacuo. The crude product was purified by column chromatography (SiO₂: ⁱPrOAc/heptane) to retrieve (E)-5-(4-chlorostyryl)-2-fluoro-4-methoxyaniline (1.14 g, 89% yield) of. MS (ESI+) m/z 278 (M+H)⁺.

### Step 4: (E)-N-(5-(4-chlorostyryl)-2-fluoro-4-methoxyphenyl) cyclopropanesulfonamide

To a stirred solution of 5-[(E)-2-(4-chlorophenyl)vinyl]-2-fluoro-4-methoxy-aniline (181 mg, 0.46 mmol) in DCM (11 mL) was added pyridine (0.18 mL, 2.3 mmol) followed by cyclopropanesulfonyl chloride (70 mg, 0.50 mmol), and the reaction mixture was stirred at RT for 4 days. The reaction was quenched with 1N HCl and then diluted with iPrOAc. The resultant white precipitate was filtered, and the filtrate was washed with water and brine, dried over Na₂SO₄, filtered, and concentrated in vacuo. The crude product was purified by column chromatography (SiO2: ⁱPrOAc/heptane) followed by reverse-phase preparative HPLC to afford 47 mg (27% yield) of the title compound as a white solid. H NMR (400 MHz, DMSO-d₆) δ 9.35 (s, 1H), 7.63-7.56 (m, 3H), 7.45-7.39 (m, 2H), 7.32 (d, J=16.5 Hz, 1H), 7.16 (d, J=16.6 Hz, 1H), 7.05 (d, J=12.1 Hz, 1H), 3.88 (s, 3H), 2.66-2.56 (m, 1H), 1.00-0.91 (m, 2H), 0.88-0.79 (m, 2H); MS (ESI+) m/z 399 (M+H)⁺.

### Example 11

### N-(4-fluoro-4′-isopropyl-6-methoxy-[1,1′-biphenyl]-3-yl) cyclopropanesulfonamide

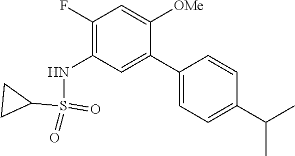 [SMILES: COc1cc(F)c(NS(=O)(=O)C2CC2)cc1-c1ccc(C(C)C)cc1]

The title compound was prepared according to the procedure of Example 10, substituting (4-isopropylphenyl)boronic acid for 2-[(E)-2-(4-chlorophenyl)vinyl]-4,4,5,5-tetramethyl-1,3,2-dioxaborolane, Example 11 (84 mg, 23%) was prepared. ¹H NMR (400 MHz, DMSO-d₆) δ 9.34 (s, 1H), 7.39-7.32 (m, 2H), 7.31-7.25 (m, 2H), 7.23 (d, J=9.0 Hz, 1H), 7.09 (d, J=12.2 Hz, 1H), 3.78 (s, 3H), 2.97-2.85 (m, 1H), 2.65-2.57 (m, 1H), 1.23 (d, J=6.8 Hz, 6H), 0.97-0.91 (m, 2H), 0.87-0.81 (m, 2H); MS (ESI+) m/z 381 (M+NH₄)⁺. HRMS (ESI−): m/z calcd for C₁₉H₂₁FNO₃S [M−H]⁻, 362.1226; found, 362.0941.

### Example 12

### N-(4-fluoro-4′-isopropyl-6-methoxy-[1,1′-biphenyl]-3-yl) methanesulfonamide

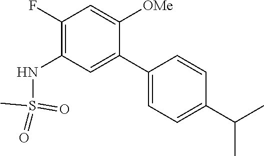 [SMILES: COc1cc(F)c(NS(C)(=O)=O)cc1-c1ccc(C(C)C)cc1]

The title compound was prepared according to the procedure of Example 10, substituting methansulfonic anhydride for cyclopropanesulfonyl chloride, Example 12 (71 mg, 42%) was prepared. ¹H NMR (400 MHz, DMSO-d₆) δ 9.35 (s, 1H), 7.40-7.18 (m, 5H), 7.11 (d, J=12.3 Hz, 1H), 3.78 (s, 3H), 2.98 (s, 3H), 2.97-2.85 (m, 1H), 1.23 (d, J=6.9 Hz, 6H); MS (ESI+) m/z 355.1 (M+NH4)⁺. HRMS (ESI−): m/z calcd for C₁₇H₁₉FNO₃S, 336.1070 [M−H]⁻; found, 336.0805.

### Example 13

### N-(5-((1R,3S)-3-(4-chlorophenyl)cyclobutyl)-6-methoxypyridin-3-yl) methanesulfonamide

The overall reaction scheme for example 13 was as follows:

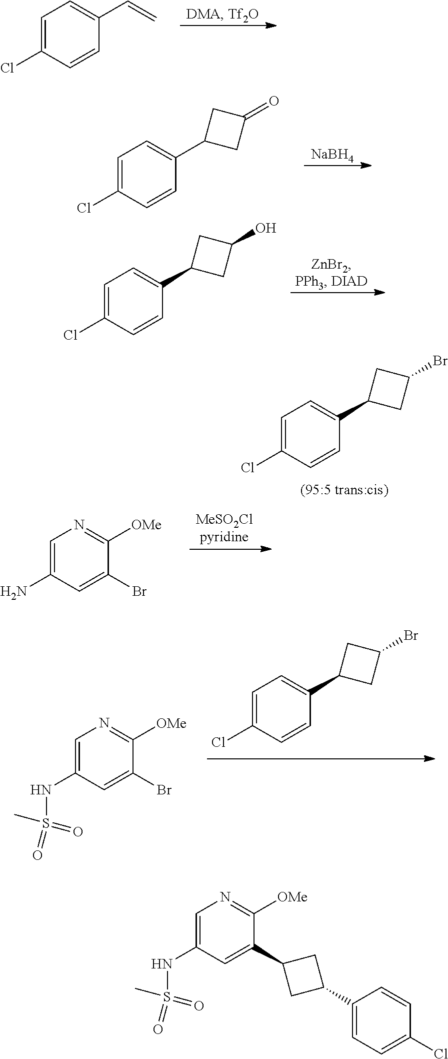

### Step 1: 3-(4-chlorophenyl)cyclobutan-1-one

A 2 L, 4 neck round-bottom flask was equipped with an argon inlet adapter, thermocouple, overhead stirrer, condenser fitted with a drying tube and an addition funnel. Dimethylacetamide (72.5 ml, 779 mmol, 1.2 eq) was added to the flask and dissolved in DCM (1.21 L). The mixture was cooled in an ice-water bath. Trifluoromethanesulfonic anhydride (153 ml, 909 mmol, 1.4 eq) was added slowly via an addition funnel while maintaining the internal temperature below 8° C. The addition took about 1.5 hours and resulted in the formation of a slurry. 4-chlorostyrene (90.0 g, 649 mmol, 1 eq) and 2,4,6-trimethylpyridine (120 ml, 909 mmol, 1.4 eq) were dissolved in DCM (180 ml). The resulting solution was then added to the reaction mixture via an addition funnel, dropwise over 2 h, while maintaining the temperature below 10° C. The reaction mixture was more readily stirred upon completing the addition. The reaction mixture was then heated to a gentle reflux for 14 hours with a heating mantle with a resulting internal temperature of around 87° C. The reaction mixture was concentrated and the residue was heated for 18 hours at reflux with CCl₄ (405 ml) and water (405 ml). The resulting multiphasic mixture, which contained a brown syrupy oily substance, was filtered through Celite®, but the oily substance still passed through the filter media. Cyclohexane (500 ml) was added and the mixture was transferred to a separatory funnel. The bottom phase was dark brown and also contained a syrupy component and the top organic phase was light yellow. The layers were separated and the aqueous phase was extracted with cyclohexane (3×). The organic extracts were combined and filtered through a pad of silica gel. The resulting filtrate was concentrated to give 3-(4-chlorophenyl)cyclobutan-1-one as light amber oil (38.3 g, 32.7% yield). ¹H NMR (400 MHz, CD₂Cl₂) δ 7.36-7.31 (m, 2H), 7.29-7.24 (m, 2H), 3.74-3.59 (m, 1H), 3.54-3.41 (m, 2H), 3.25-3.13 (m, 2H).

### Step 2: (1S,3S)-3-(4-chlorophenyl)cyclobutan-1-ol

3-(4-Chlorophenyl)cyclobutan-1-one (38.3 g, 212 mmol, 1 eq) was dissolved in MeOH (383 ml). Sodium borohydride (2.65 g, 70 mmol, 0.33 eq) was added portionwise, while maintaining the temperature between 20 to 25° C. during the addition. The reaction mixture was concentrated. Water (200 ml) and ether (300 ml) were added and the mixture was transferred to a separatory funnel. The aqueous layer was discarded and the organic layer was washed with brine, dried with sodium sulfate, filtered and concentrated to obtain (1S,3S)-3-(4-chlorophenyl)cyclobutan-1-ol as light yellow oil (36 g, 93% yield). ¹H NMR indicated good purity and a dr of 9:1 as previously reported in the literature. ¹H NMR (400 MHz, DMSO-d₆) δ 7.34 (d, J=8.4 Hz, 2H), 7.27-7.21 (m, 2H), 5.09 (d, J=7.2 Hz, 1H), 4.02 (sxt, J=7.4 Hz, 1H), 2.94-2.79 (m, 1H), 2.64-2.54 (m, 2H), 1.90-1.79 (m, 2H).

### Step 3: 1-((1R,3R)-3-bromocyclobutyl)-4-chlorobenzene (95:5 trans:cis)

(1S,3S)-3-(4-chlorophenyl)cyclobutan-1-ol (10.0 g, 54.7 mmol, 1 eq) was dissolved in anhydrous THF (400 ml). Triphenylphosphine (53.0 g, 202 mmol, 3.69 eq) was added, followed by a solution of anhydrous zinc bromide (15.2 g, 67.3 mmol, 1.23 eq) in anhydrous THF (100 ml). Finally, DIAD (39.8 ml, 3.69 equivalents) dissolved in anhydrous THF (100 ml) was added to the reaction mixture. A white solid began to form within minutes of completing the addition. The reaction mixture was allowed to stir overnight at room temperature. The resulting white solid was filtered over a plug of silica gel. The filtrate was concentrated and treated with hexane to precipitate triphenylphosphine oxide, which was removed by filtration. The resulting filtrate was concentrated and chromatographed on a silica gel column (120 g) with 100% hexanes. The impure fractions were combined and purified by column chromatography under the same conditions. All pure fractions were combined and concentrated to obtain 1-((1R,3R)-3-bromocyclobutyl)-4-chlorobenzene (95:5 trans:cis) as an oil which crystallized upon cooling (6.8 g, 50.6%, 95:5 trans:cis).

### Step 4: N-(5-bromo-6-methoxypyridin-3-yl)methanesulfonamide

To a stirred solution of 5-bromo-6-methoxy-pyridin-3-amine (20.0 g, 98.5 mmol) in DCM (100 mL) at 0° C. was added pyridine (14.3 mL, 177 mmol) followed by methanesulfonyl chloride (8.4 mL, 108.4 mmol), and the reaction mixture was stirred at room temperature for 19 h. The reaction was diluted with PrOAc. The organic phase was washed with 10% aq. HCl solution, water and brine, dried over Na₂SO₄, filtered, and concentrated in vacuo. The crude product was purified by column chromatography (SiO₂: ⁱPrOAc/heptane) and then triturated in ether to obtain N-(5-bromo-6-methoxypyridin-3-yl)methanesulfonamide (23.8 g, 86%) as a pink solid. ¹H NMR (400 MHz, CDCl₃) δ 8.02 (d, J=2.5 Hz, 1H), 7.87 (d, J=2.5 Hz, 1H), 6.43 (s, 1H), 4.01 (s, 3H), 3.02 (s, 3H); MS (ESI+) m/z 282/283 (M+H)⁺.

### Step 5: N-(5-((1R,3R)-3-(4-chlorophenyl)cyclobutyl)-6-methoxypyridin-3-yl)methanesulfonamide

An oven-dried vial was charged with N-(5-bromo-6-methoxypyridyl)methanesulfonamide (300.0 mg, 1.07 mmol), (Ir[dF(CF₃)ppy]₂(dtbpy))PF₆ (18.0 mg, 0.016 mmol), and anhydrous sodium carbonate (226.2 mg, 2.13 mmol), and purged with nitrogen for 2 min. A solution of 1-((1R,3R)-3-bromocyclobutyl)-4-chlorobenzene (95:5 trans:cis) (340.6 mg, 1.39 mmol) in anhydrous DME (7.1 mL) was then added to the vial above, followed by tris(trimethylsilyl)silane (0.34 mL, 1.07 mmol). Nitrogen was then bubbled through the resulting mixture for 5 minutes. In a separate oven-dried vial was charged with Nickel(II) chloride ethylene glycol dimethyl ether complex (12.1 mg, 0.053 mmol) and 4,4′-di-tert-butyl-2,2′-bipyridine (14.3 mg, 0.053 mmol), and the solids were purged with nitrogen for 5 minutes. Anhydrous DME (7.1 mL) was added, and nitrogen was bubbled through the reaction mixture under sonication for 5 min until formation of a green active Ni-complexed catalyzed solution. The solution was syringed out and transferred to the 1st vial, and the resulting mixture was further sonicated under nitrogen for 1 min. The reaction mixture was then stirred at room temperature and irradiated with a 34 W LED and a cooling fan for 6 h. The reaction mixture was filtered through a pad of Celite® and rinse well with DCM. The filtrate was concentrated under reduced pressure. The crude product was purified by column chromatography (SiO₂: ¹PrOAc/heptane) followed by SFC chiral separation (Chiralpak AD, isocratic 25% MeOH w/ 0.1% NH₄OH, 40° C., 2.5 min). The second peak was collected to give the title compound (81.3 mg, 20.8%) as a white solid. Chiral SFC Peak 2 (RT=0.902 min), % ee=100; ¹NMR (400 MHz, DMSO-d₆) δ 9.47 (s, 1H), 7.91 (d, J=2.5 Hz, 1H), 7.61 (dd, J=2.7, 0.9 Hz, 1H), 7.38 (s, 4H), 3.85 (s, 3H), 3.69-3.59 (m, 1H), 3.59-3.49 (m, 1H), 2.97 (s, 3H), 2.49-2.46 (m, 4H); MS (ESI+) m/z 367 (M+H)⁺.

### Examples 14 to 17

The overall reaction scheme for Examples 14 to 17 was as follows:

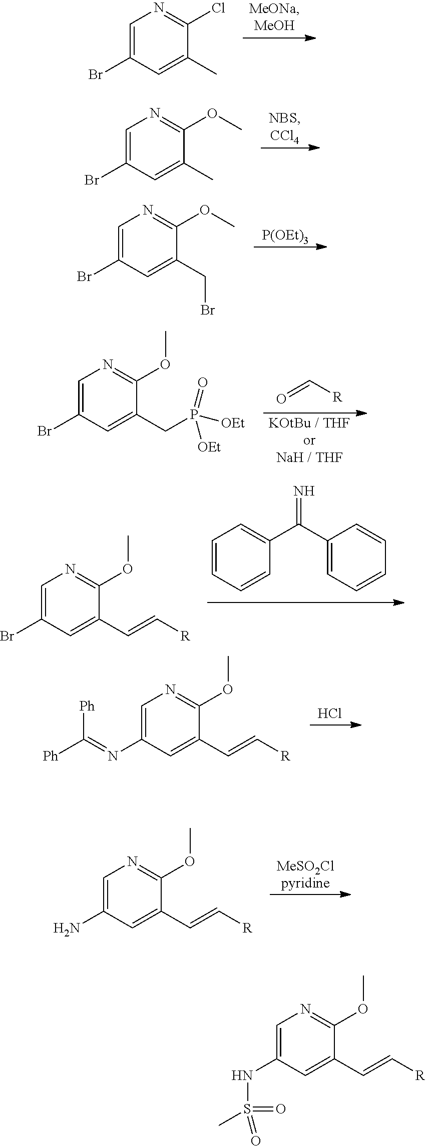

### Example 14

### (E)-N-(5-(2-(4,4-difluorocyclohexyl)vinyl)-6-methoxypyridin-3-yl) methanesulfonamide

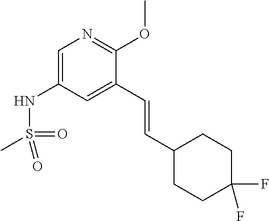 [SMILES: COc1ncc(NS(C)(=O)=O)cc1/C=C/C1CCC(F)(F)CC1]

### Step 1: 5-Bromo-2-methoxy-3-methylpyridine

To a solution of 5-bromo-2-chloro-3-methylpyridine (300 g, 1.45 mol) in MeOH (3 L) was added freshly prepared sodium methoxide (156 g, 2.9 mol), and the reaction mixture was heated to reflux and stirred overnight. The reaction mixture was quenched with acetic acid (600 mL) and concentrated under reduced pressure. The crude mixture was diluted with ethyl acetate (3 L) and washed with water (3 L). The aqueous layer was extracted with ethyl acetate (3 L) and the combined organic layers were washed with brine (3 L), dried over anhydrous MgSO₄ and evaporated under reduced pressure to provide crude 5-bromo-2-methoxy-3-methylpyridine (220 g, 75%). ¹H NMR (300 MHz, CDCl₃) δ (s, 1H), 7.47 (s, 1H), 3.93 (s, 3H), 2.05 (s, 3H).

### Step 2: 5-Bromo-3-(bromomethyl)-2-methoxypyridine

To a solution of 5-bromo-2-methoxy-3-methylpyridine (40 g, 198 mmol) in CCl₄ (400 mL) was added NBS (38.7 g, 217.4 mmol) and AIBN (1.62 g, 6.1 mmol), and the reaction mixture was heated to reflux and stirred for 2 h. The reaction mixture was concentrated under reduced pressure to give a crude residue. Petroleum ether (800 mL) was added and the reaction mixture was filtered to remove the solid. The filtrate was concentrated under reduced pressure to give a crude residue, which was triturated in petroleum ether to afford 5-bromo-3-(bromomethyl)-2-methoxypyridine as off-white solid (22 g, 40%). ¹H NMR (300 MHz, CDCl₃) δ (s, 1H), 7.73 (s, 1H), 4.43 (s, 2H), 4.00 (s, 3H).

### Step 3: Diethyl ((5-bromo-2-methoxypyridin-3-yl)methyl)phosphonate

To a solution of 5-bromo-3-(bromomethyl)-2-methoxypyridine (22 g, 78.5 mmol) in 1,4-dioxane (110 mL) was added triethyl phosphite (26 g, 217.4 mmol), and the reaction mixture was heated to reflux and stirred overnight. The reaction mixture was concentrated under reduced pressure to remove volatile solvent, and the product was distilled to afford diethyl ((5-bromo-2-methoxypyridin-3-yl)methyl)phosphonate as colorless oil (25 g, 94%). ¹H NMR (300 MHz, DMSO-d₆) δ (s, 1H), 7.70 (s, 1H), 4.09 (q, J=7.2 Hz, 4H), 3.94 (s, 3H), 3.15 (d, J=21.9 Hz, 2H), 1.27 (t, J=7.2 Hz, 6H)); MS (ESI+) m/z 337.8 (M+H)⁺.

### Step 4: (E)-5-bromo-3-(2-(4,4-difluorocyclohexyl)vinyl)-2-methoxypyridine

To a mixture of 4,4-difluorocyclohexanecarbaldehyde (1070 mg, 7.23 mmol) and 5-bromo-3-(diethoxyphosphorylmethyl)-2-methoxypyridine (820 mg, 2.41 mmol) in anhydrous THF (13.4 mL) was added potassium tert-butoxide (1910 mg, 16.9 mmol), and the reaction mixture was stirred at room temperature under N₂ for 2 h. The reaction mixture was diluted with PrOAc and water. The organic phase was washed with water and brine, dried over Na₂SO₄, filtered, and concentrated in vacuo. The crude product was purified by column chromatography (SiO₂: ⁱPrOAc/heptane) to obtain (E)-5-bromo-3-(2-(4,4-difluorocyclohexyl)vinyl)-2-methoxypyridine (258 mg, 32.2%). MS (ESI+) m/z 332/334 (M+H)⁺.

### Step 5: (E)-N-(5-(2-(4,4-difluorocyclohexyl)vinyl)-6-methoxypyridin-3-yl)-1,1-diphenylmethanimine

In a 20-mL vial was placed 5-bromo-3-[(E)-2-(4,4-difluorocyclohexyl)vinyl]-2-methoxy-pyridine (257.0 mg, 0.77 mmol), diphenylmethanimine (0.18 mL, 1.08 mmol), sodium tert-butoxide (148.7 mg, 1.55 mmol), bis(2-diphenylphosphinophenyl)ether (41.7 mg, 0.077 mmol, 41.66 mg), and tris(dibenzylidenteactone)dipalladium(0) (35.4 mg, 0.039 mmol). Degassed toluene (5.2 mL) was added. The vial was vacuum purged/back-filled with N₂ (3×) and capped. The reaction mixture was stirred at 120° C. for 40 h. The reaction mixture was diluted with ⁱPrOAc and water, and then filtered through a pad of Celite®. The biphasic layers were separated. The organic phase was washed with water and brine, dried over Na₂SO₄, filtered, and concentrated in vacuo. The crude was purified by column chromatography (SiO₂: iPrOAc/heptane) to obtain (E)-N-(5-(2-(4,4-difluorocyclohexyl)vinyl)-6-methoxypyridin-3-yl)-1,1-diphenylmethanimine (165 mg, 49.3% yield) as a yellow oil. MS (ESI+) m/z 433 (M+H)⁺.

### Step 6: (E)-5-(2-(4,4-difluorocyclohexyl)vinyl)-6-methoxypyridin-3-amine

To (E)-N-(5-(2-(4,4-difluorocyclohexyl)vinyl)-6-methoxypyridin-3-yl)-1,1-diphenylmethanimine (165 mg, 0.382 mmol) dissolved in THF (7.7 mL) was added 1N HCl (3.8 mL, 3.87 mmol), and the reaction mixture was stirred at RT for 2 h. Volatile solvent was removed under reduced pressure, and the resultant crude product was diluted with DCM and basified with 1N NaOH until pH 8. The reaction mixture was extracted with DCM (3×). The combined organic layers were washed with water and brine, dried over Na₂SO₄, filtered, and concentrated in vacuo. The crude was purified by column chromatography (SiO2: iPrOAc/heptane) to give (E)-N-(5-(2-(4,4-difluorocyclohexyl)vinyl)-6-methoxypyridin-3-yl)-1,1-diphenylmethanimine (88.4 mg, 86.1%) as a white solid. MS (ESI+) m/z 269 (M+H)⁺.

### Step 7: (E)-N-(5-(2-(4,4-difluorocyclohexyl)vinyl)-6-methoxypyridin-3-yl)methanesulfonamide

To a stirred solution of 5-[(E)-2-(4,4-difluorocyclohexyl)vinyl]-6-methoxy-pyridin-3-amine (88.4 mg, 0.33 mmol) in DCM (0.33 mL) at 0° C. was added pyridine (0.05 mL, 0.59 mmol) followed by methanesulfonyl chloride (1.100 equiv, 0.3624 mmol, 41.52 mg, 0.0281 mL) in DCM (1 mL), and the reaction mixture was stirred at room temperature for 19 h. The reaction was diluted with iPrOAc. The organic phase was washed with 10% aq. HCl solution, water and brine, dried over Na₂SO₄, filtered, and concentrated in vacuo. The crude product was purified by column chromatography (SiO2: iPrOAc/heptane) and then triturated in ether and hexane until white solid precipitated. The solid was filtered and pumped dry on high-vac to obtain the title compound (48.7 mg, 42.7%). ¹H NMR (400 MHz, DMSO-d₆) δ 9.47 (s, 1H), 7.91 (d, J=2.6 Hz, 1H), 7.64 (d, J=2.5 Hz, 1H), 6.50 (dd, J=16.3, 1.2 Hz, 1H), 6.33 (dd, J=16.2, 6.9 Hz, 1H), 3.88 (s, 3H), 2.96 (s, 3H), 2.40-2.29 (m, 1H), 2.11-1.98 (m, 2H), 1.97-1.90 (m, 1H), 1.90-1.77 (m, 3H), 1.50-1.36 (m, 2H); MS (ESI+) m/z 347.1 (M+H)⁺.

### Example 15

### (E)-N-(6-methoxy-5-(4-methylpent-1-en-1-yl)pyridin-3-yl) methanesulfonamide

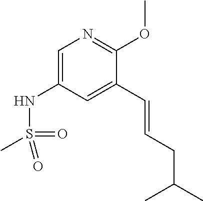 [SMILES: COc1ncc(NS(C)(=O)=O)cc1/C=C/CC(C)C]

Following the procedures of Example 14, substituting 3-methylbutanal for 4,4-difluorocyclohexane-carbaldehyde, Example 15 (68 mg, 35.2%) was prepared. ¹H NMR (400 MHz, DMSO-d₆) δ 9.46 (s, 1H), 7.90 (d, J=2.6 Hz, 1H), 7.64 (d, J=2.6 Hz, 1H), 6.50-6.41 (m, 1H), 6.33 (dt, J=15.8, 7.1 Hz, 1H), 3.88 (s, 3H), 2.96 (s, 3H), 2.13-2.07 (m, 2H), 1.77-1.65 (m, 1H), 0.91 (d, J=6.6 Hz, 6H); MS (ESI+) m/z 285.1 (M+H)⁺.

### Example 16

### N-(6-methoxy-5-((E)-2-((1R,4R)-4-(trifluoromethyl)cyclohexyl)vinyl) pyridin-3-yl)methanesulfonamide

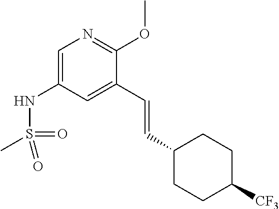 [SMILES: COc1ncc(NS(C)(=O)=O)cc1/C=C/[C@H]1CC[C@H](C(F)(F)F)CC1]

### Step 1: 5-bromo-2-methoxy-3-((E)-2-((1R,4R)-4 (trifluoromethyl) cyclohexyl)vinyl) pyridine

To a mixture of 5-bromo-3-(diethoxyphosphorylmethyl)-2-methoxy-pyridine (750 mg, 2.22 mmol) in THF (12.3 mL) was added sodium hydride (60 mass % in mineral oil) (310 mg, 7.76 mmol), and the reaction mixture was stirred at RT under N₂ for 30 min. A solution of 4-(trifluoromethyl)cyclohexanecarbaldehyde (799 mg, 4.43 mmol) dissolved in THF (5 mL) was then added, and the reaction mixture was stirred at RT for 16 h. The reaction mixture was quenched with water and poured into ⁱPrOAc. The organic layer was washed with water and brine, dried over Na₂SO₄, filtered, and concentrated in vacuo. The crude product was purified by column chromatography (SiO₂: ⁱPrOAc/heptane) to obtain 5-bromo-2-methoxy-3-((E)-2-((1R,4R)-4-(trifluoromethyl)cyclohexyl)vinyl)pyridine (694 mg, 85.9%). ¹H NMR (400 MHz, CDCl₃) δ 8.04 (d, J=2.5 Hz, 1H), 7.71 (d, J=2.3 Hz, 1H), 6.47 (dd, J=16.1, 1.3 Hz, 1H), 6.18 (dd, J=16.1, 7.0 Hz, 1H), 3.94 (s, 3H), 2.20-2.08 (m, 1H), 2.05-1.90 (m, 5H), 1.46-1.32 (m, 2H), 1.28-1.14 (m, 2H); MS (ESI+) m/z 364/365 (M+H)⁺.

Steps 2 to 4

Following the procedures for Example 14, 5-bromo-2-methoxy-3-((E)-2-((1R,4R)-4 (trifluoromethyl)cyclohexyl)vinyl)pyridine (64 mg, 55%) was prepared. ¹H NMR (400 MHz, DMSO-d₆) δ 9.47 (s, 1H), 7.90 (d, J=2.6 Hz, 1H), 7.63 (d, J=2.6 Hz, 1H), 6.46 (dd, J=16.2, 1.2 Hz, 1H), 6.28 (dd, J=16.2, 6.8 Hz, 1H), 3.88 (s, 3H), 2.95 (s, 3H), 2.31-2.10 (m, 2H), 1.96-1.92 (m, 4H), 1.41-1.18 (m, 4H); MS (ESI+) m/z 379.1 (M+H)⁺.

### Example 17

### (E)-N-(6-methoxy-5-(2-(spiro[2.3]hexan-5-yl)vinyl)pyridin-3-yl) methanesulfonamide

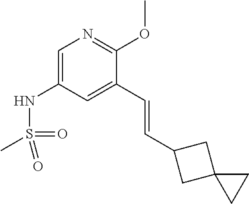 [SMILES: COc1ncc(NS(C)(=O)=O)cc1/C=C/C1CC2(CC2)C1]

Following the procedures of Example 14, substituting spiro[2.3]hexane-5-carbaldehyde for 4-(trifluoromethyl)cyclohexanecarbaldehyde, Example 17 (3.5 mg, 5%) was prepared. ¹H NMR (400 MHz, DMSO-d₆) δ 9.49 (s, 1H), 7.90 (d, J=2.5 Hz, 1H), 7.66 (d, J=2.6 Hz, 1H), 6.55 (dd, J=16.0, 7.3 Hz, 1H), 6.43 (dd, J=16.0, 1.0 Hz, 1H), 3.88 (s, 3H), 3.32-3.21 (m, 1H), 2.96 (s, 3H), 2.27-2.11 (m, 4H), 0.50-0.43 (m, 2H), 0.42-0.35 (m, 2H); MS (ESI+) m/z 309.1 (M+H)⁺.

### Example 18

The overall reaction scheme for Example 18 was as follows:

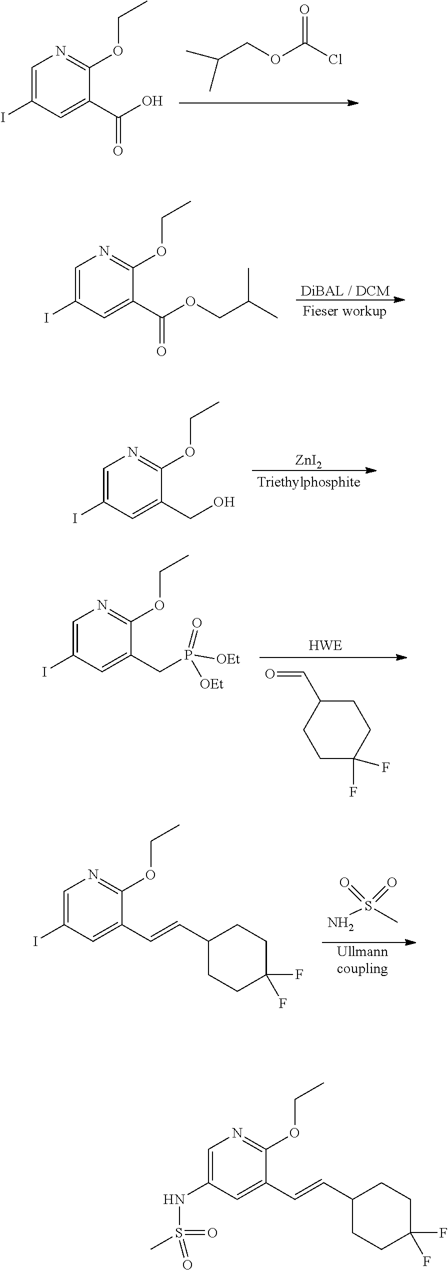

### Step 1: Isobutyl 2-ethoxy-5-iodonicotinate

To a stirred solution of 2-ethoxy-5-iodo-pyridine-3-carboxylic acid (1000 mg, 3.41 mmol), triethylamine (071 mL, 5.11 mmol), and DMAP (41.7 mg, 0.34 mmol) in anhydrous THF (13.6 mL) was added isobutyl chloroformate (536 mg, 3.92 mmol) dropwise at 0° C. under N₂. The reaction mixture was stirred at RT for 3 h. The reaction mixture was quenched with water and diluted with ⁱPrOAc. The organic layer was washed with sat. aq. NH₄Cl solution, water, sat. aq. NaHCO₃ solution, water and brine, dried over Na₂SO₄, filtered, and concentrated in vacuo. The crude product was purified by column chromatography (SiO₂: ⁱPrOAc/heptane) to obtain isobutyl 2-ethoxy-5-iodonicotinate (710 mg, 59.6%) as a white solid. ¹H NMR (400 MHz, CDCl₃) δ 8.43 (d, J=2.5 Hz, 1H), 8.34 (d, J=2.4 Hz, 1H), 4.43 (q, J=7.1 Hz, 2H), 4.08 (d, J=6.6 Hz, 2H), 2.04 (dq, J=13.3, 6.7 Hz, 1H), 1.41 (t, J=7.1 Hz, 3H), 1.01 (d, J=6.8 Hz, 6H).

### Step 2: (2-Ethoxy-5-iodopyridin-3-yl)methanol

To isobutyl 2-ethoxy-5-iodo-pyridine-3-carboxylate (710 mg, 2.03 mmol) in anhydrous DCM (20.3 mL) at −78° C. was added dropwise DIBAL (1.0 mol/L) in heptane (4.1 mL, 4.06 mmol). The reaction mixture was stirred at −78° C. for 1 h and then at RT overnight. The reaction mixture was worked up via the Fieser method to give (2-ethoxy-5-iodopyridin-3-yl)methanol (514.2, 90.6%) as a white solid. ¹H NMR (400 MHz, CDCl₃) δ 8.24 (d, J=2.3 Hz, 1H), 7.84 (dd, J=2.1, 1.1 Hz, 1H), 4.60 (dd, J=6.3, 0.7 Hz, 2H), 4.39 (q, J=7.1 Hz, 2H), 2.18 (t, J=6.4 Hz, 1H), 1.39 (t, J=7.1 Hz, 3H).

### Step 3: Diethyl ((2-ethoxy-5-iodopyridin-3-yl)methyl)phosphonate

An oven-dried flask was charged with anhydrous zinc iodide (671 mg, 2.10 mmol) and purged with N₂. Anhydrous toluene (8.7 mL) was added, followed by triethyl phosphite (0.51 mL, 2.98 mmol), and the resulting mixture was stirred at room temperature for 5 minutes. 3(2-ethoxy-5-iodopyridin-3-yl)methanol (489 mg, 1.75 mmol) dissolved in toluene (8.7 mL) and THF (1 mL for solubility purpose) was then added, and the reaction mixture was stirred under reflux condition (120° C.) for 18 h. The cooled reaction mixture was diluted with ⁱPrOAc/water and filtered through a pad of Celite® to rid the white precipitate. The organic layer from the filtrate was washed with water and brine, dried over Na₂SO₄, filtered, and concentrated in vacuo. The crude product was purified by column chromatography (SiO₂: ⁱPrOAc/heptane followed by MeOH/ⁱPrOAc) to diethyl ((2-ethoxy-5-iodopyridin-3-yl)methyl)phosphonate obtain (492 mg, 70.4%) as an oil. ¹H NMR (400 MHz, CDCl₃) δ 8.18 (t, J=2.4 Hz, 1H), 7.80 (t, J=2.6 Hz, 1H), 4.32 (q, J=7.1 Hz, 2H), 4.09-4.01 (m, 4H), 3.13 (s, 1H), 3.07 (s, 1H) 1.36 (t, J=7.0 Hz, 3H), 1.25 (t, J=7.1 Hz, 6H).

### Step 4: (E)-3-(2-(4,4-Difluorocyclohexyl)vinyl)-2-ethoxy-5-iodopyridine

To a mixture of 3-(diethoxyphosphorylmethyl)-2-ethoxy-5-iodo-pyridine (315 mg, 0.79 mmol) in THF (5 mL) was added sodium hydride (60 mass % in mineral oil) (111 mg, 2.76 mmol), and the reaction mixture was stirred at RT under N2 for 30 min. A solution of 4,4-difluorocyclohexanecarbaldehyde (234 mg, 1.58 mmol) dissolved in THF (5 mL) was then added, and the reaction mixture was stirred at RT for 4 h. The reaction mixture was quenched with sat. aq. NH₄Cl solution and poured into ⁱPrOAc. The organic layer was washed with water and brine, dried over Na₂SO₄, filtered, and concentrated in vacuo. The crude product was purified by column chromatography (SiO₂: ⁱPrOAc/heptane) to obtain (E)-3-(2-(4,4-difluorocyclohexyl)vinyl)-2-ethoxy-5-iodopyridine (304 mg, 98%). MS (ESI+) m/z 394 (M+H)⁺.

### Step 5: (E)-N-(5-(2-(4,4-Difluorocyclohexyl)vinyl)-6-ethoxypyridin-3-yl)methanesulfonamide

In a vial was placed (E)-3-(2-(4,4-difluorocyclohexyl)vinyl)-2-ethoxy-5-iodopyridine (150 mg, 0.38 mmol), methanesulfonamide (181 mg, 1.91 mmol), cuprous iodide (72.6 mg, 0.38 mmol), potassium phosphate (133 mg, 0.76 mmol), and N,N-dimethylglycine (39.7 mg, 0.38 mmol). Degassed DMA (5.5 mL) was added, and the reaction mixture was vacuum purged/back-filled with N₂ (3×) and capped. The reaction mixture was stirred at 100° C. for 3 h, diluted with ⁱPrOAc/water, and filtered through a pad of Celite®. The organic phase from the filtrate was washed with water and brine, dried over Na₂SO₄, filtered, and concentrated in vacuo. The crude product was purified by column chromatography (SiO2: ⁱPrOAc/heptane) followed by reverse-phase preparative HPLC to afford 13 mg (9.5%) of the title compound as a white solid. ¹H NMR (400 MHz, DMSO-d₆) δ 9.45 (s, 1H), 7.89 (d, J=2.6 Hz, 1H), 7.64 (d, J=2.7 Hz, 1H), 6.50 (dd, J=16.2, 1.1 Hz, 1H), 6.34 (dd, J=16.2, 7.0 Hz, 1H), 4.32 (q, J=7.0 Hz, 2H), 2.95 (s, 3H), 2.35 (d, J=9.5 Hz, 1H), 2.04 (d, J=9.6 Hz, 2H), 1.99-1.77 (m, 4H), 1.51-1.36 (m, 2H), 1.33 (t, J=7.0 Hz, 3H); MS (ESI+) m/z 361.1 (M+H)⁺.

### Example 19

### (E)-5-(4-Chlorostyryl)-N-(4-hydroxybutan-2-yl)-6-methoxynicotinamide

The overall Example 19 reaction scheme was as follows:

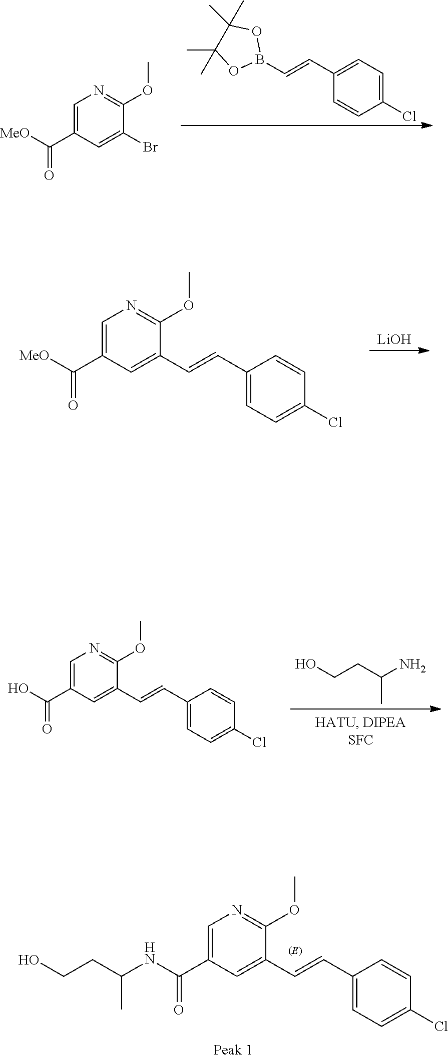

### Step 1: Methyl (E)-5-(4-chlorostyryl)-6-methoxynicotinate

A microwave vial was charged with methyl 5-bromo-6-methoxy-pyridine-3-carboxylate (600 mg, 2.44 mmol), 2-[(E)-2-(4-chlorophenyl)vinyl]-4,4,5,5-tetramethyl-1,3,2-dioxaborolane (968 mg, 3.66 mmol), Pd(dppf)Cl₂ complexed with DCM (62 mg, 0.073 mmol), sodium carbonate (440 mg, 4.14 mmol), potassium acetate (440 mg, 4.47 mmol), ACN (16 mL), and water (4 mL). The reaction mixture was vacuum purged/back-filled with N₂ (3×), and the vial was capped. The reaction mixture was microwaved at 120° C. for 40 min, diluted with iPrOAc, and filtered through a pad of Celite®. The organic phase from the filtrate was washed with water and brine, dried over Na₂SO₄, filtered, and concentrated in vacuo. The crude product was purified by column chromatography (SiO₂: ⁱPrOAc/heptane followed by MeOH/PrOAc) to give 350 mg (47.3%) of methyl (E)-5-(4-chlorostyryl)-6-methoxynicotinate. ¹H NMR (400 MHz, CDCl₃) δ 8.72 (d, J=2.2 Hz, 1H), 8.37 (d, J=2.3 Hz, 1H), 7.49-7.44 (m, 2H), 7.36-7.32 (m, 2H), 7.21 (d, J=6.2 Hz, 2H), 4.09 (s, 3H), 3.94 (s, 3H).

### Step 2: (E)-5-(4-Chlorostyryl)-6-methoxynicotinic Acid

A mixture of methyl (E)-5-(4-chlorostyryl)-6-methoxynicotinate (350 mg, 1.15 mmol) and lithium hydroxide (83 mg, 3.46 mmol) in THF (10.5 mL) and water (7.7 mL) was stirred at 40° C. for 2 h, then left stirring at RT overnight. The reaction was acidified with 1N HCl until pH 5, and the mixture was extracted with EtOAc (3×). The combined organic layers was washed with water and brine, dried over Na₂SO₄, filtered, and concentrated in vacuo to get 224 mg (67.1%) of (E)-5-(4-chlorostyryl)-6-methoxynicotinic acid as a white solid. ¹H NMR (400 MHz, DMSO-d₆) δ 13.10 (s, 1H), 8.64 (d, J=2.1 Hz, 1H), 8.44 (d, J=2.2 Hz, 1H), 7.70-7.62 (m, 2H), 7.50-7.40 (m, 3H), 7.32 (d, J=16.6 Hz, 1H), 4.04 (s, 3H).

### Step 3: (E)-5-(4-Chlorostyryl)-N-(4-hydroxybutan-2-yl)-6-methoxynicotinamide

A mixture of (E)-5-(4-chlorostyryl)-6-methoxynicotinic acid (200 mg, 0.69 mmol), 3-amino-butan-1-ol (92.3 mg, 1.04 mmol), HATU (472.4 mg, 1.24 mmol), and DIPEA (0.24 mL, 1.38 mmol) in anhydrous DMF (3.5 mL) was stirred at RT for 18 h. The reaction mixture was diluted with ⁱPrOAc. The organic phase was washed with water and brine, dried over Na₂SO₄, filtered, and concentrated in vacuo. The crude product was purified by column chromatography (SiO₂: ⁱPrOAc/heptane and MeOH/ⁱPrOAc) followed by SFC chiral separation (Chiralcel OX, isocratic 30% MeOH w/ 0.1% NH₄OH, 40° C., 2.5 min). The first peak was collected to give the title compound (101 mg, 39.4%) as a white solid. Chiral SFC Peak 1 (RT=0.695 min), % ee=100; ¹H NMR (400 MHz, DMSO-d₆) δ 8.55 (d, J=2.3 Hz, 1H), 8.41 (d, J=2.4 Hz, 1H), 8.24 (d, J=8.1 Hz, 1H), 7.69-7.62 (m, 2H), 7.50-7.43 (m, 2H), 7.40 (d, J=16.6 Hz, 1H), 7.31 (d, J=16.6 Hz, 1H), 4.43 (t, J=5.1 Hz, 1H), 4.20-4.07 (m, 1H), 4.01 (s, 3H), 3.51-3.42 (m, 2H), 1.79-1.58 (m, 2H), 1.17 (d, J=6.6 Hz, 3H); MS (ESI+) m/z 361.1 (M+H)⁺.

### Example 20

### (E)-N-(5-methoxy-4-(2-(1,4,4-trifluorocyclohexyl)vinyl)pyridin-2-yl) methanesulfonamide

The overall Example 20 reaction scheme was as follows:

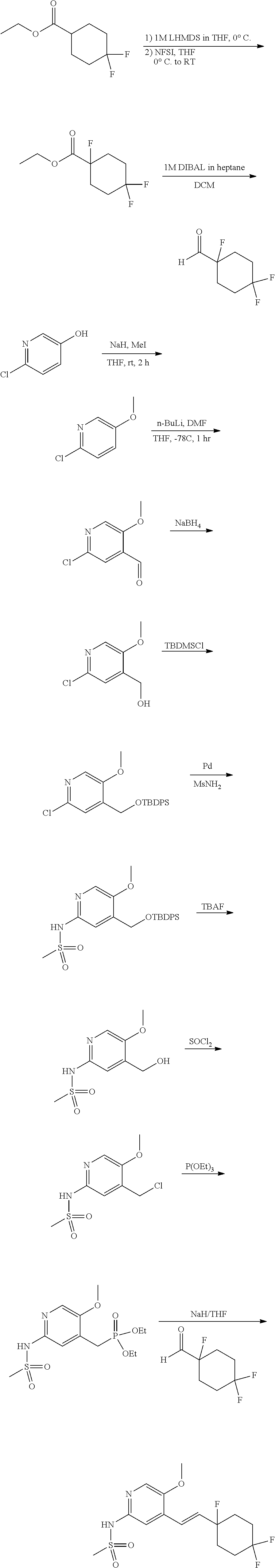

### Step 1: Ethyl 1,4,4-trifluorocyclohexane-1-carboxylate

To ethyl 4,4-difluorocyclohexanecarboxylate (1.80 g, 9.37 mmol) dissolved in anhydrous THF (19 mL) at 0° C. was added lithium bis(trimethylsilyl)amide (1 mol/L) in THF (14 mL). The resultant light yellow reaction mixture was stirred at 0° C. under N₂ for 1 h. N-Fluorobenzenesulfonimide (5.02 g, 15.9 mmol) dissolved in THF (10 mL) was then added, and the reaction mixture was stirred at RT for 3 h. The reaction mixture was quenched with 10% aq. HCl and then stirred at RT for at least 1 h. The reaction mixture was diluted with PrOAc. The organic layer was washed with water and brine, dried over Na₂SO₄, filtered, and concentrated in vacuo. The crude product was purified by column chromatography (SiO₂: iPrOAc/heptane) to collect ethyl 1,4,4-trifluorocyclohexane-1-carboxylate (1.45 g, 73.5%) as a yellow oil. ¹H NMR (400 MHz, CDCl₃) δ 4.26 (q, J=7.2 Hz, 2H), 2.26-1.98 (m, 8H), 1.32 (t, J=7.1 Hz, 3H).

### Step 2: 1,4,4-Trifluorocyclohexane-1-carbaldehyde

To ethyl 1,4,4-trifluorocyclohexanecarboxylate (500 mg, 2.38 mmol) in anhydrous DCM (20 mL) at −78° C. was added dropwise DiBAL (1.0 mol/L) in heptane (2.3 mL, 2.30 mmol). The reaction mixture was stirred at −78° C. for 2 h. The reaction was worked up via the Fieser method. The white precipitate was filtered, and the filtrate was evaporated under reduced pressure until 20 mL of solvent was left. Assumed quantitative yield and used without further purification for the next reaction step.

### Step 3: 2-Chloro-5-methoxypyridine

2-Chloro-5-hydroxypyridine (25 g, 193 mmol), potassium carbonate (53.3 g, 386 mmol), and methyl iodide (14.5 mL, 223 mmol) were combined in a flask of acetonitrile (500 mL, 0.2M) under nitrogen. The reaction mixture was stirred at room temperature overnight and then diluted with water (1 L). The reaction mixture was extracted with hexanes (3×500 mL). The combined organic layers were washed with brine, dried over sodium sulfate, and concentrated under reduced pressure. This crude residue was purified over a pad of silica eluted with hexanes (400 mL), and the filtrate was concentrated under reduced pressure to give 2-chloro-5-methoxypyridine as a yellow oil (21.7 g, 78.3%). H NMR (400 MHz, DMSO-d₆) δ 8.13 (d, J=2.9 Hz, 1H), 7.51-7.47 (m, 1H), 7.45-7.42 (m, 1H), 3.84 (s, 3H); MS (ESI+) m/z 144.1 (M+H)⁺.

### Step 4: 2-chloro-5-methoxyisonicotinaldehyde

To 2-chloro-5-methoxypyridine (10 g, 57.6 mmol) dissolved in anhydrous THF (150 mL, 0.2 M) under nitrogen was added dropwise 2.5 M n-BuLi in hexanes (42.8 mL, 108 mmol) at −78° C., taking care to keep the temperature constant. The reaction mixture was stirred at −78° C. for 30 minutes, and DMF (10.5 mL, 135 mmol) was added dropwise, maintaining the temperature at −78° C. The resulting solution was stirred for another 30 minutes at −78° C., then poured slowly into saturated ammonium chloride solution. The aqueous mixture was placed in a separatory funnel, and the organics were extracted with ethyl acetate (3×300 mL). The combined organic layers were washed with brine, dried over sodium sulfate, and concentrated under reduced pressure to give dark brown oil. The crude product was purified by column chromatography (SiO₂: EtOAc/hexanes) to obtain a beige crystalline solid (2.77 g, 22.6%). ¹H NMR (400 MHz, DMSO-d₆) δ 10.29 (s, 1H), 8.55 (s, 1H), 7.58 (s, 1H), 4.05 (s, 3H); MS (ESI+) m/z 172.2 (M+H)⁺.

### Step 5: (2-chloro-5-methoxypyridin-4-yl)methanol

To 2-chloro-5-methoxyisonicotinaldehyde (20.1 g. 117.2 mmol) in THF (585 mL, 0.2M) was added sodium borohydride (4.43 g, 117.2 mmol), and the resulting mixture was stirred for 2 h. The crude mixture was quenched with 100 ml methanol, followed by 1M HCl solution. The reaction mixture was then neutralized with sat. aqueous sodium bicarbonate solution. The aqueous mixture was placed in a separatory funnel, and the organics were extracted with EtOAc (3×600 mL). The combined organic phases were washed with brine, dried over sodium sulfate, and concentrated under reduced pressure to yield a yellow sticky solid, which was triturated with 50% DCM/hexanes (10 mL) and filtered. The filtrate was concentrated and purified by column chromatography (SiO₂: EtOAc/hexanes) to give 2-chloro-5-methoxypyridin-4-yl)methanol (8.01 g, 39.4%). ¹H NMR (400 MHz, DMSO-d₆) δ 8.07 (s, 1H), 7.38 (s, 1H), 5.46 (t, J=5.7 Hz, 1H), 4.50 (d, J=5.5 Hz, 2H), 3.89 (s, 3H); MS (ESI+) m/z 174.0 (M+H)⁺.

### Step 6: 4-(((Tert-butyldiphenylsilyl)oxy)methyl)-2-chloro-5-methoxypyridine

To a stirred solution of (2-chloro-5-methoxypyridin-4-yl)methanol (1.34 g, 7.72 mmol) in DCM (40.5 mL, 0.2M) at 0° C. was added imidazole (1.05 g, 15.4 mmol) and TBDPSCl (2.76 g, 10.0 mmol), and the reaction mixture was stirred overnight. The reaction mixture was diluted with water (20 mL) and then extracted with DCM (3×40 mL). The combined organic layers were washed with brine, dried over sodium sulfate, and concentrated under reduced pressure. The crude product was purified by column chromatography (SiO₂: EtOAc/hexanes) to give 4-(((tert-butyldiphenylsilyl)oxy)methyl)-2-chloro-5-methoxypyridine as a white solid (2.52 g, 79.4%). ¹H NMR (400 MHz, DMSO-d₆) δ 8.09 (s, 1H), 7.63 (d, J=6.9 Hz, 4H), 7.52-7.43 (m, 7H), 4.71 (s, 2H), 3.80 (s, 3H), 1.06 (s, 9H); MS (ESI+) m/z 412.0 (M+H)⁺.

### Step 7: N-(4-(((tert-butyldiphenylsilyl)oxy)methyl)-5-methoxypyridin-2-yl)methanesulfonamide

In an argon-filled flame-dried flask was charged with 4-(((tert-butyldiphenylsilyl)oxy)methyl)-2-chloro-5-methoxypyridine (4.50 g, 10.9 mmol) and 2-methy-2-butanol (90 mL, 0.12 M). The flask was then vacuum purged and filled with argon twice. Methanesulfonamide (2.08 g, 21.8 mmol), potassium phosphate (4.64 g, 21.8 mmol), and [Pd(allyl)(t-BuXPhos)]OTf (0.24 g, 0.328 mmol) were added under argon. The flask was vacuum purged and filled with argon twice, and an argon balloon was inserted in the septa. This mixture was then immersed in a pre-heated oil bath at 110° C. for 24 h, monitored by LCMS. The reaction was cooled to room temperature and quenched with sat. aqueous ammonium chloride solution (30 mL). The reaction mixture was extracted with EtOAc (3×150 mL), dried over sodium sulfate, and concentrated under reduced pressure. The crude product was purified by column chromatography (SiO₂: EtOAc/hexanes) to give N-(4-(((tert-butyldiphenylsilyl)oxy)methyl)-5-methoxypyridin-2-yl)methanesulfonamide as pink chalky solid (3.46 g, 67.3%). ¹H NMR (400 MHz, DMSO-d₆) δ 10.52-10.39 (m, 1H), 7.93 (s, 1H), 7.68-7.63 (m, 4H), 7.51-7.43 (m, 6H), 7.40 (s, 1H), 4.68 (s, 2H), 3.76 (s, 3H), 3.26 (s, 3H), 1.07 (s, 9H); MS (ESI+) m/z 471.0 (M+H)⁺.

### Step 8: N-(4-(hydroxymethyl)-5-methoxypyridin-2-yl) methanesulfonamide

To N-(4-(((tert-butyldiphenylsilyl)oxy)methyl)-5-methoxypyridin-2-yl)methanesulfonamide (2.3 g, 4.89 mmol) dissolved in THF (23 mL) was added tetrabutylammoniumfluoride (9.77 mL, 9.77 mmol, 1M in THF), and the reaction mixture was stirred at room temperature for 4 h. The reaction mixture was partitioned between EtOAc (100 mL) and water (100 mL) and separated. The aqueous phase was extracted with EtOAc (8×50 mL). The combined organic layers were dried over anhydrous Na₂SO₄, filtered and evaporated in vacuo to give an off-white solid. The crude solid was triturated with diethyl ether (5 mL) to give N-(4-(hydroxymethyl)-5-methoxypyridin-2-yl)methanesulfonamide as white solid (1 g, 88.1%). ¹H NMR (400 MHz, MeOD-d₄) δ 7.87 (s, 1H), 7.24 (s, 1H), 4.64 (s, 2H), 3.90 (s, 3H), 3.21 (s, 3H); MS (ESI+) m/z 233.1 (M+H)⁺.

### Step 9: N-(4-(chloromethyl)-5-methoxypyridin-2-yl)methanesulfonamide HCl Salt

To N-(4-(hydroxymethyl)-5-methoxypyridin-2-yl)methanesulfonamide (300 mg, 1.29 mmol) in DCM (3.0 mL) was added thionyl chloride (0.38 mL, 5.17 mmol), and the reaction mixture was stirred at room temperature for 1 h. The product precipitated and the mixture was concentrated, treated with ether, concentrated, treated with ether and concentrated again to give N-(4-(chloromethyl)-5-methoxypyridin-2-yl)methanesulfonamide as the HCl salt (371 mg, quantitative yield). ¹H NMR (400 MHz, MeOD-d₄) δ 8.03 (s, 1H), 7.41 (s, 1H), 4.74 (s, 2H), 4.00 (s, 3H), 3.28 (s, 3H).

### Step 10: N-[4-(diethoxyphosphorylmethyl)-5-methoxy-2-pyridyl]methanesulfonamide

N-(4-(chloromethyl)-5-methoxypyridin-2-yl)methanesulfonamide HCl salt (0.3 g, 1.20 mmol) and triethyl phosphite (1.03 mL, 5.98 mmol) were combined and heated at 140° C. for 6 h. The reaction mixture was evaporated under reduced pressure to yield light yellow oil. The crude oil was purified by column chromatography (SiO₂: MeOH/EtOAc) to give N-[4-(diethoxyphosphorylmethyl)-5-methoxy-2-pyridyl]methanesulfonamide as an off-white solid (0.28 g, 66.4%). ¹H NMR (400 MHz, CDCl₃) δ 10.03 (br s, 1H), 8.05 (s, 1H), 7.38 (d, J=2.8 Hz, 1H), 4.17-4.06 (m, 4H), 3.92 (s, 3H), 3.29 (s, 1H), 3.23 (s, 1H), 3.11 (s, 3H), 1.30 (t, J=7.0 Hz, 6H); MS (ESI+) m/z 353.0 (M+H)⁺.

### Step 11: (E)-N-(5-methoxy-4-(2-(1,4,4-trifluorocyclohexyl)vinyl)pyridin-2-yl)methanesulfonamide

To a mixture of N-[4-(diethoxyphosphorylmethyl)-5-methoxy-2-pyridyl]methanesulfonamide (295 mg, 0.84 mmol) in THF (16.7 mL) was added sodium hydride (60 mass % in mineral oil) (168 mg, 4.18 mmol), and the reaction mixture was stirred at RT under N₂ for 30 min. A solution of 1,4,4-trifluorocyclohexane-1-carbaldehyde in ether/DCM (395 mg, 2.38 mmol) from step 2 was then added, and the reaction mixture was stirred at RT for 4 h. The reaction mixture was quenched with sat. aq. NH₄Cl solution and poured into ⁱPrOAc. The organic layer was washed with water and brine, dried over Na₂SO₄, filtered, and concentrated in vacuo. The crude product was purified by column chromatography (SiO₂: ⁱPrOAc/heptane and MeOH/PrOAc) followed by reverse-phase preparative HPLC to afford 49.7 mg (16.3%) of the title compound as a white solid. ¹H NMR (400 MHz, DMSO-d₆) δ 10.19 (s, 1H), 8.08 (s, 1H), 7.07 (s, 1H), 6.81 (d, J=16.5 Hz, 1H), 6.74-6.62 (m, 1H), 3.88 (s, 3H), 3.22 (s, 3H), 2.13-1.94 (m, 8H); MS (ESI+) m/z 365.1 (M+H)⁺.

### Example 21

### N-(4-((E)-2-((1s,4s)-1-fluoro-4-(trifluoromethyl)cyclohexyl)vinyl)-5-methoxypyridin-2-yl)methanesulfonamide

The overall Example 21 reaction scheme was as follows:

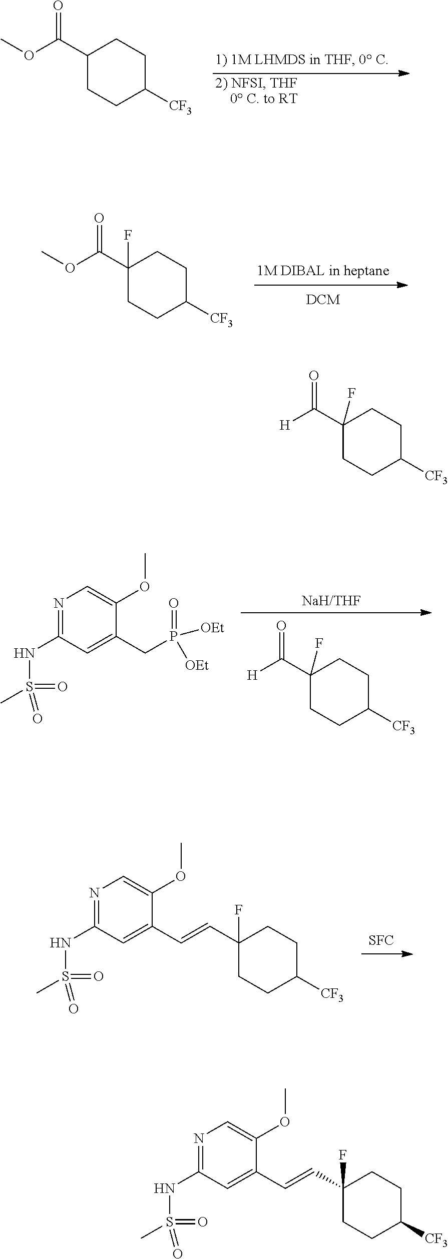

Following the procedures of Example 20, substituting methyl 4-(trifluoromethyl)cyclohexane-1-carboxylate for ethyl 4,4-difluorocyclohexanecarboxylate, racemic 21 was obtained. Chiral SFC separation (Chiralpak 1A, isocratic 10% MeOH w/ 0.1% NH₄OH, 40° C., 2.5 min) of racemic N-(4-((E)-2-(1-fluoro-4-(trifluoromethyl)cyclohexyl)vinyl)-5-methoxypyridin-2-yl)methanesulfonamide afforded the title compound (6.4 mg, 1.4%) as a white solid. Chiral SFC Peak 2 (RT=1.189 min), % ee=98.4; ¹H NMR (400 MHz, DMSO-d₆) δ 10.12 (s, 1H), 8.09 (s, 1H), 7.14 (s, 1H), 6.87 (dd, J=16.4, 2.1 Hz, 1H), 6.74 (dd, J=16.4, 15.3 Hz, 1H), 3.89 (s, 3H), 3.23 (s, 3H), 2.13-1.67 (m, 7H), 1.61-1.41 (m, 2H); MS (ESI+) m/z 397.1 (M+H)⁺.

### Example 22

### N-(1-(4-Chlorobenzyl)-3-methyl-1H-indol-6-yl)methanesulfonamide

The overall Example 22 reaction scheme was as follows:

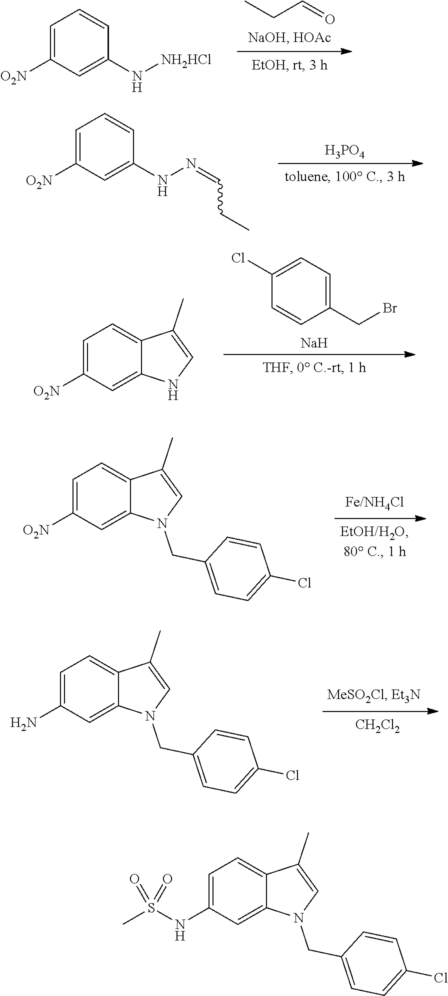

### Step 1: 1-(3-Nitrophenyl)-2-propylidenehydrazine

To a solution of 1-(3-nitrophenyl)hydrazine hydrochloride (10.0 g, 52.74 mmol) in EtOH (100 mL) was added 15% aqueous NaOH solution (50 mL) to adjust pH to 6. AcOH (24.36 mL, 421.94 mmol) and propionaldehyde (3.68 g, 63.29 mmol) were then added to the above mixture. The reaction mixture was stirred at 25° C. for 3 hours. The mixture was then poured into ice water and the precipitate was filtered, washed with water and was dried in vacuo to afford the title compound (11.5 g crude) as a yellow solid. H NMR (400 MHz, CD₃OD) δ 7.78-7.77 (m, 1H), 7.53-7.50 (m, 1H), 7.36 (t, J=8.0 Hz, 1H), 7.26-7.21 (m, 2H), 2.35-2.28 (m, 2H), 1.15 (t, J=7.6 Hz, 3H).

### Step 2: 3-Methyl-6-nitro-1H-indole

A mixture of 1-(3-nitrophenyl)-2-propylidenehydrazine (From step 1, 11.5 g, 59.5 mmol) in H₃PO₄ (100 mL) and toluene (100 mL) was stirred at 100° C. for 3 hours. The reaction mixture was diluted with water (300 mL), extracted with EtOAc (300 mL×2). The combined organic layers were washed with aqueous 10% NaOH solution (300 mL), dried over anhydrous Na₂SO₄, filtered and concentrated. The crude residue was purified by silica gel column chromatography (30% EtOAc in petroleum ether) to afford the title compound (3.5 g, 33%) as a yellow solid. ¹H NMR (400 MHz, CD₃OD) δ 8.28 (s, 1H), 7.90 (d, J=8.8 Hz, 1H), 7.57 (d, J=8.8 Hz, 1H), 7.35 (s, 1H), 2.32 (s, 3H).

### Step 3: 1-(4-Chlorobenzyl)-3-methyl-6-nitro-1H-indole

To a stirred solution of 3-methyl-6-nitro-1H-indole (From step 2, 3.5 g, 19.9 mmol) in THF (50 mL) in an ice bath was added NaH (60% in mineral oil, 1.19 g, 29.8 mmol). The reaction mixture was stirred for 30 minutes and 1-(bromomethyl)-4-chlorobenzene (6.12 g, 29.8 mmol) was added to the mixture. The reaction mixture was further stirred at 25° C. for 3 hours. Water (100 mL) was added to the reaction mixture. The mixture was extracted with EtOAc (100 mL×2). The combined organic layers were dried over anhydrous Na₂SO₄, filtered and concentrated. The crude residue was purified by silica gel column chromatography (20% EtOAc in petroleum ether) to afford the title compound (4.5 g, 75%) as a yellow solid. LCMS (ESI+) m/z 300.9 (M+H)⁺.

### Step 4: 1-(4-Chlorobenzyl)-3-methyl-1H-indol-6-amine

A mixture of 1-(4-chlorobenzyl)-3-methyl-6-nitro-1H-indole (From step 3, 4.5 g, 14.96 mmol), iron powder (4.18 g, 74.82 mmol) and NH₄Cl (4.8 g, 89.78 mmol) in EtOH (100 mL) and water (20 mL) was stirred at 80° C. for 3 hours. After being cooled to 25° C., the reaction mixture was filtered through a Celite® pad, washed with MeOH (100 mL). The filtrate was concentrated to dryness. The crude residue was purified by silica gel column chromatography (40% EtOAc in petroleum ether) to afford the title compound (3.4 g, 84%) as a yellow oil. ¹H NMR (400 MHz, CDCl₃) δ 7.35 (d, J=8.4 Hz, 1H), 7.25 (d, J=8.0 Hz, 2H), 7.02 (d, J=8.0 Hz, 2H), 6.67 (s, 1H), 6.58 (dd, J=8.0 Hz, 2.0 Hz, 1H), 6.46 (s, 1H), 5.10 (s, 2H), 3.60 (br s, 2H), 2.28 (s, 3H).

### Step 5: N-(1-(4-Chlorobenzyl)-3-methyl-1H-indol-6-yl) methanesulfonamide

To a mixture of 1-(4-chlorobenzyl)-3-methyl-1H-indol-6-amine (From step 4, 150 mg, 0.55 mmol) in pyridine (3 mL) was added methanesulfonyl chloride (0.06 mL, 0.83 mmol). The reaction mixture was stirred at 25° C. for 2 hours. The reaction mixture was diluted with water (20 mL), extracted with EtOAc (20 mL×2). The combined organic layers were dried over anhydrous Na₂SO₄, filtered and concentrated. The resulting residue was purified by reverse phase chromatography (acetonitrile 50-80/0.05% NH₄OH in water) to afford the title compound (139 mg, 72%) as a white solid. ¹H NMR (400 MHz, CD₃OD) δ 7.49 (d, J=8.0 Hz, 1H), 7.29 (d, J=8.4 Hz, 2H), 7.21 (s, 1H), 7.12 (d, J=8.4 Hz, 2H), 7.05 (s, 1H), 6.98-6.95 (m, 1H), 5.28 (s, 2H), 2.84 (s, 3H), 2.31 (s, 3H). LCMS (ESI+) m/z 348.9 (M+H)⁺.

### Example 23

### N-(1-(4-Chlorobenzyl)-3-methyl-1H-indol-6-yl)cyclopropanesulfonamide

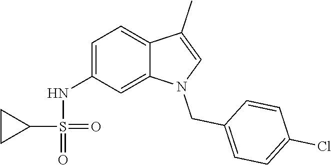 [SMILES: Cc1cn(Cc2ccc(Cl)cc2)c2cc(NS(=O)(=O)C3CC3)ccc12]

Following a similar procedure to that of step 5 of Example 22, the title compound was prepared from 1-(4-chlorobenzyl)-3-methyl-1H-indol-6-amine and cyclopropanesulfonyl chloride to furnish the title compound as a white solid (105 mg, 50%). H NMR (400 MHz, DMSO-d₆) δ 9.37 (s, 1H), 7.41 (d, J=8.4 Hz, 1H), 7.34 (d, J=8.4 Hz, 2H), 7.19-7.17 (m, 2H), 7.12 (d, J=8.4 Hz, 2H), 6.93-6.90 (m, 1H), 5.27 (s, 2H), 2.42-2.36 (m, 1H), 2.22 (s, 3H), 0.77-0.75 (m, 4H). LCMS (ESI+) m/z 375.0 (M+H)⁺.

### Example 24

### N-(3-(4-Chlorobenzyl)-1-methyl-1H-indol-5-yl)methanesulfonamide

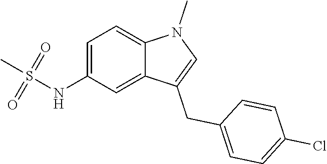 [SMILES: Cn1cc(Cc2ccc(Cl)cc2)c2cc(NS(C)(=O)=O)ccc21]

### Step 1: 3-(4-Chlorobenzyl)-1-methyl-5-nitro-1H-indole

A mixture of 1-methyl-5-nitro-indole (1.0 g, 5.7 mmol), Cu₂O (2.4 g, 17 mmol), 1-(bromomethyl)-4-chlorobenzene (1.5 g, 7.4 mmol) in acetonitrile (30 mL, 287 mmol) was stirred at 138° C. for 16 hours. The reaction mixture was filtered and concentrated. The residue was further purified by prep-HPLC (acetonitrile 0-55/0.225% FA in water) to afford the title compound (300 mg, 18%) as a yellow solid. ¹H NMR (400 MHz, CDCl₃) δ 8.47 (d, J=2.0 Hz, 1H), 8.14 (dd, J=9.2, 2.0 Hz, 1H), 7.34-7.28 (m, 2H), 7.27-7.16 (m, 3H), 6.89 (s, 1H), 4.10 (s, 2H), 3.81 (s, 3H).

### Step 2: 3-(4-Chlorobenzyl)-1-methyl-1H-indol-5-amine

Following a similar procedure to that of Step 4 of Example 22, the title compound was obtained from 3-(4-chlorobenzyl)-1-methyl-5-nitro-1H-indole as a brown solid (400 mg, 98%). ¹H NMR (400 MHz, CDCl₃) δ 7.26-7.17 (m, 4H), 7.11 (d, J=8.4 Hz, 1H), 6.78-6.67 (m, 3H), 3.99 (s, 2H), 3.69 (s, 3H).

### Step 3: N-(3-(4-Chlorobenzyl)-1-methyl-1H-indol-5-yl) methanesulfonamide

Following a similar procedure to that of Step 5 of Example 22, the title compound was prepared from 3-(4-chlorobenzyl)-1-methyl-1H-indol-5-amine (From step 2) and methanesulfonyl chloride furnish the title compound as a yellow solid (63.9 mg, 50%). ¹H NMR (400 MHz, DMSO-d₆) δ 8.02 (br s, 1H), 7.39-7.24 (m, 6H), 7.14 (s, 1H), 7.06-7.00 (m, 1H), 3.99 (s, 2H), 3.72 (s, 3H), 2.81 (s, 3H). LCMS (ESI+) m/z 348.9 (M+H)⁺.

### Example 25

### N-(4′-Isopropyl-6-methoxy-[1,1′-biphenyl]-3-yl)cyclopropane sulfonamide

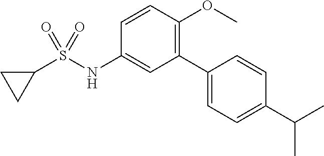 [SMILES: COc1ccc(NS(=O)(=O)C2CC2)cc1-c1ccc(C(C)C)cc1]

### Step 1: 4′-Isopropyl-6-methoxy-[1,1′-biphenyl]-3-amine

A mixture of 3-bromo-4-methoxyaniline (500 mg, 2.47 mmol), 4-isopropylphenyl boronic acid (487 mg, 2.97 mmol), Pd(dppf)Cl₂ (181 mg, 0.25 mmol) and Na₂CO₃ (787 mg, 7.42 mmol) in 1,4-dioxane (10 mL) and water (1 mL) was stirred at 100° C. for 8 hours under N₂ atmosphere. After being cooled to 25° C., the reaction mixture was diluted with water (50 mL), extracted with EtOAc (50 mL×2). The combined organic layers were dried over anhydrous Na₂SO₄, filtered and concentrated. The crude residue was purified by silica gel column chromatography (40% EtOAc in petroleum ether) to afford the title compound (510 mg, 85%) as a yellow oil. ¹H NMR (400 MHz, CDCl₃) δ 7.45 (d, J=8.0 Hz, 1H), 7.26 (d, J=8.0 Hz, 1H), 6.82 (d, J=8.4 Hz, 1H), 6.71 (d, J=2.8 Hz, 1H), 6.67-6.64 (m, 1H), 3.72 (s, 3H), 2.97-2.90 (m, 1H), 1.28 (d, J=7.2 Hz, 6H).

### Step 2: N-(4′-Isopropyl-6-methoxy-[1,1′-biphenyl]-3-yl) cyclopropanesulfonamide

Following a similar procedure to that of step 5 of Example 22, the title compound was prepared from 4′-isopropyl-6-methoxy-[1,1′-biphenyl]-3-amine and cyclopropanesulfonyl chloride which furnish the title compound as a white solid (52 mg, 30%). ¹H NMR (400 MHz, CD₃OD) δ 7.39 (d, J=8.0 Hz, 2H), 7.26-7.18 (m, 4H), 7.04 (d, J=8.0 Hz, 1H), 3.78 (s, 3H), 2.96-2.89 (m, 1H), 2.52-2.46 (m, 1H), 1.28 (d, J=6.8 Hz, 6H), 0.99-0.92 (m, 4H). LCMS (ESI+) m/z 346.0 (M+H)⁺.

### Example 26

### 4′-Cyclopropyl-N-isopropyl-6-methoxy-[1,1′-biphenyl]-3-carboxamide

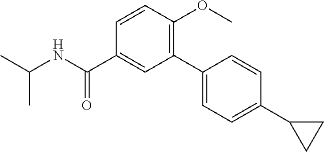 [SMILES: COc1ccc(C(=O)NC(C)C)cc1-c1ccc(C2CC2)cc1]

### Step 1: 3-Bromo-N-isopropyl-4-methoxybenzamide

A mixture of 3-bromo-4-methoxybenzoic acid (500 mg, 2.16 mmol), oxalyl chloride (0.27 mL, 3.25 mmol) and DMF (0.5 mL) in DCM (20 mL) was stirred at 0° C. for 2 hours. The reaction mixture was concentrated to afford the crude 3-bromo-4-methoxy-benzoyl chloride (500 mg, 93%) as a white solid. A mixture of the resulting 3-bromo-4-methoxy-benzoyl chloride and isopropyl amine (118 mg, 2 mmol), triethylamine (202 mg, 2 mmol) in DCM (20 mL) was stirred at 25° C. for 2 hours. Water (40 mL) was added to the reaction mixture and the mixture was extracted with DCM (40 mL). The organic layer was washed with water (50 mL×2) and was dried over anhydrous Na₂SO₄ and concentrated. The residue was purified by chromatography on silica gel (0-50% EtOAc in petroleum ether) to afford the title compound (540 mg, 99%) as a white solid. ¹H NMR (400 MHz, CDCl₃) δ 7.93 (d, J=2.4 Hz, 1H), 7.74 (dd, J=8.4, 2.4 Hz, 1H), 6.91 (d, J=8.8 Hz, 1H), 5.87 (d, J=5.2 Hz, 1H), 4.30-4.22 (m, 1H), 3.94 (s, 3H), 1.26 (d, J=6.4 Hz, 6H).

### Step 2: N-Isopropyl-4-methoxy-3-(4,4,5,5-tetramethyl-1,3,2-dioxaborolan-2-yl) benzamide

A mixture of 3-bromo-N-isopropyl-4-methoxy-benzamide (7.2 g, 26 mmol), bis(pinacolato)diboron (8.0 g, 31 mmol), 1,1′-bis(diphenylphosphino)ferrocene palladium dichloride (2.0 g, 2.65 mmol) and potassium acetate (7.8 g, 79 mmol) in 1,4-dioxane (100 mL) was stirred at 100° C. for 16 hours under N₂ atmosphere. After being cooled to 25° C., the reaction mixture was diluted with water (200 mL), extracted with EtOAc (200 mL×2). The combined organic layers were dried over anhydrous Na₂SO₄, filtered and concentrated. The crude residue was purified by silica gel column chromatography (60% EtOAc in petroleum ether) to afford the title compound (5.8 g, 69%) as a white solid. ¹H NMR (400 MHz, CDCl₃) δ 7.95-7.93 (m, 2H), 6.88 (d, J=8.8 Hz, 1H), 5.96 (d, J=7.6 Hz, 1H), 4.31-4.23 (m, 1H), 3.86 (s, 3H), 1.36 (s, 12H), 1.25 (d, J=6.4 Hz, 6H).

### Step 3: 4′-Cyclopropyl-N-isopropyl-6-methoxy-[1,1′-biphenyl]-3-carboxamide

A mixture of N-isopropyl-4-methoxy-3-(4,4,5,5-tetramethyl-1,3,2-dioxaborolan-2-yl) benzamide (150.0 mg, 0.47 mmol), 1-bromo-4-cyclopropyl-benzene (111.1 mg, 0.56 mmol), 1,1′-bis(diphenylphosphino)ferrocene palladium dichloride (34.4 mg, 0.05 mmol) and sodium carbonate (149.4 mg, 1.41 mmol) in 1,4-dioxane (5 mL) and water (1 mL) was stirred at 100° C. for 3 hours under N₂ atmosphere. After being cooled to 25° C., the reaction mixture was diluted with water (20 mL), extracted with EtOAc (20 mL×2). The combined organic layers were concentrated. The resulting residue was purified by prep-HPLC (base) to afford the title compound (55.5 mg, 38%) as a white solid. H NMR (400 MHz, DMSO-d₆) δ 8.14 (d, J=7.6 Hz, 1H), 7.84 (d, J=8.0 Hz, 1H), 7.79 (s, 1H), 7.38 (d, J=8.0 Hz, 2H), 7.18-7.07 (m, 3H), 4.17-4.00 (m, 1H), 3.80 (s, 3H), 1.97-1.91 (m, 1H), 1.15 (d, J=6.4 Hz, 6H), 0.98-0.96 (m, 2H), 0.72-0.68 (m, 2H); LCMS (ESI+) m/z 310.1 (M+H)⁺.

### Example 27

### (E)-N-(3-(2-Cyclohexylvinyl)-4-methoxyphenyl)methanesulfonamide

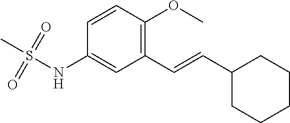 [SMILES: COc1ccc(NS(C)(=O)=O)cc1/C=C/C1CCCCC1]

### Step 1: (E)-3-(2-Cyclohexylvinyl)-4-methoxyaniline

To a solution of 3-bromo-4-methoxyaniline (150 mg, 0.74 mmol) in 1,4-dioxane (10 mL), water (1 mL) was added 1,1′-bis(diphenylphosphino)ferrocene palladium dichloride (54 mg, 0.07 mmol), 2-cyclohexylethenylboronic acid (137 mg, 0.89 mmol), sodium carbonate (236 mg, 2.23 mmol). The reaction mixture was stirred at 100° C. for 2 hours. The mixture was concentrated and the residue was purified by silica gel column chromatography (0-10% EtOAc in petroleum ether) to give the title compound (140 mg, 68%) as a yellow solid. ¹H NMR (400 MHz, CDCl₃) δ 6.83 (d, J=2.4 Hz, 1H), 6.70 (d, J=8.4 Hz, 1H), 6.63 (d, J=16.0 Hz, 1H), 6.55 (dd, J=8.4, 2.4 Hz, 1H), 6.11 (dd, J=16.0, 7.2 Hz, 1H), 3.78 (s, 3H), 2.15-2.13 (m, 1H), 1.86-1.72 (m, 5H), 1.32-1.14 (m, 5H).

### Step 2: (E)-N-(3-(2-Cyclohexylvinyl)-4-methoxyphenyl) methanesulfonamide

Following a similar procedure to that of Example 22, the title compound was prepared from (E)-3-(2-cyclohexylvinyl)-4-methoxyaniline and methanesulfonyl chloride as a white solid (126.6 mg, 79%). ¹H NMR (400 MHz, CDCl₃) δ 7.30 (s, 1H), 7.11 (d, J=8.4 Hz, 1H), 6.82 (d, J=8.8 Hz, 1H), 6.63 (d, J=16.4 Hz, 1H), 6.18 (dd, J=16.0, 7.2 Hz, 1H), 3.84 (s, 3H), 2.97 (s, 3H), 2.16-2.13 (m, 1H), 1.83-1.67 (m, 5H), 1.35-1.13 (m, 5H).

### Example 28

### (E)-N-(3-(2-Cyclohexylvinyl)-4-methoxyphenyl)cyclopropane sulfonamide

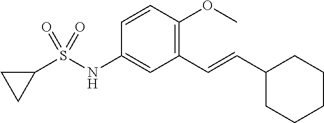 [SMILES: COc1ccc(NS(=O)(=O)C2CC2)cc1/C=C/C1CCCCC1]

Following a similar procedure to that of Example 22, the title compound was prepared from (E)-3-(2-cyclohexylvinyl)-4-methoxyaniline and cyclopropanesulfonyl chloride as a white solid (117.9 mg, 68%). ¹H NMR (400 MHz, CDCl₃) δ 7.33 (d, J=2.4 Hz, 1H), 7.12 (dd, J=8.8, 2.4 Hz, 1H), 6.80 (d, J=8.8 Hz, 1H), 6.63 (d, J=16.0 Hz, 1H), 6.17 (dd, J=16.0, 6.8 Hz, 1H), 3.84 (s, 3H), 2.49-2.38 (m, 1H), 2.20-2.09 (m, 1H), 1.85-1.70 (m, 5H), 1.35-1.17 (m, 5H), 1.15-1.10 (m, 2H), 0.99-0.90 (m, 2H).

### Example 29

### (E)-3-(2-Cyclohexylvinyl)-N-isopropyl-4-methoxybenzamide

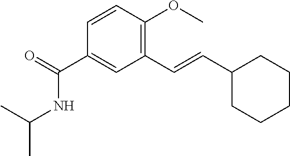 [SMILES: COc1ccc(C(=O)NC(C)C)cc1/C=C/C1CCCCC1]

Following a similar procedure to that of Example 28, the title compound was prepared from 3-bromo-N-isopropyl-4-methoxybenzamide and 2-cyclohexylethenyl boronic acid which furnish the title compound as a yellow solid (82.3 mg, 74%). ¹H NMR (400 MHz, CDCl₃) δ 7.82 (d, J=2.4 Hz, 1H), 7.58 (dd, J=8.4, 2.4 Hz, 1H), 6.85 (d, J=8.4 Hz, 1H), 6.68 (d, J=16.4 Hz, 1H), 6.26 (dd, J=16.0, 6.8 Hz, 1H), 5.85 (d, J=7.2 Hz, 1H), 4.35-4.23 (m, 1H), 3.88 (s, 3H), 2.17-2.14 (m, 1H), 1.84-1.60 (m, 5H), 1.31-1.18 (m, 11H). LCMS (ESI+) m/z 302.0 (M+H)⁺.

### Example 30

### (E)-N-Isopropyl-4-methoxy-3-(3-phenylprop-1-en-1-yl)benzamide

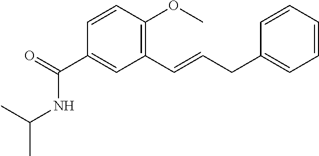 [SMILES: COc1ccc(C(=O)NC(C)C)cc1/C=C/Cc1ccccc1]

Following a similar procedure to that of Example 28, the title compound was prepared from 3-bromo-N-isopropyl-4-methoxybenzamide and (E)-(3-phenylprop-1-en-1-yl)boronic acid to furnish the title compound as a white solid (69.7 mg, 72%). ¹H NMR (400 MHz, CDCl₃) δ 7.77 (d, J=2.0 Hz, 1H), 7.62 (dd, J=8.4, 2.4 Hz, 1H), 7.33-7.20 (m, 5H), 6.86 (d, J=8.4 Hz, 1H), 6.78 (d, J=16.0 Hz, 1H), 6.47-6.39 (m, 1H), 5.83 (s, 1H), 4.30-4.22 (m, 1H), 3.88 (s, 3H), 3.58 (d, J=6.8 Hz, 2H), 1.24 (d, J=6.4 Hz, 6H); LCMS (ESI+) m/z 310.0 (M+H)⁺.

### Example 31

### (E)-3-(2-Cyclohexylvinyl)-4-methoxybenzamide

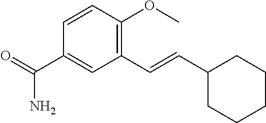 [SMILES: COc1ccc(C(N)=O)cc1/C=C/C1CCCCC1]

### Step 1: 3-Bromo-4-methoxybenzamide

Following a similar procedure to that of Example 26, the title compound was prepared from 3-bromo-4-methoxybenzoic acid and ammonia in THF to furnish the title compound as a white solid (3.6 g, 98%). ¹H NMR (400 MHz, DMSO-d₆) δ 8.10 (d, J=2.0 Hz, 1H), 7.97 (s, 1H), 7.89 (dd, J=8.8, 2.0 Hz, 1H), 7.35 (s, 1H), 7.17 (d, J=8.8 Hz, 1H), 3.89 (s, 3H). LCMS (ESI+) m/z 229.9 (M+H)⁺.

### Step 2: (E)-3-(2-Cyclohexylvinyl)-4-methoxybenzamide

Following a similar procedure to that of Example 27, the title compound was prepared from 3-bromo-4-methoxybenzamide (From step 1) and 2-cyclohexylethenyl boronic acid to furnish the title compound as a white solid (55.6 mg, 49%). ¹H NMR (400 MHz, CDCl₃) δ 7.91 (d, J=2.0 Hz, 1H), 7.65 (dd, J=8.4, 2.0 Hz, 1H), 6.87 (d, J=8.4 Hz, 1H), 6.66 (d, J=16.0 Hz, 1H), 6.25 (dd, J=16.4, 7.2 Hz, 1H), 6.17-5.47 (m, 2H), 3.89 (s, 3H), 2.16 (d, J=7.4 Hz, 1H), 1.84-1.69 (m, 5H), 1.38-1.14 (m, 5H). LCMS (ESI+) m/z 260.0 (M+H)⁺.

### Example 32

### (E)-4-Methoxy-3-(3-phenylprop-1-en-1-yl)benzamide

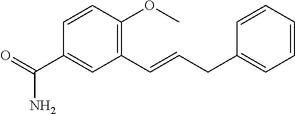 [SMILES: COc1ccc(C(N)=O)cc1/C=C/Cc1ccccc1]

Following a similar procedure to that of Example 27, the title compound was prepared from 3-bromo-4-methoxybenzamide and (E)-(3-phenylprop-1-en-1-yl)boronic acid to furnish the title compound as a white solid (67.4 mg, 58%). ¹H NMR (400 MHz, CDCl₃) δ 7.88 (d, J=2.0 Hz, 1H), 7.68 (dd, J=8.4, 2.0 Hz, 1H), 7.36-7.19 (m, 5H), 6.89 (d, J=8.8 Hz, 1H), 6.79 (d, J=16.0 Hz, 1H), 6.48-6.40 (m, 1H), 6.20-5.60 (br s, 2H), 3.91 (s, 3H), 3.59 (d, J=7.2 Hz, 2H). LCMS (ESI+) m/z 267.9 (M+H)⁺.

### Example 33

### (E)-N-(4-Methoxy-3-(3-phenylprop-1-en-1-yl)phenyl)cyclopropane sulfonamide

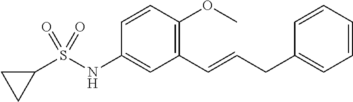 [SMILES: COc1ccc(NS(=O)(=O)C2CC2)cc1/C=C/Cc1ccccc1]

### Step 1: (E)-4-Methoxy-3-(3-phenylprop-1-en-1-yl)aniline

Following a similar procedure to that of Example 27, the title compound was prepared from 3-bromo-4-methoxy aniline and trans-3-phenyl propen-1-yl-boronic acid as a yellow oil. ¹H NMR (400 MHz, CDCl₃) δ 7.33-7.21 (m, 5H), 6.81-6.70 (m, 3H), 6.58 (dd, J=8.4, 2.4 Hz, 1H), 6.33-6.27 (m, 1H), 3.78 (s, 3H), 3.57 (d, J=7.2 Hz, 2H).

### Step 2: (E)-N-(4-Methoxy-3-(3-phenylprop-1-en-1-yl)phenyl) cyclopropanesulfonamide

Following a similar procedure to that of Example 22, the title compound was obtained from (E)-4-Methoxy-3-(3-phenylprop-1-en-1-yl)aniline as a white solid (131 mg, 76%). ¹HNMR (400 MHz, DMSO-d₆) δ 9.32 (s, 1H), 7.32-7.21 (m, 6H), 7.11-7.08 (m, 1H), 6.95 (d, J=8.0 Hz, 1H), 6.67 (d, J=16.0 Hz, 1H), 6.31-6.27 (m, 1H), 3.77 (s, 3H), 3.53 (d, J=7.2 Hz, 2H), 2.52-2.50 (m, 1H), 0.88-0.82 (m, 4H).

### Example 34

### (E)-N-(3-(4-Chlorostyryl)-4-methoxyphenyl)ethenesulfonamide

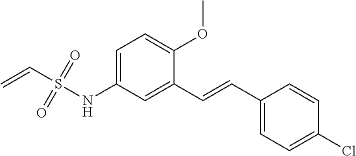 [SMILES: C=CS(=O)(=O)Nc1ccc(OC)c(/C=C/c2ccc(Cl)cc2)c1]

### Step 1: (E)-3-(4-Chlorostyryl)-4-methoxyaniline

Following a similar procedure to that of Example 27, the title compound was prepared from 3-bromo-4-methoxyphenylamine and E-2-(4-chlorophenyl)vinylboronic acid as a white solid (6.3 g, 98%). ¹H NMR (400 MHz, DMSO-d₆) δ 7.55 (d, J=8.4 Hz, 2H), 7.46-7.31 (m, 3H), 7.02 (d, J=16.4 Hz, 1H), 6.89 (d, J=2.8 Hz, 1H), 6.77 (d, J=8.8 Hz, 1H), 6.55 (dd, J=8.8, 2.4 Hz, 1H), 4.68 (s, 2H), 3.72 (s, 3H).

### Step 2: (E)-N-(3-(4-Chlorostyryl)-4-methoxyphenyl)ethenesulfonamide

Following a similar procedure to that of Example 22, the title compound was prepared from (E)-3-(4-chlorostyryl)-4-methoxyaniline (From step 1) and ethenesulfonyl chloride as a yellow solid (50.6 mg, 38%). ¹HNMR (400 MHz, DMSO-d₆) δ 9.65 (s, 1H), 7.60 (d, J=8.4 Hz, 2H), 7.46-7.31 (m, 4H), 7.13-7.05 (m, 2H), 7.02-6.96 (m, 1H), 6.78-6.71 (m, 1H), 6.08-5.94 (m, 2H), 3.82 (s, 3H).

### Example 35

The overall Example 35 reaction scheme was as follows:

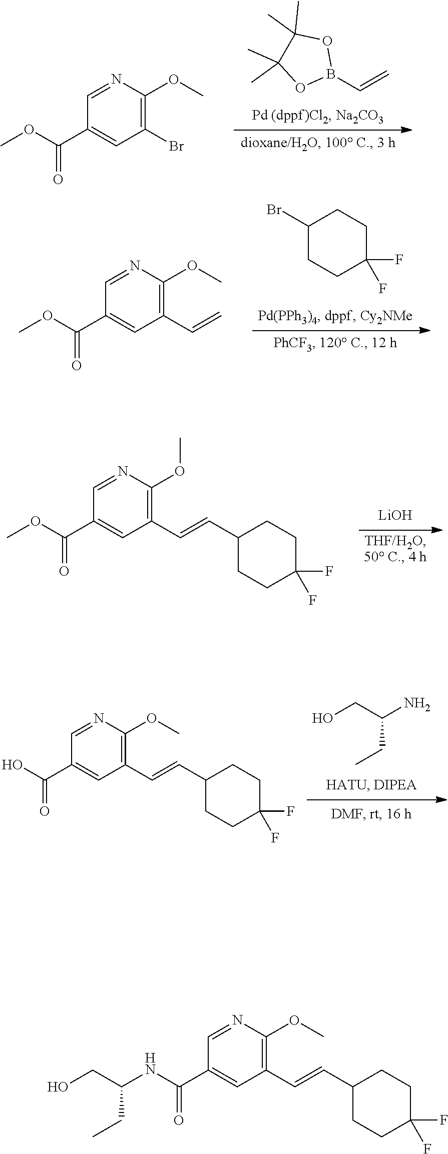

### Step 1: Methyl 6-methoxy-5-vinylnicotinate

A mixture of methyl 5-bromo-6-methoxy-pyridine-3-carboxylate (0.5 g, 2.03 mmol) and 1,1′-bis(diphenylphosphino)ferrocene palladium dichloride (0.07 g, 0.10 mmol), vinyl boronic acid pinacol ester (344 mg, 2.24 mmol), sodium carbonate (0.65 g, 6.1 mmol) in 1,4-dioxane (5 mL), water (1 mL) was stirred at 100° C. under N₂ for 3 hours. The reaction mixture was concentrated and the residue was purified by column chromatography on silica gel (0-33% EtOAc in petroleum ether) to afford the title compound (220 mg, 56%) as a white solid. LCMS (ESI+) m/z 194.0 (M+H)⁺.

### Step 2: (E)-Methyl 5-(2-(4,4-difluorocyclohexyl)vinyl)-6-methoxynicotinate

A mixture of 1,1′-bis(diphenylphosphino)ferrocene (57 mg, 0.10 mmol), N,N-dicyclohexylmethylamine (606 mg, 3.11 mmol), 4-bromo-1,1-difluoro-cyclohexane (412 mg, 2.07 mmol), Pd(PPh₃)₄ (119 mg, 0.10 mmol) and methyl 6-methoxy-5-vinyl-pyridine-3-carboxylate (200 mg, 1.04 mmol) in (trifluoromethyl)benzene (5 mL) was stirred at 120° C. for 12 hours under N₂ atmosphere. The reaction mixture was concentrated and purified by prep-TLC (20% EtOAc in petroleum) to afford the title compound (60 mg, 19%) as a white solid. ¹H NMR (400 MHz, CDCl₃) δ 8.68 (d, J=2.0 Hz, 1H), 8.21 (d, J=2.0 Hz, 1H), 6.57 (d, J=16.0 Hz, 1H), 6.29 (dd, J=16.0, 7.2 Hz, 1H), 4.04 (s, 3H), 3.92 (s, 3H), 2.28-2.26 (m, 1H), 2.20-2.08 (m, 2H), 1.94-1.86 (m, 2H), 1.85-1.70 (m, 2H), 1.64-1.54 (m, 2H).

### Step 3: (E)-5-(2-(4,4-Difluorocyclohexyl)vinyl)-6-methoxynicotinic Acid

To a solution of (E)-methyl 5-(2-(4,4-difluorocyclohexyl)vinyl)-6-methoxynicotinate (60 mg, 0.19 mmol) in water (1 mL), methanol (1 mL) and THF (1 mL) was added lithium hydroxide (23 mg, 0.96 mmol). The mixture was stirred for 5 hours at 25° C. The reaction mixture was acidified with aqueous 2 N hydrochloric acid to pH=5 and the mixture was extracted with EtOAc (50 mL×3). The combined organic layers were dried over anhydrous Na₂SO₄ and concentrated to afford the title compound (50 mg, 87%) as a brown solid. LCMS (ESI+) m/z 298.0 (M+H)⁺.

### Step 4: (R,E)-5-(2-(4,4-Difluorocyclohexyl)vinyl)-N-(1-hydroxybutan-2-yl)-6-methoxynicotinamide

To a solution of (E)-5-(2-(4,4-difluorocyclohexyl)vinyl)-6-methoxynicotinic acid (50 mg, 0.17 mmol), (R)-2-amino-1-butanol (18 mg, 0.20 mmol) and HATU (77 mg, 0.20 mmol) in DMF (0.50 mL) was added N,N-diisopropylethylamine (0.08 mL, 0.50 mmol) and the reaction mixture was stirred for 2 hours at 15° C. The reaction mixture was diluted with water (20 mL), extracted with DCM (20 mL×2). The combined organic layers were dried over anhydrous Na₂SO₄, filtered and concentrated. The crude residue was purified by prep-HPLC (acetonitrile 30-60/0.1% NH₄HCO₃ in water) to afford the title compound (12.1 mg, 20%) as a white solid. ¹H NMR (400 MHz, CDCl₃) δ 8.42 (d, J=2.4 Hz, 1H), 8.06 (d, J=2.4 Hz, 1H), 6.57 (d, J=16.0 Hz, 1H), 6.31 (dd, J=16.0, 6.8 Hz, 1H), 6.24 (d, J=8.0 Hz, 1H), 4.15-4.05 (m, 1H), 4.02 (s, 3H), 3.87-3.68 (m, 2H), 2.60-2.58 (m, 1H), 2.28-2.27 (m, 1H), 2.21-2.08 (m, 2H), 1.94-1.64 (m, 6H), 1.61-1.49 (m, 2H), 1.03 (t, J=7.2 Hz, 3H). LCMS (ESI+) m/z 369.1 (M+H)⁺.

### Example 36

### (E)-N-(5-(2-(3,3-Dimethylcyclobutyl)vinyl)-6-methoxypyridin-3-yl)methanesulfonamide

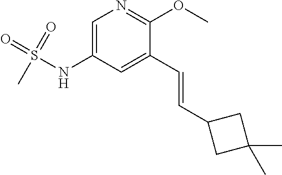 [SMILES: COc1ncc(NS(C)(=O)=O)cc1/C=C/C1CC(C)(C)C1]

### Step 1: 3,3-Dimethylcyclobutanecarbaldehyde

To a stirred solution of methyl 3,3-dimethylcyclobutanecarboxylate (250 mg, 1.76 mmol) in DCM (2 mL) was added DIBAL (1.0 M in toluene, 1.6 mL, 1.6 mmol) at −78° C. The reaction mixture was stirred at −78° C. for 2 hours. Water (1 mL) was added to the reaction mixture. The mixture was dried over anhydrous MgSO₄, filtered and concentrated to remove the low boiling solvents. This afforded the title compound as a 10% solution in toluene, which was used directly in the next step without further purification. ¹H NMR (400 MHz, CDCl₃) δ 9.73 (d, J=2.0 Hz, 1H), 3.11-3.03 (m, 1H), 2.06-1.92 (m, 4H), 1.20 (s, 3H), 1.08 (s, 3H).

### Step 2: (E)-5-Bromo-3-(2-(3,3-dimethylcyclobutyl)vinyl)-2-methoxypyridine

To a solution of diethyl ((5-bromo-2-methoxypyridin-3-yl)methyl)phosphonate (0.4 g, 1.18 mmol) in toluene (2 mL) at 0° C. was added sodium tert-pentoxide (0.13 g, 1.18 mmol) and the mixture was stirred for 20 minutes at 0° C. 3,3-Dimethylcyclobutane carbaldehyde (Resulting from step 1, toluene solution, about 1.4 mmol) was added dropwise and the reaction mixture was stirred for 1.5 hours at 0° C. The reaction mixture was poured into saturated aqueous NH₄Cl solution (10 mL) and was extracted with EtOAc (10 mL×2). The combined organic layers were washed with brine (10 mL), dried over anhydrous Na₂SO₄ and concentrated and the residue was purified by column chromatography on silica gel (0-10% EtOAc in petroleum ether) to afford the title compound (250 mg, 71%) as a colorless oil. ¹H NMR (400 MHz, CD₃OD) δ 8.03 (d, J=2.4 Hz, 1H), 7.87 (d, J=2.4 Hz, 1H), 6.54 (dd, J=16.0, 6.8 Hz, 1H), 6.41 (d, J=16.0 Hz, 1H), 3.95 (s, 3H), 3.15-2.96 (m, 1H), 2.09-1.96 (m, 2H), 1.85-1.74 (m, 2H), 1.23 (s, 3H), 1.11 (s, 3H).

### Step 3: (E)-N-(5-(2-(3,3-Dimethylcyclobutyl)vinyl)-6-methoxypyridin-3-yl)methanesulfonamide

A mixture of (E)-5-bromo-3-(2-(3,3-dimethylcyclobutyl)vinyl)-2-methoxy pyridine (From step 2, 100 mg, 0.34 mmol), allylpalladium(II) chloride dimer (12 mg, 0.03 mmol), 2-di-tert-butylphosphino-2′,4′,6′-triisopropylbiphenyl (28 mg, 0.07 mmol), methane sulfonamide (64 mg, 0.68 mmol), potassium carbonate (140 mg, 1.01 mmol) in 1,4-dioxane (5 mL) was stirred at 100° C. for 16 hours under N₂ atmosphere. The reaction mixture was filtered and the filtrate was concentrated. The residue was further purified by prep-HPLC(acetonitrile 55-75/0.1% NH₄HCO₃ in water) to afford the title compound (13.8 mg, 13%) as a white solid. H NMR (400 MHz, DMSO-d₆) δ 9.33 (br s, 1H), 7.88 (d, J=2.4 Hz, 1H), 7.63 (d, J=2.4 Hz, 1H), 6.47 (dd, J=16.0, 6.8 Hz, 1H), 6.38 (d, J=16.0 Hz, 1H), 3.87 (s, 3H), 3.13-3.02 (m, 1H), 2.95 (s, 3H), 1.98-1.91 (m, 2H), 1.76-1.69 (m, 2H), 1.17 (s, 3H), 1.06 (s, 3H); LCMS (ESI+) m/z 311.0 (M+H)⁺.

### Example 37

### N-(6-Methoxy-5-((E)-2-(trans-3-(trifluoromethyl)cyclobutyl)vinyl)pyridin-3-yl)methanesulfonamide

The overall Example 37 reaction scheme was as follows:

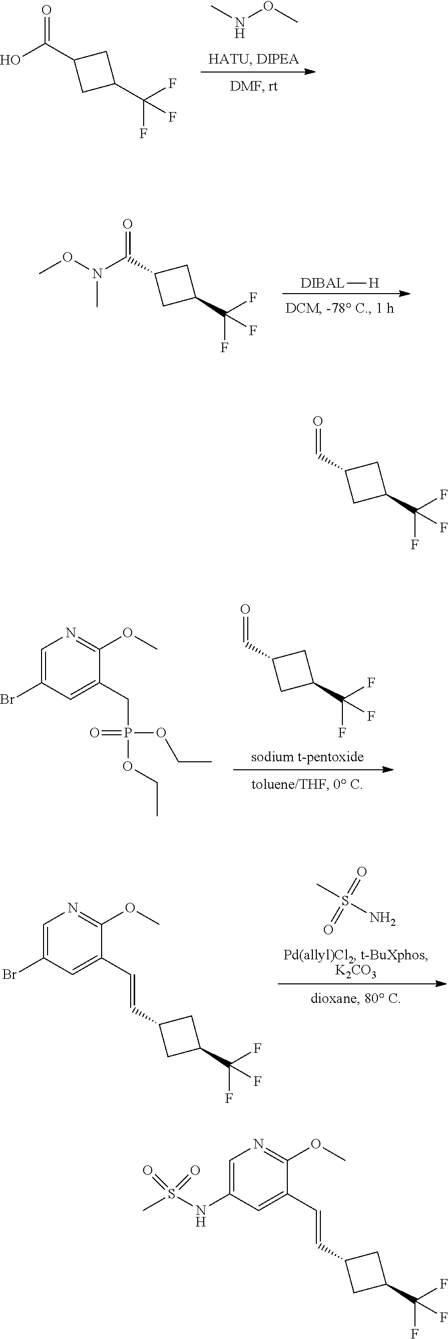

### Step 1: Trans-N-methoxy-N-methyl-3-(trifluoromethyl) cyclobutanecarboxamide

A mixture of 3-(trifluoromethyl)cyclobutanecarboxylic acid (500 mg, 2.97 mmol), DIPEA (1.3 mL, 7.44 mmol) and HATU (1.47 g, 3.87 mmol) in DMF (10 mL) was stirred at 17° C. for 0.5 hours. N,O-Dimethylhydroxylamine hydrochloride (377 mg, 3.87 mmol) was added and the mixture was stirred at 17° C. for 1 hour. The mixture was concentrated and the residue was diluted with EtOAc (30 mL), washed with water (30 mL) and brine (30 mL). The organic phase was dried over anhydrous Na₂SO₄, concentrated and purified by column chromatography on silica gel (50% EtOAc in petroleum ether) to afford trans-N-methoxy-N-methyl-3-(trifluoromethyl)cyclobutane carboxamide (200 mg, 32%) and cis-N-methoxy-N-methyl-3-(trifluoromethyl) cyclobutanecarboxamide (400 mg, 64%) both as colorless oil. cis isomer: ¹H NMR (400 MHz, CDCl₃) δ 3.67 (s, 3H), 3.40-3.29 (m, 1H), 3.19 (s, 3H), 2.96-2.80 (m, 1H), 2.55-2.41 (m, 2H), 2.35-2.23 (m, 2H). trans isomer: ¹H NMR (400 MHz, CDCl₃) δ 3.66 (s, 3H), 3.56-3.55 (m, 1H), 3.20 (s, 3H), 3.04-2.87 (m, 1H), 2.56-2.52 (m, 2H), 2.43-2.31 (m, 2H).

### Step 2: trans-3-(Trifluoromethyl)cyclobutanecarbaldehyde

To a stirred solution of trans-N-methoxy-N-methyl-3-(trifluoromethyl)cyclobutane carboxamide (From step 1, 180 mg, 0.85 mmol) in DCM (4 mL) was added DIBAL (1 M in toluene, 0.85 mL, 0.85 mmol) at −78° C. The reaction mixture was stirred at −78° C. for 2 hours. water (0.5 mL) was added to the reaction mixture. The reaction mixture was dried over anhydrous MgSO₄ and concentrated to afford the title compound (500 mg in 0.5 mL toluene) which was used directly in the next step. ¹H NMR (400 MHz, CDCl₃) δ 9.79 (s, 1H), 3.26-3.25 (m, 1H), 2.90-2.83 (m, 2H), 2.51-2.46 (m, 2H).

### Step 3: 5-Bromo-2-methoxy-3-((E)-2-(trans-3-(trifluoromethyl) cyclobutyl)vinyl) pyridine

Following a similar procedure to that of Example 36, The title compound was prepared from trans-3-(trifluoromethyl)cyclobutane carbaldehyde and diethyl ((5-bromo-2-methoxypyridin-3-yl)methyl)phosphonate. ¹H NMR (400 MHz, CDCl₃) δ 8.07 (d, J=2.4 Hz, 1H), 7.74 (d, J=2.4 Hz, 1H), 6.49-6.38 (m, 2H), 3.96 (s, 3H), 3.30-3.24 (m, 1H), 2.98-2.88 (m, 1H), 2.51-2.45 (m, 2H), 2.25-2.20 (m, 2H).

### Step 4: N-(6-methoxy-5-((E)-2-(trans-3-(trifluoromethyl)cyclobutyl) vinyl)pyridin-3-yl)methanesulfonamide

Following a similar procedure to that of Example 36, the title compound was prepared from 5-bromo-2-methoxy-3-((E)-2-(trans-3-(trifluoromethyl)cyclobutyl) vinyl)pyridine (From step 3) and methanesulfonamide as a white solid (25 mg, 24%). ¹H NMR (400 MHz, CD₃OD) δ 7.91 (d, J=2.8 Hz, 1H), 7.72 (d, J=2.8 Hz, 1H), 6.58-6.49 (m, 2H), 3.95 (s, 3H), 3.27-3.20 (m, 1H), 3.07-2.98 (m, 1H), 2.94 (s, 3H), 2.47-2.41 (m, 2H), 2.29-2.22 (m, 2H). LCMS (ESI+) m/z 350.9 (M+H)⁺.

### Example 38

### (E)-N-(5-(2-Cyclohexylvinyl)-2-fluoro-6-methoxypyridin-3-yl) methanesulfonamide

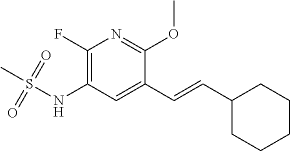 [SMILES: COc1nc(F)c(NS(C)(=O)=O)cc1/C=C/C1CCCCC1]

### Step 1: 5-Bromo-6-methoxy-3-nitropyridin-2-amine

To the solution of 6-methoxy-3-nitro-2-pyridinamine (3.5 g, 20.7 mmol) in DMF (40 mL) was added N-bromosuccinimide (4.05 g, 22.7 mmol) portion wise at 0° C. The reaction mixture was stirred at 20° C. for 2 hours. The reaction mixture was poured into water (300 mL). The resulting precipitate was filtered and dried in vacuo to give the title compound (4.5 g, 88%) as a yellow solid. ¹H NMR (400 MHz, CDCl₃) δ 8.50 (s, 1H), 7.90 (br s, 1H), 5.72 (br s, 1H), 3.98 (s, 3H).

### Step 2: 3-Bromo-6-fluoro-2-methoxy-5-nitropyridine

5-Bromo-6-methoxy-3-nitro-pyridin-2-amine (26.0 g, 104.8 mmol) was slowly added to a solution of HF/pyridine (200 mL) at 0° C. Sodium nitrite (7.1 g, 102.7 mmol) was added slowly portion wise to the reaction mixture and the reaction mixture was stirred for 1 hour. The mixture was poured into ice water (1 L) and extracted with EtOAc (500 mL×2). The organic layers were combined and washed successively with 1 N NaOH solution (900 mL×2), saturated NaHCO₃ solution (800 mL), and brine (800 mL). The organic layer was separated, dried over anhydrous Na₂SO₄, filtered and concentrated under vacuum. The residue was purified with column chromatography on silica gel (0-10% EtOAc in petroleum ether) to afford the title compound (19 g, 72%) as a light yellow solid. ¹H NMR (400 MHz, CDCl₃) δ 8.67 (d, J=8.0 Hz, 1H), 4.12 (s, 3H).

### Step 3: 5-Bromo-2-fluoro-6-methoxypyridin-3-amine

Following a similar procedure to that of Example 22, the title compound was prepared from 5-bromo-2-fluoro-6-methoxy-3-nitro-pyridine as a yellow solid (5.6 g, 64%). LCMS (ESI+) m/z 222.8 (M+H)⁺.

### Step 4: N-(5-Bromo-2-fluoro-6-methoxypyridin-3-yl) methanesulfonamide

Following a similar procedure to that of Example 22, the title compound was prepared from 5-bromo-2-fluoro-6-methoxy-pyridin-3-amine and methanesulfonyl chloride as a white solid (7.2 g, 95%). ¹H NMR (400 MHz, CDCl₃) δ 8.06 (d, J=8.8 Hz, 1H), 6.30 (s, 1H), 3.98 (s, 3H), 3.02 (s, 3H).

### Step 5: N-(2-Fluoro-6-methoxy-5-vinylpyridin-3-yl)methanesulfonamide

Following a similar procedure to that of Example 35, the title compound was prepared from N-(5-bromo-2-fluoro-6-methoxy-3-pyridyl)methanesulfonamide (From step 4) and vinylboronic acid pinacolester as a white solid (800 mg, 81%). H NMR (400 MHz, CDCl₃) δ 7.92 (d, J=9.6 Hz, 1H), 6.76 (dd, J=18.0, 11.2 Hz, 1H), 6.18 (s, 1H), 5.79 (d, J=18.0 Hz, 1H), 5.36 (d, J=11.2 Hz, 1H), 3.95 (s, 3H), 3.00 (s, 3H).

### Step 6: (E)-N-(5-(2-Cyclohexylvinyl)-2-fluoro-6-methoxypyridin-3-yl) methanesulfonamide

Following a similar procedure to that of Example 35, the title compound was prepared from N-(2-fluoro-6-methoxy-5-vinyl-3-pyridyl)methanesulfonamide and iodocyclohexane as a white solid (26.1 mg, 39%). ¹H NMR (400 MHz, CDCl₃) δ 7.88 (d, J=9.6 Hz, 1H), 6.41 (d, J=16.4 Hz, 1H), 6.20 (dd, J=16.4, 6.8 Hz, 1H), 6.08 (s, 1H), 3.94 (s, 3H), 3.00 (s, 3H), 2.21-2.05 (m, 1H), 1.83-1.59 (m, 5H), 1.37-1.08 (m, 5H). LCMS (ESI+) m/z 329.2 (M+H)⁺.

### Example 39

### (E)-N-(5-(2-(4,4-Difluorocyclohexyl)vinyl)-2-fluoro-6-methoxypyridin-3-yl)methanesulfonamide

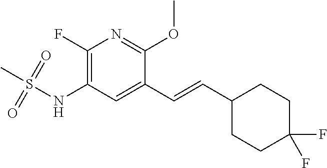 [SMILES: COc1nc(F)c(NS(C)(=O)=O)cc1/C=C/C1CCC(F)(F)CC1]

Following a similar procedure to that of Example 27, the title compound was prepared from N-(2-fluoro-6-methoxy-5-bromo-3-pyridyl)methanesulfonamide and (E)-3-cyclopentylprop-1-enyl]boronic acid as a white solid (74.7 mg, 68%). ¹H NMR (400 MHz, DMSO-d₆) δ 9.44 (s, 1H), 7.85 (d, J=9.6 Hz, 1H), 6.54-6.21 (m, 2H), 3.89 (s, 3H), 3.02 (s, 3H), 2.20 (t, J=6.8 Hz, 2H), 1.98-1.91 (m, 1H), 1.79-1.66 (m, 2H), 1.62-1.40 (m, 4H), 1.27-1.08 (m, 2H); LCMS (ESI+) m/z 329.3 (M+H)⁺.

### Example 40

### (E)-N-(5-(3-Cyclopentylprop-1-en-1-yl)-2-fluoro-6-methoxypyridin-3-yl) methanesulfonamide

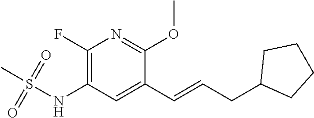 [SMILES: COc1nc(F)c(NS(C)(=O)=O)cc1/C=C/CC1CCCC1]

Following a similar procedure to that of Example 27, the title compound was prepared from N-(2-fluoro-6-methoxy-5-bromo-3-pyridyl)methanesulfonamide and (E)-3-cyclopentylprop-1-enyl]boronic acid as a white solid (74.7 mg, 68%). ¹H NMR (400 MHz, DMSO-d₆) δ 9.44 (s, 1H), 7.85 (d, J=9.6 Hz, 1H), 6.54-6.21 (m, 2H), 3.89 (s, 3H), 3.02 (s, 3H), 2.20 (t, J=6.8 Hz, 2H), 1.98-1.91 (m, 1H), 1.79-1.66 (m, 2H), 1.62-1.40 (m, 4H), 1.27-1.08 (m, 2H); LCMS (ESI+) m/z 329.3 (M+H)⁺.

### Example 41

### (R,E)-4-(2-(4,4-Difluorocyclohexyl)vinyl)-N-(1-hydroxybutan-2-yl)-5-methoxy picolinamide

The overall Example 40 reaction scheme was as follows:

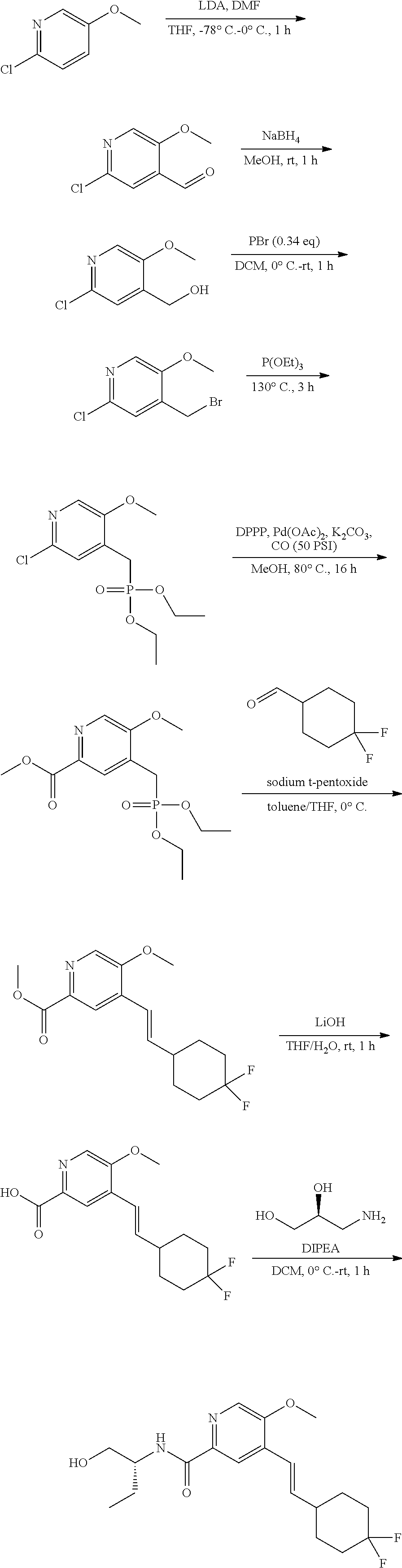

### Step 1: 2-Chloro-5-methoxyisonicotinaldehyde

To a mixture of 2-chloro-5-methoxy-pyridine (6.0 g, 42 mmol) in THF (50 mL) was added lithium diisopropylamide (2.0 M in THF, 42 mL, 83.6 mmol) dropwise at −78° C. The reaction mixture was stirred at −78° C. for 1 hour. N,N-dimethylformamide (5.0 mL, 83.6 mmol) was added to the reaction mixture at −78° C., and the mixture was stirred for 1 hour. Saturated NH₄Cl solution (100 mL) was added to the reaction mixture. The solution was extracted with EtOAc (200 mL×2). The combined organic layers were dried over anhydrous Na₂SO₄ and concentrated. The residue was purified by column chromatography on silica gel (0-25% EtOAc in petroleum ether) to afford the title compound (5.5 g, 77%) as a white solid. ¹H NMR (400 MHz, CDCl₃) δ 10.41 (s, 1H), 8.27 (s, 1H), 7.59 (s, 1H), 4.03 (s, 3H).

### Step 2: (2-Chloro-5-methoxypyridin-4-yl)methanol

To a mixture of 2-chloro-5-methoxy-pyridine-4-carbaldehyde (6.0 g, 35.0 mmol) in methanol (50 mL) was added sodium borohydride (1.59 g, 42.0 mmol) at 15° C. The reaction mixture was stirred at 15° C. for 1 hour. Water (50 mL) was added to the reaction mixture and the mixture was concentrated. The residue was diluted with water (200 mL), extracted with EtOAc (200 mL×2). The combined organic layers were dried over anhydrous Na₂SO₄, filtered and concentrated to afford the title compound (6.0 g, 99%) as a white solid which was used for the next step directly. ¹H NMR (400 MHz, CDCl₃) δ 7.91 (s, 1H), 7.38 (s, 1H), 4.69 (s, 2H), 3.90 (s, 3H).

### Step 3: 4-(Bromomethyl)-2-chloro-5-methoxypyridine

To a mixture of (2-chloro-5-methoxy-4-pyridyl)methanol (3.0 g, 17.3 mmol) in dichloromethane (30 mL) was added phosphorous tribromide (560 uL, 5.9 mmol) at 0° C. The reaction mixture was stirred at 15° C. for 2 hours. The solution was concentrated and the residue was purified by column chromatography on silica gel (0-20% EtOAc in petroleum ether) to afford the title compound (1.9 g, 47%) as a white solid. ¹H NMR (400 MHz, CDCl₃) δ 7.99 (s, 1H), 7.29 (s, 1H), 4.38 (s, 2H), 3.97 (s, 3H).

### Step 4: Diethyl ((2-chloro-5-methoxypyridin-4-yl)methyl)phosphonate

A mixture of 4-(bromomethyl)-2-chloro-5-methoxypyridine (1.9 g, 10.9 mmol) in triethyl phosphite (10 mL) was stirred at 130° C. for 3 hours. The reaction mixture was concentrated to give the title compound (2.4 g, 75%) as a colorless oil which was used for next step without purification. LCMS (ESI+) m/z 293.9 (M+H)⁺.

### Step 5: Methyl 4-((diethoxyphosphoryl)methyl)-5-methoxypicolinate

A mixture of diethyl ((2-chloro-5-methoxypyridin-4-yl)methyl)phosphonate (2.4 g, 8.2 mmol), potassium carbonate (2.3 g, 16.3 mmol), palladium acetate (183 mg, 0.8 mmol), and 1,3-bis(diphenylphosphino)propane (674 mg, 1.6 mmol) in methanol (50 mL) was heated at 80° C. under CO atmosphere (50 psi) for 16 hours. The reaction mixture was filtered and concentrated. The residue was purified by column chromatography on silica gel (0-10% MeOH in DCM) to afford the title compound (1.7 g, 66%) as a light yellow oil. ¹H NMR (400 MHz, CDCl₃) δ 8.32 (s, 1H), 8.08 (d, J=2.4 Hz, 1H), 4.06 (q, J=7.2 Hz, 4H), 4.01 (s, 3H), 3.96 (s, 3H), 3.30-3.20 (m, 2H), 1.26 (t, J=7.2 Hz, 6H).

### Step 6: (E)-Methyl 4-(2-(4,4-difluorocyclohexyl)vinyl)-5-methoxypicolinate

Following a similar procedure to that of Example 36, the title compound was prepared from methyl 4-((diethoxyphosphoryl)methyl)-5-methoxypicolinate and 4,4-difluorocyclohexane carboxaldehyde as a white solid (30 mg, 61%). LCMS (ESI+) m/z 312.1 (M+H)⁺.

### Step 7: (E)-4-(2-(4,4-Difluorocyclohexyl)vinyl)-5-methoxypicolinic Acid

Following a similar procedure to that of Example 35, the title compound was prepared from (E)-methyl 4-(2-(4,4-difluorocyclohexyl)vinyl)-5-methoxypicolinate as a white solid (30 mg, 90%). LCMS (ESI+) m/z 298.0 (M+H)⁺.

### Step 8: (R,E)-4-(2-(4,4-Difluorocyclohexyl)vinyl)-N-(1-hydroxybutan-2-yl)-5-methoxy picolinamide

Following a similar procedure to that of Example 35, the title compound was prepared from (E)-4-(2-(4,4-difluorocyclohexyl)vinyl)-5-methoxypicolinic acid and (R)-2-amino-1-butanol (13 mg, 0.15 mmol) as a white solid (8.1 mg, 22%). ¹H NMR (400 MHz, CD₃OD) δ 8.29 (s, 1H), 8.11 (s, 1H), 6.74 (d, J=16.0 Hz, 1H), 6.55 (dd, J=16.0, 6.8 Hz, 1H), 4.01 (s, 3H), 3.99-3.93 (m, 1H), 3.67-3.58 (m, 2H), 2.36-2.32 (m, 1H), 2.08-2.05 (m, 2H), 1.93-1.85 (m, 3H), 1.81-1.66 (m, 2H), 1.62-1.51 (m, 3H), 0.95 (t, J=7.2 Hz, 3H). LCMS (ESI+) m/z 369.1 (M+H)⁺.

### Example 42

### N—((R)-1-Hydroxybutan-2-yl)-5-methoxy-4-((E)-2-(trans-4-(trifluoromethyl)cyclohexyl)vinyl)picolinamide

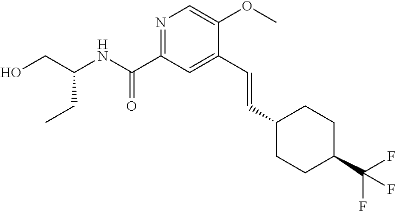 [SMILES: CC[C@H](CO)NC(=O)c1cc(/C=C/[C@H]2CC[C@H](C(F)(F)F)CC2)c(OC)cn1]

### Step 1: trans-N-Methoxy-N-methyl-4-(trifluoromethyl) cyclohexanecarboxamide

A mixture of trans-4-(trifluoromethyl)cyclohexane carboxylic acid (4.5 g, 22.94 mmol), N,O-dimethyl hydroxylamine hydrochloride (2.68 g, 27.53 mmol), HATU (10.47 g, 27.53 mmol) and DIPEA (11.32 mL, 68.82 mmol) in DMF (60 mL) was stirred at 15° C. for 3 hours. The reaction mixture was diluted in EtOAc (100 mL), washed with brine (100 mL×3). The organic layer was dried over anhydrous Na₂SO₄, filtered and concentrated. The crude residue was purified by silica gel column chromatography (40% EtOAc in petroleum ether) to afford the title compound (3.9 g, 71%) as a solid. ¹H NMR (400 MHz, CDCl₃) δ 3.71 (s, 3H), 3.19 (s, 3H), 2.71-2.69 (m, 1H), 2.08-2.02 (m, 3H), 1.94-1.90 (m, 2H), 1.56-1.53 (m, 2H), 1.39-1.36 (m, 2H).

### Step 2: trans-4-(Trifluoromethyl)cyclohexanecarbaldehyde

Following a similar procedure to that of Example 36, the title compound was prepared from trans-N-methoxy-N-methyl-4-(trifluoromethyl)cyclohexane carboxamide as a light yellow oil (1.2 g, 80%). ¹H NMR (400 MHz, CDCl₃) δ 9.65 (s, 1H), 2.24-2.00 (m, 6H), 1.40-1.27 (m, 4H).

### Step 3: Methyl 5-methoxy-4-((E)-2-(trans-4-(trifluoromethyl)cyclohexyl) vinyl) picolinate

Following a similar procedure to that of Example 36, the title compound was prepared from methyl 4-((diethoxyphosphoryl)methyl)-5-methoxypicolinate and trans-4-(trifluoromethyl)cyclohexanecarbaldehyde as a white solid (250 mg, 77%). ¹H NMR (400 MHz, CDCl₃) δ 8.32 (s, 1H), 8.16 (s, 1H), 6.65 (d, J=16.4 Hz, 1H), 6.50 (dd, J=16.0, 6.8 Hz, 1H), 4.02 (s, 3H), 3.99 (s, 3H), 2.22-2.20 (m, 1H), 2.06-1.98 (m, 5H), 1.45-1.41 (m, 2H), 1.29-1.25 (m, 2H).

### Step 4: 5-Methoxy-4-((E)-2-(trans-4-(trifluoromethyl)cyclohexyl) vinyl)picolinic Acid

Following a similar procedure to that of Example 35, the title compound was prepared from methyl 5-methoxy-4-((E)-2-(trans-4-(trifluoromethyl)cyclohexyl) vinyl)picolinate as a white solid (200 mg, 83%). LCMS (ESI+) m/z 330.1 (M+H)⁺.

### Step 5: N—((R)-1-Hydroxybutan-2-yl)-5-methoxy-4-((E)-2-(trans-4-(trifluoro-methyl)cyclohexyl)vinyl)picolinamide

Following a similar procedure to that of Example 35, the title compound was prepared from 5-methoxy-4-((E)-2-(trans-4-(trifluoromethyl)cyclohexyl)vinyl)picolinic acid and (R)-2-amino-1-butanol (32 mg, 0.36 mmol) as a light yellow solid (55 mg, 45%). ¹H NMR (400 MHz, CD₃OD) δ 8.30 (s, 1H), 8.12 (s, 1H), 6.71 (d, J=16.0 Hz, 1H), 6.57 (dd, J=16.0, 6.8 Hz, 1H), 4.03 (s, 3H), 4.00-3.95 (m, 1H), 3.69-3.61 (m, 2H), 2.26-2.10 (m, 2H), 2.03-1.95 (m, 4H), 1.80-1.58 (m, 2H), 1.48-1.27 (m, 4H), 0.99 (t, J=7.2 Hz, 3H). LCMS (ESI+) m/z 401.1 (M+H)⁺.

### Example 43

### (S,E)-N-(2,3-Dihydroxypropyl)-5-methoxy-4-(2-(spiro[2.3]hexan-5-yl) vinyl)picolinamide

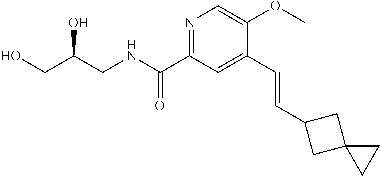 [SMILES: COc1cnc(C(=O)NC[C@H](O)CO)cc1/C=C/C1CC2(CC2)C1]

### Step 1: Methyl spiro[2.3]hexane-5-carboxylate

To a solution of Et₂Zn (11.89 mL, 11.89 mmol) in DCM (10 mL) was added a solution of TFA (0.88 mL, 11.89 mmol) in DCM (10 mL) dropwise at 0° C. for 30 minutes. A solution of CH₂I2 (0.96 mL, 11.89 mmol) in DCM (10 mL) was added dropwise at 0° C. for 45 minutes. The reaction mixture was stirred at 0° C. for 1 hour. A solution of methyl 3-methylenecyclobutanecarboxylate (500 mg, 3.96 mmol) in DCM (5 mL) was added to the reaction mixture. The reaction mixture was allowed to warm to 15° C. for 16 hours. Saturated NH₄Cl solution (50 mL) was added to the reaction mixture and the mixture was extracted with DCM (50 mL×2). The combined organic layers were dried over anhydrous Na₂SO₄, filtered and concentrated. The crude residue was purified by silica gel column chromatography (10% EtOAc in petroleum ether) to afford the title compound (400 mg, 72%) as a yellow oil. ¹H NMR (400 MHz, CDCl₃) δ 3.71 (s, 3H), 3.34-3.27 (m, 1H), 2.53-2.48 (m, 2H), 2.26-2.20 (m, 2H), 0.49-0.42 (m, 4H).

### Step 2: Spiro[2.3]hexane-5-carbaldehyde

Following a similar procedure to that of Example 36, the title compound was prepared from spiro[2.3]hexane-5-carboxylate as a light yellow oil (220 mg, 70%). ¹H NMR (400 MHz, CDCl₃) δ 9.83 (s, 1H), 3.31-3.24 (m, 1H), 2.44-2.39 (m, 2H), 2.31-2.26 (m, 2H), 0.50-0.40 (m, 4H).

### Step 3: (E)-Methyl 5-methoxy-4-(2-(spiro[2.3]hexan-5-yl) vinyl)picolinate

Following a similar procedure to that of Example 36, the title compound was prepared from methyl 4-(diethoxyphosphorylmethyl)-5-methoxy-pyridine-2-carboxylate and spiro[2.3]hexane-5-carbaldehyde as a light yellow oil (150 mg, 58%). LCMS (ESI+) m/z 274.0 (M+H)⁺.

### Step 4: (E)-5-Methoxy-4-(2-(spiro[2.3]hexan-5-yl)vinyl)picolinic acid

Following a similar procedure to that of Example 35, the title compound was prepared from (E)-Methyl 5-methoxy-4-(2-(spiro[2.3]hexan-5-yl)vinyl)picolinate as a light yellow solid (100 mg, 70%). LCMS (ESI+) m/z 260.0 (M+H)⁺.

### Step 5: (S,E)-N-(2,3-Dihydroxypropyl)-5-methoxy-4-(2-(spiro[2.3]hexan-5-yl)vinyl)picolinamide

Following a similar procedure to that of Example 35, the title compound was prepared from (E)-5-Methoxy-4-(2-(spiro[2.3]hexan-5-yl)vinyl)picolinic acid (From step 4) and (S)-3-amino-1,2-propanediol as a light yellow solid (20 mg, 31%). ¹H NMR (400 MHz, CD₃OD) δ 8.28 (s, 1H), 8.14 (s, 1H), 6.84 (dd, J=16.0, 6.8 Hz, 1H), 6.64 (d, J=16.0 Hz, 1H), 4.02 (s, 3H), 3.82-3.80 (m, 1H), 3.65-3.55 (m, 3H), 3.45-3.43 (m, 1H), 3.33-3.31 (m, 1H), 2.31-2.18 (m, 4H), 0.51-0.40 (m, 4H). LCMS (ESI+) m/z 333.1 (M+H)⁺.

### Example 44

### (R,E)-N-(1-Hydroxybutan-2-yl)-5-methoxy-4-(2-(spiro[2.3]hexan-5-yl)vinyl)picolinamide

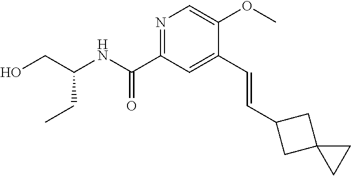 [SMILES: CC[C@H](CO)NC(=O)c1cc(/C=C/C2CC3(CC3)C2)c(OC)cn1]

Following a similar procedure to that of Example 35, the title compound was prepared from (E)-5-Methoxy-4-(2-(spiro[2.3]hexan-5-yl)vinyl)picolinic acid and (R)-2-amino-1-butanol. ¹HNMR (400 MHz, CD₃OD) δ 8.29 (s, 1H), 8.15 (s, 1H), 6.84 (dd, J=16.0, 6.8 Hz, 1H), 6.65 (d, J=16.0 Hz, 1H), 4.03-3.97 (m, 4H), 3.69-362 (m, 2H), 3.35-3.33 (m, 1H), 2.31-2.18 (m, 4H), 1.75-1.60 (m, 2H), 1.00-0.96 (m, 3H), 0.51-0.40 (m, 4H). LCMS (ESI⁺) m/z 333.1 (M+H)⁺.

### Example 45

### N-(6-Methoxy-5-((E)-2-(trans-4-(trifluoromethyl)cyclohexyl)vinyl) pyridazin-3-yl)methanesulfonamide

The overall Example 45 reaction scheme was as follows:

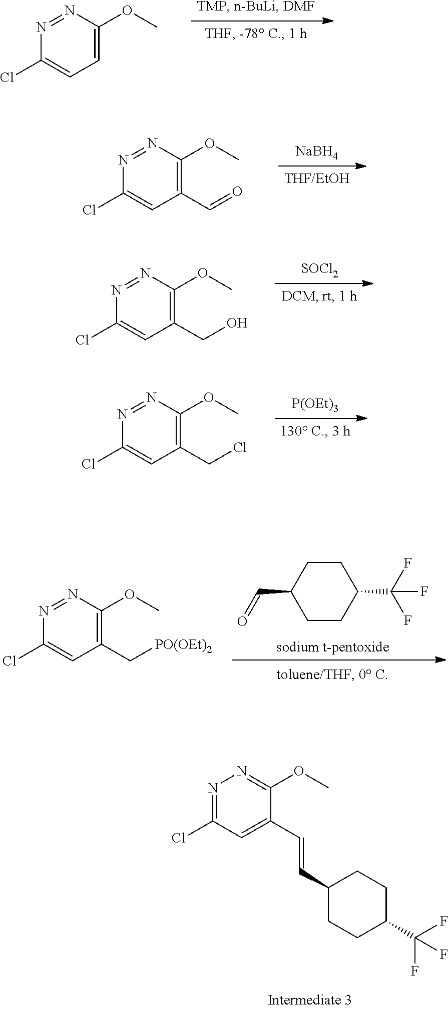

### Step 1: 6-Chloro-3-methoxypyridazine-4-carbaldehyde

To a solution of 2,2,6,6-tetramethylpiperidine (2.6 mL, 15.2 mol) in tetrahydrofuran (25 mL) was added n-butyllithium (2.5 M, 6.1 mL, 15.2 mmol) at −78° C. The reaction mixture was stirred at 0° C. for 30 minutes. Then the mixture was cooled to −78° C. A solution (pre-cooled to −78° C.) of 3-chloro-6-methoxypyridazine (2.0 g, 13.8 mmol) in THF (10 mL) was added dropwise. The resulting mixture was stirred at −78° C. for 30 minutes. N,N-dimethylformamide (1.33 mL, 41.5 mmol, pre-cooled to −78° C.) was added dropwise. The reaction mixture was stirred at −78° C. for 90 minutes. To the reaction mixture was added a pre-mixed solution of conc. HCl (10 mL), EtOH (15 mL) and THF (20 mL). The reaction mixture was warmed to 15° C., and extracted with EtOAc (100 mL×2). The combined organic layers were dried over anhydrous Na₂SO₄, filtered and concentrated to afford the title compound (2.3 g, 96%) as a light brown oil which was used for next step immediately without further purification.

### Step 2: (6-Chloro-3-methoxypyridazin-4-yl)methanol

To the mixture of 6-chloro-3-methoxy-pyridazine-4-carbaldehyde (From step 1, 2.3 g, 13.3 mmol) in methanol (30 mL) was added sodium borohydride (0.61 g, 16.0 mmol) at 15° C. The reaction was stirred at 15° C. for 16 hours. To the reaction mixture was added water (20 mL) and the mixture was concentrated to remove organic solvent. The remaining solution was extracted with EtOAc (100 mL×2). The combined organic layers were dried over anhydrous Na₂SO₄, concentrated and purified by column (0-30% EtOAc in petroleum ether) to afford the title compound (1.9 g, 86% purity) as a white solid. ¹H NMR (400 MHz, CDCl₃) δ 7.58 (t, J=1.6 Hz, 1H), 4.75 (d, J=5.6 Hz, 2H), 4.13 (s, 3H), 2.60 (t, J=5.6 Hz, 1H).

### Step 3: 6-Chloro-4-(chloromethyl)-3-methoxypyridazine

To the mixture of (6-chloro-3-methoxy-pyridazin-4-yl)methanol (From step 2, 1.0 g, 5.7 mmol) in dichloromethane (15 mL) was added thionyl chloride (1.7 mL, 22.9 mmol) at 0° C., The mixture was stirred at 0° C. for 1 hour. The mixture was concentrated to afford the title compound (1.1 g, 99%) as a black solid. The crude product was used for next step immediately without further purification.

### Step 4: Diethyl ((6-chloro-3-methoxypyridazin-4-yl)methyl)phosphonate

Following a similar procedure to that of Example 41, the title compound was prepared from 6-chloro-4-(chloromethyl)-3-methoxy-pyridazine and triethyl phosphite as a light brown oil (1.3 g, 77% purity). ¹H NMR (400 MHz, CDCl₃) δ 7.40 (s, 1H), 4.13 (s, 3H), 4.10 (q, J=7.2 Hz, 4H), 3.17-3.09 (m, 2H), 1.30 (t, J=7.2 Hz, 6H).

### Step 5: 6-Chloro-3-methoxy-4-((E)-2-(trans-4-(trifluoromethyl) cyclohexyl)vinyl)pyridazine

Following a similar procedure to that of step 2 of Example 36, the title compound was prepared from diethyl ((6-chloro-3-methoxypyridazin-4-yl)methyl)phosphonate and 4-(trifluoromethyl)cyclohexane carbaldehyde as a colorless oil (220 mg, 40%). ¹H NMR (400 MHz, CDCl₃) δ 7.34 (s, 1H), 6.57 (dd, J=16.0, 6.8 Hz, 1H), 6.47-6.40 (d, J=16.0 Hz, 1H), 4.15 (s, 3H), 2.30-2.17 (m, 1H), 2.11-1.92 (m, 5H), 1.50-1.32 (m, 2H), 1.31-1.16 (m, 2H). LCMS (ESI+) m/z 321.0 (M+H)⁺.

### Step 6: N-(6-Methoxy-5-((E)-2-(trans-4-(trifluoromethyl)cyclohexyl) vinyl)pyridazin-3-yl)methanesulfonamide

Following a similar procedure to that of Example 36, the title compound was prepared from 6-chloro-3-methoxy-4-[(E)-2-[trans-4-(trifluoromethyl)cyclohexyl]vinyl]pyridazine and methane sulfonamide as a white solid (16.8 mg, 14%). ¹H NMR (400 MHz, CD₃OD) δ 7.60 (s, 1H), 6.71 (dd, J=16.0, 6.8 Hz, 1H), 6.48 (d, J=16.0 Hz, 1H), 3.97 (s, 3H), 3.05 (s, 3H), 2.30-2.19 (m, 1H), 2.18-2.06 (m, 1H), 2.02-1.88 (m, 4H), 1.46-1.28 (m, 4H). LCMS (ESI+) m/z 380.1 (M+H)⁺.

### Example 46

### N—((R)-1-Hydroxybutan-2-yl)-6-methoxy-5-((E)-2-(trans-4-(trifluoromethyl)cyclohexyl)vinyl)pyridazine-3-carboxamide

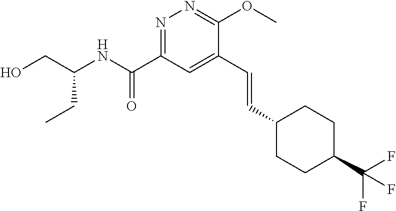 [SMILES: CC[C@H](CO)NC(=O)c1cc(/C=C/[C@H]2CC[C@H](C(F)(F)F)CC2)c(OC)nn1]

A mixture of 6-chloro-3-methoxy-4-[(E)-2-[trans-4-(trifluoromethyl) cyclohexyl]vinyl] pyridazine (130 mg, 0.4 mmol), (R)-2-amino-1-butanol (108 mg, 1.2 mmol), palladium acetate (9 mg, 0.04 mmol), 1,3-bis(diphenylphosphino) propane (33.4 mg, 0.08 mmol) and potassium carbonate (168 mg, 1.22 mmol) in N,N-dimethylformamide (5 mL) was heated at 80° C. under CO atmosphere (50 psi) for 16 hours. The solution was diluted with EtOAc (20 mL), washed with water (5 mL×3). The organic layer was dried over anhydrous Na₂SO₄, filtered and concentrated. The residue was purified by preparative-TLC (10% MeOH in DCM) to afford the crude product (30 mg) which was further purified by reverse phase chromatography (Phenomenex Gemini 150*25 mm*10 um, water (0.05% ammonia hydroxide v/v)-ACN, 49%-79%) to afford the title compound (7.4 mg, 5%) as a white solid. ¹H NMR (400 MHz, CD₃OD) δ 8.12 (s, 1H), 6.74 (dd, J=16.0, 7.2 Hz, 1H), 6.56 (d, J=16.0 Hz, 1H), 4.19 (s, 3H), 4.06-3.97 (m, 1H), 3.66-3.64 (m, 2H), 2.26-2.30 (m, 1H), 2.18-2.07 (m, 1H), 1.99-1.95 (m, 4H), 1.81-1.54 (m, 2H), 1.45-1.26 (m, 4H), 0.97 (t, J=7.6 Hz, 3H). LCMS (ESI+) m/z 402.1 (M+H)⁺.

### Example 47

### (R,E)-N-(1-hydroxybutan-2-yl)-6-methoxy-5-(2-(spiro[2.3]hexan-5-yl) vinyl)pyridazine-3-carboxamide

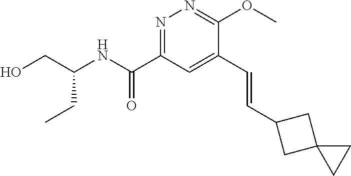 [SMILES: CC[C@H](CO)NC(=O)c1cc(/C=C/C2CC3(CC3)C2)c(OC)nn1]

### Step 1: (E)-6-chloro-3-methoxy-4-(2-(spiro[2.3]hexan-5-yl)vinyl) pyridazine

Following a similar procedure to that of Example 45, the title compound was prepared from diethyl ((6-chloro-3-methoxypyridazin-4-yl)methyl)phosphonate and spiro[2.3]hexane-5-carbaldehyde as a light yellow oil (0.25 g, 49%). ¹H NMR (400 MHz, CDCl₃) δ 7.38 (s, 1H), 6.89 (dd, J=15.6, 7.6 Hz, 1H), 6.40 (d, J=16.0 Hz, 1H), 4.16 (s, 3H), 3.39-3.29 (m, 1H), 2.32-2.27 (m, 2H), 2.22-2.17 (m, 2H), 0.50-0.41 (m, 4H).

### Step 2: (R,E)-N-(1-hydroxybutan-2-yl)-6-methoxy-5-(2-(spiro[2.3]hexan-5-yl) vinyl)pyridazine-3-carboxamide

Following a similar procedure to that of Example 46, the title compound was prepared from (E)-6-chloro-3-methoxy-4-(2-(spiro[2.3]hexan-5-yl)vinyl)pyridazine and (R)-(−)-2-amino-1-butanol as a light yellow solid (50 mg, 25%). ¹H NMR (400 MHz, CD₃OD) δ 8.15 (s, 1H), 7.05 (dd, J=15.6, 7.6 Hz, 1H), 6.52 (d, J=16.0 Hz, 1H), 4.21 (s, 1H), 4.08-4.02 (m, 1H), 3.68-3.67 (m, 2H), 3.38-3.31 (m, 1H), 2.32-2.22 (m, 4H), 1.82-1.71 (m, 1H), 1.68-1.57 (m, 1H), 1.01-0.98 (t, J=7.2 Hz, 3H), 0.51-0.41 (m, 4H). LCMS (ESI+) m/z 332.0 (M+H)⁺.

### Example 48

### (E)-N-(5-methoxy-4-(2-(4-(trifluoromethyl)cyclohexyl)vinyl)pyridin-2-yl)methanesulfonamide

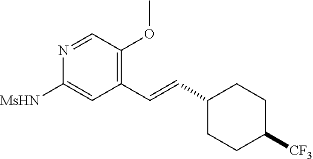

### Step 1: (E)-N-(5-methoxy-4-(2-(4-(trifluoromethyl)cyclohexyl)vinyl) pyridin-2-yl)methanesulfonamide

Diethyl ((5-methoxy-2-(methylsulfonamido)pyridin-4-yl)methyl)phosphonate (59 mg, 0.167 mmol) was dissolved in THF (0.5 mL) and cooled to 0° C. Followed by NaH (10 mg, 0.251 mmol, 1.5 eq., 60%) was added under argon atmosphere and the mixture was stirred at 0° C. for 30 min. 4-(Trifluoromethyl)cyclohexane-1-carbaldehyde (30.2 mg, 0.167) was added to the reaction at 0° C. and the mixture was stirred at rt until consumption of starting material had been observed. The reaction mixture was poured into ice water and the compound was extracted into ethyl acetate (3×2 mL) and the combined organic layer was dried using anhydrous Na₂SO₄, filtered and concentrated under vacuum to yield oil. The crude was purified by using flash chromatography using DCM and MeOH as gradients to yield off white solid (39 mg, 61.5%). LCMS (ESI+) m/z 379.0. ¹H NMR (400 MHz, DMSO-d₆) δ=10.10 (br s, 1H), 8.02 (br s, 1H), 7.02 (s, 1H), 6.58-6.48 (m, 1H), 6.44-6.31 (m, 1H), 3.86 (s, 3H), 3.21 (s, 3H), 2.32-2.13 (m, 2H), 1.96-1.81 (m, 4H), 1.40-1.20 (m, 4H).

### Example 49

### N-[3-[(E)-2-(4-chlorophenyl)vinyl]-4-methoxy-phenyl]cyclopropanesulfonamide

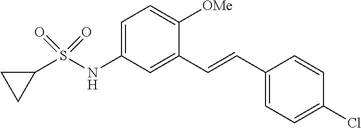 [SMILES: COc1ccc(NS(=O)(=O)C2CC2)cc1/C=C/c1ccc(Cl)cc1]

### Step 1: (E)-3-(4-chlorostyryl)-4-methoxyaniline

To a 100 mL screw top vial was added 3-bromo-4-methoxy-aniline (1 g, 4.9 mmol) phosphate tribasic (2 equiv., 9.8985 mmol), 4-chloro-beta-styrylboronic acid pinacol ester (1.5 equiv., 7.4239 mmol), chloro(2-dicyclohexylphosphino-2′,6′-dimethoxy-1,1′-biphenyl)(2′-amino-1,1′-biphenyl-2-yl) palladium(II) (0.05 equiv., 0.24746 mmol), dicyclohexyl((2-[(2,6-dimethoxyphenyl)methyl]phenyl)methyl)phosphane (0.05 equiv., 0.24746 mmol), and 10:1 Tol/Water (0.25 M, 20 mL). The reaction was the then heated to 80° C. overnight. The reaction was cooled to rt, filtered with Celite®, dried with MgSO4, and concentrated. The organics where then purified by silica get chromatography from 0% Heptane to 100% iPrOAc to yield 3-[(E)-2-(4-chlorophenyl)vinyl]-4-methoxy-aniline (1 g, 3.851 mmol) 77% Yield. LCMS (ESI+) m/z 259.9 (M+H)^{+ 1}H NMR (400 MHz, DMSO-d6) δ=7.58-7.53 (m, 2H), 7.42-7.38 (m, 2H), 7.34 (d, J=16.5 Hz, 1H), 7.02 (d, J=16.5 Hz, 1H), 6.88 (d, J=2.7 Hz, 1H) 6.77 (d, J=8.7 Hz, 1H), 6.54 (dd, J=8.6, 2.7 Hz, 1H), 3.72 (s, 3H).

### Step 2: N-[3-[(E)-2-(4-chlorophenyl)vinyl]-4-methoxy-phenyl]cyclopropanesulfonamide

To a 100 ml vial was added 3-[(I)-2-(4-chlorophenyl)vinyl]-4-methoxy-aniline (100 mg, 0.39 mmol), N,N-diisopropylethylamine (4 equiv., 1.54 mmol), cyclopropanesulfonyl chloride (1.5 equiv., 0.5776 mmol), and dichloromethane (0.2 M, 2 mL). The reaction was stirred until LC-MS indicated that the starting material was consumed then filtered through a 0.45 μM filter and concentrated. The organics purified by reverse phase HPLC (0.1% Formic Acid in water/Acetonitrile 40-80, Gemini-NX C18 10 uM) to yield N-[3-[(E)-2-(4-chlorophenyl)vinyl]-4-methoxy-phenyl]cyclopropanesulfonamide (53.5 mg, 0.147 mmol, 38.2%) LCMS (ESI+) m/z 364.0 (M)^{+ 1}H NMR (400 MHz, DMSO-d6) δ 9.39 (s, 1), 7.65-7.54 (m, 2H), 7.51-7.34 (m, 4H), 7.21-6.99 (m, 3H), 3.84 (s, 3H) 2.59-2.53 (m, 1H), 1.00-0.82 (m, 4H).

### Example 50

### (E)-2-cyano-N-(5-(2-(4,4-difluorocyclohexyl)vinyl)-6-methoxypyridin-3-yl)-2-methylpropane-1-sulfonamide

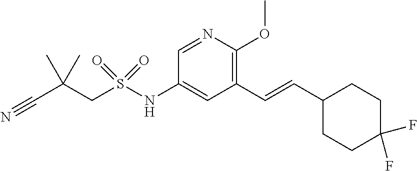 [SMILES: COc1ncc(NS(=O)(=O)CC(C)(C)C#N)cc1/C=C/C1CCC(F)(F)CC1]

5-[(E)-2-(4,4-Difluorocyclohexyl)vinyl]-6-methoxy-pyridin-3-amine (50 mg, 0.18 mmol), was diluted in pyridine (0.2 M, 1 mL), and then 2-cyano-2-methyl-propane-1-sulfonyl chloride (36 mg, 1.1 equiv., 0.2050 mmol) was added. The reaction was stirred till LC-MS indicated that starting material was consumed. The reaction mixture was then filtered through a 0.45 uM filter and concentrated. The reaction were purified by SFC (5-60%, 1% NH₄OH in water, Pyridyl Amide) to furnish 2-cyano-N-[5-[(E)-2-(4,4-difluorocyclohexyl)vinyl]-6-methoxy-3-pyridyl]-2-methyl-propane-1-sulfonamide (31.4 mg, 0.0759 mmol, 41%). LCMS (ESI+) m/z 414.1 (M+H)^{+ 1}H NMR (400 MHz, DMSO-d6) δ 9.75 (s, 1H), 8.27 (d, J=2.5 Hz, 1H), 7.98 (d, J=2.5 Hz, 1H), 6.53 (dd, J=163, 1.2 Hz, 1H), 6.32 (dd, J=16.2, 6.9 Hz, 1H), 3.90 (s, 3H), 3.34 (s, 2H), 2.41-2.29 (m, 1H), 2.16-1.77 (m, 6H), 1.52 (m 8H).

### Example 51

### (E)-3-(4-Chlorostyryl)-N-(4-hydroxybutan-2-yl)-4-methoxybenzamide

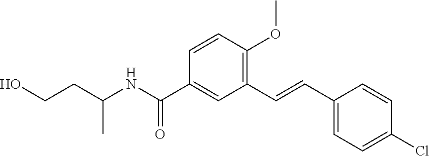 [SMILES: COc1ccc(C(=O)NC(C)CCO)cc1/C=C/c1ccc(Cl)cc1]

### Step 1: ethyl 3-formyl-4-hydroxybenzoate

To a solution of ethyl 4-hydroxybenzoate (100 g, 0.6 mol) and Et₃N (450 mL, 3.6 mol) in DCE (1 L) was added MgCl₂ (285 g, 3 mol) and the mixture was stirred for 1 h at 40° C. Paraformaldehyde (180 g, 6 mol) was added and the mixture was stirred for 3 h at 70° C. After cooling to 0° C., 1M HCl (3 L) was added. The mixture was filtered, and washed with DCM (170 mL). The organic layer was separated, washed with 1M HCl (170 mL) and brine (170 mL), dried over anhydrous Na₂SO₄ and concentrated in vacuo. The crude product was purified by silica column chromatography to afford the desired product (80 g, 68%). ¹H NMR (300 MHz, CDCl₃): δ 11.40 (s, 1H), 9.97 (s, 1H), 8.34 (s, 1H), 8.21 (d, J=10.8 Hz, 1H), 7.05 (d, J=8.7 Hz, 1H), 4.41 (q, J=7.2 Hz, 2H), 1.42 (t, J=7.2 Hz, 2H).

### Step 2: ethyl 3-formyl-4-methoxybenzoate

To a solution of ethyl 3-formyl-4-hydroxybenzoate (155 g, 0.8 mol, 1 eq) in acetone (1.5 L) were added K₂CO3 (144 g, 1.04 mol, 1.3 eq) and Me₂CO₃ (86.4 g, 0.96 mol, 1.2 eq). The resultant mixture was stirred at reflux for 1 h. After cooling, the insoluble was filtered and cake was washed with EtOAc (100 mL×3). The filtrate was diluted with EtOAc (1 L) and aqueous NaHCO₃ (1 L). The organic layer was separated and the aqueous layer was extracted with EtOAc (3×1 L). The combined organic layers were washed with H₂O (2×1 L) and dried over MgSO₄. The solvent was evaporated to get the crude product ethyl 3-formyl-4-methoxybenzoate which was purified on silica column affording the desired product (115 g, 69%). ¹HNMR (300 MHz, CDCl₃): □□ 10.47 (s, 1H), 8.51 (s, 1H), 8.26 (d, J=10.8 Hz, 1H), 7.05 (d, J=8.7 Hz, 1H), 4.38 (q, J=7.2 Hz, 2H), 4.02 (s, 3H), 1.41 (t, J=7.2 Hz, 2H).

### Step 3: ethyl (E)-3-(4-chlorostyryl)-4-methoxybenzoate

To a solution of diethyl (4-chlorobenzyl)phosphonate (160 g, 0.61 mol) in toluene (1.5 L) at 0° C. was added sodium tertpentoxide (89 g, 0.87 mol) and the mixture was stirred for 20 min at 0° C. Then a solution of ethyl 3-formyl-4-methoxybenzoate (120 g, 0.58 mol, 1 eq) in THF (500 mL) was added dropwise over 20 min and the reaction mixture was stirred for 1.5 h at 0° C. The reaction mixture was poured into saturated aqueous solution of NH₄Cl (3 L) and extracted with EtOAc (2 L×2). The organic layers were combined, washed with brine (2 L), dried over anhydrous Na₂SO₄, filtered, and concentrated in vacuo to give crude ethyl (E)-3-(4-chlorostyryl)-4-methoxybenzoate (204 g). The crude product obtained was used in next step without purification.

### Step 4 (E)-3-(4-chlorostyryl)-4-methoxybenzoic Acid

To a solution of crude ethyl (E)-3-(4-chlorostyryl)-4-methoxybenzoate (160 g) in MeOH (1 L) was added 20% aqueous solution of KOH (260 mL). The mixture was stirred for 2 h at 65° C., and then cooled to 0° C. The reaction mixture was adjusted to pH=3 by addition of 1 M HCl. The resulting precipitate was filtered to afford desired product (E)-3-(4-chlorostyryl)-4-methoxybenzoic acid (104 g). LCMS (ESI+) m/z 286.7 (M−H)^{- 1}H NMR (300 MHz, DMSO-d₆): δ 12.77 (br, 1H), 8.21 (s, 1H), 7.89 (d, J=8.7 Hz, 1H), 7.66-7.63 (m, 2H), 7.46-7.41 (m, 3H), 7.27-7.17 (m, 1H), 7.15 (d, J=8.7 Hz, 1H), 3.94 (s, 3H).

### Step 5: ((E)-3-(4-chlorostyryl)-N-(4-hydroxybutan-2-yl)-4-methoxybenzamide

The 3-[(E)-2-(4-chlorophenyl)vinyl]-4-methoxy-benzoic acid (100 mg, 0.3464 mmol) was added to a 20 mL vial, then DMF (0.2 M, 1.2 mL), then triethylamine (0.193 mL, 1.4 mmol), then Pybop (270 mg, 0.51 mmol) were added. The reaction was stirred for 30 min and then the 3-amino-butan-1-ol (62 mg, 0.6928 mmol) was added. The reaction was quenched with sat. aq. sodium carbonate, extracted with iPrOAc, dried with MgSO4, filtered and concentrated. The crude product was purified by chiral SFC (25% MeOH w/ 0.1% NH4OH, Chiralpak IC, Peak 2 (RT=1.14 min)) to furnish the desired product in 20% Yield (25.2 mg). LCMS (ESI+) m/z 360.1 (M+1)^{+ 1}H NMR (400 MHz, DMSO-d6) δ 8.16-8.07 (m, 2), 7.80 (dd, J=8.7, 2.2 Hz, 1H), 7.67-7.58 (m, 2H), 7.48-7.38 (m, 3H), 7.28 (d, J=16.5 Hz, 1H), 7.11 (d, J=8.7 Hz, 1H), 4.43 (t, J=5.1 Hz, 1H), 4.13 (m, 1H), 3.91 (s, 3H), 3.46 (m, 2H), 1.79-1.57 (m, 2H) 1.17 (d, J=6.6 Hz, 3H).

### Example 52

### (E)-N-(6-cyclopropyl-5-(2-(4,4-difluorocyclohexyl)vinyl)pyridin-3-yl) methanesulfonamide

The overall Example 52 reaction scheme was as follows:

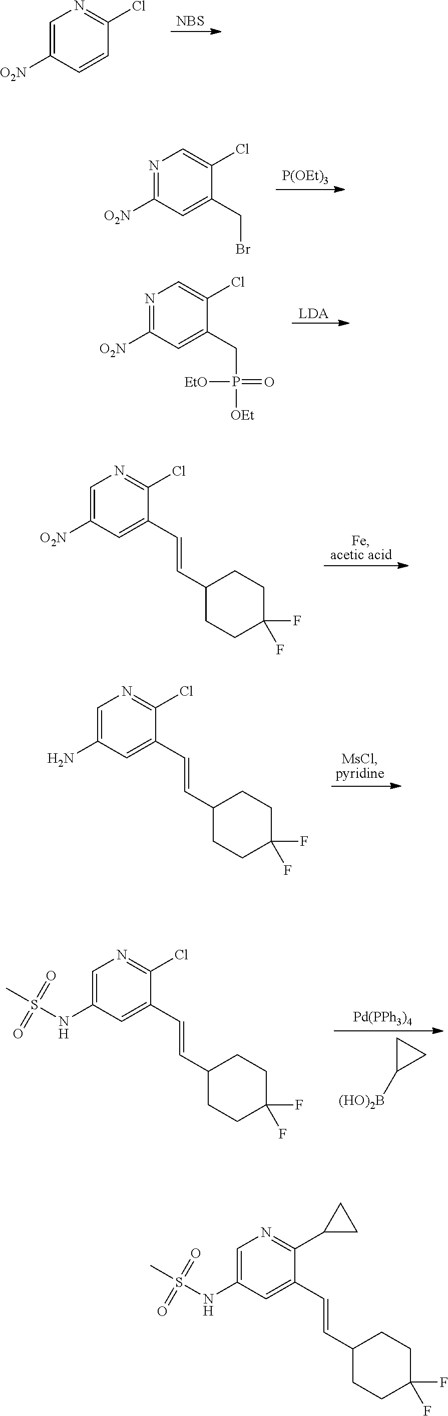

### Step 1: 3-(bromomethyl)-2-chloro-5-nitropyridine

To a solution of 2-chloro-3-methyl-5-nitropyridine (1.72 g, 10 mmol) in CCl₄ (20 mL) was added NBS (1.96 g, 11 mmol), and AIBN (164 mg, 1 mmol). The reaction mixture was heated to 120° C. in a sealed tube and stirred for 2 h. Then the reaction was cooled to room temperature and was concentrated under reduced pressure to give a crude residue. 20 mL of water was added and the organics were extracted with EtOAc (20 mL×3). The combined organic layers were dried over Na₂SO₄, filtrated and concentrated to give a crude residue. The residue was purified on silica gel chromatography to furnish the title compound (900 mg, 36%). ¹H NMR (300 MHz, CDCl₃): δ 9.19 (s, 1H), 8.61 (s, 1H), 4.63 (s, 2H).

### Step 2: diethyl ((2-chloro-5-nitropyridin-3-yl)methyl)phosphonate

To a solution of 3-(bromomethyl)-2-chloro-5-nitropyridine (900 mg, 3.6 mmol) in 1,4-dioxane (10 mL) was added triethyl phosphite (12 g, 7.2 mmol). The reaction mixture was heated to reflux and stirred overnight. The reaction mixture was concentrated under reduced pressure to give a crude residue, which was purified on silica gel chromatography to afford the title compound (300 ng, 27%). ¹H NMR (300 MHz, CDCl₃): δ 9.11 (s, 1H), 8.56 (s, 1H), 4.15-4.11 (m, 4H), 3.44 (d, J=21.9 Hz, 2H), 1.33-1.11 (m, 6H).

### Step 3: (E)-2-chloro-3-(2-(4,4-difluorocyclohexyl)vinyl)-5-nitropyridine

A solution of diisopropylamine (0.4 mL, 2.8 mmol) in THF (15 mL) was cooled to −78° C. under an atmosphere of argon. N-butyllithium (1.12 mL, 2.5 M in hexane) was added slowly and then a solution of diethyl ((2-chloro-5-nitropyridin-3-yl)methyl)phosphonate (862 mg, 2.8 mmol) in THF (10 mL) was added. The mixture was warmed to 0° C. and then stirred for 1 hour. A solution of 4,4-difluorocyclohexane-1-carbaldehyde (370 mg, 2.5 mmol) in dry THF (15 mL) was added dropwise. The reaction was kept at 0° C. for 2 hours, then allowed to warm to room temperature and stirred for 24 hours. The reaction was quenched with a saturated aqueous NH₄Cl solution and extracted with EtOAc. The combined organic phases were dried, filtered and concentrated to dryness. Silica gel chromatography afforded the title compound (250 mg, 29%). ¹H NMR (300 MHz, CDCl₃) δ 9.08 (s, 1H), 8.57 (s, 1H), 6.75 (d, J=15.6 Hz, 1H), 6.44-6.36 (m, 1H), 2.42-2.40 (m, 1H), 2.20-2.18 (m, 2H), 1.94-1.58 (m, 6H).

### Step 4: (E)-6-chloro-5-(2-(4,4-difluorocyclohexyl)vinyl)pyridin-3-amine

To a stirred solution of (E)-2-chloro-3-(2-(4,4-difluorocyclohexyl)vinyl)-5-nitropyridine (600 mg, 2 mmol) in acetic acid (3 mL) was added iron powder (560 mg, 10 mmol). The reaction was heated to 80° C. and stirred for 2 hours. At which point the reaction was filtered through Celite® and washed with acetic acid. The filtrate was evaporated to dryness and then the solution was adjusted to pH8 with a saturated aqueous solution of sodium bicarbonate. The organics were extracted with dichloromethane (5 mL×5). The combined organic material was washed with brine, dried over Na₂SO₄, filtered and concentrated to afford the title compound which was used directly in next step without further purification (450 mg, crude). ¹H NMR (300 MHz, CDCl₃): δ 7.79 (s, 1H), 7.15 (s, 1H), 6.65 (d, J=15.6 Hz, 1H), 6.15-6.07 (m, 1H), 3.53 (br, 2H), 2.32-2.30 (m, 1H), 2.17-2.14 (m, 2H), 1.92-1.57 (m, 6H).

### Step 5: (E)-N-(6-chloro-5-(2-(4,4-difluorocyclohexyl)vinyl)pyridin-3-yl) methanesulfonamide

A solution of (E)-6-chloro-5-(2-(4,4-difluorocyclohexyl)vinyl)pyridin-3-amine (450 mg, 1.65 mmol) and pyridine (156.4 mg, 1.98 mmol, 1.2 eq) in DCM (3 mL) was cooled to 10° C. To the solution was added methanesulphonyl chloride (225.7 mg, 1.98 mmol) dropwise. The reaction mixture was allowed to warm to room temperature and stirred for an additional 20 hours. The reaction mixture was then diluted with DCM (5 mL) and washed with water (10 mL) and brine. The organic layer was dried over anhydrous sodium sulfate and concentrated to give a crude residue. The organic material was purified on silica gel chromatography to furnish the title compound (210 mg, 36% total for steps 4&5). ¹H NMR (300 MHz, CDCl₃): δ 8.19 (s, 1H), 7.92-7.86 (m, 2H), 6.66 (d, J=15.6 Hz, 1H), 6.28-6.21 (m, 1H), 3.09 (s, 3H), 2.34-2.30 (m, 1H), 2.14-2.07 (m, 2H), 1.89-1.57 (m, 6H).

### Step 6: (E)-N-(6-cyclopropyl-5-(2-(4,4-difluorocyclohexyl)vinyl)pyridin-3-yl)methanesulfonamide

(E)-N-(6-chloro-5-(2-(4,4-difluorocyclohexyl)vinyl)pyridin-3-yl)methanesulfonamide (150 mg, 0.4276 mmol), tetrakis(triphenylphosphine)palladium(0) (49 mg, 0.043 mmol), potassium carbonate (236 mg, 1.71 mmol) and cyclopropyl boronic acid (116 mg, 1.28 mmol) were charged into a 10 ml seal tube. Then 1,4-dioxane (0.85 mL) and water (0.09 mL) were added and the solution was degassed with nitrogen for 5 minutes then sealed. The reaction was then heated to 110° C. for 72 hours, at which point it was cooled to room temperature. The organic material was filtered through a 0.45 uM filter and concentrated. Purification by achiral SFC (5-15% CO₂/0.1% NH₄OH in MeOH, Torus DEA column) yielded the title compound (10.3 mg, 7% yield). LCMS (ESI+) m/z 357.2 (M+1)^{+ 1}H NMR (400 MHz, DMSO-d6) δ 9.69 (s, 1H), 8.15 (d, J=2.4 Hz, 1H), 7.53 (d, J=2.4 Hz, 1H), 6.85 (dd, J=16.0, 1.3 Hz, 1H), 6.15 (dd, J=16.0, 6.8 Hz, 1H), 2.99 (s, 3H), 2.45-2.31 (m, 1H), 2.23 (m, 1H), 2.06 (m, 2H), 1.99-1.78 (m, 4H), 1.57-1.38 (m, 2H), 0.95-0.85 (m, 4H).

### Example 53

### Example 53A: Preparation of 4,4-Difluorocyclohexanecarbaldehyde, and Intermediate of the Structure

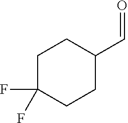

To a mixture of ethyl 4,4-difluorocyclohexanecarboxylate (1.0 g, 5.2 mmol) in dichloromethane (20 mL) was added DIBAL-H (4.8 mL, 4.8 mmol) at −78° C. The reaction was stirred at −78° C. for 1 h. The reaction was quenched with NH₄Cl (5 mL). The mixture was dried over MgSO₄, filtered and concentrated to afford the title compound (1 g, 75% purity) as a colorless oil, which was used for the next step directly without further purification.

### Example 53B: Preparation of 4-(Trifluoromethyl)cyclohexanecarbaldehyde

The overall Example 53B reaction scheme was as follows:

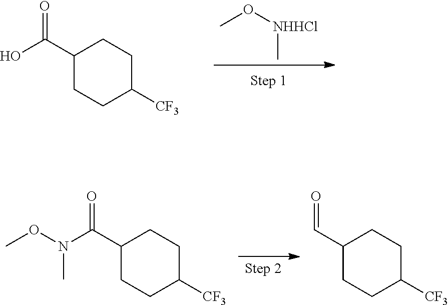

### Step 1: N-Methoxy-N-methyl-4-(trifluoromethyl)cyclohexanecarboxamide

A mixture of HATU (11.7 g, 30.59 mmol), N,O-dimethylhydroxylamine hydrochloride (3 g, 30.6 mmol) and 4-(trifluoromethyl)cyclohexanecarboxylic acid (5 g, 25.5 mmol) was dissolved in DMF (50 mL). N,N-Diisopropylethylamine (17.8 mL, 102.0 mmol) was then added. The mixture was stirred at 20° C. for 1 h. The resulting solution was extracted with ethyl acetate (20 mL×2) and the organic layers were combined. The combined organic layer was dried over anhydrous sodium sulfate and concentrated under vacuum. The residue was purified by flash chromatography on silica gel eluting with methyl MeOH/DCM (1:15) to afford the title compound (5.7 g, 93%) as a white solid. ¹H NMR (400 MHz, CDCl₃): δ 3.69 (s, 3H), 3.19 (s, 3H), 2.97-2.85 (m, 1H), 2.12-2.06 (m, 1H), 2.04-1.88 (m, 4H), 1.78-1.66 (m, 2H), 1.63-1.54 (m, 2H).

### Step 2: 4-(Trifluoromethyl)cyclohexanecarbaldehyde

A mixture of N-methoxy-N-methyl-4-(trifluoromethyl)cyclohexanecarboxamide (3.8 g, 16 mmol) in dichloromethane (62 mL) was was combined with DIBAL-H (47.65 mL, 47.65 mmol) at −78° C. The reaction was stirred at −78° C. for 1 h. The reaction was quenched with saturated NH₄Cl (5 mL). The mixture was then dried over MgSO₄, filtered and concentrated to afford the title compound (1 g, 70% purity) as a colorless oil, which was subsequently directly used without further purification.

### Example 54

### (E)-5-(2-(4,4-difluorocyclohexyl)vinyl)-6-methoxy-N-(3-(2-(methylamino)-2-oxoethyl)benzyl)nicotinamide

The overall Example 54 reaction scheme was as follows:

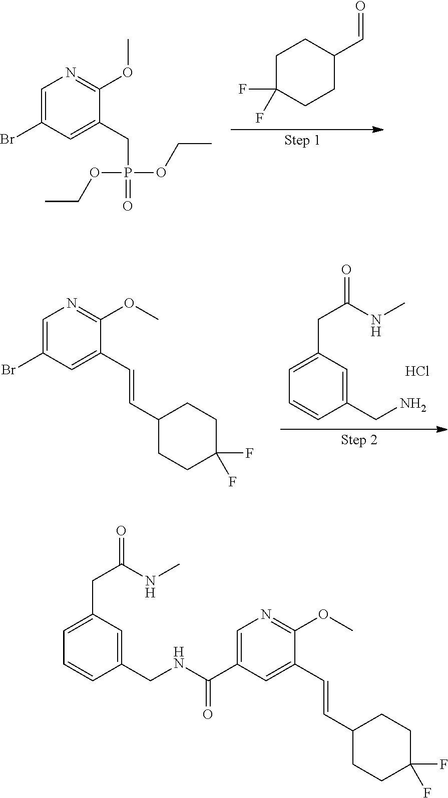

### Step 1: (E)-5-Bromo-3-(2-(4,4-difluorocyclohexyl)vinyl)-2-methoxypyridine

To a solution of 5-bromo-3-(diethoxyphosphorylmethyl)-2-methoxy-pyridine (0.2 g, 0.59 mmol) in toluene (10 mL) at 0° C. was added sodium tert-pentoxide (0.07 g, 0.65 mmol) and the mixture was stirred for 20 min at 0° C. A solution of 4,4-difluorocyclohexane carboxaldehyde (0.11 g, 0.56 mmol, 75% purity) in THF (2 mL) was then added dropwise and the reaction mixture was stirred for 1.5 h at 0° C. The reaction mixture was poured into saturated aqueous NH₄Cl solution (50 mL) and extracted with EtOAc (100 mL×2). The organic layer was dried over Na₂SO₄, filtered and concentrated to dryness. The residue was purified by column chromatography on silica gel (0-10% EtOAc in petroleum ether) to afford the title compound (180 mg, 91%) as a colorless oil. ¹H NMR (400 MHz, CDCl₃): δ 8.05 (d, J=2.4 Hz, 1H), 7.71 (d, J=2.4 Hz, 1H), 6.51 (d, J=15.6 Hz, 1H), 6.21 (dd, J=15.6, 7.2 Hz, 1H), 3.95 (s, 3H), 2.30-2.04 (m, 3H), 1.94-1.71 (m, 4H), 1.60-1.57 (m, 2H); LCMS (ESI): m/z 332.0 (M+H)⁺.

### Step 2: (E)-5-(2-(4,4-difluorocyclohexyl)vinyl)-6-methoxy-N-(3-(2-(methylamino)-2-oxoethyl)benzyl)nicotinamide

A mixture of 5-bromo-3-[(E)-2-(4,4-difluorocyclohexyl)vinyl]-2-methoxy-pyridine (100 mg, 0.30 mmol), Pd(OAc)₂ (7 mg, 0.03 mmol), XantPhos (35 mg, 0.06 mmol), Na₂CO₃ (160 mg, 1.51 mmol) and 2-[3-(aminomethyl)phenyl]-N-methyl-acetamide hydrochloride (130 mg, 0.60 mmol) in toluene (2 mL) and DMF (2 mL) was stirred at 80° C. for 12 hours under CO atmosphere (50 psi). The resulting solution was extracted with dichloromethane (20 mL×2) and water (20 mL). The organic layers were combined, dried over anhydrous sodium sulfate and concentrated under vacuum. The residue was purified by flash chromatography on silica gel eluting with methanol/dichloromethane (1:15) to afford the title compound (16.1 mg, 12%) as a brown solid. ¹H NMR (400 MHz, CDCl₃): δ 8.44 (d, J=2.0 Hz, 1H), 8.10 (d, J=2.0 Hz, 1H), 7.39-7.29 (m, 2H), 7.26-7.16 (m, 2H), 6.58 (d, J=16.0 Hz, 1H), 6.50 (t, J=5.6 Hz, 1H, NH), 6.31 (dd, J=16.0, 6.8 Hz, 1H), 5.44 (br s, 1H), 4.65 (d, J=5.6 Hz, 2H), 4.02 (s, 3H), 3.56 (s, 2H), 2.77 (d, J=4.8 Hz, 3H), 2.29-2.26 (m, 1H), 2.20-2.06 (m, 2H), 1.95-1.70 (m, 4H), 1.60-1.50 (m, 2H); LCMS (ESI): m/z 458.2 (M+H)⁺.

### Example 55

### (E)-4-(2-(4,4-Difluorocyclohexyl)vinyl)-5-methoxy-N-(3-(2-(methylamino)-2-oxoethyl)benzyl)picolinamide

The overall Example 55 reaction scheme was as follows:

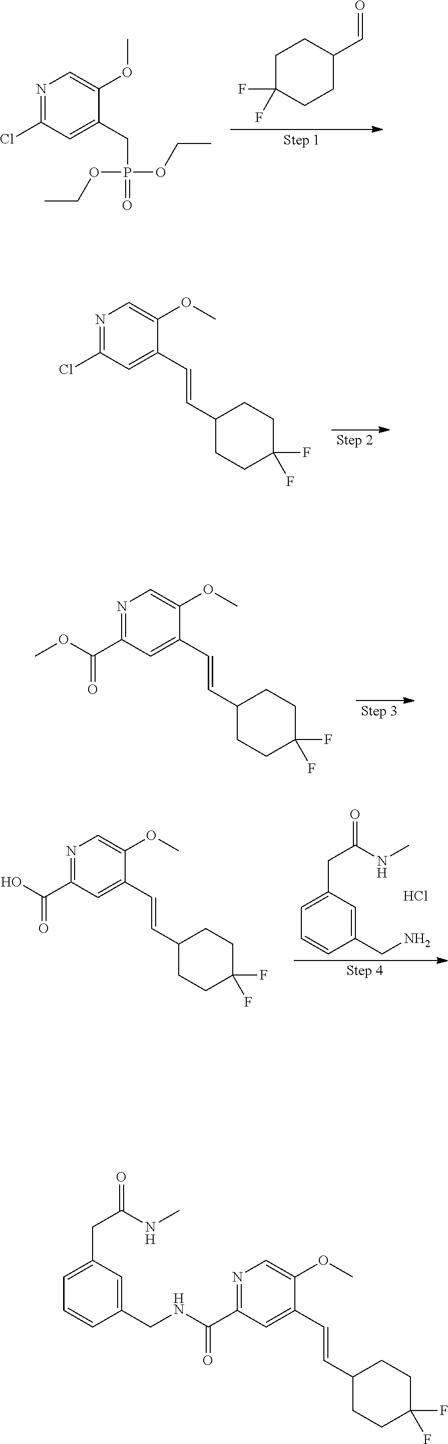

### Step 1: (E)-2-Chloro-4-(2-(4,4-difluorocyclohexyl)vinyl)-5-methoxypyridine

To a solution of 2-chloro-4-(diethoxyphosphorylmethyl)-5-methoxy-pyridine (0.3 g, 1.02 mmol) in toluene (10 mL) at 0° C. was added sodium tert-pentoxide (0.12 g, 1.12 mmol) and the mixture was stirred for 20 min at 0° C. Then a solution of 4,4-difluorocyclohexane carboxaldehyde (0.19 g, 1.25 mmol) in THF (15 mL) was added dropwise and the reaction mixture was stirred at 0° C. for 1.5 h. The reaction mixture was poured into saturated aqueous NH₄Cl solution (50 mL) and extracted with EtOAc (100 mL×2). The organic layers were combined, washed with brine (50 mL), dried over Na₂SO₄ and concentrated. The residue was purified by chromatography on silica gel (0-10% EtOAc in petroleum ether) to afford the title compound (140 mg, 48%) as a colorless oil. ¹H NMR (400 MHz, CDCl₃): δ 7.99-7.94 (m, 1H), 7.29 (s, 1H), 6.62 (d, J=16.4 Hz, 1H), 6.38 (dd, J=16.4, 6.8 Hz, 1H), 3.92 (s, 3H), 2.32-2.28 (m, 1H), 2.22-2.10 (m, 2H), 1.92-1.90 (m, 1H), 1.86-1.72 (m, 2H), 1.67-1.52 (m, 3H); LCMS (ESI): m/z 288.1 (M+H)⁺.

### Step 2: (E)-Methyl 4-(2-(4,4-difluorocyclohexyl)vinyl)-5-methoxypicolinate

A mixture of 2-chloro-4-[(E)-2-(4,4-difluorocyclohexyl)vinyl]-5-methoxy-pyridine (0.14 g, 0.49 mmol), potassium carbonate (0.13 g, 0.97 mmol), Pd(OAc)₂ (11 mg, 0.05 mmol), and 1,3-bis(diphenylphosphino)propane (41 mg, 0.10 mmol) in methanol (3 mL) was heated at 80° C. under CO atmosphere (50 psi) for 16 h. The solution was filtered be celite and the filtrate was concentrated. The residue was purified by TLC (50% EtOAc in petroleum ether, R_{f}=0.4) to afford the title compound methyl 4-[(E)-2-(4,4-difluorocyclohexyl)vinyl]-5-methoxy-pyridine-2-carboxylate (0.10 g, 66%) as a light yellow oil. LCMS (ESI): m/z 312.1 (M+H)⁺.

### Step 3: (E)-4-(2-(4,4-Difluorocyclohexyl)vinyl)-5-methoxypicolinic acid

A mixture of LiOH.H₂O (68 mg, 1.61 mmol) and methyl 4-[(E)-2-(4,4-difluorocyclohexyl)vinyl]-5-methoxy-pyridine-2-carboxylate (0.1 g, 0.32 mmol) in methanol (6 mL), water (6 mL) was stirred at 15° C. for 16 hours. The reaction mixture was concentrated to remove methanol and adjusted pH to 6 with 1 N HCl. The resulting solution was extracted with EtOAc (30 mL×2), and the organic layer was dried over Na₂SO₄ and concentrated to give the title compound (90 mg, 94%) as a brown solid. LCMS (ESI) m/z 297.9 (M+H)⁺.

### Step 4: (E)-4-(2-(4,4-Difluorocyclohexyl)vinyl)-5-methoxy-N-(3-(2-(methylamino)-2-oxoethyl)benzyl)picolinamide

To a mixture of 4-[(E)-2-(4,4-difluorocyclohexyl)vinyl]-5-methoxy-pyridine-2-carboxylic acid (90 mg, 0.34 mmol), 2-[3-(aminomethyl)phenyl]-N-methyl-acetamide hydrochloride (87 mg, 0.40 mmol) in DMF (2 mL) was added N,N-diisopropylethylamine (0.3 mL, 1.68 mmol). HATU (255 mg, 0.67 mmol) was then added. The reaction mixture was stirred at 15° C. for 16 h. Water (30 mL) was added to quench and the solution was extracted with EtOAc (30 mL×2). The organic layer was dried over Na₂SO₄ and concentrated. The residue was purified by TLC (13% MeOH in DCM, R_{f}=0.6) to afford the title compound (73.2 mg, 48%) as a white solid. ¹H NMR (400 MHz, CDCl₃): δ 8.44-8.08 (m, 3H), 7.34-7.32 (m, 2H), 7.23-7.20 (m, 1H), 6.84-6.50 (m, 2H), 5.61 (s, 1H), 4.69 (s, 2H), 4.04 (s, 3H), 3.59 (s, 2H), 2.80 (s, 3H), 2.38-2.35 (m, 1H), 2.20-2.17 (m, 2H), 2.03-1.75 (m, 4H), 1.65-1.63 (m, 2H); LCMS (ESI): m/z 458.2 (M+H)⁺.

### Example 56

### 6-Methoxy-N-(3-(2-(methylamino)-2-oxoethyl)benzyl)-5-((E)-2-(cis-4-(trifluoro methyl)cyclohexyl)vinyl)nicotinamide and 6-methoxy-N-(3-(2-(methylamino)-2-oxoethyl) benzyl)-5-((E)-2-(trans-4-(trifluoromethyl)cyclohexyl)vinyl) nicotinamide

The overall Example 56 reaction scheme was as follows:

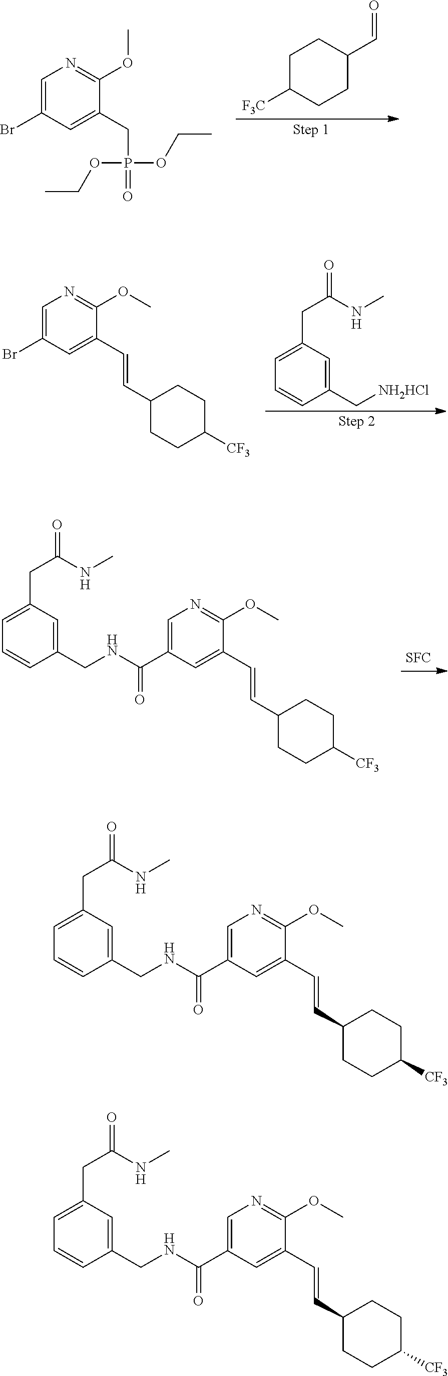

### Step 1: (E)-5-Bromo-2-methoxy-3-(2-(4-(trifluoromethyl)cyclohexyl)vinyl) pyridine

To a solution of 5-bromo-3-(diethoxyphosphorylmethyl)-2-methoxy-pyridine (0.5 g, 1.48 mmol) in toluene (7.5 mL) at 0° C. was added sodium tert-pentoxide (0.2 g, 1.77 mmol) and the mixture was stirred for 20 min at 0° C. Then a solution of 4-(trifluoromethyl) cyclohexanecarbaldehyde (0.64 g, 3.54 mmol) in tetrahydrofuran (7.5 mL) was added dropwise and the reaction mixture was stirred for 1.5 h at 0° C. The reaction mixture was poured into saturated aqueous solution of NH₄Cl (50 mL) and extracted with EtOAc (100 mL×2). The organic layers were combined, washed with brine (50 mL), dried over Na₂SO₄ and concentrated. The residue was purified by chromatography on silica gel (0-10% EtOAc in petroleum ether) to afford the title compound (100 mg, 19%) as colorless oil. LCMS (ESI): m/z 364.0 (M+H)⁺.

### Step 2: (E)-6-Methoxy-N-(3-(2-(methylamino)-2-oxoethyl)benzyl)-5-(2-(4-(trifluoromethyl)cyclohexyl)vinyl)nicotinamide

To a mixture of 2-[3-(aminomethyl)phenyl]-N-methyl-acetamide hydrochloride (354 mg, 1.65 mmol), 4,5-bis(diphenylphosphino)-9,9-dimethylxanthene (96 mg, 0.16 mmol), 5-bromo-2-methoxy-3-[(E)-2-[4-(trifluoromethyl)cyclohexyl]vinyl]pyridine (300 mg, 0.82 mmol) in N,N-dimethylformamide (7.5 mL) and toluene (7.5 mL) was added Pd(OAc)₂ (32 mg, 0.08 mmol). The reaction was stirred at 80° C. for 16 h under CO atmosphere (50 psi). Water (30 mL) was added into it and the solution was extracted with EtOAc (30 mL×2). The organic layer was dried over Na₂SO₄, filtered and concentrated. The residue was purified by TLC (13% MeOH in DCM, R_{f}=0.6) to afford the title compound (50 mg, 12%) as a white solid. LCMS (ESI): m/z 490.1 (M+H)⁺.

### Step 3: 6-Methoxy-N-(3-(2-(methylamino)-2-oxoethyl)benzyl)-5-((E)-2-(cis-4-(trifluoromethyl)cyclohexyl)vinyl)nicotinamide and 6-methoxy-N-(3-(2-(methylamino)-2-oxoethyl)benzyl)-5-((E)-2-(trans-4-(trifluoromethyl)cyclohexyl)vinyl)nicotinamide

6-Methoxy-N-[[3-[2-(methylamino)-2-oxo-ethyl]phenyl]methyl]-5-[(E)-2-[4-(trifluoromethyl)cyclohexyl]vinyl]pyridine-3-carboxamide (50 mg, 0.10 mmol) was separated by SFC (DAICEL CHIRALPAK IC (250 mm*30 mm, 5 um)), (0.1% NH₃H₂O in EtOH, 40%) to afford 6-methoxy-N-(3-(2-(methylamino)-2-oxoethyl)benzyl)-5-((E)-2-(cis-4-(trifluoromethyl) cyclohexyl)vinyl)nicotinamide (6.9 mg, 19%) and 6-methoxy-N-(3-(2-(methylamino)-2-oxoethyl)benzyl)-5-((E)-2-(trans-4-(trifluoromethyl)cyclohexyl)vinyl) nicotinamide (7.2 mg, 14%) as both white solids.

6-Methoxy-N-(3-(2-(methylamino)-2-oxoethyl)benzyl)-5-((E)-2-(cis-4-(trifluoromethyl) cyclohexyl)vinyl)nicotinamide. H NMR (400 MHz, CDCl₃): δ 8.44 (s, 1H), 8.12 (s, 1H), 7.40-7.30 (m, 2H), 7.24-7.19 (m, 2H), 6.65-6.55 (m, 1H), 6.54-6.39 (m, 2H), 5.44 (s, 1H), 4.66 (d, J=5.2 Hz, 2H), 4.03 (s, 3H), 3.56 (s, 2H), 2.77 (d, J=4.0 Hz, 3H), 2.59 (m, 1H), 2.13 (m, 1H), 1.92-1.68 (m, 8H).

6-Methoxy-N-(3-(2-(methylamino)-2-oxoethyl)benzyl)-5-((E)-2-(trans-4-(trifluoromethyl) cyclohexyl)vinyl)nicotinamide. H NMR (400 MHz, CDCl₃): δ 8.44 (s, 1H), 8.10 (s, 1H), 7.38-7.28 (m, 2H), 7.25-7.15 (m, 2H), 6.64-6.49 (m, 2H), 6.28 (dd, J=16.0, 6.8 Hz, 1H), 5.50 (s, 1H), 4.63 (d, J=4.8 Hz, 2H), 4.02 (s, 3H), 3.54 (s, 2H), 2.77 (d, J=4.0 Hz, 3H), 2.18-2.15 (m, 1H), 2.07-1.91 (m, 5H), 1.46-1.32 (m, 2H), 1.29-1.15 (m, 2H).

### Example 57

### 5-Methoxy-N-(3-(2-(methylamino)-2-oxoethyl)benzyl)-4-((E)-2-(cis-4-(trifluoro methyl)cyclohexyl)vinyl)picolinamide and 5-methoxy-N-(3-(2-(methylamino)-2-oxoethyl) benzyl)-4-((E)-2-(trans-4-(trifluoromethyl)cyclohexyl)vinyl) picolinamide

The overall Example 57 reaction scheme was as follows:

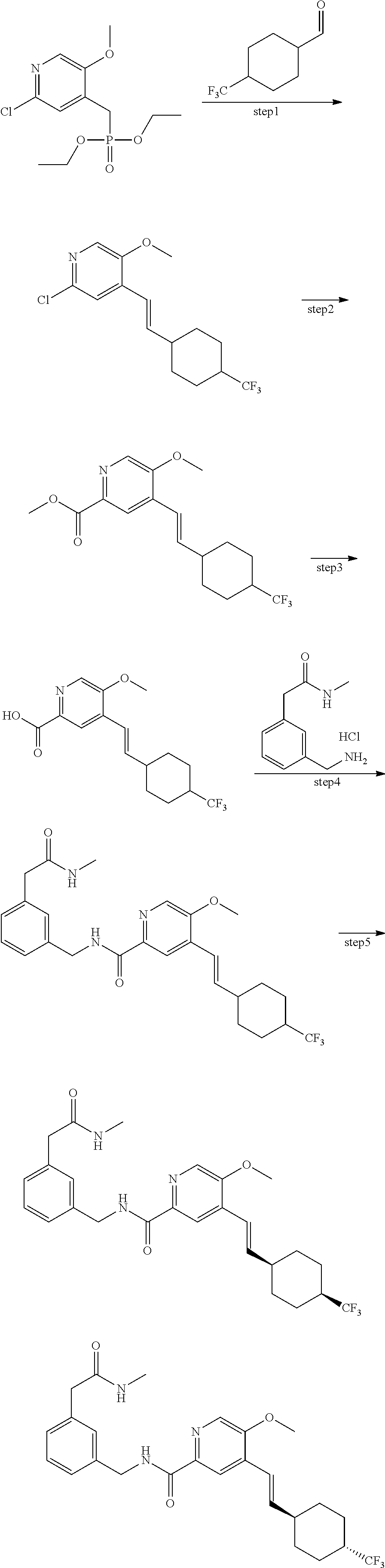

### Step 1: (E)-2-Chloro-5-methoxy-4-(2-(4-(trifluoromethyl)cyclohexyl)vinyl) pyridine

To a solution of 2-chloro-4-(diethoxyphosphorylmethyl)-5-methoxy-pyridine (1.0 g, 3.15 mmol) in toluene (18 mL) at 0° C. was added sodium tert-pentoxide (0.42 g, 3.78 mmol) and the mixture was stirred for 20 min at 0° C. A solution of 4-(trifluoro methyl)cyclohexanecarbaldehyde (1.62 g, 6.3 mmol, 70% purity) in THF (18 mL) was then added dropwise, and the reaction mixture was stirred at 0° C. for 1.5 h. The reaction mixture was poured into saturated aqueous NH₄Cl solution (50 mL) and extracted with EtOAc (100 mL×2). The organic layers were combined, washed with brine (50 mL), dried over Na₂SO₄ and concentrated. The residue was purified by column chromatography on silica gel (0-10% EtOAc in petroleum ether) to afford the title compound (340 mg, 34%) as a colorless oil. LCMS (ESI): m/z 320.1 (M+H)⁺.

### Step 2: (E)-Methyl 5-methoxy-4-(2-(4-(trifluoromethyl)cyclohexyl)vinyl) picolinate

A mixture of 2-chloro-5-methoxy-4-[(E)-2-[4-(trifluoromethyl)cyclohexyl]vinyl] pyridine (0.32 g, 1 mmol), potassium carbonate (0.28 g, 2 mmol), Pd(OAc)₂ (23 mg, 0.10 mmol) and 1,3-bis(diphenylphosphino)propane (83 mg, 0.20 mmol) in MeOH (10 mL) and DMF (10 mL) was heated at 80° C. under CO atmosphere (50 psi) for 16 h. The solution was extracted with EtOAc (30 mL×2) and washed with water (30 mL). The organic layer was dried over Na₂SO₄ and concentrated. The residue was purified by TLC (50% EtOAc in petroleum ether, R_{f}=0.4) to afford the title compound (0.31 g, 90%) as a light yellow oil. LCMS (ESI): m/z 344.1 (M+H)⁺.

### Step 3: (E)-5-Methoxy-4-(2-(4-(trifluoromethyl)cyclohexyl)vinyl)picolinic Acid

To a mixture of LiOH H₂O (190 mg, 4.5 mmol) and methyl 5-methoxy-4-[(E)-2-[4-(trifluoromethyl)cyclohexyl]vinyl]pyridine-2-carboxylate (0.31 g, 0.90 mmol) in water (15 mL), MeOH (15 mL) and tetrahydrofuran (3 mL) was stirred at 15° C. for 16 hours. The reaction solution was concentrated to remove organic solvent and adjusted pH to 6 with aq. 1 N HCl solution. Then the solution was extracted with EtOAc (30 mL×2) and washed with water (30 mL). The organic layer was dried over Na₂SO₄ and concentrated to afford the title compound (200 mg, 67%) as a white solid. LCMS (ESI): m/z 330.1 (M+H)⁺.

### Step 4: (E)-5-Methoxy-N-(3-(2-(methylamino)-2-oxoethyl)benzyl)-4-(2-(4-(trifluoro methyl)cyclohexyl)vinyl)picolinamide

To a solution of 5-methoxy-4-[(E)-2-[4-(trifluoromethyl)cyclohexyl]vinyl]pyridine-2-carboxylic acid (200 mg, 0.61 mmol) and 2-[3-(aminomethyl)phenyl]-N-methyl-acetamide hydrochloride (157 mg, 0.73 mmol), N,N-diisopropylethylamine (0.54 mL, 3.04 mmol) in N,N-dimethylformamide (5 mL) was added HATU (462 mg, 1.21 mmol). The reaction was stirred at 15° C. for 16 h. Water (30 mL) was added and the solution was extracted with EtOAc (30 mL×2). The organic layer was dried over Na₂SO₄, filtered and concentrated to dryness. The residue was purified by prep-TLC (10% MeOH in DCM, R_{f}=0.6) to afford the title compound (130 mg, 44%) as a white solid. LCMS (ESI): m/z 490.1 (M+H)⁺.

### Step 5: 5-Methoxy-N-(3-(2-(methylamino)-2-oxoethyl)benzyl)-4-((E)-2-(cis-4-(trifluoromethyl)cyclohexyl)vinyl)picolinamide and 5-methoxy-N-(3-(2-(methylamino)-2-oxoethyl)benzyl)-4-((E)-2-(trans-4-(trifluoromethyl)cyclohexyl)vinyl)picolinamide

5-Methoxy-N-[[3-[2-(methylamino)-2-oxo-ethyl]phenyl]methyl]-4-[(E)-2-[4-(trifluoromethyl)cyclohexyl]vinyl]pyridine-2-carboxamide (130 mg, 0.27 mmol) was separated by SFC (DAICEL CHIRALPAK AS-H (250 mm*30 mm, 5 um)), (0.1% NH₃H₂O in IPA, 50%) to afford 5-methoxy-N-(3-(2-(methylamino)-2-oxoethyl)benzyl)-4-((E)-2-(cis-4-(trifluoromethyl)cyclohexyl)vinyl)picolinamide (79.2 mg, 61%) and 5-methoxy-N-(3-(2-(methylamino)-2-oxoethyl)benzyl)-4-((E)-2-(trans-4-(trifluoromethyl)cyclohexyl)vinyl) picolinamide (28.9 mg, 22%), both as white solids.

5-Methoxy-N-(3-(2-(methylamino)-2-oxoethyl)benzyl)-4-((E)-2-(cis-4-(trifluoro methyl)cyclohexyl)vinyl)picolinamide. H NMR (400 MHz, CD₃OD): δ 8.27 (s, 1H), 8.16 (s, 1H), 7.95 (br s, 1H), 7.31-7.21 (m, 3H), 7.20 (d, J=6.8 Hz, 1H), 6.79-6.64 (m, 2H), 4.58 (s, 2H), 4.01 (s, 3H), 3.47 (s, 2H), 2.73-2.66 (m, 3H), 2.64-2.55 (m, 1H), 2.29-2.13 (m, 1H), 1.86-1.83 (m, 2H), 1.79-1.58 (m, 6H).

5-Methoxy-N-(3-(2-(methylamino)-2-oxoethyl)benzyl)-4-((E)-2-(trans-4-(trifluoro methyl)cyclohexyl)vinyl)picolinamide. H NMR (400 MHz, CD₃OD): δ 8.28 (s, 1H), 8.13 (s, 1H), 7.33-7.21 (m, 3H), 7.18 (d, J=6.8 Hz, 1H), 6.69 (d, J=16.0 Hz, 1H), 6.53 (dd, J=16.0, 6.8 Hz, 1H), 4.58 (s, 2H), 4.01 (s, 3H), 3.47 (s, 2H), 2.69 (s, 3H), 2.27-2.07 (m, 2H), 2.05-1.89 (m, 4H), 1.49-1.25 (m, 4H).

### Example 58

### (E)-N-(5-(2-(4,4-Difluorocyclohexyl)vinyl)-6-methoxypyridin-3-yl)ethenesulfonamide

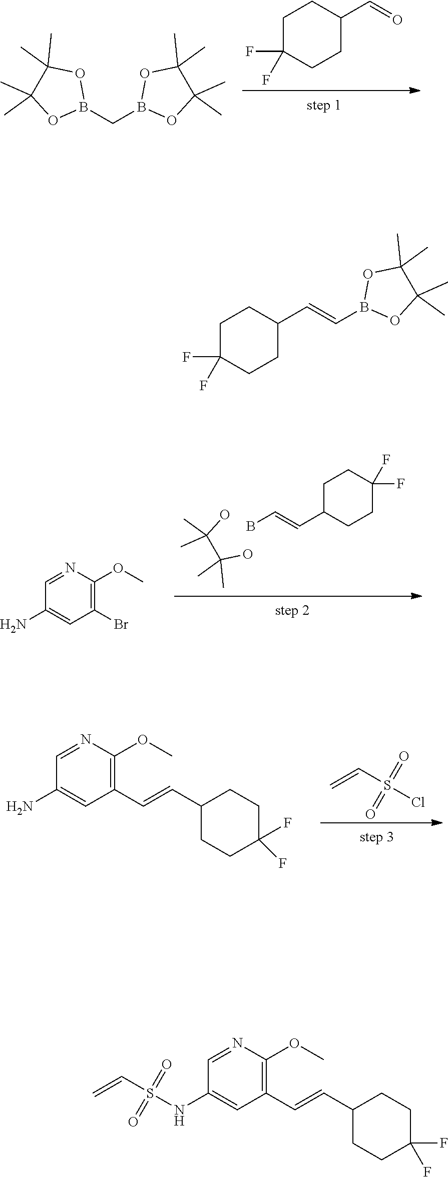

### Step 1: (E)-2-(2-(4,4-Difluorocyclohexyl)vinyl)-4,4,5,5-tetramethyl-1,3,2-dioxaborolane

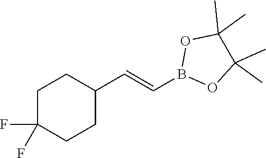 [SMILES: CC1(C)OB(/C=C/C2CCC(F)(F)CC2)OC1(C)C]

In the glove box, lithium 2,2,6,6-tetramethylpiperidin-1-ide (1.0 M in THF, 8.10 mL, 8.10 mmol) was transferred to a 50 mL flask. The flask was sealed, and removed from the glove box. THF (5.0 mL) was added to the flask at 0° C. and a solution of bis(4,4,5,5-tetramethyl-1,3,2-dioxaborolan-2-yl)methane (2.20 g, 8.1 mmol) in THF (5.0 mL) was added. The reaction was then stirred for 10 minutes and then cooled to −78° C. A solution of 4,4-difluorocyclohexanecarbaldehyde (10.0 g, 6.7 mmol) in THF (20.0 mL) was added. The reaction was stirred at −78° C. for additional 4 hours. The reaction mixture was quenched with saturated NH₄Cl (50 mL). The solution was extracted with EtOAc (50 mL×3). The organic layers were washed with water (50 mL), dried over Na₂SO₄ and concentrated. The residue was purified by column chromatography on silica gel (0-2% EtOAc in petroleum ether) to afford the title compound (300 mg, 16%) as a white solid. ¹H NMR (400 MHz, CDCl₃): δ 6.56 (dd, J=18.0, 6.0 Hz, 1H), 5.46 (dd, J=18.0, 1.2 Hz, 1H), 2.21-2.03 (m, 3H), 1.89-1.67 (m, 4H), 1.57-1.42 (m, 2H), 1.28 (s, 12H).

### Step 2: (E)-5-(2-(4,4-Difluorocyclohexyl)vinyl)-6-methoxypyridin-3-amine

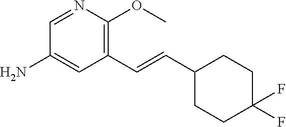 [SMILES: COc1ncc(N)cc1/C=C/C1CCC(F)(F)CC1]

To a solution of 5-bromo-6-methoxy-pyridin-3-amine (100 mg, 0.49 mmol) in dioxane (5.0 mL) and water (1.0 mL) were added Pd(dppf)Cl₂ (36 mg, 0.050 mmol), (E)-2-(2-(4,4-difluorocyclohexyl)vinyl)-4,4,5,5-tetramethyl-1,3,2-dioxaborolane (200 mg, 0.74 mmol) and Na₂CO₃ (160 mg, 1.5 mmol). Then the reaction mixture was placed under nitrogen atmosphere and stirred at 100° C. for 16 h. The reaction mixture was concentrated. The residue was purified by prep-TLC (50% EtOAc in petroleum ether) to afford the title compound (120 mg, 90%) as a white solid. ¹H NMR (400 MHz, DMSO-d₆): δ 7.40 (d, J=2.4 Hz, 1H), 7.11 (d, J=2.8 Hz, 1H), 6.45 (d, J=16.0 Hz, 1H), 6.16 (dd, J=16.0, 7.2 Hz, 1H), 4.71 (s, 2H), 3.79-3.70 (m, 3H), 2.33-2.24 (m, 1H), 2.09-1.99 (m, 2H), 1.96-1.76 (m, 4H), 1.48-1.31 (m, 2H).

### Step 3: (E)-N-(5-(2-(4,4-Difluorocyclohexyl)vinyl)-6-methoxypyridin-3-yl)ethenesulfonamide

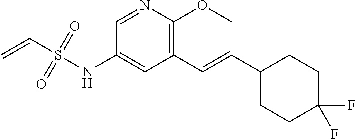 [SMILES: C=CS(=O)(=O)Nc1cnc(OC)c(/C=C/C2CCC(F)(F)CC2)c1]

To a mixture of (E)-5-(2-(4,4-Difluorocyclohexyl)vinyl)-6-methoxypyridin-3-amine (70 mg, 0.26 mmol) in pyridine (3.0 mL) was added ethenesulfonyl chloride (0.04 mL, 0.32 mmol) at 0° C. The reaction mixture was stirred at room temperature for 2 h. The mixture was concentrated and the residue was purified by prep-HPLC (Boston Green ODS 150*30 mm*5 um, acetonitrile 50-80/water (0.225% FA)) to afford the title compound (19.93 mg, 21%) as a white solid. ¹H NMR (400 MHz, CDCl₃): δ 7.84 (d, J=2.4 Hz, 1H), 7.61 (d, J=2.4 Hz, 1H), 6.61-6.44 (m, 2H), 6.28-6.15 (m, 2H), 6.09 (s, 1H), 5.99 (dd, J=10.0, 2.0 Hz, 1H), 3.97 (s, 3H), 2.35-2.25 (m, 1H), 2.19-2.10 (m, 2H), 1.92-1.72 (m, 4H), 1.65-1.56 (m, 2H). LCMS (ESI): m/z 359.0 (M+H)⁺.

### Example 59

A TEAD lipid assay was done according to the following method. His-tagged TEAD proteins were pre-incubated with a Bodipy-C16 FP probe (Life Technologies Cat #D3821) for 30 minutes at room temperature to pre-form TEAD-probe complex. The relatively large size of the TEAD-probe complex resulted in slower tumbling of the probe, yielding high fluorescence polarization value (mP). Addition of compounds that are TEAD lipid pocket binders resulted in the displacement of the probe from TEAD and decreased fluorescence polarization (mP) values. After 60 minutes incubation of compounds with the TEAD Bodipy-C16 complex, fluorescence polarization values were measured on an EnVision multi-label plate reader (Perkin Elmer Cat #2104-0010A.) The free probe resulted in faster tumbling or low fluorescence polarization values. Duplicate 10 point dose response curves were generated for each test compound. The potency of compounds as TEAD lipid pocket binders was determined by IC₅₀ value generated using a non-linear 4 parameter curve fit.

A Detroit 562 reporter assay was done as follows. For stable reporter line generation and maintenance, Detroit 562 cells (ATCC Cat #CCL-138) were transfected with a reporter plasmid containing a Nano-Luciferase reporter element under control for the hippo pathway response element TEAD. As a counter-screen, the plasmid also contained firefly luciferase under control of the PGK promoter which is unrelated to the hippo pathway. Following transfection and dilution cloning, individual clones were selected and characterized. Clones were grown and maintained in RPMI 1640, 10% fetal bovine serum, 2 mM L-glutamine, 50 ug/mL Zeocin (Invitrogen #R25005). For reporter assay with test compounds, cells were plated (Day 1) in 384 well tissue culture treated assay plates and incubated overnight. Two cell plates were prepared for each compound plate. The following day (day 2), the cells were treated with compounds and incubated overnight. On day three, cell plates were incubated with either NanoGlo nanoluciferase reagent (Promega Cat #N1150) for on-target determination of pathway inhibition, or Firefly luciferase reagent (Promega Cat #E8150), for determination of off target activity of compounds. Luminesence measurements were measured on an EnVision multi-label plate reader (Perkin Elmer Cat #2104-0010A.) Duplicate 10 point dose response curves were generated for each test compound. The potency of compounds as TEAD lipid pocket binders was determined by IC₅₀ value generated using a non-linear 4 parameter curve fit.

The results for compounds referenced in Tables 1 to 3 (with the exception of compounds 54, 55, 56A, 56B, 57A and 57B) are presented in Table 4 below.

TABLE 4

| | Lipid FP | Lipid FP |
|---|---|---|
| | TEAD3 | TEAD2 |
| Compound | IC₅₀ [uM] | IC₅₀ [uM] |
| 1B | 1.8 | 1 |
| 2 | 1.5 | 1 |
| 3 | 1.8 | 0.58 |
| 4 | 1.3 | 0.96 |
| 5 | 0.77 | 0.6 |
| 6A | 1.7 | 0.96 |
| 7A | 1.7 | 0.91 |
| 8A | 1.4 | 0.74 |
| 9A | 1.6 | 0.78 |
| 10 | 1.9 | 1.1 |
| 11 | 0.63 | 0.56 |
| 12 | 0.86 | 0.59 |
| 13 | 1.8 | 0.73 |
| 14 | 0.43 | 0.38 |
| 15 | 2 | 1.2 |
| 16 | 1.2 | 0.69 |
| 17 | 1.8 | 0.81 |
| 18 | 1.2 | 1.9 |
| 19 | 1.6 | 1.9 |
| 20 | 0.76 | 2 |
| 21 | 0.63 | 0.86 |
| 22 | 1.3 | 0.93 |
| 23 | 0.83 | 1.2 |
| 24 | 0.78 | 0.36 |
| 25 | 1.2 | 0.38 |
| 26 | 1.7 | 2 |
| 27 | 1.2 | 1 |
| 28 | 1 | 0.89 |
| 29 | 1.1 | 1 |
| 30 | 0.62 | 0.72 |
| 31 | 0.97 | 0.78 |
| 32 | 1.4 | 0.61 |
| 33 | 1.2 | 0.84 |
| 34 | 1.2 | 0.96 |
| 35 | 1.4 | 1.9 |
| 36 | 1.4 | 0.95 |
| 37 | 1.3 | 0.75 |
| 38 | 2 | 1.1 |
| 39 | 0.97 | 0.55 |
| 40 | 0.65 | 0.64 |
| 41 | 1.1 | 1.8 |
| 42 | 0.78 | 0.53 |
| 43 | 1.7 | 0.98 |
| 44 | 0.7 | 0.83 |
| 45 | 1.7 | 1.6 |
| 46 | 1.6 | 1.6 |
| 47 | 0.86 | 1.8 |
| 48 | 1.6 | 1.1 |
| 49 | 1.9 | 0.75 |
| 50 | 1.1 | 1.3 |
| 51 | 2 | 0.96 |
| 52 | 13 | 1.6 |

The results for compounds 54, 55, 56A, 56B, 57A, 57B and 58 referenced in Table 1 are presented in Table 5 below.

TABLE 5

| | Lipid | Lipid | Lipid | Lipid |
|---|---|---|---|---|
| | HTRF | HTRF | HTRF | HTRF |
| | TEAD1 | TEAD2 | TEAD3 | TEAD4 |
| Compound | IC₅₀ [uM] | IC₅₀ [uM] | IC₅₀ [uM] | IC₅₀ [uM] |
| 54 | 0.318 | 0.146 | 4.00 | 0.224 |
| 55 | 0.198 | 0.089 | 0.230 | 0.073 |
| 56A | 0.708 | 0.079 | 15.17 | 0.355 |
| 56B | 0.193 | 0.031 | 2.089 | 0.039 |
| 57A | 0.389 | 0.046 | 0.867 | 0.131 |
| 57B | 0.128 | 0.017 | 0.298 | 0.019 |
| 58 | 0.034 | 0.010 | 0.034 | 0.008 |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

It is to be understood that the invention is not limited to the particular embodiments and aspects of the disclosure described above, as variations of the particular embodiments and aspects may be made and still fall within the scope of the appended claims. All documents cited to or relied upon herein are expressly incorporated by reference.

1. A compound of formula (I) or a pharmaceutically acceptable salt thereof: 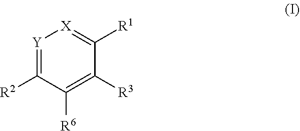
wherein
(i) R¹ is selected from —C₁₋₆ alkyl, —C₃₋₈ cycloalkyl, —C₁₋₆ alkyl-C₃₋₈ cycloalkyl, —C₁₋₆ haloalkyl, —O—C₁₋₆ alkyl, —O—C₃₋₈ cycloalkyl, —O—C₁₋₆ alkyl-C₃₋₈ cycloalkyl and —O—C₁₋₆ haloalkyl;
(ii) R² is selected from —C(O)—N(R^{a})(R^{b}) and —N(R^{c})—S(O)₂(R^{a}),
wherein each R^{a}, R^{b}, R^{c} and R^{d} is independently selected from —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₁₋₆ alkyl-C₅₋₂₀ aryl, —C₃₋₈ heterocyclyl, —C₆₋₂₀ aryl and —C₁₋₂₀ heteroaryl, wherein each —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₁₋₆ alkyl-C₅₋₂₀ aryl, —C₃₋₈ heterocyclyl, —C₆₋₂₀ aryl, —C₁₋₂₀ heteroaryl is independently optionally substituted with at least one of oxo, —CN, —C₁₋₁₂ alkyl, —C₁₋₁₂ haloalkyl, halo, —NO₂, —N(R^{e})(R^{f}), —C₁₋₆ alkyl-C(O)—N(R^{e})(R^{f}), and —OR^{e}, wherein each R^{a}, R^{b} and R^{c} may further optionally be independently selected from hydrogen,
wherein each R^{e} and R^{f} is independently selected from hydrogen, —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃₋₈ heterocyclyl, —C₆₋₂₀ aryl and —C₁₋₂ heteroaryl, wherein each —C₂₋₁₂ alkenyl, —C₂₋₁₂ alkynyl, —C₃₋₈ cycloalkyl, —C₁₋₆ alkyl-C₃₋₈ cycloalkyl, —C₁₋₁₂ haloalkyl, —C₃₋₈ heterocyclyl, —C₆₋₂₀ aryl, —C₁₋₂₀ heteroaryl is independently optionally substituted with at least one of oxo, —CN, —C₁₋₁₂ alkyl, —C₁₋₁₂ haloalkyl, halo, —NO₂, —O—C₁₋₁₂ alkyl and —OH;
(iii) R³ is -(A)ₙ-R⁵ wherein
A is selected from a bond, —C₁₋₁₂ alkyl-, —C₃₋₈ cycloalkyl- and —C₂₋₁₂ alkenyl-;
R⁵ is selected from hydrogen, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl, and —C₅₋₁₃ spirocycle,
wherein for A and R⁵ each —C₁₋₁₂ alkyl-, —C₃— cycloalkyl-, —C₂₋₁₂ alkenyl-, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl and —C₅₋₁₃ spirocycle is independently optionally substituted with at least one of oxo, —CN, —C₁₋₁₂ alkyl, —C₁₋₁₂haloalkyl, C-₃₋₈ cycloalkyl, halo, —NO₂, —N(R^{e})(R^{f}), and —OR^{e}, and
n is 0 or 1;
(iv) each X and Y is independently selected from CR⁴ and N; and
(v) each R⁴ and R⁶ is independently selected from hydrogen, halogen, —C₁₋₆ haloalkyl, and CN
wherein when X and Y are each CR⁴ and when R² is —C(O)—N(R^{a})(R^{b}), A is selected from optionally substituted —C₁₋₁₂ alkyl-, —C₃₋₈ cycloalkyl- and —C₃₋₁₂ alkenyl- and R⁵ is selected from hydrogen, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl, and —C₅₋₁₃ spirocycle,
wherein for A and R⁵, each —C₁₋₁₂ alkyl-, —C₃₋₈ cycloalkyl-, —C₃₋₁₂ alkenyl-, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl and —C₅₋₁₃ spirocycle is independently optionally substituted with at least one of oxo, —CN, —C₁₋₁₂ alkyl, —C₁₋₁₂haloalkyl, C-₃₋₈ cycloalkyl, halo, —NO₂, —N(R^{e})(R^{f}), and —OR^{e}.

2. The compound of claim 1 wherein:
(i) R¹ is —O—C₁₋₆ alkyl;
(ii) R^{a} and R^{b} are independently selected from hydrogen and —C₁₋₁₂ alkyl, wherein —C₁₋₁₂ alkyl is optionally substituted with at least one —OH;
(iii) R^{c} is hydrogen and R^{d} is selected from —C₁₋₁₂ alkyl, —C₂₋₁₂ alkenyl and —C₃₋₈ cycloalkyl, wherein —C₁₋₁₂ alkyl is optionally substituted with —CN;
(iv) R⁵ is selected from hydrogen, —C₃₋₈ cycloalkyl, —C₆₋₂₀ aryl and —C₅₋₁₃ spirocycle wherein each —C₃₋₈ cycloalkyl, —C₆₋₂₀ aryl and —C₅₋₁₃ spirocycle is independently optionally substituted with at least one of C₁₋₁₂ alkyl, C₁₋₁₂ haloalkyl, halo and C-₃₋₈ cycloalkyl;
(v) each R⁴ is independently selected from hydrogen and halo; and
(vi) R⁶ is hydrogen.

3. The compound of claim 1 wherein:
(i) R¹ is selected from —O—C₁₋₄ alkyl, —O—C₁₋₂ alkyl and —O—CH₃;
(ii)(a) R² is —C(O)—N(R^{a})(R^{b}), R^{a} is hydrogen, and R^{b} is selected from hydrogen, C₁₋₆ alkyl, C₁₋₄ alkyl and C₂₋₄ alkyl, wherein said alkyl is optionally substituted with at least one —OH,
(ii)(b) R² is —C(O)—N(R^{a})(R^{b}), R^{a} is hydrogen, and R^{b} is C₁₋₃-alkyl-C₅₆ aryl wherein the C₅₋₆ aryl is substituted with —C₁₋₃ alkyl-C(O)—N(R^{e})(R^{f}) wherein R^{e} is H and R^{f} is C₁₋₃ alkyl, or
(ii)(c) R² is —N(R^{c})—S(O)₂(R^{d}), R^{c} is hydrogen, and R^{d} is selected from (1) C₁₋₄ alkyl, C₁₋₂ alkyl, —C₃₋₆ cycloalkyl or —CH₃, (2) C₂₋₄ alkenyl or C₂ alkenyl, (3) —C₁₋₆ alkyl-CN or —C₁₋₄ alkyl-CN, and (4) C₃₋₈ cycloalkyl, C₃₋₆ cycloalkyl or C₃ cycloalkyl;
(iii) A is selected from (1) —C₃₋₈ cycloalkyl-, —C₃₋₅ cycloalkyl- or —C₃₋₄ cycloalkyl- and (2) —C₂₋₆ alkenyl-, —C₂₋₄ alkenyl- or —C₂₋₃ alkenyl-; and
(iv) R⁵ is selected from (1) hydrogen, (2) —C₃₋₈ cycloalkyl, —C₃₋₆ cycloalkyl or —C₄₋₆ cycloalkyl, wherein each said cycloalkyl is optionally substituted with one or more halo, —C₁₋₄ alkyl, —C₁₋₃ alkyl, —CH₃, —C₁₋₄ haloalkyl, —C₁₋₂ haloalkyl, or —C₁ haloalkyl, (3) C₅₋₆ aryl or C₆ aryl, wherein each said aryl is optionally substituted with one or more halo, —C₁₋₄ alkyl, —C₃ alkyl, —CH₃, —C₃₋₆ cycloalkyl, or —C₃ cycloalkyl, and (4) C₅₋₁₂ spirocycle, C₅₋₈ spirocycle, or C₆ spirocycle.

4. The compound of claim 1, wherein X is CH.

5. The compound of claim 1, wherein X is N.

6. The compound of any preceding claim wherein Y is CH.

7. The compound of claim 1, wherein Y is CF.

8. The compound of claim 1, wherein Y is N.

9. The compound of claim 1, wherein halo is selected from F and Cl.

10. The compound of claim 1, wherein haloalkyl is selected from —CHF₂ and —CF₃.

11. The compound of claim 1, wherein R² is selected from:

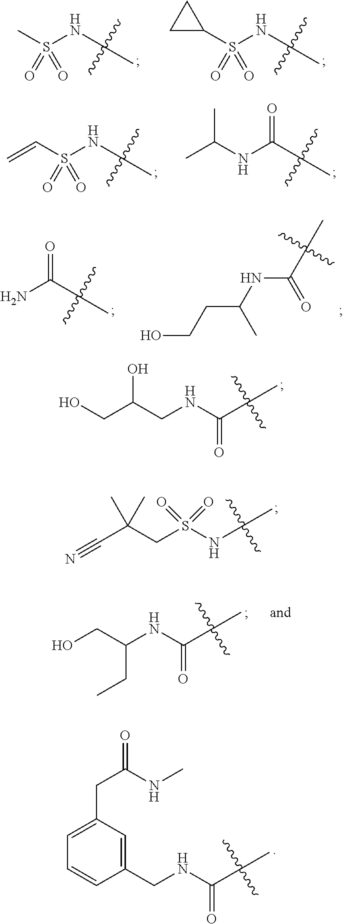

12. The compound of claim 1, wherein -(A)ₙ-R⁵ is selected from:

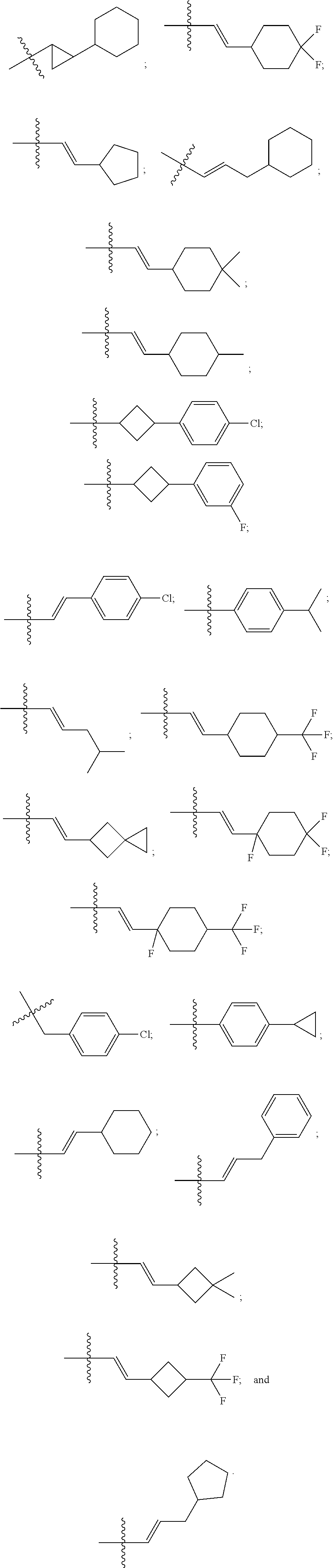

13. The compound of claim 1, wherein the compound is selected from:

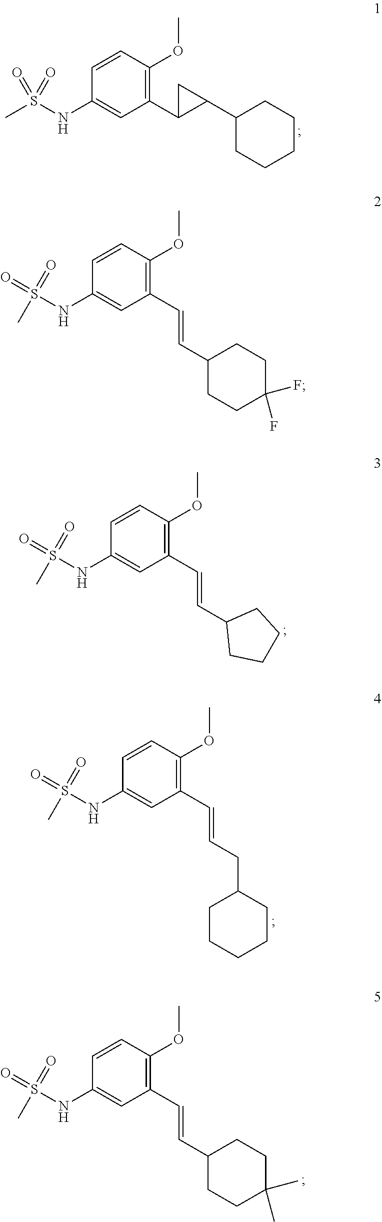 [SMILES: COc1ccc(NS(C)(=O)=O)cc1C1CC1C1CCCCC1 ; COc1ccc(NS(C)(=O)=O)cc1/C=C/C1CCC(F)(F)CC1 ; COc1ccc(NS(C)(=O)=O)cc1/C=C/C1CCCC1 ; COc1ccc(NS(C)(=O)=O)cc1/C=C/CC1CCCCC1 ; COc1ccc(NS(C)(=O)=O)cc1/C=C/C1CCC(C)(C)CC1]

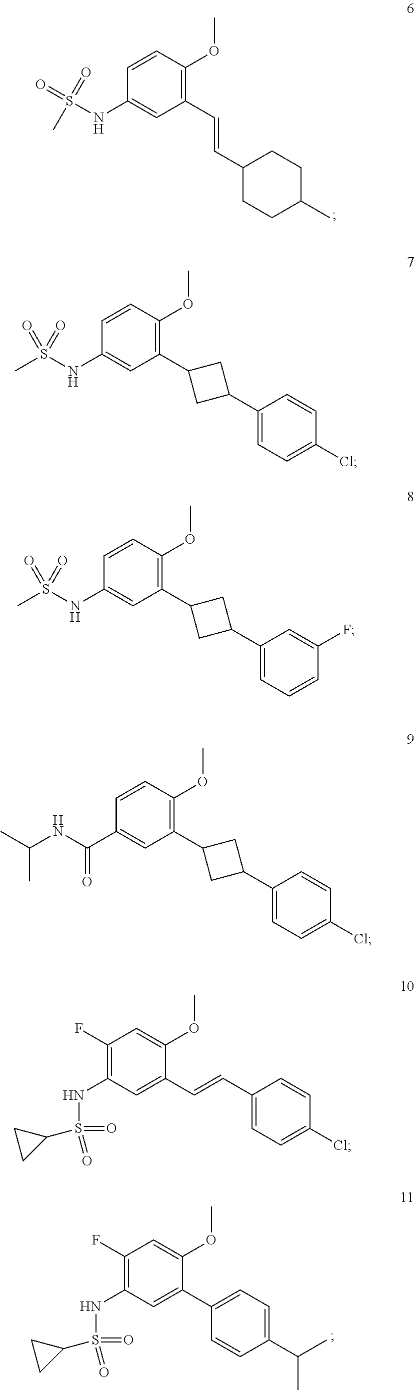

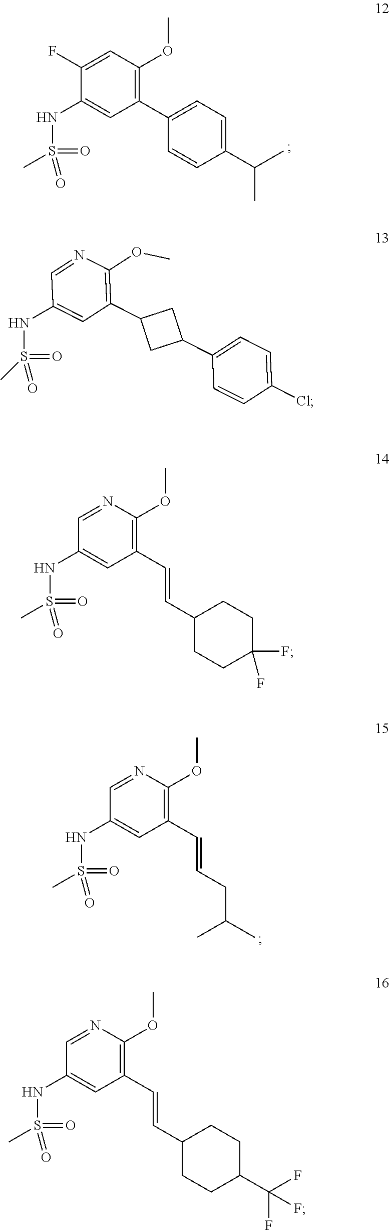

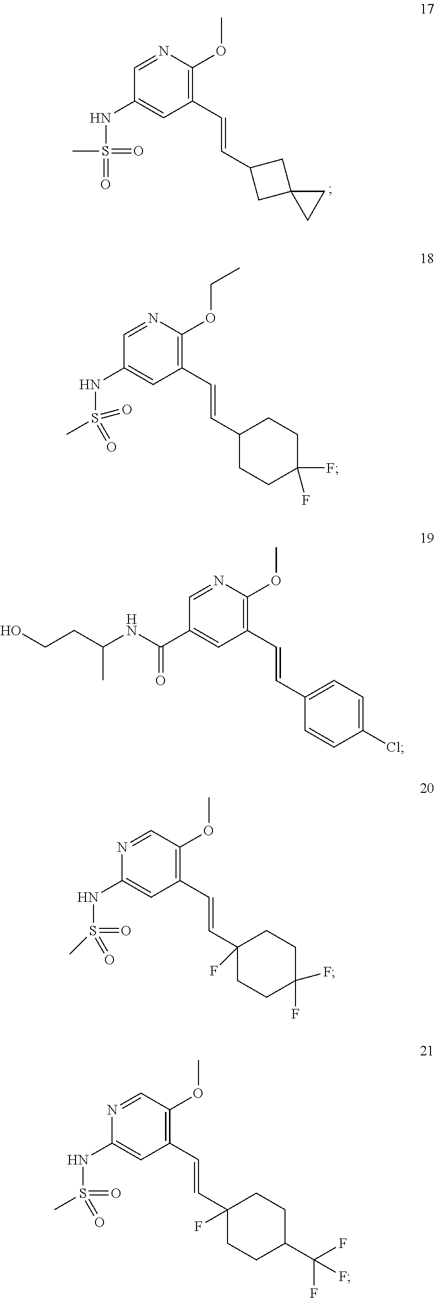 [SMILES: COc1ncc(NS(C)(=O)=O)cc1/C=C/C1CC2(CC2)C1 ; CCOc1ncc(NS(C)(=O)=O)cc1/C=C/C1CCC(F)(F)CC1 ; COc1ncc(C(=O)NC(C)CCO)cc1/C=C/c1ccc(Cl)cc1 ; COc1cnc(NS(C)(=O)=O)cc1/C=C/C1(F)CCC(F)(F)CC1 ; COc1cnc(NS(C)(=O)=O)cc1/C=C/C1(F)CCC(C(F)(F)F)CC1]

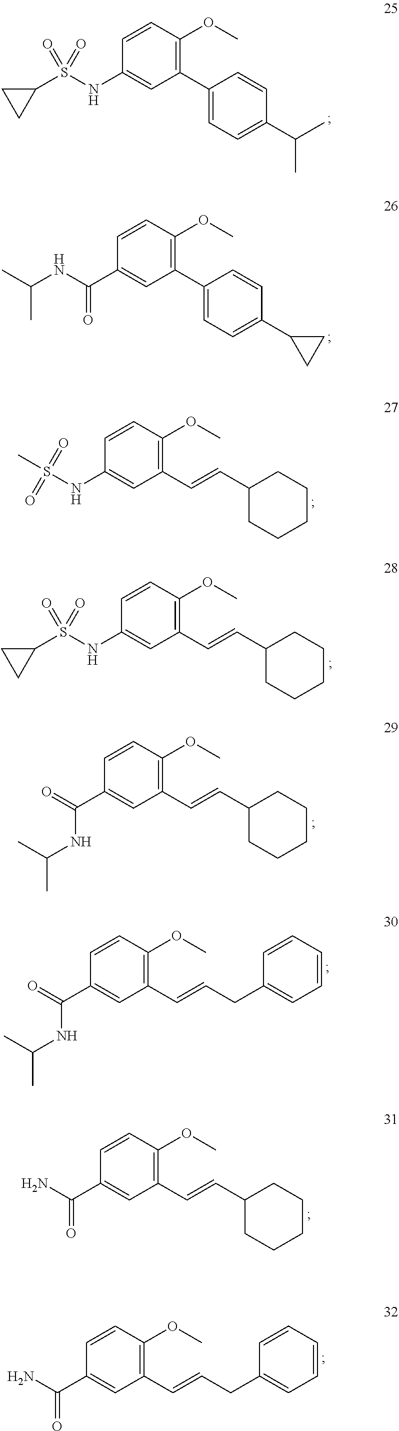

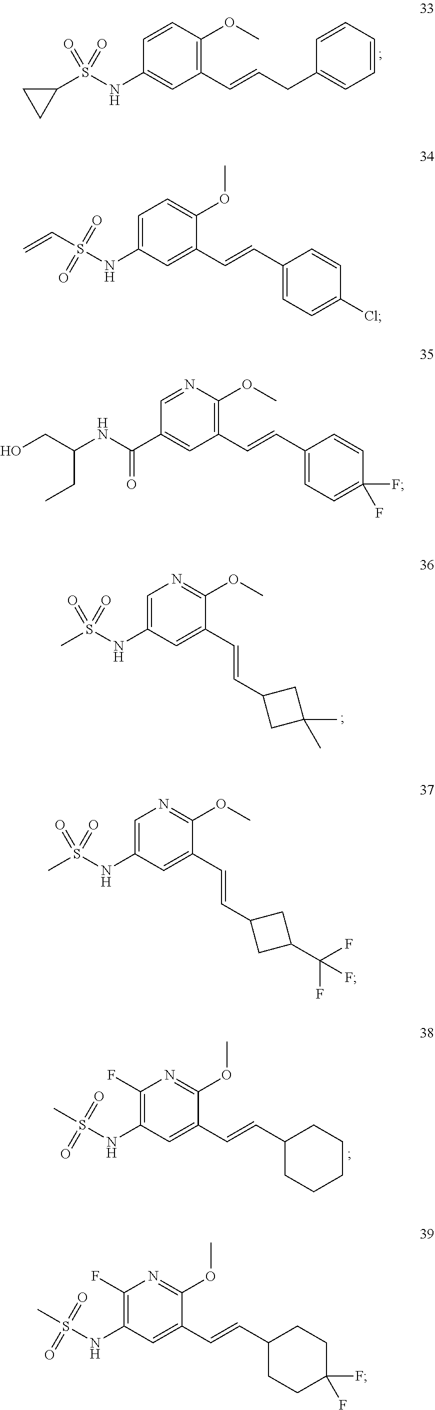

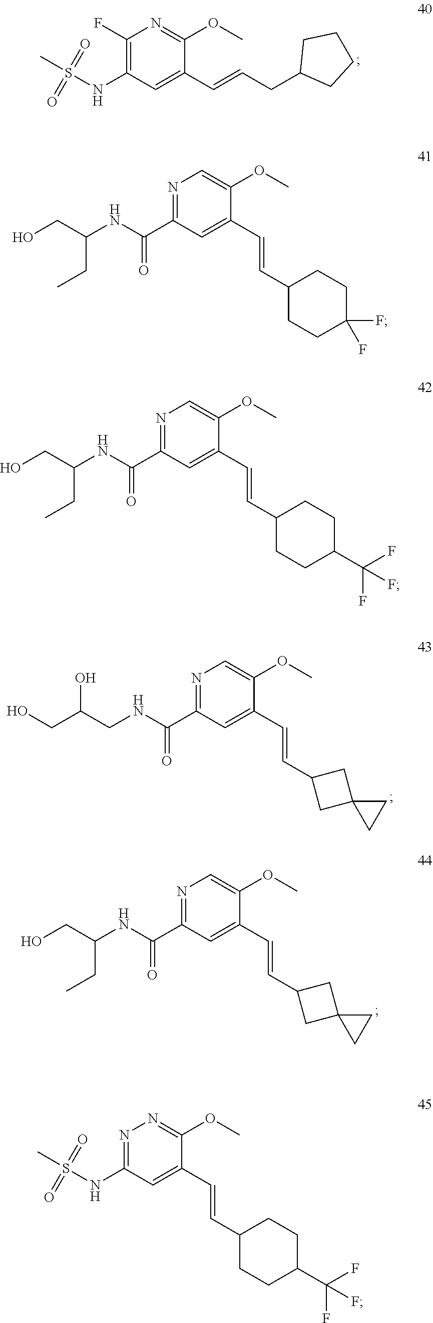 [SMILES: COc1nc(F)c(NS(C)(=O)=O)cc1/C=C/CC1CCCC1 ; CCC(CO)NC(=O)c1cc(/C=C/C2CCC(F)(F)CC2)c(OC)cn1 ; CCC(CO)NC(=O)c1cc(/C=C/C2CCC(C(F)(F)F)CC2)c(OC)cn1 ; COc1cnc(C(=O)NCC(O)CO)cc1/C=C/C1CC2(CC2)C1 ; CCC(CO)NC(=O)c1cc(/C=C/C2CC3(CC3)C2)c(OC)cn1 ; COc1nnc(NS(C)(=O)=O)cc1/C=C/C1CCC(C(F)(F)F)CC1]

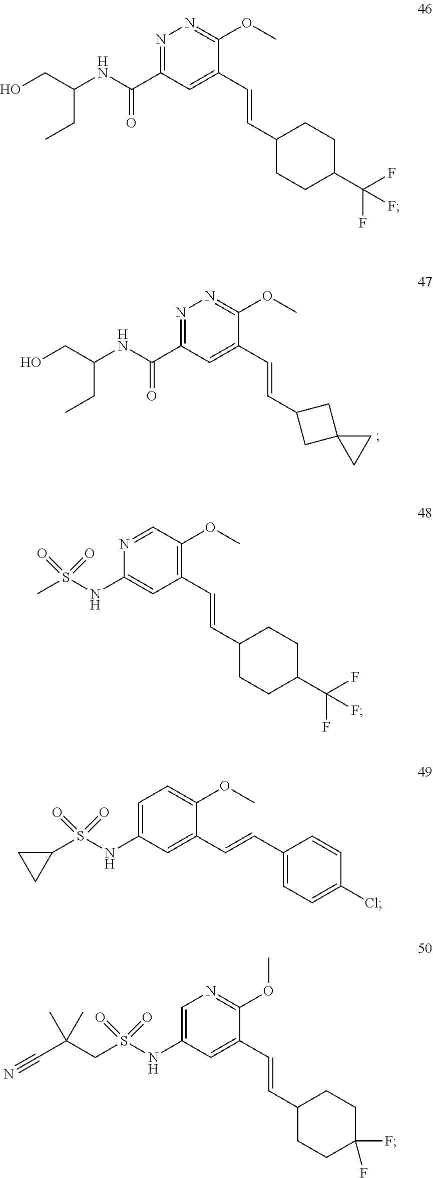 [SMILES: CCC(CO)NC(=O)c1cc(/C=C/C2CCC(C(F)(F)F)CC2)c(OC)nn1 ; CCC(CO)NC(=O)c1cc(/C=C/C2CC3(CC3)C2)c(OC)nn1 ; COc1cnc(NS(C)(=O)=O)cc1/C=C/C1CCC(C(F)(F)F)CC1 ; COc1ccc(NS(=O)(=O)C2CC2)cc1/C=C/c1ccc(Cl)cc1 ; COc1ncc(NS(=O)(=O)CC(C)(C)C#N)cc1/C=C/C1CCC(F)(F)CC1]

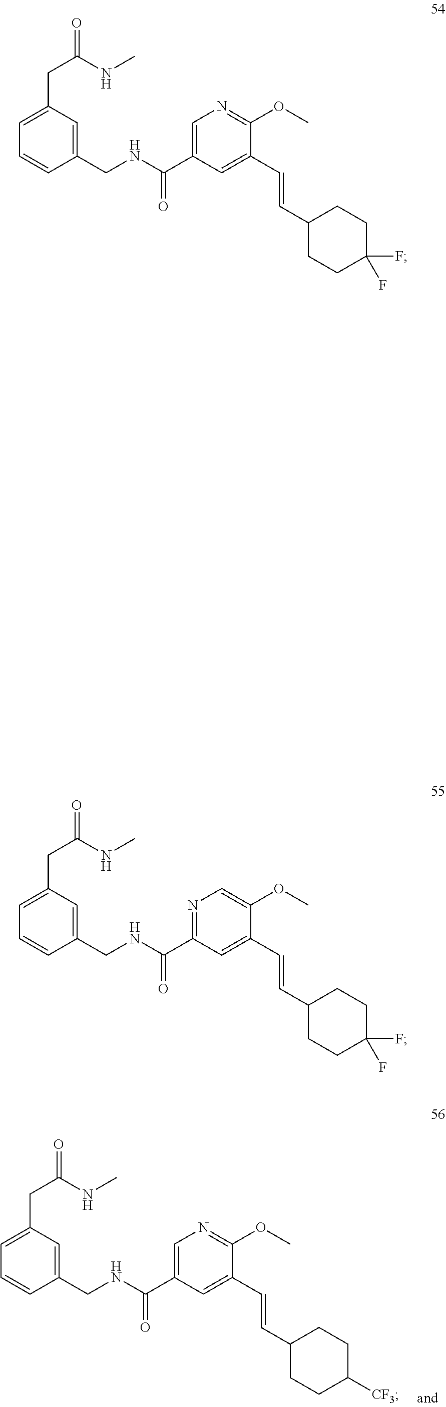 [SMILES: CNC(=O)Cc1cccc(CNC(=O)c2cnc(OC)c(/C=C/C3CCC(F)(F)CC3)c2)c1 ; CNC(=O)Cc1cccc(CNC(=O)c2cc(/C=C/C3CCC(F)(F)CC3)c(OC)cn2)c1 ; CNC(=O)Cc1cccc(CNC(=O)c2cnc(OC)c(/C=C/C3CCC(C(F)(F)F)CC3)c2)c1]

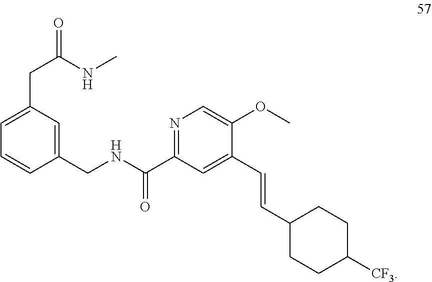 [SMILES: CNC(=O)Cc1cccc(CNC(=O)c2cc(/C=C/C3CCC(C(F)(F)F)CC3)c(OC)cn2)c1]

14. The compound of claim 13, wherein the compound is selected from:

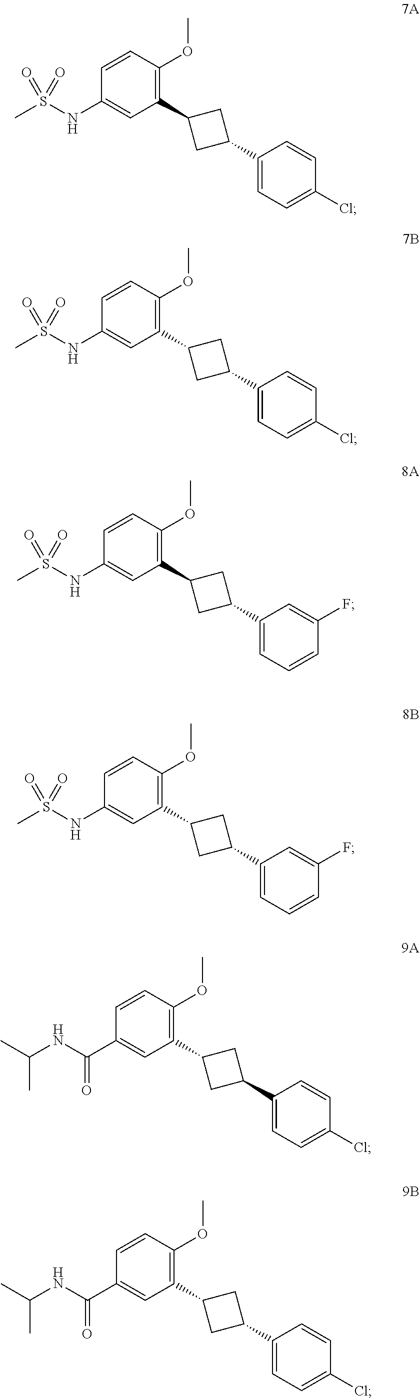 [SMILES: COc1ccc(NS(C)(=O)=O)cc1[C@H]1C[C@H](c2ccc(Cl)cc2)C1 ; COc1ccc(NS(C)(=O)=O)cc1[C@H]1C[C@@H](c2ccc(Cl)cc2)C1 ; COc1ccc(NS(C)(=O)=O)cc1[C@H]1C[C@H](c2cccc(F)c2)C1 ; COc1ccc(NS(C)(=O)=O)cc1[C@H]1C[C@@H](c2cccc(F)c2)C1 ; COc1ccc(C(=O)NC(C)C)cc1[C@H]1C[C@H](c2ccc(Cl)cc2)C1 ; COc1ccc(C(=O)NC(C)C)cc1[C@H]1C[C@@H](c2ccc(Cl)cc2)C1]

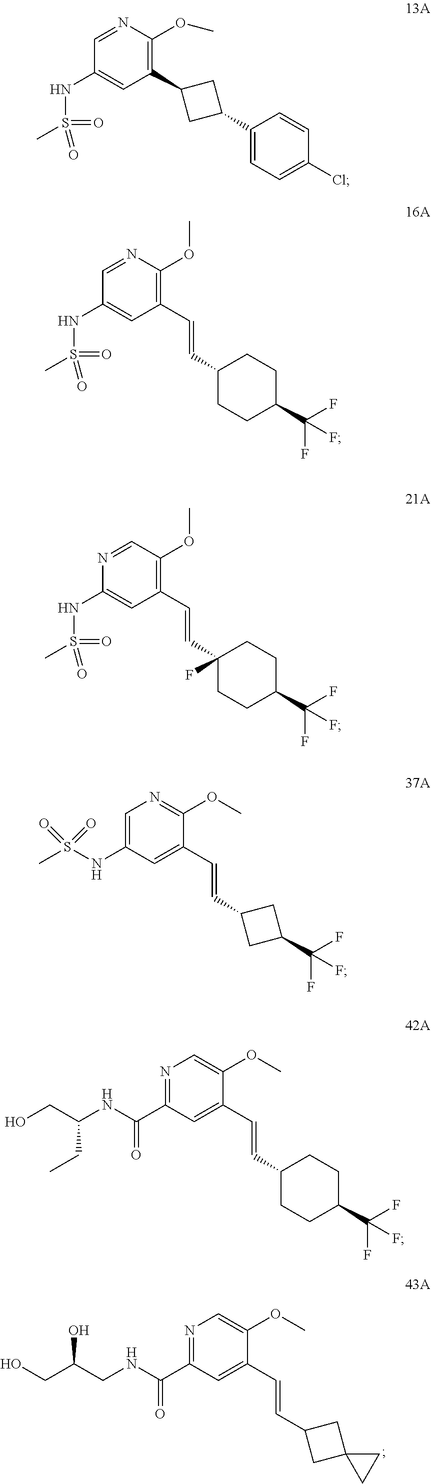 [SMILES: COc1ncc(NS(C)(=O)=O)cc1[C@H]1C[C@H](c2ccc(Cl)cc2)C1 ; COc1ncc(NS(C)(=O)=O)cc1/C=C/[C@H]1CC[C@H](C(F)(F)F)CC1 ; COc1cnc(NS(C)(=O)=O)cc1/C=C/[C@]1(F)CC[C@@H](C(F)(F)F)CC1 ; COc1ncc(NS(C)(=O)=O)cc1/C=C/[C@H]1C[C@H](C(F)(F)F)C1 ; CC[C@H](CO)NC(=O)c1cc(/C=C/[C@H]2CC[C@H](C(F)(F)F)CC2)c(OC)cn1 ; COc1cnc(C(=O)NC[C@H](O)CO)cc1/C=C/C1CC2(CC2)C1]

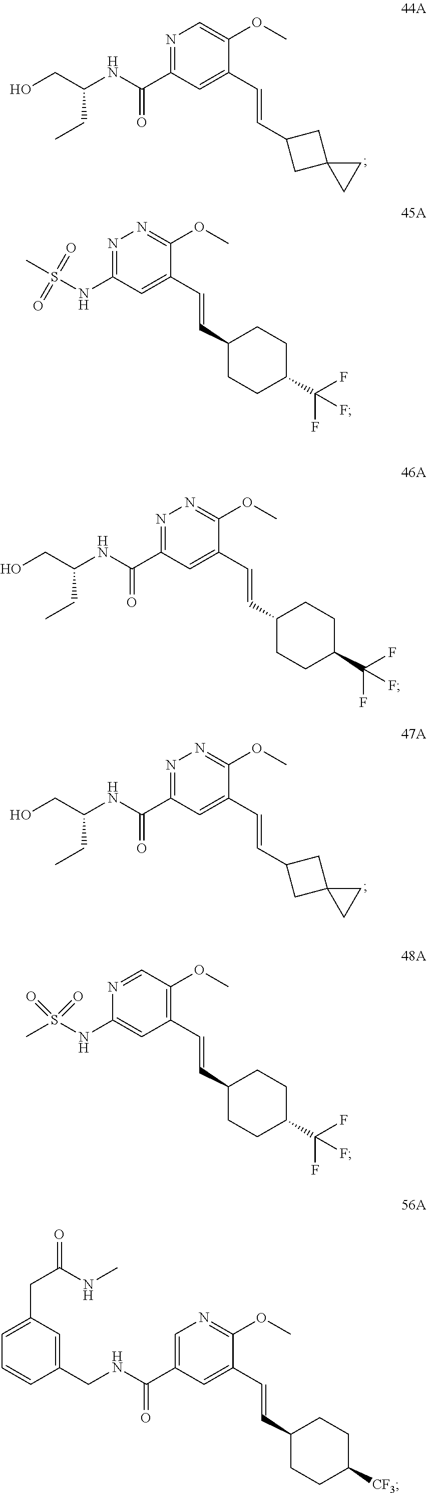 [SMILES: CC[C@H](CO)NC(=O)c1cc(/C=C/C2CC3(CC3)C2)c(OC)cn1 ; COc1nnc(NS(C)(=O)=O)cc1/C=C/[C@H]1CC[C@H](C(F)(F)F)CC1 ; CC[C@H](CO)NC(=O)c1cc(/C=C/[C@H]2CC[C@H](C(F)(F)F)CC2)c(OC)nn1 ; CC[C@H](CO)NC(=O)c1cc(/C=C/C2CC3(CC3)C2)c(OC)nn1 ; COc1cnc(NS(C)(=O)=O)cc1/C=C/[C@H]1CC[C@H](C(F)(F)F)CC1 ; CNC(=O)Cc1cccc(CNC(=O)c2cnc(OC)c(/C=C/[C@H]3CC[C@@H](C(F)(F)F)CC3)c2)c1]

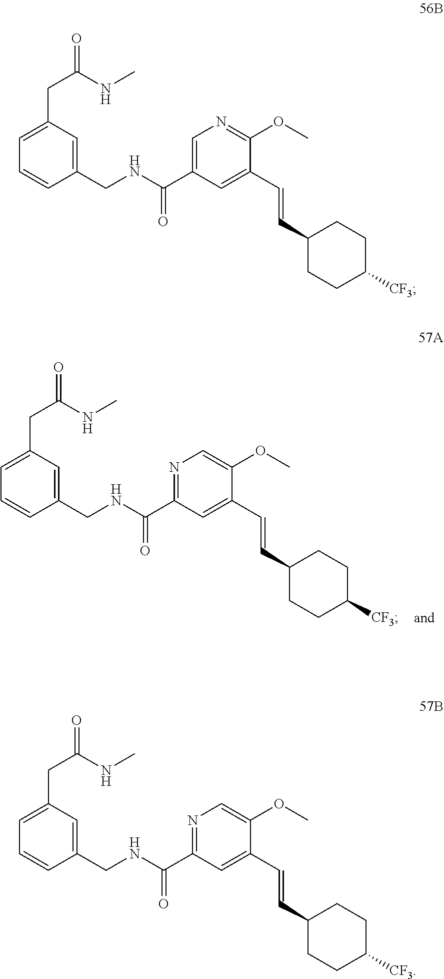 [SMILES: CNC(=O)Cc1cccc(CNC(=O)c2cnc(OC)c(/C=C/[C@H]3CC[C@H](C(F)(F)F)CC3)c2)c1 ; CNC(=O)Cc1cccc(CNC(=O)c2cc(/C=C/[C@H]3CC[C@@H](C(F)(F)F)CC3)c(OC)cn2)c1 ; "and" ; CNC(=O)Cc1cccc(CNC(=O)c2cc(/C=C/[C@H]3CC[C@H](C(F)(F)F)CC3)c(OC)cn2)c1]

15. A compound of formula (II) or a pharmaceutically acceptable salt thereof: 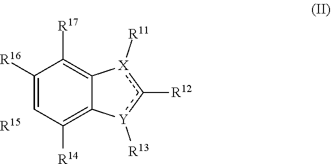
wherein
(i) R¹¹ is selected from hydrogen, —C₁₋₆ alkyl, —C₃₋₈ cycloalkyl, —C₁₋₆ alkyl-C₃₋₈ cycloalkyl, and —C₁₋₆ haloalkyl;
(ii) R¹⁵ is —C(O)—N(R^{g})(R^{h}) or —N(Rⁱ)—S(O)₂(R^{j}),
wherein each R^{g}, R^{h}, Rⁱ and R^{j} is independently selected from —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃₋₈ heterocyclyl, —C₆₋₂₀ aryl and —C₁₋₂₀ heteroaryl, and wherein each —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃₋₈ heterocyclyl, —C₆₋₂₀ aryl and —C₁₋₂₀ heteroaryl is independently optionally substituted with at least one of oxo, —CN, —C₁₋₁₂ alkyl, —C₁₋₁₂ haloalkyl, halo, —NO₂, —N(R^{k})(R^{l}), and —OR^{k}, wherein R^{g}, R^{h} and Rⁱ may be further independently selected from H,
wherein each R^{k} and R^{l} is independently selected from hydrogen, —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃₋₈ heterocyclyl, —C₆₋₂₀ aryl and —C₁₋₂₀ heteroaryl, wherein each —C₁₋₁₂ alkyl, —C₂₋₁₂ alkenyl, —C₂₋₁₂ alkynyl, —C₃₋₈ cycloalkyl, —C₁₋₆ alkyl-C₃₋₈ cycloalkyl, —C₃— heterocyclyl, —C₆₋₂₀ aryl, —C₁₋₂₀ heteroaryl is independently optionally substituted with at least one of oxo, —CN, —C₁₋₁₂ alkyl, —C₁₋₁₂ haloalkyl, halo, —NO₂, —O—C₁₋₁₂ alkyl and —OH;
(iii) R¹³ is -(A)ₙ-R¹⁸ wherein
A is selected from —C₁₋₁₂ alkyl-, —C₃₋₈ cycloalkyl- and —C₂₋₁₂ alkenyl-,
R¹⁸ is selected from hydrogen, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl and —C₅₋₁₃ spirocycle,
wherein for A and R¹⁸ each —C₁₋₁₂ alkyl-, —C₃₋₈ cycloalkyl-, —C₂₋₁₂ alkenyl-, —C₃₋₈ cycloalkyl, —C₁₋₆ alkyl-C₃₋₈ cycloalkyl, —C₃₋₈ heterocyclyl, —C₆₋₂₀ aryl, —C₁₋₂₀ heteroaryl and —C₅₋₁₃ spirocycle is independently optionally substituted with at least one of oxo, —CN, —C₁₋₁₂ alkyl, —C₁₋₁₂haloalkyl, —C₃₋₈ cycloalkyl, halo, —NO₂, —N(R^{k})(R^{l}), and —OR^{k}, and
n is 0 or 1;
(iv) the dashed lines represent optional double bonds wherein (a) X is C, Y is N, the bond between X and the ring carbon atom bearing R¹² is a double bond, and the bond between Y and the ring carbon atom bearing R¹² is a single bond, or (b) X is N, Y is C, the bond between X and the ring carbon atom bearing R¹² is a single bond, and the bond between Y and the ring carbon atom bearing R¹² is a double bond; and
(v) each R¹², R¹⁴, R¹⁶ and R¹⁷ is independently selected from hydrogen, halogen, —C₁₋₆ alkyl and —C₁₋₆ haloalkyl.

16. The compound of claim 15 wherein:
(i) R¹¹ is —C₁₋₆ alkyl;
(ii) R^{g} and R^{h} are independently selected from hydrogen, —C₁₋₁₂ alkyl and —C₃₋₈ cycloalkyl, wherein said —C₁₋₁₂ alkyl and —C₃₋₈ cycloalkyl are independently optionally substituted with at least one —OH;
(iii) Rⁱ is hydrogen and R^{j} is selected from —C₁₋₁₂ alkyl, —C₂₋₁₂ alkenyl and —C₃₋₈ cycloalkyl, wherein —C₁₋₁₂ alkyl is optionally substituted with —CN;
(iv) R¹⁸ is selected from hydrogen, —C₃₋₈ cycloalkyl, —C₆₋₂₀ aryl and —C₅₋₁₃ spirocycle wherein each —C₃₋₈ cycloalkyl, —C₆₋₂₀ aryl and —C₅₋₁₃ spirocycle is independently optionally substituted with at least one of C₁₋₁₂ alkyl, C₁₋₁₂ haloalkyl, halo and C-₃₋₈ cycloalkyl; and
(v) each of R¹², R¹⁴, R¹⁶ and R¹⁷ is hydrogen.

17. The compound of claim 15, wherein:
(i) R¹¹ is selected from C₁₋₄ alkyl, C₁₋₂ alkyl and —CH₃;
(ii) R¹⁵ is —N(Rⁱ)—S(O)₂(R^{j}), Rⁱ is hydrogen, and R^{j} is selected from C₁₋₄ alkyl, C₁₋₂ alkyl and —CH₃;
(iii) A is selected from —C₁₋₆ alkyl-, —C₁₋₄ alkyl-, —C₁₋₂ alkyl- or —CH₂—; and
(iv) R¹⁸ is C₅₋₆ aryl or C₆ aryl, wherein said aryl is optionally substituted with one or more halo.

18. The compound of claim 15, wherein X is C, and Y is N.

19. The compound of claim 15, wherein X is N and Y is C.

20. The compound of claim 15, wherein halo is Cl.

21. The compound of claim 15, wherein R¹⁵ is selected from

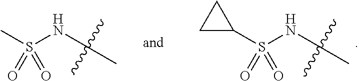

22. The compound of claim 15, wherein -(A)ₙ-R¹⁸ is

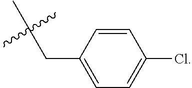 [SMILES: *Cc1ccc(Cl)cc1]

23. The compound of claim 1, wherein the compound is selected from:

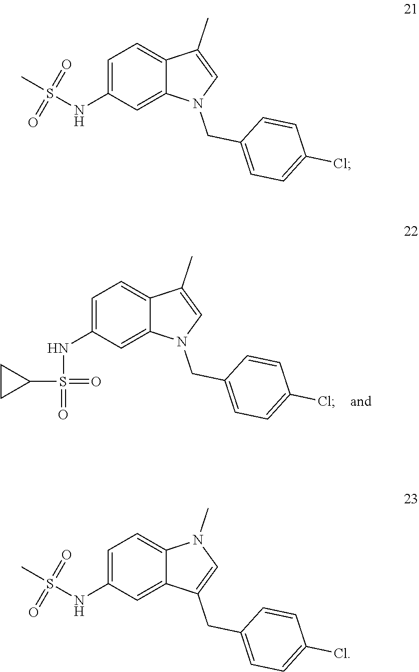 [SMILES: Cc1cn(Cc2ccc(Cl)cc2)c2cc(NS(C)(=O)=O)ccc12 ; Cc1cn(Cc2ccc(Cl)cc2)c2cc(NS(=O)(=O)C3CC3)ccc12 ; "and" ; Cn1cc(Cc2ccc(Cl)cc2)c2cc(NS(C)(=O)=O)ccc21]

24. The compound of claim 1, wherein the compound is selected from:

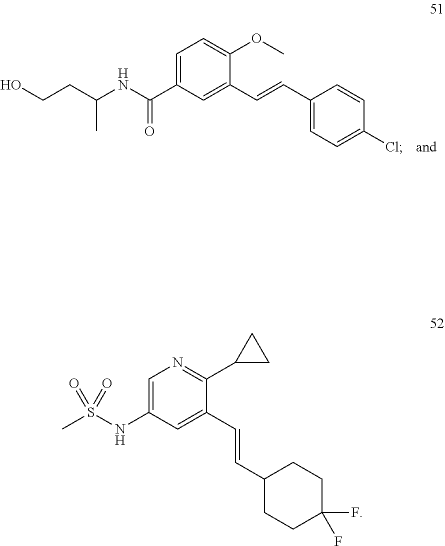 [SMILES: COc1ccc(C(=O)NC(C)CCO)cc1/C=C/c1ccc(Cl)cc1 ; "and" ; CS(=O)(=O)Nc1cnc(C2CC2)c(/C=C/C2CCC(F)(F)CC2)c1]

25. A pharmaceutical composition, comprising a compound of claim 1, or a pharmaceutically acceptable salt thereof, and a pharmaceutically acceptable carrier, diluent or excipient.

26-27. (canceled)

28. A method for treating a disease or condition in a mammal comprising, administering a compound of claim 1, or a pharmaceutically acceptable salt thereof to the mammal.

29-30. (canceled)

31. The method of claim 28 wherein the disease or condition is acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

32-33. (canceled)

34. A method for modulating TEAD activity, comprising contacting TEAD with a compound as described in claim 1 or a salt thereof.

35. A method for treating a disease or condition mediated by TEAD activity in a mammal, comprising administering a compound of claim 1 or a pharmaceutically acceptable salt thereof to the mammal.

36. The method of claim 35 wherein the disease or condition is acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

37. The compound of claim 1, wherein the compound of formula I, or a pharmaceutically acceptable salt thereof, is a compound of formula IA, or a pharmaceutically acceptable salt thereof:

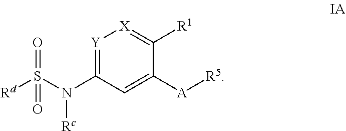

38. The compound of claim 1, wherein the compound of formula I, or a pharmaceutically acceptable salt thereof, is a compound of formula IB, or a pharmaceutically acceptable salt thereof:

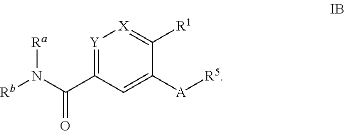

39. A process for the preparation of a compound of claim 1.

40. The compound of claim 1, said compound obtained by the process of claim 39.

41. (canceled)


SEQUENCE LISTING (18 sequences)

SEQ ID NO: 1 - protein, 440 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 447 aa, Artificial Sequence
  IgG4-kappa humanized monoclonal antibody against PD-1 receptor
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 218 aa, Artificial Sequence
  IgG4-kappa humanized monoclonal antibody against PD-1 receptor
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 10 aa, Artificial Sequence
  monoclonal antibody of IgG1 isotype against the protein programmed cell death-ligand 1
GFTFSDSWIH

SEQ ID NO: 6 - protein, 18 aa, Artificial Sequence
  monoclonal antibody of IgG1 isotype against the protein programmed cell death-ligand 1
AWISPYGGSTYYADSVKG

SEQ ID NO: 7 - protein, 9 aa, Artificial Sequence
  monoclonal antibody of IgG1 isotype against the protein programmed cell death-ligand 1
RHWPGGFDY

SEQ ID NO: 8 - protein, 11 aa, Artificial Sequence
  monoclonal antibody of IgG1 isotype against the protein programmed cell death-ligand 1
RASQDVSTAVA

SEQ ID NO: 9 - protein, 7 aa, Artificial Sequence
  monoclonal antibody of IgG1 isotype against the protein programmed cell death-ligand 1
SASFLYS

SEQ ID NO: 10 - protein, 9 aa, Artificial Sequence
  monoclonal antibody of IgG1 isotype against the protein programmed cell death-ligand 1
QQYLYHPAT

SEQ ID NO: 11 - protein, 118 aa, Artificial Sequence [sequence omitted]

SEQ ID NO: 12 - protein, 108 aa, Artificial Sequence
  monoclonal antibody of IgG1 isotype against the protein programmed cell death-ligand 1
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 13 - protein, 447 aa, Artificial Sequence
  monoclonal antibody of IgG1 isotype against the protein programmed cell death-ligand 1
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 214 aa, Artificial Sequence
  monoclonal antibody of IgG1 isotype against the protein programmed cell death-ligand 1
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 449 aa, Homo sapiens
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 216 aa, Homo sapiens
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 17 - protein, 450 aa, Homo sapiens
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 215 aa, Homo sapiens
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC